(19) United States
(12) Patent Application Publication
(10) Pub. No.: US 2025/0382340 A1
(43) Pub. Date: Dec. 18, 2025

(54) DNA CONSTRUCTS FOR IMPROVED T CELL IMMUNOTHERAPY

(71) Applicants: The Regents of the University of California, Oakland, CA (US); The J. David Gladstone Institutes, a testamentary trust established under the Will of J. David Glads, San Francisco, CA (US)

(72) Inventors: Franziska Blaeschke, San Francisco, CA (US); Theodore Lee Roth, San Francisco, CA (US); Alexander Marson, San Francisco, CA (US); Yan Yi Chen, San Francisco, CA (US); Ryan Apathy, San Francisco, CA (US)

(73) Assignees: The Regents of The University of California, Oakland, CA (US); The J. David Gladstone Institutes, a testamentary trust established under the will of J. David Glads, San Francisco, CA (US)

(21) Appl. No.: 18/837,837

(22) PCT Filed: Feb. 14, 2023
(86) PCT No.: PCT/US2023/062603

§ 371 Date: Aug. 12, 2024

Related U.S. Application Data
(60) Provisional application No. 63/309,938, filed on Feb. 14, 2022.

(51) Int. Cl. C07K 14/47; A61K 40/11; A61K 40/32; A61K 40/41; A61K 40/42; C07K 14/725; C12N 5/0783; C12Q 1/6869

(52) U.S. Cl. CPC C07K 14/4702; A61K 40/11; A61K 40/32; A61K 40/416; A61K 40/42; C07K 14/7051; C12N 5/0636; C12Q 1/6869; C12N 2510/00

(57) ABSTRACT

Provided herein are methods and compositions for modifying the genome of human T cells. Further, the compositions and methods described herein can be used to generate human T cells with altered specificity and functionality, while limiting the side effects associated with T cell therapies. Provided herein is a human T cell that heterologously expresses one or more polypeptides. In some embodiment, the one or more polypeptides, for example, two or more polypeptides, are encoded by a nucleic acid construct inserted into the TCR locus of the cell.

### CROSS-REFERENCES TO RELATED APPLICATIONS

This application is a U.S. National Phase application Under Section 371 of PCT/US2023/062603, filed Feb. 14, 2023, which claims the benefit of and priority to U.S. Provisional Application No. 63/309,938, filed on Feb. 14, 2022, which are hereby incorporated by reference in its entirety.

### SEQUENCE LISTING

The instant application contains a Sequence Listing which has been submitted electronically in XML format and is hereby incorporated by reference in its entirety. Said XML copy, created on Mar. 27, 2023, is named 081906-1372820-247510PC_SL.xml and is 232,000 bytes in size.

### BACKGROUND OF THE INVENTION

Current techniques for modification of ex vivo or intravitally gene edited cells for therapeutic use have focused on correction of an existing mutation, limiting therapeutic applicability to conditions caused by a single mutation resulting in a misfunctioning gene, or on integrating an entirely new synthetic gene, requiring extensive research and development into creating a new therapeutically useful synthetic DNA sequence. Therefore, there are limited options for genomic modifications. Given the importance of T cells in adoptive cellular therapeutics, the ability to obtain human T cells and modify them to produce edited T cells with desirable function(s) could be beneficial in the development and application of adoptive T cell therapies.

### BRIEF SUMMARY OF THE INVENTION

The present disclosure is directed to compositions and methods for modifying the genome of a T cell. The inventors have discovered that human T cells can be modified to alter T cell specificity and function. By inserting a nucleic acid encoding one or more polypeptides, and a heterologous T cell receptor (TCR) or a synthetic antigen receptor (e.g., a chimeric antigen receptor (CAR)) into a specific endogenous site in the genome of the T cell, (e.g., a TCR locus), human T cells having the desired antigen specificity of the TCR or CAR and the function of the polypeptide can be made. Further, the compositions and methods described herein can be used to generate human T cells with altered specificity and functionality, while limiting the side effects associated with T cell therapies.

Provided herein is a human T cell that heterologously expresses one or more polypeptides. In some embodiment, the one or more polypeptides, for example, two or more polypeptides, are encoded by a nucleic acid construct inserted into the TCR locus of the cell.

In some embodiments, the human T cell heterologously expresses: a polypeptide comprising a TFAP4 protein, and a polypeptide comprising a BATF protein; a polypeptide comprising a BATF3 protein, and a polypeptide comprising a TFAP4 protein; a polypeptide comprising a FOXJ2 protein, and a polypeptide comprising a RARA protein; a polypeptide comprising a ID3 protein, and a polypeptide comprising TFAP4 protein; a polypeptide comprising a BATF protein, and a polypeptide comprising IRF2 protein; a polypeptide comprising a NANOG protein, and a polypeptide comprising a TFAP4 protein; a polypeptide comprising a MAFF protein, and a polypeptide comprising SATB1 protein; a polypeptide comprising a BATF protein, and a polypeptide comprising IRF1 protein; a polypeptide comprising a TFAP4 protein, and a polypeptide comprising HOPX protein; a polypeptide comprising a BATF protein, and a polypeptide comprising HES2 protein; a polypeptide comprising a ATF2 protein, and a polypeptide comprising BATF protein; a polypeptide comprising a TIGIT protein, and a polypeptide comprising ICOS protein; a polypeptide comprising a LTBR protein, and a polypeptide comprising a 4-1BB protein; a polypeptide comprising a DR5 protein, and a polypeptide comprising an ICOS protein; a polypeptide comprising a CCR4 protein; a polypeptide comprising a BATF3 protein, and a polypeptide comprising SMAD4 protein; a polypeptide comprising a FOXP3 protein, and a polypeptide comprising RELA protein; a polypeptide comprising a ID3 protein, and a polypeptide comprising BATF3 protein; a polypeptide comprising an ID2 protein, a polypeptide comprising a TP73 protein; a polypeptide comprising a SMAD3 protein, or a polypeptide comprising a BATF3 protein.

In some embodiments, the human T cell expresses a polypeptide comprising a human TFAP4 protein, and a polypeptide comprising a human TIM3 extracellular domain or a portion thereof linked to a human 4-1BB intracellular domain (and optionally about 1-15 (e.g., 12) amino acids of the human 4-1BB extracellular domain) via a transmembrane domain.

In some embodiments, the human T cell expresses a polypeptide comprising a human SOX5 protein, and a truncated TIGIT protein comprising the human TIGIT extracellular domain or a portion thereof, the TIGIT transmembrane domain and about 1-10 (e.g., 7) amino acids of the human TIGIT intracellular domain.

In some embodiments, the human T cell expresses a polypeptide comprising a human MYC protein, and a polypeptide comprising a human TGFbR2 extracellular domain or a portion thereof linked to amino acids 41-142 of human MyD88 (and optionally 1-10 (e.g. 7) amino acids of the TGFbR2 intracellular domain) via a transmembrane domain.

In some embodiments, the human T cell expresses a polypeptide comprising a BATF3 protein, and a polypeptide comprising 4-1BB protein.

In some embodiments, the human T cell expresses a polypeptide comprising a TFAP4 protein, and a polypeptide comprising a human LTBR extracellular domain or a portion thereof linked to the intracellular domain of human IL-4R (and optionally 1-10 (e.g. 7) amino acids of the LTBR intracellular domain) via a transmembrane domain.

In some embodiments, the human T cell expresses a polypeptide comprising a ATF1 protein, and a polypeptide comprising a human IL4RA extracellular domain or a portion thereof linked to the intracellular domain of human 4-1BB.

In some embodiments, the human T cell expresses a polypeptide comprising a BATF protein, and a polypeptide comprising a human 2B4 extracellular domain or a portion thereof linked to the intracellular domain of human 4-1BB, and optionally about 1-15 (e.g., 12) amino acids of the human 4-1BB extracellular domain.

In some embodiments, the human T cell expresses a polypeptide comprising a RORC protein, and a polypeptide comprising a VISTA extracellular domain or a portion thereof linked to the intracellular domain of human CD28, and optionally about 1-15 (e.g., 12) amino acids of the human CD28 extracellular domain.

In some embodiments, the human T cell expresses a polypeptide comprising a BATF protein and a polypeptide comprising CXCR 1 protein

In some embodiments, the human T cell expresses a polypeptide comprising a MAFF protein and a polypeptide comprising IL2RA protein.

In some embodiments, the human T cell expresses a polypeptide comprising a ATF1 protein, and a polypeptide comprising a human TNFRSF12 extracellular domain or a portion thereof linked to the intracellular domain of human OX40 (and optionally 1-10 (e.g. 7) amino acids of the TNFRSF12 intracellular domain) via a transmembrane domain.

In some embodiments, the human T cell expresses a polypeptide comprising a human TIM3 extracellular domain or a portion thereof linked to the intracellular domain of human 4-1BB (and optionally about 1-15 (e.g., 12) amino acids of the human 4-1BB extracellular domain) via a transmembrane domain.

In some embodiments, the human T cell expresses a polypeptide comprising a human BATF2 extracellular domain or a portion thereof and a truncated PD-1 comprising the human PD-1 extracellular domain or a portion thereof, the PD-1 transmembrane domain and about 1-12 (e.g., 10) amino acids of the human PD-1 intracellular domain.

In some embodiments, the human T cell expresses a polypeptide comprising a human TCF7 protein, and a polypeptide comprising a human LATI protein.

In some embodiments, the human T cell expresses a polypeptide comprising a human STAT5 protein, and a human IL-2RA protein.

In some embodiments, the human T cell expresses a polypeptide comprising a human FOXJ2 protein, and a polypeptide comprising a LTBR extracellular domain or a portion thereof linked to the intracellular domain of human CD28 (and optionally 1-10 (e.g. 7) amino acids of the LTBR intracellular domain) via a transmembrane domain.

In some embodiments, the human T cell expresses a polypeptide comprising a human FOXJ2 protein, and a polypeptide comprising a LTBR extracellular domain or a portion thereof linked to amino acids 41-142 of human MyD88 (and optionally 1-10 (e.g. 7) amino acids of the LTBR intracellular domain) via a transmembrane domain.

In some embodiments, the human T cell expresses a polypeptide comprising a human HOPX protein, and a polypeptide comprising an IL4RA extracellular domain or a portion thereof linked to the intracellular domain of human 41-BB via a transmembrane domain,

In some embodiments, the human T cell expresses a polypeptide comprising an ID2 protein, and a polypeptide comprising a BLTA extracellular domain or a portion thereof linked to the intracellular domain of human 4-1BB, and optionally about 1-15 (e.g., 12) amino acids of the human 4-1BB extracellular domain.

In some embodiments, the human T cell expresses a polypeptide comprising a human SMAD1 protein, and a polypeptide comprising a MCT4 protein.

In some embodiments, the human T cell expresses a polypeptide comprising a human SMAD1 protein, and a polypeptide comprising a LTBR extracellular domain or a portion thereof linked to the intracellular domain of human IL-4R (and optionally 1-10 (e.g. 7) amino acids of the LTBR intracellular domain) via a transmembrane domain.

In some embodiments, the human T cell expresses a polypeptide comprising a DR5 extracellular domain or a portion thereof linked to a polypeptide comprising about 1-12 amino acids (e.g., 7 amino acids) of the DR5 intracellular domain and a polypeptide comprising the intracellular domain of human ICOS via a transmembrane domain.

In some embodiments, the human T cell expresses a polypeptide comprising a TIGIT extracellular domain or a portion thereof linked to the intracellular domain of human ICOS (and optionally about 1-15 (e.g., 12) amino acids of the human ICOS extracellular domain) via a transmembrane domain.

In some embodiments, the human T cell expresses a polypeptide comprising a LTBR extracellular domain or a portion thereof linked to the intracellular domain of human 4-1BB (and optionally about 1-15 (e.g., 7) amino acids of the human LTBR intracellular domain) via a transmembrane domain. In some embodiments, the polypeptide further comprises about 1-12 amino acids (e.g., 7 amino acids) of the intracellular domain of 4-1BB linked to the intracellular domain of 4-1BB.

In some embodiments, the human T cell expresses a polypeptide comprising CCR4.

In some embodiments, the human T cell expresses a polypeptide comprising a full-length 4-1BB protein, FOXJ2 protein, HES2 protein, or a fragment thereof.

In some embodiments, the T cell heterologously expresses two or more polypeptides, wherein each polypeptide comprises an amino acid sequence that is at least 95% identical to an amino acid sequence selected from the group consisting of SEQ ID NO: 30, 31, 32, 33, 34, 35, 36, 37, 38, 39, 40, 41, 42, 43, 44, 45, 46, 47, 48, 49, 50, 51, 52, 53, 54, 55, 56, 57, 58, 69, 70, 71, 72, 73, 74, 75, 76, 77, 78, 79, 80, 81, 82, 83, 84, 85, 160, 161, and 162 (i.e., SEQ ID NOs: 30-58, 69-85, 160, 161 and 162).

In some embodiments, the heterologous nucleic acid construct comprises two or more nucleic acid sequences, wherein each nucleic acid sequence is at least 95% identical to a nucleic acid sequence selected from the group consisting of SEQ ID NO: 1-29, and 59-68.

In some embodiments, the T cell expresses an antigen-specific T-cell receptor (TCR) or synthetic antigen receptor that recognizes a target antigen. In some embodiments, the T cell is a regulatory T cell, effector T cell, a memory T cell or naïve T cell. In some embodiments, the effector T cell is a CD8+ T cells or a CD4+ T cell. In some embodiments, the effector T cell is a CD8+ CD4+ T cell. In some embodiments, the T cell is a primary cell.

In some embodiments, the target insertion site is in exon 1 of a TCR-alpha subunit constant gene (TRAC). In some embodiments, the target insertion site is in exon 1 of a TCR-beta subunit constant gene (TRBC).

In some embodiments, the heterologous nucleic acid inserted into the human T cell encodes, in the following order, (i) a first self-cleaving peptide sequence; (ii) a first heterologous TCR subunit chain, wherein the TCR subunit chain comprises a variable region and a constant region of the TCR subunit; (iii) a second self-cleaving peptide sequence; (iv) one or more polypeptides as described herein; (v) a third self-cleaving peptide sequence; (vi) a variable region of a second heterologous TCR subunit chain; and (vii) a portion of the N-terminus of the endogenous TCR subunit, wherein, if the endogenous TCR subunit of the cell is a TCR-alpha (TCR-α) subunit, the first heterologous TCR subunit chain is a heterologous TCR-beta (TCR-β) subunit chain and the second heterologous TCR subunit chain is a heterologous TCR-α subunit chain, and wherein if the endogenous TCR subunit of the cell is a TCR-β subunit, the first heterologous TCR subunit chain is a heterologous TCR-α subunit chain and the second heterologous TCR subunit chain is a heterologous TCR-β subunit chain. Optionally, in any of the constructs described herein, the construct encodes two, three, four or more heterologous polypeptides.

In some embodiments, the heterologous nucleic acid inserted into the human T cell encodes, in the following order, (i) a first self-cleaving peptide sequence; (ii) one or more heterologous polypeptides as described herein; (iii) a second self-cleaving peptide sequence; (iv) a first heterologous TCR subunit chain, wherein the TCR subunit chain comprises a variable region and a constant region of the TCR subunit; (v) a third self-cleaving peptide sequence; (vi) a variable region of a second heterologous TCR subunit chain; and (vii) a portion of the N-terminus of the endogenous TCR subunit, wherein, if the endogenous TCR subunit of the cell is a TCR-alpha (TCR-α) subunit, the first heterologous TCR subunit chain is a heterologous TCR-beta (TCR-β) subunit chain and the second heterologous TCR subunit chain is a heterologous TCR-α subunit chain, and wherein if the endogenous TCR subunit of the cell is a TCR-β subunit, the first heterologous TCR subunit chain is a heterologous TCR-α subunit chain and the second heterologous TCR subunit chain is a heterologous TCR-β subunit chain.

In some embodiments, the nucleic acid construct encodes, in the following order, (i) a first self-cleaving peptide sequence; (ii) a synthetic antigen receptor; (iii) a second self-cleaving peptide sequence; (iv) one or more heterologous polypeptides described herein; and (v) a third self-cleaving peptide sequence or a polyA sequence.

In some embodiments, the nucleic acid construct encodes, in the following order, (i) a first self-cleaving peptide sequence; (ii) one or more heterologous polypeptides described herein; (iii) a second self-cleaving peptide sequence; (iv) a synthetic antigen receptor; and (v) a third self-cleaving peptide sequence or a polyA sequence.

In some embodiments, the heterologous nucleic acid construct comprises two or more nucleic acid sequences, wherein each nucleic acid is at least 95% identical to a nucleic acid sequence selected from the consisting of SEQ ID NO: 1-29, and 59-68.

Also provided is a method of modifying a human T cell comprising (a) introducing into the human T cell (i) a targeted nuclease that cleaves a target region in the TCR locus of a human T cell to create a target insertion site in the genome of the cell; and (ii) a nucleic acid construct encoding a polypeptide comprising a TFAP4 protein and a BATF protein; a polypeptide comprising a BATF3 protein and a TFAP4 protein; a polypeptide comprising a FOXJ2 protein and a RARA protein; a polypeptide comprising a ID3 protein and a TFAP4 protein; a polypeptide comprising a BATF protein and a IRF2 protein; a polypeptide comprising a NANOG protein and a TFAP4 protein; a polypeptide comprising a MAFF protein and a SATB1 protein; a polypeptide comprising a BATF protein and a IRF1 protein; a polypeptide comprising a TFAP4 protein and a HOPX protein; a polypeptide comprising a BATF protein and a HES2 protein; a polypeptide comprising a ATF2 protein and a BATF protein; a polypeptide comprising a BATF3 protein and a SMAD4 protein; a polypeptide comprising a FOXP3 protein and a RELA protein; a polypeptide comprising a ID3 protein and a BATF3 protein; a polypeptide comprising an ID2 protein and a polypeptide comprising a TP73 protein; a polypeptide comprising a SMAD3 protein, or a polypeptide comprising a BATF3 protein; a polypeptide comprising a HES2 protein; a polypeptide comprising a FOXJ2 protein; a polypeptide comprising (a) a human TFAP4 protein, and (b) a polypeptide comprising a human TIM3 extracellular domain or a portion thereof linked to a human 4-1BB intracellular domain (and optionally about 1-15 (e.g., 12) amino acids of the human 4-1BB extracellular domain) via a transmembrane domain; a polypeptide comprising (a) a human SOX5 protein, and (b) a truncated TIGIT protein comprising the human TIGIT extracellular domain or a portion thereof, the TIGIT transmembrane domain and about 1-10 (e.g., 7) amino acids of the human TIGIT intracellular domain; a polypeptide comprising (a) a human MYC protein, and (b) a polypeptide comprising a human TGFbR2 extracellular domain or a portion thereof linked to amino acids 41-142 of human MyD88 (and optionally 1-10 (e.g. 7) amino acids of the TGFbR2 intracellular domain) via a transmembrane domain; a polypeptide comprising a BATF3 protein and a 4-1BB protein; a polypeptide comprising (a) a TFAP4 protein, and (b) a polypeptide comprising a human LTBR extracellular domain or a portion thereof linked to the intracellular domain of human IL-4R (and optionally 1-10 (e.g. 7) amino acids of the LTBR intracellular domain) via a transmembrane domain; a polypeptide comprising (a) a ATF1 protein, and (b) a polypeptide comprising a human IL4RA extracellular domain or a portion thereof linked to the intracellular domain of human 4-1BB; a polypeptide comprising (a) a BATF protein, and (b) a polypeptide comprising a human 2B4 extracellular domain or a portion thereof linked to the intracellular domain of human 4-1BB, and optionally about 1-15 (e.g., 12) amino acids of the human 4-1BB extracellular domain; a polypeptide comprising (a) a RORC protein, and (b) a polypeptide comprising a VISTA extracellular domain or a portion thereof linked to the intracellular domain of human CD28, and optionally about 1-15 (e.g., 12) amino acids of the human CD28 extracellular domain; a polypeptide comprising a BATF protein and a CXCR 1 protein; a polypeptide comprising a MAFF protein and a IL2RA protein; a polypeptide comprising (a) a ATF1 protein, and (b) a polypeptide comprising a human TNFRSF12 extracellular domain or a portion thereof linked to the intracellular domain of human OX40 (and optionally 1-10 (e.g. 7) amino acids of the TNFRSF12 intracellular domain) via a transmembrane domain; a polypeptide comprising a human TIM3 extracellular domain or a portion thereof linked to the intracellular domain of human 4-1BB (and optionally about 1-15 (e.g., 12) amino acids of the human 4-1BB extracellular domain) via a transmembrane domain; a polypeptide comprising a human BATF2 extracellular domain or a portion thereof linked to a truncated PD-1 comprising the human PD-1 extracellular domain or a portion thereof, the PD-1 transmembrane domain and about 1-12 (e.g., 10) amino acids of the human PD-1 intracellular domain; a polypeptide comprising a human TCF7 protein and a human LATI protein; a polypeptide comprising a human 4-1BB protein; a polypeptide comprising a human STAT5 protein and a human IL-2RA protein; a polypeptide comprising (a) a human FOXJ2 protein and (b) a polypeptide comprising a LTBR extracellular domain or a portion thereof linked to the intracellular domain of human CD28 (and optionally 1-10 (e.g. 7) amino acids of the LTBR intracellular domain) via a transmembrane domain; a polypeptide comprising (a) a human FOXJ2 protein and (b) a polypeptide comprising a LTBR extracellular domain or a portion thereof linked to amino acids 41-142 of human MyD88 (and optionally 1-10 (e.g. 7) amino acids of the LTBR intracellular domain) via a transmembrane domain; a polypeptide comprising (a) a human HOPX protein and (b) a polypeptide comprising a IL4RA extracellular domain or a portion thereof linked to the intracellular domain of human 41-BB via a transmembrane domain; a polypeptide comprising (a) a ID2 protein, and (b) a polypeptide comprising a BLTA extracellular domain or a portion thereof linked to the intracellular domain of human 4-1BB, and optionally about 1-15 (e.g., 12) amino acids of the human 4-1BB extracellular domain; a polypeptide comprising a human SMAD1 protein and a MCT4 protein; and a polypeptide comprising (a) a human SMAD1 protein and (b) a LTBR extracellular domain or a portion thereof linked to amino acids the intracellular domain of human IL-4R (and optionally 1-10 (e.g. 7) amino acids of the LTBR intracellular domain) via a transmembrane domain; and (b) allowing recombination to occur, thereby inserting the nucleic acid construct in the target insertion site to generate a modified human T cell. In any of the compositions and methods provided herein, the nucleic acid construct can include nucleic acid sequences encoding two or more polypeptides, wherein the two or more polypeptides are the same polypeptide, for example, a nucleic acid construct encoding two or more transcription factors, wherein both transcription factors are the same (e.g., an IDE polypeptide and an ID3 polypeptide; an EOMES polypeptide and an EOMES polypeptide; a TFAP4 polypeptide and a TFAP 4 polypeptide, or a BATF polypeptide and a BATF polypeptide).

In some methods, the polypeptide comprises an amino acid sequence that is at least 95% identical to an amino acid sequence selected from the group consisting of SEQ ID NO: 30-SEQ ID NO: 58, SEQ ID NO: 69-85, SEQ ID NO: 160, SEQ ID NO: 161 and SEQ ID NO: 162.

In some methods, the target insertion site is in exon 1 of a TCR-alpha subunit constant gene (TRAC) or in exon 1 of a TCR-beta subunit constant gene (TRBC).

In some methods, the nucleic acid construct is inserted by introducing a viral vector comprising the nucleic acid construct into the cell. In some embodiments, the targeted nuclease is selected from the group consisting of an RNA-guided nuclease domain, a transcription activator-like effector nuclease (TALEN), a zinc finger nuclease (ZFN) and a megaTAL.

In some methods, the targeted nuclease, a guide RNA and the DNA template are introduced into the cell as a ribonucleoprotein complex (RNP)-DNA template complex, wherein the RNP-DNA template complex comprises: (i) the RNP, wherein the RNP comprises the targeted nuclease and the guide RNA; and (ii) the nucleic acid construct.

In some methods, the T cell expresses an antigen-specific T-cell receptor (TCR) or synthetic antigen receptor that recognizes a target antigen. In some embodiments, the T cell is a regulatory T cell, effector T cell, a memory T cell or naïve T cell. In some embodiments, the effector T cell is a CD8+ T cells or a CD4+ T cell. In some embodiments, the effector T cell is a CD8+CD4+ T cell. In some embodiments, the T cell is a primary cell.

Also provided are modified T cells produced by any of the methods described herein.

Further provided is a method of enhancing an immune response in a human subject comprising administering any of the T cells described herein. In some embodiments, the T cell expresses an antigen-specific TCR that recognizes a target antigen in the subject. In some embodiments, the human subject has cancer and the target antigen is a cancer-specific antigen. In some embodiments, the human subject has an autoimmune disorder or an allergic disorder and the antigen is an antigen associated with the autoimmune disorder or the allergic disorder. In some embodiments, the subject has an infection and the target antigen is an antigen associated with the infection. In some embodiments, the T-cell is autologous. In some embodiments, the T-cell is allogenic. In some embodiments, the T cell is an induced pluripotent stem cell (iPSC)-derived T cell.

### BRIEF DESCRIPTION OF THE DRAWINGS

The present application includes the following figures. The figures are intended to illustrate certain embodiments and/or features of the compositions and methods, and to supplement any description(s) of the compositions and methods. The figures do not limit the scope of the compositions and methods, unless the written description expressly indicates that such is the case.

FIGS. 1A-C is a schematic outlining an exemplary pooled assembly approach described herein. (A) Schematic illustration showing the pooled assembly approach that was used to generate a combinatorial library of the tonic signaling GD2 CAR plus 100 transcription factors (TFs)×100 transcription factors resulting in a >10,000-member library. The constructs (and the order of integration) was identified by amplicon sequencing of the fusion region between the two transcription factors (3′ barcode of TF1 and 5′ barcode of TF2). (B) Schematic illustration of the amplicon sequencing strategy used to detect the barcodes. (C) Representation of combinatorial constructs was analyzed in the plasmid pool and the cell pool on day 4 after electroporation and showed >99% representation of the >10,000 constructs.

FIGS. 2A-B show that the TF×TF combinatorial library was knocked into primary human T cells. As the GD2 CAR provides tonic signaling/chronic stimulation, T cells were cultured without addition of target cells. Cells were sorted on day 16 and day 4 after electroporation and the log 2 fold change (log 2FC) was calculated (day 16/day 4). Log 2FC is shown and highlights that combinations of TFAP4 and BATF performed best.

FIGS. 3A-B show the construction of a second combinatorial library. (A) A second combinatorial library was generated using the tonic signaling GD2 CAR plus 100 TFs×129 therapeutic constructs (TCs) (synthetic receptors, chemokine receptors etc.) resulting in a 13,362-member library. Again, constructs were identified by amplicon sequencing of the fusion regions. (B) Representation of combinatorial constructs was analyzed in the plasmid pool and the cell pool on day 4 after electroporation and showed >99% representation of the >10,000 constructs.

FIG. 4 shows the hits from the TF×TC screen. Screens were performed as described in FIG. 2. Top hits TFAP4_TIM-3/4-1BB and SOX5_TIGIT_trunc are highlighted. Many of the combinatorial constructs with highest Log 2FC have either TFAP4 or BATF/BATF3 as the first insert in their combinatorial construct. Looking at the fusion receptors in the TC part of the construct, 4-1BB intracellular domains was over-represented compared to CD28 intracellular domains in the top hits.

FIG. 5A-D provide validation analysis. (A) For validation analyses, single knock-ins of the combinatorial constructs (including a GD2 CAR) were performed. 50%/50% co-culture analyses of different combinatorial constructs are shown and highlight that adding TFAP4 to the constructs seems to have a stronger effect than adding BATF. (B) Activation marker expression was analyzed on CAR T cells 8 days after electroporation and showed increased levels of CD25 in TFAP4-overexpressing CAR T cells. (C) Exemplary flow cytometry plots are shown for phenotypic marker analyses 14 days after electroporation. Addition of BATF, but especially TFAP4 increases the percentage of CD62L-positive T cells. (D) Phenotypic analysis of the different combinatorial constructs 14 days after electroporation.

FIGS. 6A-D show differential expression of genes. Differentially expressed genes in the BATF-TFAP4 constructs compared to mCherry-NGFR control constructs were analyzed by RNA-seq 14 days after electroporation. A Volcano plot shows differentially expressed genes. The most differentially expressed gene was TFAP4. The color indicates if the respective gene was also found under the most differentially expressed genes when comparing TFAP4-mCherry vs control, BATF-mCherry vs control or in both of these conditions. Highlighted in yellow are genes that only show up as differentially expressed when comparing BATF-TFAP4 vs control. (B) Log 2 fold changes between the tested condition and control (mCherry-NGFR) were correlated between BATF-TFAP4 and the other constructs. Correlation analyses indicated that BATF-TFAP4 share most changes in gene expression with TFAP4-mCherry and BATF-mCherry while the correlation between BATF-TFAP4 and mCherry-JUN is lower. (C) Gene set enrichment analysis of BATF-TFAP4 vs mCherry-NGFR cells on day 14 (baseline) highlight increased expression of genes involved in cell cycle regulation. (D) After stimulation with target cells, expression of genes involved in glycolysis, oxidative phosphorylation and fatty acid metabolism was increased in BATF-TFAP4 cells while expression of genes involved in apoptosis was decreased.

FIGS. 7A-B show the killing capacity of combinatorial constructs. (A) Combinatorial constructs were co-cultured with Nalm-6/GD2 target cells and target cell killing was analyzed. BATF-TFAP4 combinatorial constructs outcompete other constructs in terms of killing capacity. (B) This finding was confirmed across multiple effector: target (E:T) ratios.

FIGS. 8A-B show in vivo results. To analyze in vivo functionality, a xenograft model of adoptive T cell transfer was used. NSG mice were injected with 0.5M Nalm-6/GFP/Luc/GD2 cells IV on day 0 followed by injection of 1M GD2 CAR T cells IV three days later. Leukemic load was determined by bioluminescence imaging. (B) Leukemia growth analyses after injection of combinatorial T cells from two different T cell donors are shown.

FIG. 9 is a schematic illustration of an exemplary GD2 CAR combinatorial construct. Figure discloses “SGSG” as SEQ ID NO: 175 and “GSGS” as SEQ ID NO: 176.

FIGS. 10A-D show that combinatorial pooled knockin screens uncovered efficient transcription factor combinations. (A) The ModPoKI platform was further advanced to screen pairwise combinations of transcription factors together with the GD2 CAR. In the combinatorial functional module, 100 TFs were combined with 100 TFs (in addition to controls) resulting in ˜10,000 different TF combinations. They were combined with the GD2 CAR resulting in a knockin size range of ˜3.3 to ˜8.2 kb. (B) Barcode sequencing of the TF×TF combinatorial plasmid library showed size-dependent representation, but confirmed that >99% of constructs were represented after pooled assembly. Statistics were done using linear regression (Im function in R studio). (C) Knockin percentage of combinatorial constructs was analyzed in the cell pool on day 4 after electroporation by amplicon sequencing and showed >99% representation of the ˜10,000 constructs. N=2 individual donors. Statistics were done using linear regression (Im function in R studio). (D) The TF×TF combinatorial library was knocked into primary human T cells. As the GD2 CAR provides tonic stimulation, T cells were cultured without addition of target cells. Cells were sorted on day 16 and day 4 after electroporation and the log 2 fold change (log 2FC) was calculated (day 16/day 4). Log 2FC for the combinatorial TF×TF constructs is shown and highlights that combinations of TFAP4 and BATF performed best. N=2 individual donors. Statistics were calculated using DESeq2. To create the volcano plot, the two possible construct orientations (e.g. BATF-TFAP4 and TFAP4-BATF) were combined to one dataset. The right panel shows barcode representation of the two construct orientations×two donors.

FIGS. 11A-E show the combinatorial knockin strategy and plasmid representation of TF×TF library, related to FIG. 10. (A-B) Schematic illustration showing the pooled assembly approach used to generate a combinatorial library of the tonic signaling GD2 CAR plus 100 transcription factors (TFs)×100 TFs resulting in a ˜10,000-member library. The inserts for TF position 1 and 2 were separately generated by pooled PCRs off of the existing TF library. The backbone (consisting of the GD2 CAR plus homology arms) and the two inserts were assembled in a pooled Hifi DNA assembly reaction resulting in the combinatorial ModPoKI plasmid library. Double-stranded HDR template was generated by pooled PCR followed by non-viral pooled knockin into primary human T cells. (C) The resulting fusion region between the two transcription factors (combinatorial barcoded multicistronic adaptor) consisted of both the barcode of the insert in position 1 and the barcode of the insert in position 2. To read out the barcode region. mRNA was isolated, reverse transcribed into cDNA and amplified using the 5′ and 3′ sequencing adaptors to add Illumina Read 1 and Read 2. The indexing sequences were added in a second PCR step. Amplicon sequencing was performed and mRNA/cDNA combinatorial barcode counts were calculated. (D) Exemplary knockin of a control construct containing the GD2 CAR with tNGFR and RFP including the new combinatorial barcoded multicistronic adaptor sequences into primary human T cells. (E) The TF×TF combinatorial library was knocked into primary human T cells. As the GD2 CAR provides tonic signaling. T cells were cultured without addition of target cells. Cells were sorted on day 16 and day 4 after electroporation and the log 2 fold change (log 2FC) was calculated (day 16/day 4). Log 2FC for the top 10 combinatorial TF×TF constructs is shown and compared to controls. N=2 individual donors. Barcode representation of the two construct orientations×two donors is shown.

FIGS. 12A-G show combinatorial knockin of TFAP4 and BATF induces favorable transcriptional programs in therapeutic T Cells. (A) Competitive fitness assays with specific combinatorial knockin constructs (including the GD2 CAR) were performed. ˜ 50%/50% co-culture analyses of different combinatorial constructs are shown. N=2 individual donors in technical triplicates. Mean+SEM shown. Unpaired t test on day 4 was performed to determine statistical significance. (B) Activation marker expression was analyzed on CAR T cells 8 days after electroporation and showed increased levels of CD25 in TFAP4 KI and BATF-TFAP dual KI CAR T cells compared to BATF KI CAR T cells. N=2 individual donors in technical duplicates. Mean+SEM shown. 2-way ANOVA with Holm-Sidak's multiple comparisons correction was performed to determine statistical significance. (C) Exemplary flow cytometry plots are shown for phenotypic markers 14 days after electroporation. (D) Phenotypic analysis of the different combinatorial KI CAR T cells 14 days after electroporation. N=2 individual donors in technical duplicates. Mean+SEM shown. 2-way ANOVA with Holm-Sidak's multiple comparisons correction was performed to determine statistical significance. (E) Differentially expressed genes in the BATF-TFAP4 KI CAR T cells compared to RFP-tNGFR control KI CAR T cells were analyzed by RNA-seq 14 days after electroporation, shown in a volcano plot. The most differentially expressed gene was TFAP4 (not shown, log 2FC=5.0, padj=6.03E-77). The color indicates if the respective gene was also found among the most differentially expressed genes when comparing TFAP4-RFP KI CAR T cells vs control KI CAR T cells, BATF-RFP KI CAR T cells vs control KI CAR T cells or in both of these comparisons. Highlighted in yellow are genes that were differentially expressed selectively in BATF-TFAP4 vs RFP-tNGFR KI CAR T cells. N=2 individual donors. (F) Combinatorial KI CAR T cells were co-cultured with Nalm-6/GFP/Luc/GD2 target cells and target cell killing was analyzed. N=2 individual donors in technical triplicates. Mean+SEM shown. 2-way ANOVA with Holm-Sidak's multiple comparisons correction was performed to determine statistical significance. (G) To assess in vivo function, NSG mice were injected with 0.5e6 Nalm-6/GFP/Luc/GD2 cells IV on day 0 followed by injection of 1e6 GD2 CAR KI T cells IV three days later. Leukemic load was determined by bioluminescence imaging. N=2 individual T cell donors with 2-5 mice per donor per group. The two different donors are shown separately in FIG. 14B. Mean+SEM shown. 2-way ANOVA with Holm Sidak's multiple comparisons test was performed to compare all constructs against the control (RFP-tNGFR) (both donors combined).

FIGS. 13A-E show transcriptomic changes driven by combinatorial BATF-TFAP4 knockin, related to FIG. 12. (A) Different combinatorial validation constructs were knocked into primary human T cells as described in FIG. 6. Bulk RNA-seq was performed 14 days after electroporation. Differentially expressed genes between BATF-TFAP4 KI GD2 CAR T cells and RFP-tNGFR KI GD2 CAR T cells are plotted. N=2 individual donors. (B) Log 2 fold changes in gene expression between the tested KI condition and control KI (RFP-tNGFR) were correlated between BATF-TFAP4 and the other constructs. Correlation analyses indicated that BATF-TFAP4 KIs were most similar in gene expression with RFP-TFAP4 and BATF-RFP, while the correlation between BATF-TFAP4 KI cells and RFP-JUN KI cells was lower. N=2 individual donors. Statistics were done using linear regression (Im function in R studio). (C-E) Gene set enrichment analysis of the BATF-TFAP4 combinatorial construct compared to RFP-tNGFR (C), RFP-TFAP4 (D) or BATF-RFP (E) without and with addition of GD2+ target cells for 24 h is shown (day 14 vs day 15 after electroporation, respectively). Notably, after stimulation with target cells, expression of genes involved in fatty acid metabolism, glycolysis, oxidative phosphorylation and IL2/STAT5 signaling was increased in BATF-TFAP4 cells compared to RFP-tNGFR cells. N=2 individual donors.

FIGS. 14A-B show the functional capacity of combinatorial BATF-TFAP4 Knockin in Vitro and in Vivo, related to FIG. 12. Combinatorial KI GD2 CAR T cells were co-cultured with Nalm-6/GFP/Luc/GD2 target cells and target cell killing was analyzed via Incucyte. BATF-TFAP4 combinatorial KI GD2 CAR T cells trended to outperform other combinatorial KI GD2 CAR T cells in terms of in vitro killing capacity. N=2 individual donors in technical triplicates. 2-way ANOVA with Holm Sidak's multiple comparisons test was performed to compare all constructs against the control (RFP-tNGFR). Mean+SEM shown. (B) Data from FIG. 12 divided by donors. Mean+SEM shown.

FIG. 15 shows the results of a CD25 sorted screen. The GD2 CAR single insert TF library was knocked into three individual primary human T cell donors. Cells were sorted for CD25 high vs low expression on day 8 after electroporation. Log 2FC of insert representation in the CD25 high vs low bin is shown. Data was normalized to controls (GFP/RFP) and to fit on a −1/1 axis. Donor-to-donor variability was observed for some constructs (mean+SEM shown).

FIG. 16 shows the results of single stimulation screens (A375/CD19 addition vs unstim condition) and repetitive stimulation screens (5× addition of A375/CD19) in a complementary model using a CD19/28z CAR in combination with the full TF and SR single insert library described in Example III. Single knockin TF and SR libraries in combination with a CD19/28z CAR were knocked into two-three individual primary human T cell donors. Input population as well as single stimulation populations (unstim vs A375/CD19 stim for 4 days) and repetitive stimulation populations (consecutive stims with A375/CD19 for 2 days×5) were harvested and changes in construct abundance were analyzed using a Log 2FC over input population. N=3 individual donors for repetitive stimulation, n=2 individual donors for single stimulation. Data was normalized to controls (GFP/RFP) and to fit on a −1/1 axis.

### DEFINITIONS

Unless defined otherwise, all technical and scientific terms used herein have the same meaning as commonly understood by one of ordinary skill in the art to which this disclosure belongs. All patents, patent applications and publications referred to throughout the disclosure herein are incorporated by reference in their entirety.

As used in the specification and the appended claims, the singular forms “a,” “an,” and “the” include plural referents unless the context clearly dictates otherwise. For example, reference to “a transcript” or “the transcript” may include a plurality of transcripts.

The use of any and all examples or exemplary language (e.g., “such as”) provided herein, is intended merely to better illustrate the invention and does not pose a limitation on the scope of the invention unless otherwise claimed.

The terms “may,” “may be,” “can,” and “can be,” and related terms are intended to convey that the subject matter involved is optional (that is, the subject matter is present in some examples and is not present in other examples), not a reference to a capability of the subject matter or to a probability, unless the context clearly indicates otherwise.

The terms “optional” and “optionally” mean that the subsequently described event, circumstance, or material may or may not occur or be present, and that the description includes instances where the event, circumstance, or material occurs or is present as well as instances where it does not occur or is not present.

The use herein of the terms “including,” “comprising,” or “having,” and variations thereof, is meant to encompass the elements listed thereafter and equivalents thereof as well as additional elements. Embodiments recited as “including,” “comprising,” or “having” certain elements are also contemplated as “consisting essentially of” and “consisting of” those certain elements. As used herein, “and/or” refers to and encompasses any and all possible combinations of one or more of the associated listed items, as well as the lack of combinations where interpreted in the alternative (“or”).

As used herein, the transitional phrase “consisting essentially of” (and grammatical variants) is to be interpreted as encompassing the recited materials or steps “and those that do not materially affect the basic and novel characteristic(s)” of the claimed invention. See, In re Herz, 537 F.2d 549, 551-52, 190 U.S.P.Q. 461, 463 (CCPA 1976) (emphasis in the original); see also MPEP § 2111.03. Thus, the term “consisting essentially of” as used herein should not be interpreted as equivalent to “comprising.”

The term “nucleic acid” or “nucleotide” refers to deoxyribonucleic acids (DNA) or ribonucleic acids (RNA) and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)).

The term “gene” can refer to the segment of DNA involved in producing or encoding a polypeptide chain. It may include regions preceding and following the coding region (leader and trailer) as well as intervening sequences (introns) between individual coding segments (exons). Alternatively, the term “gene” can refer to the segment of DNA involved in producing or encoding a non-translated RNA, such as an rRNA, tRNA, guide RNA (e.g., a single guide RNA), or micro RNA.

As used herein, the term “endogenous” with reference to a nucleic acid, for example, a gene, or a protein in a cell is a nucleic acid or protein that occurs in that particular cell as it is found in nature, for example, at its natural genomic location or locus. Moreover, a cell “endogenously expressing” a nucleic acid or protein expresses that nucleic acid or protein as it is found in nature.

As used herein the phrase “heterologous” refers to what is not normally found in nature. The term “heterologous nucleotide sequence” refers to a nucleotide sequence not normally found in a given cell in nature. As such, a heterologous nucleotide sequence may be: (a) foreign to its host cell (i.e., is exogenous to the cell); (b) naturally found in the host cell (i.e., endogenous) but present at an unnatural quantity in the cell (i.e., greater or lesser quantity than naturally found in the host cell); or (c) be naturally found in the host cell but positioned outside of its natural locus.

A “promoter” is defined as one or more a nucleic acid control sequences that direct transcription of a nucleic acid. As used herein, a promoter includes necessary nucleic acid sequences near the start site of transcription, such as, in the case of a polymerase II type promoter, a TATA element. A promoter also optionally includes distal enhancer or repressor elements, which can be located as much as several thousand base pairs from the start site of transcription.

A nucleic acid is “operably linked” when it is placed into a functional relationship with another nucleic acid sequence. For example, a promoter or enhancer is operably linked to a coding sequence if it affects the transcription of the sequence; or a ribosome binding site is operably linked to a coding sequence if it is positioned so as to facilitate translation.

“Polypeptide,” “peptide,” and “protein” are used interchangeably herein to refer to a polymer of amino acid residues. As used herein, the terms encompass amino acid chains of any length, including full-length proteins, wherein the amino acid residues are linked by covalent peptide bonds.

As used herein, the term “complementary” or “complementarity” refers to specific base pairing between nucleotides or nucleic acids. Complementary nucleotides are, generally, A and T (or A and U), and G and C. The guide RNAs described herein can comprise sequences, for example, DNA targeting sequences that are perfectly complementary or substantially complementary (e.g., having 1-4 mismatches) to a genomic sequence.

The “CRISPR/Cas” system refers to a widespread class of bacterial systems for defense against foreign nucleic acid. CRISPR/Cas systems are found in a wide range of eubacterial and archaeal organisms. CRISPR/Cas systems include type I, II, and III sub-types. Wild-type type II CRISPR/Cas systems utilize an RNA-mediated nuclease, for example, Cas9, in complex with guide and activating RNA to recognize and cleave foreign nucleic acid. Guide RNAs having the activity of both a guide RNA and an activating RNA are also known in the art. In some cases, such dual activity guide RNAs are referred to as a single guide RNA (sgRNA).

Cas9 homologs are found in a wide variety of eubacteria, including, but not limited to bacteria of the following taxonomic groups: Actinobacteria, Aquificae, Bacteroidetes-Chlorobi, Chlamydiae-Verrucomicrobia, Chlroflexi, Cyanobacteria, Firmicutes, Proteobacteria, Spirochaetes, and Thermotogae. An exemplary Cas9 protein is the Streptococcus pyogenes Cas9 protein. Additional Cas9 proteins and homologs thereof are described in, e.g., Chylinksi, et al., RNA Biol. 2013 May 1; 10 (5): 726-737; Nat. Rev. Microbiol. 2011 June; 9 (6): 467-477; Hou, et al., Proc Natl Acad Sci USA. 2013 Sep. 24; 110 (39): 15644-9; Sampson et al., Nature. 2013 May 9; 497 (7448): 254-7; and Jinek, et al., Science. 2012 Aug. 17; 337 (6096): 816-21. Variants of any of the Cas9 nucleases provided herein can be optimized for efficient activity or enhanced stability in the host cell. Thus, engineered Cas9 nucleases are also contemplated. See, for example, “Slaymaker et al., “Rationally engineered Cas9 nucleases with improved specificity,” Science 351 (6268): 84-88 (2016)).

As used herein, the term “Cas9” refers to an RNA-mediated nuclease (e.g., of bacterial or archeal origin, or derived therefrom). Exemplary RNA-mediated nucleases include the foregoing Cas9 proteins and homologs thereof. Other RNA-mediated nucleases include Cpf1 (See, e.g., Zetsche et al., Cell, Volume 163, Issue 3, p759-771, 22 Oct. 2015) and homologs thereof. As used herein, the term “ribonucleoprotein” complex and the like refers to a complex between a targeted nuclease, for example, Cas9, and a crRNA (e.g., guide RNA or single guide RNA), the Cas9 protein and a trans-activating crRNA (tracrRNA), the Cas9 protein and a guide RNA, or a combination thereof (e.g., a complex containing the Cas9 protein, a tracrRNA, and a crRNA guide RNA). It is understood that in any of the embodiments described herein, a Cas9 nuclease can be substituted with a Cpf1 nuclease or any other guided nuclease.

As used herein, the phrase “modifying” in the context of modifying a genome of a cell refers to inducing a structural change in the sequence of the genome at a target genomic region. For example, the modifying can take the form of inserting a nucleotide sequence into the genome of the cell. For example, a nucleotide sequence encoding one or more polypeptides can be inserted into the genomic sequence the TCR locus of a T cell. As used throughout a “TCR locus” is a location in the genome where the gene encoding a TCRα subunit, a TCRβ subunit, a TCRγ subunit, or a TCRδ subunit is located.

Such modifying can be performed, for example, by inducing a double stranded break within a target genomic region, or a pair of single stranded nicks on opposite strands and flanking the target genomic region. Methods for inducing single or double stranded breaks at or within a target genomic region include the use of a Cas9 nuclease domain, or a derivative thereof, and a guide RNA, or pair of guide RNAs, directed to the target genomic region.

As used herein, the phrase “introducing” in the context of introducing a nucleic acid or a complex comprising a nucleic acid, for example, an RNP-DNA template complex, refers to the translocation of the nucleic acid sequence or the RNP-DNA template complex from outside a cell to inside the cell. In some cases, introducing refers to translocation of the nucleic acid or the complex from outside the cell to inside the nucleus of the cell. Various methods of such translocation are contemplated, including but not limited to, electroporation, contact with nanowires or nanotubes, receptor mediated internalization, translocation via cell penetrating peptides, liposome mediated translocation, and the like.

As used herein, the term “selectable marker” refers to a gene which allows selection of a host cell, for example, a T cell, comprising a marker. The selectable markers may include, but are not limited to: fluorescent markers, luminescent markers and drug selectable markers, cell surface receptors, and the like. In some embodiments, the selection can be positive selection; that is, the cells expressing the marker are isolated from a population, e.g. to create an enriched population of cells expressing the selectable marker. Separation can be by any convenient separation technique appropriate for the selectable marker used. For example, if a fluorescent marker is used, cells can be separated by fluorescence activated cell sorting, whereas if a cell surface marker has been inserted, cells can be separated from the heterogeneous population by affinity separation techniques, e.g. magnetic separation, affinity chromatography, “panning” with an affinity reagent attached to a solid matrix, fluorescence activated cell sorting or other convenient technique.

As used herein, a “cell” can be a human T cell or a cell capable of differentiating into a T cell, for example, a T cell that expresses a TCR receptor molecule. These include hematopoietic stem cells and cells derived from hematopoietic stem cells.

As used herein, the phrase “hematopoietic stem cell” refers to a type of stem cell that can give rise to a blood cell. Hematopoietic stem cells can give rise to cells of the myeloid or lymphoid lineages, or a combination thereof. Hematopoietic stem cells are predominantly found in the bone marrow, although they can be isolated from peripheral blood, or a fraction thereof. Various cell surface markers can be used to identify, sort, or purify hematopoietic stem cells. In some cases, hematopoietic stem cells are identified as c-kit⁺ and lin⁻. In some cases, human hematopoietic stem cells are identified as CD34⁺, CD59⁺, Thy1/CD90⁺, CD38^{lo/-}, C-kit/CD117⁺, lin⁻. In some cases, human hematopoietic stem cells are identified as CD34⁻, CD59⁺, Thy1/CD90⁺, CD38^{lo/-}, C-kit/CD117⁺, lin⁻. In some cases, human hematopoietic stem cells are identified as CD133⁺, CD59⁺, Thy1/CD90⁺, CD38^{lo/-}, C-kit/CD117⁺, lin⁻. In some cases, mouse hematopoietic stem cells are identified as CD34^{lo/-}, SCA-1⁺, Thy1^{+/lo}, CD38⁺, C-kit⁺, lin⁻. In some cases, the hematopoietic stem cells are CD150⁺CD48⁻CD244⁻.

As used herein, the phrase “hematopoietic cell” refers to a cell derived from a hematopoietic stem cell. The hematopoietic cell may be obtained or provided by isolation from an organism, system, organ, or tissue (e.g., blood, or a fraction thereof). Alternatively, an hematopoietic stem cell can be isolated and the hematopoietic cell obtained or provided by differentiating the stem cell. Hematopoietic cells include cells with limited potential to differentiate into further cell types. Such hematopoietic cells include, but are not limited to, multipotent progenitor cells, lineage-restricted progenitor cells, common myeloid progenitor cells, granulocyte-macrophage progenitor cells, or megakaryocyte-erythroid progenitor cells. Hematopoietic cells include cells of the lymphoid and myeloid lineages, such as lymphocytes, erythrocytes, granulocytes, monocytes, and thrombocytes. In some embodiments, the hematopoietic cell is an immune cell, such as a T cell, B cell, macrophage, a natural killer (NK) cell or dendritic cell. In some embodiments the cell is an innate immune cell.

As used herein, the phrase “T cell” refers to a lymphoid cell that expresses a T cell receptor molecule. T cells include human alpha beta (αβ) T cells and human gamma delta (γδ) T cells. T cells include, but are not limited to, naïve T cells, stimulated T cells, primary T cells (e.g., uncultured), cultured T cells, immortalized T cells, helper T cells, cytotoxic T cells, memory T cells, regulatory T cells, natural killer T cells, combinations thereof, or sub-populations thereof. T cells can be CD4⁺, CD8⁺, or CD4⁺ and CD8⁺. T cells can also be CD4⁻, CD8⁻, or CD4⁻ and CD8⁻. T cells can be helper cells, for example helper cells of type T_{H}1, T_{H}2, T_{H}3, T_{H}9, T_{H}17, or T_{FH}. T cells can be cytotoxic T cells. Regulatory T cells can be FOXP3⁺ or FOXP3⁻. T cells can be alpha/beta T cells or gamma/delta T cells. In some cases, the T cell is a CD4⁺CD25^{hi}CD127^{lo} regulatory T cell. In some cases, the T cell is a regulatory T cell selected from the group consisting of type 1 regulatory (Tr1), T_{H}3, CD8+CD28−, Treg17, and Qa-1 restricted T cells, or a combination or sub-population thereof. In some cases, the T cell is a FOXP3⁺ T cell. In some cases, the T cell is a CD4⁺CD25^{lo}CD127^{hi} effector T cell. In some cases, the T cell is a CD4⁺CD25^{lo}CD127^{hi}CD45RA^{hi}CD45RO⁻ naïve T cell. A T cell can be a recombinant T cell that has been genetically manipulated.

As used herein, the phrase “primary” in the context of a primary cell is a cell that has not been transformed or immortalized. Such primary cells can be cultured, sub-cultured, or passaged a limited number of times (e.g., cultured 0, 1, 2, 3, 4, 5, 6, 7, 8, 9, 10, 11, 12, 13, 14, 15, 16, 17, 18, 19, or 20 times). In some cases, the primary cells are adapted to in vitro culture conditions. In some cases, the primary cells are isolated from an organism, system, organ, or tissue, optionally sorted, and utilized directly without culturing or sub-culturing. In some cases, the primary cells are stimulated, activated, or differentiated. For example, primary T cells can be activated by contact with (e.g., culturing in the presence of) CD3, CD28 agonists, IL-2, IFN-γ, or a combination thereof.

“Treating” refers to any indicia of success in the treatment or amelioration or prevention of the disease, condition, or disorder, including any objective or subjective parameter such as abatement; remission; diminishing of symptoms or making the disease condition more tolerable to the patient; slowing in the rate of degeneration or decline; or making the final point of degeneration less debilitating.

As used herein, the term “homology directed repair” or HDR refers to a cellular process in which cut or nicked ends of a DNA strand are repaired by polymerization from a homologous template nucleic acid. Thus, the original sequence is replaced with the sequence of the template. In some cases, an exogenous template nucleic acid, for example, a DNA template, can be introduced to obtain a specific HDR-induced change of the sequence at a target site. In this way, specific mutations can be introduced at a cut site, for example, a cut site created by a targeted nuclease. A single-stranded DNA template or a double-stranded DNA template can be used by a cell as a template for editing or modifying the genome of a cell, for example, by HDR. Generally, the single-stranded DNA template or a double-stranded DNA template has at least one region of homology to a target site. In some cases, the single-stranded DNA template or double-stranded DNA template has two homologous regions, for example, a 5′ end and a 3′ end, flanking a region that contains the DNA template to be inserted at a target cut or insertion site.

The term “substantial identity” or “substantially identical,” as used in the context of polynucleotide or polypeptide sequences, refers to a sequence that has at least 60% sequence identity to a reference sequence. Alternatively, percent identity can be any integer from 60% to 100%. Exemplary embodiments include at least: 60%, 65%, 70%, 75%, 80%, 85%, 90%, 91%, 92%, 93%, 94%, 95%, 96%, 97%, 98%, or 99%, as compared to a reference sequence using the programs described herein; preferably BLAST using standard parameters, as described below. One of skill will recognize that these values can be appropriately adjusted to determine corresponding identity of proteins encoded by two nucleotide sequences by taking into account codon degeneracy, amino acid similarity, reading frame positioning and the like.

For sequence comparison, typically one sequence acts as a reference sequence to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window,” as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison may be conducted by the local homology algorithm of Smith and Waterman Add. APL. Math. 2:482 (1981), by the homology alignment algorithm of Needleman and Wunsch J. Mol. Biol. 48:443 (1970), by the search for similarity method of Pearson and Lipman Proc. Natl. Acad. Sci. (U.S.A.) 85:2444 (1988), by computerized implementations of these algorithms (e.g., BLAST), or by manual alignment and visual inspection.

Algorithms that are suitable for determining percent sequence identity and sequence similarity are the BLAST and BLAST 2.0 algorithms, which are described in Altschul et al. (1990) J. Mol. Biol. 215:403-410 and Altschul et al. (1977) Nucleic Acids Res. 25:3389-3402, respectively. Software for performing BLAST analyses is publicly available through the National Center for Biotechnology Information (NCBI) web site. The algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al, supra). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word size (W) of 28, an expectation (E) of 10, M=1, N=−2, and a comparison of both strands. For amino acid sequences, the BLASTP program uses as defaults a word size (W) of 3, an expectation (E) of 10, and the BLOSUM62 scoring matrix (see Henikoff & Henikoff, Proc. Natl. Acad. Sci. USA 89:10915 (1989)).

The BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P (N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.01, more preferably less than about 10⁻⁵, and most preferably less than about 10⁻²⁰.

### DETAILED DESCRIPTION OF THE INVENTION

The following description recites various aspects and embodiments of the present compositions and methods. No particular embodiment is intended to define the scope of the compositions and methods. Rather, the embodiments merely provide non-limiting examples of various compositions and methods that are at least included within the scope of the disclosed compositions and methods. The description is to be read from the perspective of one of ordinary skill in the art; therefore, information well known to the skilled artisan is not necessarily included.

The present disclosure is directed to compositions and methods for modifying the genome of a T cell. The inventors have discovered that human T cells can be modified to alter T cell specificity and function. In some embodiments, the human T cells are modified to heterologous express one or more transcription factors to alter T cell function, for example, to increase the tumor cell killing properties of the T cell. In some embodiments, the human T cells are modified to heterologous express two or more transcription factors (e.g., a combination of two or more transcription factors) to alter T cell function, for example, to increase the tumor cell killing properties of the T cell. In some embodiments, the human T cells are modified to heterologously express a combination of a transcription factor and a switch receptor (i.e, a chimeric molecule or fusion protein that combines a ligand binding domain with a signaling domain) to alter T cell function, for example, to increase the tumor cell killing properties of the T cell.

### Compositions

Provided herein is a human T cell that heterologously expresses one or more polypeptides, wherein the one or more polypeptides are encoded by a nucleic acid construct inserted into the TCR locus of the cell. Any of the polypeptides described herein can be heterologously expressed in a human T cell. In some examples, two or more, three or more, four or more or five or more polypeptides described herein are heterologously expressed in a human T cell. In some examples the one or more, two or more, three or more polypeptides etc., are encoded by one or more nucleic acid constructs.

Exemplary polypeptides include, but are not limited to, the amino acid sequences set forth in Table 1 and Table 2, for example, SEQ ID NOs: 30-58 and 69-85, 160, 161, and 162. A polypeptide comprising an amino acid sequence that is at least 80%, 85%, 90%, 99%, or 100% identical to any one of SEQ ID NOs: 30-58 and 69-85, 160, 161, and 162 can also be expressed in a human T cell. Other polypeptides that can be heterologously expressed include polypeptides comprising the amino acid sequences set forth in Table 3. A polypeptide comprising an amino acid sequence that is at least 80%, 85%, 90%, 99%, or 100% identical to any one of the amino acid sequences set forth in Table 3 can also be heterologously expressed in a human T cell.

In some embodiments, the nucleic acid construct inserted into the human T cell encodes two or more polypeptides. In some embodiments, the nucleic acid construct can comprise two or more nucleic acid sequences in a multicistronic construct. It is understood that the two or more nucleic acid sequences encoding the two or more polypeptides can be in any order in the nucleic acid construct.

In some embodiments, the nucleic acid construct inserted into the human T cell encodes a TFAP polypeptide comprising an amino acid sequence at least 95% identical to SEQ ID NO: 30, and a BATF polypeptide comprising an amino acid sequence at least 95% identical to SEQ ID NO: 31.

In some embodiments, the nucleic acid construct inserted into the human T cell encodes a BATF3 polypeptide comprising an amino acid sequence at least 95% identical to SEQ ID NO: 32, and a TFAP4 polypeptide comprising an amino acid sequence at least 95% identical to SEQ ID NO: 30.

In some embodiments, the nucleic acid construct inserted into the human T cell encodes a FOXJ2 polypeptide comprising an amino acid sequence at least 95% identical to SEQ ID NO: 33, and a RARA polypeptide comprising an amino acid sequence at least 95% identical to SEQ ID NO: 34.

In some embodiments, the nucleic acid construct inserted into the human T cell encodes a ID3 polypeptide comprising an amino acid sequence at least 95% identical to SEQ ID NO: 35, and a TFAP4 polypeptide comprising an amino acid sequence at least 95% identical to SEQ ID NO: 30.

In some embodiments, the nucleic acid construct inserted into the human T cell encodes a BATF polypeptide comprising an amino acid sequence at least 95% identical to SEQ ID NO: 31, and a IRF2 polypeptide comprising an amino acid sequence at least 95% identical to SEQ ID NO: 36.

In some embodiments, the nucleic acid construct inserted into the human T cell encodes a NANOG polypeptide comprising an amino acid sequence at least 95% identical to SEQ ID NO: 37, and a TFAP4 polypeptide comprising an amino acid sequence at least 95% identical to SEQ ID NO: 30.

In some embodiments, the nucleic acid construct inserted into the human T cell encodes a MAFF polypeptide comprising an amino acid sequence at least 95% identical to SEQ ID NO: 38, and a SATB1 polypeptide comprising an amino acid sequence at least 95% identical to SEQ ID NO: 39.

In some embodiments, the nucleic acid construct inserted into the human T cell encodes a BATF polypeptide comprising an amino acid sequence at least 95% identical to SEQ ID NO: 32, and a IRF1 polypeptide comprising an amino acid sequence at least 95% identical to SEQ ID NO: 36.

In some embodiments, the nucleic acid construct inserted into the human T cell encodes a TFAP4 polypeptide comprising an amino acid sequence at least 95% identical to SEQ ID NO: 39, and a HOPX polypeptide comprising an amino acid sequence at least 95% identical to SEQ ID NO: 51.

In some embodiments, the nucleic acid construct inserted into the human T cell encodes a TFAP4 polypeptide comprising an amino acid sequence at least 95% identical to SEQ ID NO: 30, and a HOPX polypeptide comprising an amino acid sequence at least 95% identical to SEQ ID NO: 42.

In some embodiments, the nucleic acid construct inserted into the human T cell encodes a BATF polypeptide comprising an amino acid sequence at least 95% identical to SEQ ID NO: 31, and a HES2 polypeptide comprising an amino acid sequence at least 95% identical to SEQ ID NO: 43.

In some embodiments, the nucleic acid construct inserted into the human T cell encodes a ATF2 polypeptide comprising an amino acid sequence at least 95% identical to SEQ ID NO: 47, and a BATF polypeptide comprising an amino acid sequence at least 95% identical to SEQ ID NO: 31.

In some embodiments, the nucleic acid construct inserted into the human T cell encodes a BATF3 polypeptide comprising an amino acid sequence at least 95% identical to SEQ ID NO:

32, and a SMAD4 polypeptide comprising an amino acid sequence at least 95% identical to SEQ ID NO: 48.

In some embodiments, the nucleic acid construct inserted into the human T cell encodes a FOXP3 polypeptide comprising an amino acid sequence at least 95% identical to SEQ ID NO: 49, and a RELA polypeptide comprising an amino acid sequence at least 95% identical to SEQ ID NO: 50.

In some embodiments, the nucleic acid construct inserted into the human T cell encodes a ID3 polypeptide comprising an amino acid sequence at least 95% identical to SEQ ID NO: 35, and a BATF3 polypeptide comprising an amino acid sequence at least 95% identical to SEQ ID NO: 32.

In some embodiments, the nucleic acid construct inserted into the human T cell encodes a SMAD3 polypeptide comprising an amino acid sequence at least 95% identical to SEQ ID NO: 46, and a BATF3 polypeptide comprising an amino acid sequence at least 95% identical to SEQ ID NO: 32.

In some embodiments, the nucleic acid construct inserted into the human T cell encodes an ID2 polypeptide comprising an amino acid sequence at least 95% identical to SEQ ID NO: 44, and a TP73 polypeptide comprising an amino acid sequence at least 95% identical to SEQ ID NO: 45.

In some embodiments, the nucleic acid construct inserted into the human T cell encodes a TFAP4 polypeptide comprising an amino acid sequence at least 95% identical to SEQ ID NO: 30, and a TIM-3/4-1BB polypeptide comprising an amino acid sequence at least 95% identical to SEQ ID NO: 69.

In some embodiments, the nucleic acid construct inserted into the human T cell encodes a SOX5 polypeptide comprising an amino acid sequence at least 95% identical to SEQ ID NO: 51, and a TIM-3/4-1BB polypeptide comprising an amino acid sequence at least 95% identical to SEQ ID NO: 69.

In some embodiments, the nucleic acid construct inserted into the human T cell encodes a MYC polypeptide comprising an amino acid sequence at least 95% identical to SEQ ID NO: 53, and a TGFbR2/MyD88 polypeptide comprising an amino acid sequence at least 95% identical to SEQ ID NO: 70.

In some embodiments, the nucleic acid construct inserted into the human T cell encodes a BATF3 polypeptide comprising an amino acid sequence at least 95% identical to SEQ ID NO: 32, and a 4-1BB polypeptide comprising an amino acid sequence at least 95% identical to SEQ ID NO: 55.

In some embodiments, the nucleic acid construct inserted into the human T cell encodes a TFAP4 polypeptide comprising an amino acid sequence at least 95% identical to SEQ ID NO: 30, and a LTBR-IL-4R polypeptide comprising an amino acid sequence at least 95% identical to SEQ ID NO: 71.

In some embodiments, the nucleic acid construct inserted into the human T cell encodes a ATF1 polypeptide comprising an amino acid sequence at least 95% identical to SEQ ID NO: 58, and a IL4RA/4-1BB polypeptide comprising an amino acid sequence at least 95% identical to SEQ ID NO: 72.

In some embodiments, the nucleic acid construct inserted into the human T cell encodes a BATF polypeptide comprising an amino acid sequence at least 95% identical to SEQ ID NO: 31, and a 2B4/4-1BB polypeptide comprising an amino acid sequence at least 95% identical to SEQ ID NO: 73.

In some embodiments, the nucleic acid construct inserted into the human T cell encodes a RORC polypeptide comprising an amino acid sequence at least 95% identical to SEQ ID NO: 79, and a VISTA/CD28 polypeptide comprising an amino acid sequence at least 95% identical to SEQ ID NO: 74.

In some embodiments, the nucleic acid construct inserted into the human T cell encodes a BATF polypeptide comprising an amino acid sequence at least 95% identical to SEQ ID NO: 31, and a CXCR1 polypeptide comprising an amino acid sequence at least 95% identical to SEQ ID NO: 57.

In some embodiments, the nucleic acid construct inserted into the human T cell encodes a MAFF polypeptide comprising an amino acid sequence at least 95% identical to SEQ ID NO: 38, and a IL-2RA polypeptide comprising an amino acid sequence at least 95% identical to SEQ ID NO: 54.

In some embodiments, the nucleic acid construct inserted into the human T cell encodes a ATF1 polypeptide comprising an amino acid sequence at least 95% identical to SEQ ID NO: 58, and a TNFRSF12-OX40 polypeptide comprising an amino acid sequence at least 95% identical to SEQ ID NO: 75.

In some embodiments, the nucleic acid construct inserted into the human T cell encodes a TIM-3/4-1BB polypeptide comprising an amino acid sequence at least 95% identical to SEQ ID NO: 69.

In some embodiments, the nucleic acid construct inserted into the human T cell encodes a BATF2 polypeptide comprising an amino acid sequence at least 95% identical to SEQ ID NO: 80 and a truncated PD1 polypeptide comprising an amino acid sequence at least 95% identical to SEQ ID NO: 81.

In some embodiments, the nucleic acid construct inserted into the human T cell encodes a TCF7 polypeptide comprising an amino acid sequence at least 95% identical to SEQ ID NO: 82, and a LAT polypeptide comprising an amino acid sequence at least 95% identical to SEQ ID NO: 83.

In some embodiments, the nucleic acid construct inserted into the human T cell encodes a STAT5 polypeptide comprising an amino acid sequence at least 95% identical to SEQ ID NO: 83, and a IL-2RA polypeptide comprising an amino acid sequence at least 95% identical to SEQ ID NO: 54.

In some embodiments, the nucleic acid construct inserted into the human T cell encodes a FOXJ2 polypeptide comprising an amino acid sequence at least 95% identical to SEQ ID NO: 33, and a LTBR-CD28 polypeptide comprising an amino acid sequence at least 95% identical to SEQ ID NO: 76.

In some embodiments, the nucleic acid construct inserted into the human T cell encodes a FOXJ2 polypeptide comprising an amino acid sequence at least 95% identical to SEQ ID NO: 33, and a LTBR-MyD88 polypeptide comprising an amino acid sequence at least 95% identical to SEQ ID NO: 77.

In some embodiments, the nucleic acid construct inserted into the human T cell encodes a HOPX polypeptide comprising an amino acid sequence at least 95% identical to SEQ ID NO: 42, and a IL4RA/4-1BB polypeptide comprising an amino acid sequence at least 95% identical to SEQ ID NO: 72.

In some embodiments, the nucleic acid construct inserted into the human T cell encodes a ID2 polypeptide comprising an amino acid sequence at least 95% identical to SEQ ID NO: 44, and a BTLA/4-1BB polypeptide comprising an amino acid sequence at least 95% identical to SEQ ID NO: 85.

In some embodiments, the nucleic acid construct inserted into the human T cell encodes a SMAD1 polypeptide comprising an amino acid sequence at least 95% identical to SEQ ID NO: 56, and a MCT4 polypeptide comprising an amino acid sequence at least 95% identical to SEQ ID NO: 84.

In some embodiments, the nucleic acid construct inserted into the human T cell encodes a SMAD1 polypeptide comprising an amino acid sequence at least 95% identical to SEQ ID NO: 56, and a LTBR-IL-4R polypeptide comprising an amino acid sequence at least 95% identical to SEQ ID NO: 71.

In some embodiments, the nucleic acid construct inserted into the human T cell encodes a TIGIT polypeptide comprising an amino acid sequence at least 95% identical to SEQ ID NO: 121, and an ICOS polypeptide comprising an amino acid sequence at least 95% identical to SEQ ID NO: 159;

In some embodiments, the nucleic acid construct inserted into the human T cell encodes a LTBR polypeptide comprising an amino acid sequence at least 95% identical to SEQ ID NO: 123, and an 4-1BB polypeptide comprising an amino acid sequence at least 95% identical to SEQ ID NO: 146;

In some embodiments, the nucleic acid construct inserted into the human T cell encodes a DR5 polypeptide comprising an amino acid sequence at least 95% identical to SEQ ID NO: 133, and an ICOS polypeptide comprising an amino acid sequence at least 95% identical to SEQ ID NO: 159.

In some embodiments, the polypeptide comprises a full-length HES2 protein, a FOXJ2 protein, or a 41-BB protein.

One or more nucleic acid sequences described herein, for example, one or more of SEQ ID Nos: 1-29, and 59-68 can be inserted into the TCR locus of a T cell. Nucleic acid sequences encoding two or more polypeptides described herein can also be inserted into the TCR locus of a T cell. In some embodiments, a nucleic acid sequence encoding one or more sequences selected from the group consisting of SEQ ID NO: 30-SEQ ID NO: 58, SEQ ID NO: 69-85, SEQ ID NO: 160, SEQ ID NO: 161 and SEQ ID NO: 162, is inserted into the TCR locus of the T cell. In some embodiments, one or more nucleic acid sequences that are at least 80%, 85%, 90%, 99%, or 100% identical to any one of the nucleic acid sequences set forth as SEQ ID Nos: 1-29, and 59-68, or a nucleic acid sequence that encodes two or more sequences selected from the group consisting of SEQ ID NO: 30-SEQ ID NO: 58, SEQ ID NO: 69-85, SEQ ID NO: 160, SEQ ID NO: 161 and SEQ ID NO: 162 is inserted into the TCR locus of the T cell.

Any polypeptide sequence or combinations of polypeptide sequences; nucleic acid sequence or combinations of nucleic acid sequences; T cell comprising one or more polypeptides; T cell comprising one or more nucleic acid sequences; or a method that uses a T cell, polypeptide or nucleic acid sequence described herein can be claimed.

Insertion of a heterologous coding sequence into the TCR locus means that the expression of one or more heterologous proteins will be controlled by the endogenous TCR promoter and in some embodiments will be expressed as part of a larger fusion protein with a TCR polypeptide that is subsequently cleaved to form separate TCR and heterologous polypeptides. The TCR polypeptide can be endogenous or also added to the TCR locus to provide a novel TCR affinity (for example, but not limited to, to a cancer antigen) to the T-cell. In some embodiments, the nucleic acid construct is inserted in a target insertion site in exon 1 of a TCR-alpha subunit constant gene (TRAC). In some embodiments, the nucleic acid construct is inserted in a target insertion site in exon 1 of a TCR-beta subunit constant gene (TRBC), for example, in exon 1 of a TRBC1 gene or exon1 of a TRBC2 gene. Upon insertion of the nucleic acid construct into the TCR locus of a cell, the construct is under the control of an endogenous TCR promoter, for example a TRACI promoter or a TRBC promoter. As set forth below, the nucleic acid constructs provided herein encode a TCR or synthetic antigen receptor that is co-expressed with the polypeptide. Once the construct is incorporated into the genome of the T cell by HDR, and under the control of the endogenous promoter, the T cells can be cultured under conditions that allow transcription of the inserted construct into a single mRNA sequence encoding a fusion polypeptide that is then processed into separate heterologous polypeptides (e.g., for example by cleavage of a peptide sequence linking the polypeptides). Insertion of any of the nucleic acid constructs described herein encoding the components of a heterologous T cell receptor and one or more heterologous polypeptides will produce a T cell with the specificity of the heterologous TCR receptor and the function of the heterologous polypeptide(s). In some embodiments, the T cell expresses an antigen-specific TCR that recognizes a target antigen. Similarly, insertion of any of the nucleic acid constructs described herein encoding a synthetic antigen receptor and one or more heterologous polypeptides will produce a T cell with the specificity of the synthetic antigen receptor and the function of the heterologous polypeptide. In some embodiments, the T cell expresses a synthetic antigen receptor that recognizes a target antigen.

In some embodiments, the heterologous nucleic acid inserted into the human T cell encodes, in the following order, (i) a first self-cleaving peptide sequence; (ii) a first heterologous TCR subunit chain, wherein the TCR subunit chain comprises a variable region and a constant region of the TCR subunit; (iii) a second self-cleaving peptide sequence; (iv) one or more heterologous polypeptides as described herein; (v) a third self-cleaving peptide sequence; (vi) a variable region of a second heterologous TCR subunit chain; and (vii) a portion of the N-terminus of the endogenous TCR subunit, wherein, if the endogenous TCR subunit of the cell is a TCR-alpha (TCR-α) subunit, the first heterologous TCR subunit chain is a heterologous TCR-beta (TCR-β) subunit chain and the second heterologous TCR subunit chain is a heterologous TCR-α subunit chain, and wherein if the endogenous TCR subunit of the cell is a TCR-β subunit, the first heterologous TCR subunit chain is a heterologous TCR-α subunit chain and the second heterologous TCR subunit chain is a heterologous TCR-β subunit chain.

In some embodiments, the heterologous nucleic acid inserted into the human T cell encodes, in the following order, (i) a first self-cleaving peptide sequence; (ii) one or more heterologous polypeptides as described herein; (iii) a second self-cleaving peptide sequence; (iv) a first heterologous TCR subunit chain, wherein the TCR subunit chain comprises a variable region and a constant region of the TCR subunit; (v) a third self-cleaving peptide sequence; (vi) a variable region of a second heterologous TCR subunit chain; and (vii) a portion of the N-terminus of the endogenous TCR subunit, wherein, if the endogenous TCR subunit of the cell is a TCR-alpha (TCR-α) subunit, the first heterologous TCR subunit chain is a heterologous TCR-beta (TCR-β) subunit chain and the second heterologous TCR subunit chain is a heterologous TCR-α subunit chain, and wherein if the endogenous TCR subunit of the cell is a TCR-β subunit, the first heterologous TCR subunit chain is a heterologous TCR-α subunit chain and the second heterologous TCR subunit chain is a heterologous TCR-β subunit chain.

In the compositions and methods described herein, if the endogenous TCR subunit is a TCR-alpha (TCR-α) subunit, the first heterologous TCR subunit chain is a heterologous TCR-beta (TCR-β) subunit chain and the second heterologous TCR subunit chain is a heterologous TCR-α subunit chain. In some methods, if the endogenous TCR subunit is a TCR-β subunit, the first heterologous TCR subunit chain is a heterologous TCR-α subunit chain and the second heterologous TCR subunit chain is a heterologous TCR-β subunit chain.

As used throughout, the term “endogenous TCR subunit” is the TCR subunit, for example, TCR-α or TCR-β that is endogenously expressed by the cell that the nucleic acid construct is introduced into. As set forth above, the nucleic acid constructs described herein encode multiple amino acid sequences that are expressed as a multicistronic sequence that is processed, i.e., self-cleaved, to produce two or more amino acid sequences, for example, a TCR-α subunit, a TCR-β subunit and the polypeptide encoded by the construct, or a synthetic antigen receptor (e.g. a CAR (See, for example, Guedan et al. “Engineering and Design of Chimeric Antigen Receptors,” Mol. Ther. Methods & Clinical Development 12:145-156 (2019)) or SynNotch receptor (See, for example, Cho et al. “Engineering Axl specific CAR and SynNotch receptor for cancer therapy,” Nature Scientific Reports 8, Article No: 3846 (2018)) and the polypeptide encoded by the construct.

In some nucleic acid constructs, the size of the nucleic acid encoding the N-terminal portion of the endogenous TCR subunit will depend on the number of nucleotides in the endogenous TRAC or TRBC nucleic acid sequence between the start of TRAC exon 1 or TRBC exon 1 and the targeted insertion site. For example, if the number of nucleotides between the start of TRAC exon 1 and the insertion site is less than or greater than 25 nucleotides, a nucleic acid of less than or greater than 25 nucleotides encoding the N-terminal portion of the endogenous TCR-a subunit can be in the construct.

In the examples above, translation of the mRNA sequence transcribed from the construct results in expression of one protein that self-cleaves into four or more separate polypeptide sequences, i.e., an inactive, endogenous variable region peptide lacking a transmembrane domain, (which can be, e.g., degraded in the endoplasmic reticulum or secreted following translation), a full-length heterologous antigen-specific TCR-β chain or TCR-α chain, one or more polypeptide sequence as described herein, and a full length heterologous antigen-specific TCR-α chain or TCR-βchain. The full-length antigen specific TCR-β chain and the full length antigen-specific TCR-α chain form a TCR with desired antigen-specificity. In some embodiments, the polypeptide enhances or imparts a desired function(s) in the T cell. mRNA transcribed from any of the other nucleic acid constructs described herein are similarly processed in a T cell.

In some embodiments, the nucleic acid construct encodes, in the following order, (i) a first self-cleaving peptide sequence; (ii) a first heterologous TCR subunit chain, wherein the TCR subunit chain comprises the variable region and the constant region of the TCR subunit; (iii) a second self-cleaving peptide sequence; (iv) a second heterologous TCR subunit chain, wherein the TCR subunit chain comprises the variable region and the constant region of the TCR subunit; (v) a third self-cleaving peptide sequence; (vi) one or more heterologous polypeptides described herein; and (vii) a fourth self-cleaving peptide sequence or a poly A sequence, wherein if the endogenous TCR subunit is a TCR-alpha (TCR-α) subunit, the first heterologous TCR subunit chain is a heterologous TCR-beta (TCR-β) subunit chain and the second heterologous TCR subunit chain is a heterologous TCR-α subunit chain, and wherein if the endogenous TCR subunit is a TCR-β subunit, the first heterologous TCR subunit chain is a heterologous TCR-α subunit chain and the second heterologous TCR subunit chain is a heterologous TCR-β subunit chain.

In some embodiments, the nucleic acid construct encodes, in the following order, (i) a first self-cleaving peptide sequence; (ii) a synthetic antigen receptor; (iii) a second self-cleaving peptide sequence; (iv) one or more heterologous polypeptide described herein; and (v) a third self-cleaving peptide sequence or a polyA sequence.

In some embodiments, the nucleic acid construct encodes, in the following order, (i) a first self-cleaving peptide sequence; (ii) one or more heterologous polypeptide; (iii) a second self-cleaving peptide sequence; (iv) a synthetic antigen receptor; and (v) a third self-cleaving peptide sequence or a polyA sequence. It is understood that in any of the nucleic acid constructs described herein, the one or more heterologous polypeptides can be separated by self-cleaving peptides such that the one or more polypeptides are self-cleaved into one or more separate polypeptides in the human T cell, as described above.

Examples of self-cleaving peptides include, but are not limited to, self-cleaving viral 2A peptides, for example, a porcine teschovirus-1 (P2A) peptide, a Thosea asigna virus (T2A) peptide, an equine rhinitis A virus (E2A) peptide, or a foot-and-mouth disease virus (F2A) peptide. Self-cleaving 2A peptides allow expression of multiple gene products from a single construct. (See, for example, Chng et al. “Cleavage efficient 2A peptides for high level monoclonal antibody expression in CHO cells,” MAbs 7 (2): 403-412 (2015)). In some embodiments, the nucleic acid construct comprises two or more self-cleaving peptides. In some embodiments, the two or more self-cleaving peptides are all the same. In other embodiments, at least one of the two or more self-cleaving peptides is different.

In some embodiments, one or more linker sequences separate the components of the nucleic acid construct. The linker sequence can be two, three, four, five, six, seven, eight, nine, ten amino acids or greater in length.

In some embodiments, the nucleic acid construct comprises flanking homology arm sequences having homology to a human TCR locus. In the compositions and methods described herein, the length of one or both homology arm sequences is at least about 50, 100, 150, 200, 250, 300, 350, 400 or 450 nucleotides. In some cases, a nucleotide sequence that is homologous to a genomic sequence is at least 80%, 90%, 95%, 99% or 100% complementary to the genomic sequence. In some embodiments, one or both homology arm sequences optionally comprises a mismatched nucleotide sequence compared to a homologous sequence in the genomic sequence in the TCR locus flanking the insertion site in the TCR locus.

In some embodiments, the nucleic acid construct optionally encodes a selectable marker that can be used to separate or isolate subpopulations of modified T cells. In some embodiments, the nucleic acid construct optionally comprises one or more barcode sequences that indicate the identity of the one or more polypeptides encoded by the construct.

Any of the polypeptides described herein can be encoded by any of the nucleic acid constructs described herein. In some embodiments, the polypeptide sequence encoded by the heterologous nucleic acid construct is at least 95% identical to an amino acid sequence selected from the group consisting of SEQ ID NO: 30-58 and 69-85. In some embodiments, the heterologous nucleic acid construct encodes two or more polypeptides, wherein each polypeptide is at least 95% identical to an amino acid sequence selected from the group consisting of SEQ ID NO: 30-58 and 69-85.

Also provided are polypeptides that are at least 95% identical to SEQ ID NO: 30-58 and 69-85. Nucleic acids encoding these polypeptides are also provided herein.

Also provided is a human T cell comprising any of the nucleic acid sequences described herein. Populations (e.g., a plurality) of human T cells comprising any of the nucleic acid sequences described herein are also provided.

Any of the nucleic acid constructs encoding any of the polypeptides described herein can be used to make modified T cells. In some embodiments, the method comprises (a) introducing into the human T cell (i) a targeted nuclease that cleaves a target region in the TCR locus of a human T cell to create a target insertion site in the genome of the cell; and (ii) a nucleic acid construct encoding one or more of the polypeptides described herein, for example, a polypeptide comprising a TFAP4 protein and a BATF protein; a polypeptide comprising a BATF3 protein and a TFAP4 protein; a polypeptide comprising a FOXJ2 protein and a RARA protein; a polypeptide comprising a ID3 protein and a TFAP4 protein; a polypeptide comprising a BATF protein and a IRF2 protein; a polypeptide comprising a NANOG protein and a TFAP4 protein; a polypeptide comprising a MAFF protein and a SATB1 protein; a polypeptide comprising a BATF protein and a IRF1 protein; a polypeptide comprising a TFAP4 protein and a HOPX protein; a polypeptide comprising a BATF protein and a HES2 protein; a polypeptide comprising a ATF2 protein and a BATF protein; a polypeptide comprising a BATF3 protein and a SMAD4 protein; a polypeptide comprising a FOXP3 protein and a RELA protein; a polypeptide comprising a ID3 protein and a BATF3 protein; a polypeptide comprising a EOMES protein; a polypeptide comprising a HES2 protein; a polypeptide comprising a FOXJ2 protein; a polypeptide comprising (a) a human TFAP4 protein, and (b) a polypeptide comprising a human TIM3 extracellular domain or a portion thereof linked to a human 4-1BB intracellular domain (and optionally about 1-15 (e.g., 12) amino acids of the human 4-1BB extracellular domain) via a transmembrane domain; a polypeptide comprising (a) a human SOX5 protein, and (b) a truncated TIGIT protein comprising the human TIGIT extracellular domain or a portion thereof, the TIGIT transmembrane domain and about 1-10 (e.g., 7) amino acids of the human TIGIT intracellular domain; a polypeptide comprising (a) a human MYC protein, and (b) a polypeptide comprising a human TGFbR2 extracellular domain or a portion thereof linked to amino acids 41-142 of human MyD88 (and optionally 1-10 (e.g. 7) amino acids of the TGFbR2 intracellular domain) via a transmembrane domain; a polypeptide comprising a BATF3 protein and a 4-1BB protein; a polypeptide comprising (a) a TFAP4 protein, and (b) a polypeptide comprising a human LTBR extracellular domain or a portion thereof linked to the intracellular domain of human IL-4R (and optionally 1-10 (e.g. 7) amino acids of the LTBR intracellular domain) via a transmembrane domain; a polypeptide comprising (a) a ATF1 protein, and (b) a polypeptide comprising a human IL4RA extracellular domain or a portion thereof linked to the intracellular domain of human 4-1BB; a polypeptide comprising (a) a BATF protein, and (b) a polypeptide comprising a human 2B4 extracellular domain or a portion thereof linked to the intracellular domain of human 4-1BB, and optionally about 1-15 (e.g., 12) amino acids of the human 4-1BB extracellular domain; a polypeptide comprising (a) a RORC protein, and (b) a polypeptide comprising a VISTA extracellular domain or a portion thereof linked to the intracellular domain of human CD28, and optionally about 1-15 (e.g., 12) amino acids of the human CD28 extracellular domain; a polypeptide comprising a BATF protein and a CXCR 1 protein; a polypeptide comprising a MAFF protein and a IL2RA protein; a polypeptide comprising (a) a ATF1 protein, and (b) a polypeptide comprising a human TNFRSF12 extracellular domain or a portion thereof linked to the intracellular domain of human OX40 (and optionally 1-10 (e.g. 7) amino acids of the TNFRSF12 intracellular domain) via a transmembrane domain; a polypeptide comprising a human TIM3 extracellular domain or a portion thereof linked to the intracellular domain of human 4-1BB (and optionally about 1-15 (e.g., 12) amino acids of the human 4-1BB extracellular domain) via a transmembrane domain; a polypeptide comprising a human BATF2 extracellular domain or a portion thereof linked to a truncated PD-1 comprising the human PD-1 extracellular domain or a portion thereof, the PD-1 transmembrane domain and about 1-12 (e.g., 10) amino acids of the human PD-1 intracellular domain; a polypeptide comprising a human TCF7 protein and a human LATI protein; a polypeptide comprising a human 4-1BB protein; a polypeptide comprising a human STAT5 protein and a human IL-2RA protein; a polypeptide comprising (a) a human FOXJ2 protein and (b) a polypeptide comprising a LTBR extracellular domain or a portion thereof linked to the intracellular domain of human CD28 (and optionally 1-10 (e.g. 7) amino acids of the LTBR intracellular domain) via a transmembrane domain; a polypeptide comprising (a) a human FOXJ2 protein and (b) a polypeptide comprising a LTBR extracellular domain or a portion thereof linked to amino acids 41-142 of human MyD88 (and optionally 1-10 (e.g. 7) amino acids of the LTBR intracellular domain) via a transmembrane domain; a polypeptide comprising (a) a human HOPX protein and (b) a polypeptide comprising a IL4RA extracellular domain or a portion thereof linked to the intracellular domain of human 41-BB via a transmembrane domain; a polypeptide comprising (a) a ID2 protein, and (b) a polypeptide comprising a BLTA extracellular domain or a portion thereof linked to the intracellular domain of human 4-1BB, and optionally about 1-15 (e.g., 12) amino acids of the human 4-1BB extracellular domain; a polypeptide comprising a human SMAD1 protein and a MCT4 protein; or a polypeptide comprising (a) a human SMAD1 protein and (b) a LTBR extracellular domain or a portion thereof linked to amino acids the intracellular domain of human IL-4R (and optionally 1-10 (e.g. 7) amino acids of the LTBR intracellular domain) via a transmembrane domain; and (b) allowing recombination to occur, thereby inserting the nucleic acid construct in the target insertion site to generate a modified human T cell.

In some embodiments, the nucleic acid is inserted into a T cell by introducing into the T cell, (a) a targeted nuclease that cleaves a target region in exon 1 of a TCR-α subunit constant gene (TRAC) to create an insertion site in the genome of the T cell; and (b) the nucleic acid construct, wherein the nucleic acid construct is incorporated into the insertion site by homology directed repair (HDR). In some embodiments, the nucleic acid construct is inserted into a T cell by introducing into the T cell, (a) a targeted nuclease that cleaves a target region in exon 1 of a TCR-β subunit constant gene (TRBC), for example, TRBC1 or TRBC 2, to create an insertion site in the genome of the T cell; and (b) the nuclei acid construct, wherein the nucleic acid sequence is incorporated into the insertion site by homology directed repair (HDR).

In some embodiments, the nucleic acid construct is inserted by introducing a viral vector comprising the nucleic acid construct into the cell. Examples of viral vectors include, but are not limited to, adeno-associated viral (AAV) vectors, retroviral vectors or lentiviral vectors. In some embodiments, the lentiviral vector is an integrase-deficient lentiviral vector.

In some embodiments, the nucleic acid construct is inserted by introducing a non-viral vector comprising the nucleic acid construct into the cell. In non-viral delivery methods, the nucleic acid can be naked DNA, or in a non-viral plasmid or vector. For non-viral delivery methods, the DNA template can be inserted using a non-viral genome targeting protocol based on a Cas9 shuttle system and an anionic polymer. Transposon-based gene transfer can also be used. See, for example, Tipanee et al. “Preclinical and clinical advances in transposon-based gene therapy.” Biosci Rep. 37 (6): BSR20160614 (2017).

In some cases, the nucleic acid sequence is introduced into the cell as a linear DNA template. In some cases, the nucleic acid sequence is introduced into the cell as a double-stranded DNA template. In some cases, the DNA template is a single-stranded DNA template. In some cases, the single-stranded DNA template is a pure single-stranded DNA template. As used herein, by “pure single-stranded DNA” is meant single-stranded DNA that substantially lacks the other or opposite strand of DNA. By “substantially lacks” is meant that the pure single-stranded DNA lacks at least 100-fold more of one strand than another strand of DNA. In some cases, the DNA template is a double-stranded or single-stranded plasmid or mini-circle.

In some embodiments, the targeted nuclease is selected from the group consisting of an RNA-guided nuclease domain, a transcription activator-like effector nuclease (TALEN), a zinc finger nuclease (ZFN) and a megaTAL (See, for example, Merkert and Martin “Site-Specific Genome Engineering in Human Pluripotent Stem Cells,” Int. J. Mol. Sci. 18 (7): 1000 (2016)). In some embodiments, the RNA-guided nuclease is a Cas9 nuclease and the method further comprises introducing into the cell a guide RNA that specifically hybridizes to a target region in the genome of the cell, for example, a target region in exon 1 of the TRAC gene in a T cell. In other embodiments, the RNA-guided nuclease is a Cas9 nuclease and the method further comprises introducing into the cell a guide RNA that specifically hybridizes to a target region in exon 1 of the TRBC gene.

As used throughout, a guide RNA (gRNA) sequence is a sequence that interacts with a site-specific or targeted nuclease and specifically binds to or hybridizes to a target nucleic acid within the genome of a cell, such that the gRNA and the targeted nuclease co-localize to the target nucleic acid in the genome of the cell. Each gRNA includes a DNA targeting sequence or protospacer sequence of about 10 to 50 nucleotides in length that specifically binds to or hybridizes to a target DNA sequence in the genome. For example, the DNA targeting sequence is about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length. In some embodiments, the gRNA comprises a crRNA sequence and a transactivating crRNA (tracrRNA) sequence. In some embodiments, the gRNA does not comprise a tracrRNA sequence.

Generally, the DNA targeting sequence is designed to complement (e.g., perfectly complement) or substantially complement the target DNA sequence. In some cases, the DNA targeting sequence can incorporate wobble or degenerate bases to bind multiple genetic elements. In some cases, the 19 nucleotides at the 3′ or 5′ end of the binding region are perfectly complementary to the target genetic element or elements. In some cases, the binding region can be altered to increase stability. For example, non-natural nucleotides, can be incorporated to increase RNA resistance to degradation. In some cases, the binding region can be altered or designed to avoid or reduce secondary structure formation in the binding region. In some cases, the binding region can be designed to optimize G-C content. In some cases, G-C content is preferably between about 40% and about 60% (e.g., 40%, 45%, 50%, 55%, 60%).

In some embodiments, the Cas9 protein can be in an active endonuclease form, such that when bound to target nucleic acid as part of a complex with a guide RNA or part of a complex with a DNA template, a double strand break is introduced into the target nucleic acid. In the methods provided herein, a Cas9 polypeptide or a nucleic acid encoding a Cas9 polypeptide can be introduced into the cell. The double strand break can be repaired by HDR to insert the DNA template into the genome of the cell. Various Cas9 nucleases can be utilized in the methods described herein. For example, a Cas9 nuclease that requires an NGG protospacer adjacent motif (PAM) immediately 3′ of the region targeted by the guide RNA can be utilized. Such Cas9 nucleases can be targeted to, for example, a region in exon 1 of the TRAC or exon 1 of the TRAB that contains an NGG sequence. As another example, Cas9 proteins with orthogonal PAM motif requirements can be used to target sequences that do not have an adjacent NGG PAM sequence. Exemplary Cas9 proteins with orthogonal PAM sequence specificities include, but are not limited to those described in Esvelt et al., Nature Methods 10:1116-1121 (2013).

In some cases, the Cas9 protein is a nickase, such that when bound to target nucleic acid as part of a complex with a guide RNA, a single strand break or nick is introduced into the target nucleic acid. A pair of Cas9 nickases, each bound to a structurally different guide RNA, can be targeted to two proximal sites of a target genomic region and thus introduce a pair of proximal single stranded breaks into the target genomic region, for example exon 1 of a TRAC gene or exon 1 of a TRBC gene. Nickase pairs can provide enhanced specificity because off-target effects are likely to result in single nicks, which are generally repaired without lesion by base-excision repair mechanisms. Exemplary Cas9 nickases include Cas9 nucleases having a D10A or H840A mutation (See, for example, Ran et al. “Double nicking by RNA-guided CRISPR Cas9 for enhanced genome editing specificity,” Cell 154 (6): 1380-1389 (2013)).

In some embodiments, the Cas9 nuclease, the guide RNA and the nucleic acid sequence are introduced into the cell as a ribonucleoprotein complex (RNP)-nucleic acid sequence (e.g. a DNA template) complex, wherein the RNP-nucleic acid sequence complex comprises: (i) the RNP, wherein the RNP comprises the Cas9 nuclease and the guide RNA; and (ii) the nucleic acid sequence or construct.

In some embodiments, the molar ratio of RNP to DNA template can be from about 3:1 to about 100:1. For example, the molar ratio can be from about 5:1 to 10:1, from about 5:1 to about 15:1, 5:1 to about 20:1; 5:1 to about 25:1; from about 8:1 to about 12:1; from about 8:1 to about 15:1, from about 8:1 to about 20:1, or from about 8:1 to about 25:1.

In some embodiments, the DNA template in the RNP-DNA template complex is at a concentration of about 2.5 pM to about 25 pM. In some embodiments, the amount of DNA template is about 1 μg to about 10 μg.

In some cases, the RNP-DNA template complex is formed by incubating the RNP with the DNA template for less than about one minute to about thirty minutes, at a temperature of about 20° C. to about 25° C. In some embodiments, the RNP-DNA template complex and the cell are mixed prior to introducing the RNP-DNA template complex into the cell.

In some embodiments the nucleic acid sequence or the RNP-DNA template complex is introduced into the cells by electroporation. Methods, compositions, and devices for electroporating cells to introduce a RNP-DNA template complex can include those described in the examples herein. Additional or alternative methods, compositions, and devices for electroporating cells to introduce a RNP-DNA template complex can include those described in WO/2006/001614 or Kim, J. A. et al. Biosens. Bioelectron. 23, 1353-1360 (2008). Additional or alternative methods, compositions, and devices for electroporating cells to introduce a RNP-DNA template complex can include those described in U.S. Patent Appl. Pub. Nos. 2006/0094095; 2005/0064596; or 2006/0087522. Additional or alternative methods, compositions, and devices for electroporating cells to introduce a RNP-DNA template complex can include those described in Li, L. H. et al. Cancer Res. Treat. 1, 341-350 (2002); U.S. Pat. Nos. 6,773,669; 7,186,559; 7,771,984; 7,991,559; 6,485,961; 7,029,916; and U.S. Patent Appl. Pub. Nos: 2014/0017213; and 2012/0088842. Additional or alternative methods, compositions, and devices for electroporating cells to introduce a RNP-DNA template complex can include those described in Geng, T. et al., J. Control Release 144, 91-100 (2010); and Wang, J., et al. Lab. Chip 10, 2057-2061 (2010).

In some embodiments, the RNP is delivered to the cells in the presence of an anionic polymer. In some embodiments, the anionic polymer is an anionic polypeptide or an anionic polysaccharide. In some embodiments, the anionic polymer is an anionic polypeptide (e.g., a polyglutamic acid (PGA), a polyaspartic acid, or polycarboxyglutamic acid). In some embodiments, the anionic polymer is an anionic polysaccharide (e.g., hyaluronic acid (HA), heparin, heparin sulfate, or glycosaminoglycan). In some embodiments, the anionic polymer is poly(acrylic acid) (PAA), poly(methacrylic acid) (PMAA), poly(styrene sulfonate), or polyphosphate. In some embodiments, the anionic polymer has a molecular weight of at least 15 kDa (e.g., between 15 kDa and 50 kDa). In some embodiments, the anionic polymer and the Cas protein are in a molar ratio of between 10:1 and 120:1, respectively (e.g., 10:1, 20:1, 30:1, 40:1, 50:1, 60:1, 70:1, 80:1, 90:1, 100:1, 110:1, or, 120:1). In some embodiments of this aspect, the molar ratio of sgRNA:Cas protein is between 0.25:1 and 4:1 (e.g., 0.25:1, 0.5:1, 1:1, 1.2:1, 1.4:1, 1.6:1, 1.8:1, 2:1, 2.2:1, 2.4:1, 2.6:1, 2.8:1, 3:1, 3.2:1, 3.4:1, 3.6:1, 3.8:1, or 4:1).

In some embodiments, the donor template comprises a homology directed repair (HDR) template and one or more DNA-binding protein target sequences. In some embodiments, the donor template has one DNA-binding protein target sequence and one or more protospacer adjacent motif (PAM). The complex containing the DNA-binding protein (e.g., a RNA-guided nuclease), the donor gRNA, and the donor template can shuttle the donor template, without cleavage of the DNA-binding protein target sequence, to the desired intracellular location (e.g., the nucleus) such that the HDR template can integrate into the cleaved target nucleic acid. In some embodiments, the DNA-binding protein target sequence and the PAM are located at the 5′ terminus of the HDR template. Particularly, in some embodiments, the PAM can be located at the 5′ terminus of the DNA-binding protein target sequence. In other embodiments, the PAM can be located at the 3′ terminus of the DNA-binding protein target sequence. In some embodiments, the DNA-binding protein target sequence and the PAM are located at the 3′ terminus of the HDR template. Particularly, in some embodiments, the PAM can be located at the 5′ terminus of the DNA-binding protein target sequence. In other embodiments, the PAM is located at the 3′ terminus of the DNA-binding protein target sequence. In some embodiments, the donor template has two DNA-binding protein target sequences and two PAMs. Particularly, in some embodiments, a first DNA-binding protein target sequence and a first PAM are located at the 5′ terminus of the HDR template and a second DNA-binding protein target sequence and a second PAM are located at the 3′ terminus of the HDR template. In some embodiments, the first PAM is located at the 5′ terminus of the first DNA-binding protein target sequence and the second PAM is located at the 5′ of the second DNA-binding protein target sequence. In other embodiments, the first PAM is located at the 5′ terminus of the first DNA-binding protein target sequence and the second PAM is located at the 3′ of the second DNA-binding protein target sequence. In yet other embodiments, the first PAM is located at the 3′ terminus of the first DNA-binding protein target sequence and the second PAM is located at the 5′ of the second DNA-binding protein target sequence. In yet other embodiments, the first PAM is located at the 3′ terminus of the first DNA-binding protein target sequence and the second PAM is located at the 3′ of the second DNA-binding protein target sequence.

In some embodiments, the nucleic acid sequence or RNP-DNA template complex are introduced into about 1×10⁵ to about 2× 10⁶ cells T cells. For example, the nucleic acid sequence or RNP-DNA template complex can be introduced into about 1× 10⁵ cells to about 5× 10⁵ cells, about 1× 10⁵ cells to about 1× 10⁶ cells, 1× 10⁵ cells to about 1.5×10⁶ cells, 1×10⁵ cells to about 2× 10⁶ cells, about 1× 10⁶ cells to about 1.5×10⁶ cells or about 1×10⁶ cells to about 2× 10⁶ cells.

In the methods and compositions provided herein, the human T cells can be primary T cells. In some embodiments, the T cell is a regulatory T cell, an effector T cell, a memory T cell or a naïve T cell. In some embodiments, the effector T cell is a CD8 T cell. In some embodiments, the T cell is an CD4+ cell. In some embodiments, the T cell is a CD4 CD8 T cell. In some embodiments, the T cell is a CD4 CD8-T cell. In some embodiments, the T cell is a T cell that expresses a TCR receptor or differentiates into a T cell that expresses a TCR receptor.

### Methods of Treatment

Any of the methods and compositions described herein can be used to modify T cells obtained from a human subject. Any of the methods and compositions described herein can be used to modify T cells obtained from a human subject to enhance an immune response in the subject. Any of the methods and compositions described herein can be used to modify T cells obtained from a human subject to treat or prevent a disease (e.g., cancer, an infectious disease, an autoimmune disease, transplantation rejection, graft vs. host disease or other inflammatory disorder in a subject).

As used herein by subject is meant an individual. The subject can be an adult subject or a pediatric subject. Pediatric subjects include subjects ranging in age from birth to eighteen years of age.

Provided herein is a method of enhancing an immune response in a human subject comprising administering any of the modified T cells described herein, i.e., T cells that heterologously express a polypeptide described herein, for example, a polypeptide comprising a TFAP4 protein and a BATF protein; a polypeptide comprising a BATF3 protein and a TFAP4 protein; a polypeptide comprising a FOXJ2 protein and a RARA protein; a polypeptide comprising a ID3 protein and a TFAP4 protein; a polypeptide comprising a BATF protein and a IRF2 protein; a polypeptide comprising a NANOG protein and a TFAP4 protein; a polypeptide comprising a MAFF protein and a SATB1 protein; a polypeptide comprising a BATF protein and a IRF 1 protein; a polypeptide comprising a TFAP4 protein and a HOPX protein; a polypeptide comprising a BATF protein and a HES2 protein; a polypeptide comprising a ATF2 protein and a BATF protein; a polypeptide comprising a BATF3 protein and a SMAD4 protein; a polypeptide comprising a FOXP3 protein and a RELA protein; a polypeptide comprising a ID3 protein and a BATF3 protein; a polypeptide comprising a EOMES protein; a polypeptide comprising a HES2 protein; a polypeptide comprising a FOXJ2 protein;
a polypeptide comprising (a) a human TFAP4 protein, and (b) a polypeptide comprising a human TIM3 extracellular domain or a portion thereof linked to a human 4-1BB intracellular domain (and optionally about 1-15 (e.g., 12) amino acids of the human 4-1BB extracellular domain) via a transmembrane domain;
a polypeptide comprising (a) a human SOX5 protein, and (b) a truncated TIGIT protein comprising the human TIGIT extracellular domain or a portion thereof, the TIGIT transmembrane domain and about 1-10 (e.g., 7) amino acids of the human TIGIT intracellular domain;
a polypeptide comprising (a) a human MYC protein, and (b) a polypeptide comprising a human TGFbR2 extracellular domain or a portion thereof linked to amino acids 41-142 of human MyD88 (and optionally 1-10 (e.g. 7) amino acids of the TGFbR2 intracellular domain) via a transmembrane domain;
a polypeptide comprising a BATF3 protein and a 4-1BB protein;
a polypeptide comprising (a) a TFAP4 protein, and (b) a polypeptide comprising a human LTBR extracellular domain or a portion thereof linked to the intracellular domain of human IL-4R (and optionally 1-10 (e.g. 7) amino acids of the LTBR intracellular domain) via a transmembrane domain;
a polypeptide comprising (a) a ATF1 protein, and (b) a polypeptide comprising a human IL4RA extracellular domain or a portion thereof linked to the intracellular domain of human 4-1BB;
a polypeptide comprising (a) a BATF protein, and (b) a polypeptide comprising a human 2B4 extracellular domain or a portion thereof linked to the intracellular domain of human 4-1BB, and optionally about 1-15 (e.g., 12) amino acids of the human 4-1BB extracellular domain;
a polypeptide comprising (a) a RORC protein, and (b) a polypeptide comprising a VISTA extracellular domain or a portion thereof linked to the intracellular domain of human CD28, and optionally about 1-15 (e.g., 12) amino acids of the human CD28 extracellular domain;
a polypeptide comprising a BATF protein and a CXCR 1 protein;
a polypeptide comprising a MAFF protein and a IL2RA protein;
a polypeptide comprising (a) a ATF1 protein, and (b) a polypeptide comprising a human TNFRSF12 extracellular domain or a portion thereof linked to the intracellular domain of human OX40 (and optionally 1-10 (e.g. 7) amino acids of the TNFRSF12 intracellular domain) via a transmembrane domain;
a polypeptide comprising a human TIM3 extracellular domain or a portion thereof linked to the intracellular domain of human 4-1BB (and optionally about 1-15 (e.g., 12) amino acids of the human 4-1BB extracellular domain) via a transmembrane domain;
a polypeptide comprising a human BATF2 extracellular domain or a portion thereof linked to a truncated PD-1 comprising the human PD-1 extracellular domain or a portion thereof, the PD-1 transmembrane domain and about 1-12 (e.g., 10) amino acids of the human PD-1 intracellular domain;
a polypeptide comprising a human TCF7 protein and a human LATI protein;
a polypeptide comprising a human 4-1BB protein;
a polypeptide comprising a human STAT5 protein and a human IL-2RA protein;
a polypeptide comprising (a) a human FOXJ2 protein and (b) a polypeptide comprising a LTBR extracellular domain or a portion thereof linked to the intracellular domain of human CD28 (and optionally 1-10 (e.g. 7) amino acids of the LTBR intracellular domain) via a transmembrane domain;
a polypeptide comprising (a) a human FOXJ2 protein and (b) a polypeptide comprising a LTBR extracellular domain or a portion thereof linked to amino acids 41-142 of human MyD88 (and optionally 1-10 (e.g. 7) amino acids of the LTBR intracellular domain) via a transmembrane domain;
a polypeptide comprising (a) a human HOPX protein and (b) a polypeptide comprising a IL4RA extracellular domain or a portion thereof linked to the intracellular domain of human 41-BB via a transmembrane domain;
a polypeptide comprising (a) a ID2 protein, and (b) a polypeptide comprising a BLTA extracellular domain or a portion thereof linked to the intracellular domain of human 4-1BB, and optionally about 1-15 (e.g., 12) amino acids of the human 4-1BB extracellular domain;
a polypeptide comprising a human SMAD1 protein and a MCT4 protein; or a polypeptide comprising (a) a human SMAD1 protein and (b) a LTBR extracellular domain or a portion thereof linked to amino acids the intracellular domain of human IL-4R (and optionally 1-10 (e.g. 7) amino acids of the LTBR intracellular domain) via a transmembrane domain.

In some embodiments, T cells are obtained from the subject and modified using any of the methods provided herein to express an antigen-specific TCR or synthetic antigen receptor, prior to administering the modified T cells to the subject. In some embodiments, the subject has cancer and the target antigen is a cancer-specific antigen. In some embodiments, the subject has an autoimmune disorder and the antigen is an antigen associated with the autoimmune disorder. In some embodiments, the subject has an infection and target antigen is an antigen associated with the infection.

Also provided is a method for treating cancer in a human subject comprising: a) obtaining T cells from the subject; b) modifying the T cells using any of the methods provided herein to express an antigen-specific TCR or a synthetic antigen receptor that recognizes a target antigen in the subject; and c) administering the modified T cells to the subject, wherein the human subject has cancer and the target antigen is a cancer-specific antigen. As used throughout, the phrase “cancer-specific antigen” means an antigen that is unique to cancer cells or is expressed more abundantly in cancer cells than in in non-cancerous cells. In some embodiments, the cancer-specific antigen is a tumor-specific antigen.

As used herein, cancer is a disease characterized by the rapid and uncontrolled growth of aberrant cells. Cancer cells can spread locally or through the bloodstream and lymphatic system to other parts of the body. In some embodiments, the cancer is a solid tumor. In some embodiments, the cancer is a blood or hematological cancer. Exemplary cancers include, but are not limited to, breast cancer, prostate cancer, ovarian cancer, glioblastoma, cervical cancer, skin cancer, pancreatic cancer, colorectal cancer, bladder cancer, endometrial cancer, renal cancer, liver cancer, brain cancer, lymphoma, leukemia (for example, acute myeloid leukemia), myeloma, lung cancer, and the like. It is understood that the methods provided herein can also be used to target circulating cancer cells, for example, cells shed by a solid tumor into the bloodstream of a subject.

In some embodiments, tumor infiltrating lymphocytes, a heterogeneous and cancer-specific T-cell population, are obtained from a cancer subject and expanded ex vivo. The characteristics of the patient's cancer determine a set of tailored cellular modifications, and these modifications are applied to the tumor infiltrating lymphocytes using any of the methods described herein.

Also provided herein is a method of treating an autoimmune disease, an allergic disorder or transplant rejection in a human subject comprising: a) obtaining T cells from the subject; b) modifying the T cells using any of the methods provided herein to express an antigen-specific TCR or synthetic antigen receptor that recognizes a target antigen in the subject; and c) administering the modified T cells to the subject, wherein the human subject has an autoimmune disorder and the target antigen is antigen associated with the autoimmune disorder. In some embodiments, the T cells are regulatory T cells.

As used herein, an autoimmune disease is a disease where the immune system cannot differentiate between a subject's own cells and foreign cells, thus causing the immune system to mistakenly attack healthy cells in the body. Examples of autoimmune disorders include, but are not limited to, inflammatory bowel disease, multiple sclerosis, psoriasis, rheumatoid arthritis, systemic lupus erythematosus, Graves' disease, type 1 diabetes, Sjogren's syndrome, autoimmune thyroid disease, and celiac disease.

Also provided herein is a method of treating an infection in a human subject comprising: a) obtaining T cells from the subject; b) modifying the T cells using any of the methods provided herein to express an antigen-specific TCR or a synthetic antigen receptor that recognizes a target antigen in the subject; and c) administering the modified T cells to the subject, wherein the subject has an infection and the target antigen is an antigen associated with the infection in the subject.

In some embodiments, the T cell is autologous (i.e, from the same subject who will receive the modified cells) or allogenic (i.e., from a subject other than the subject who will receive the modified cells). In some examples, the T cell is an iPSC-derived T cell. See, for example, Nagano et al. Mol. Therapy Methods & Clinical Development 16:126-135 (2020). Any of the methods of treatment provided herein can further comprise expanding the population of T cells before the T cells are modified. Any of the methods of treatment provided herein can further comprise expanding the population of T cells after the T cells are modified and prior to administration to the subject.

### Screening Methods

Also provided are methods for identifying combinations of heterologous polypeptides, for example, combinations of two or transcription factors, or combinations of a transcription factor a switch receptor, that can alter one or more properties of a human T cell, for example, enhance the cell killing properties of a human T cell or a population of human T cells. Provided herein is A method for identifying a targeted insertion in the genome of a cell comprising: (a) introducing into a population of cells (i) a targeted nuclease that cleaves a target region in the genome of the cell to create a target insertion site; and (ii) a plurality of DNA templates that are different by sequence from each other, wherein each DNA template comprises: i. two or more heterologous coding or noncoding nucleic acid sequences; and ii. a unique barcode nucleotide sequence that indicates the identity of each heterologous coding or noncoding nucleic acid sequence; (b) allowing recombination to occur, thereby creating a population of modified cells; (c) (i) isolating genomic DNA; or (ii) isolating mRNA from the cells and generating cDNA from the mRNA; and (d) sequencing the genomic DNA or the cDNA to identify a DNA template inserted into the target insertion site for a cell. In some methods, the step of isolating mRNA further comprises digesting DNA from the cells. In some methods, the barcode sequences in the genomic DNA or cDNA are sequenced to identify the two or more heterologous coding or noncoding nucleic acid sequences.

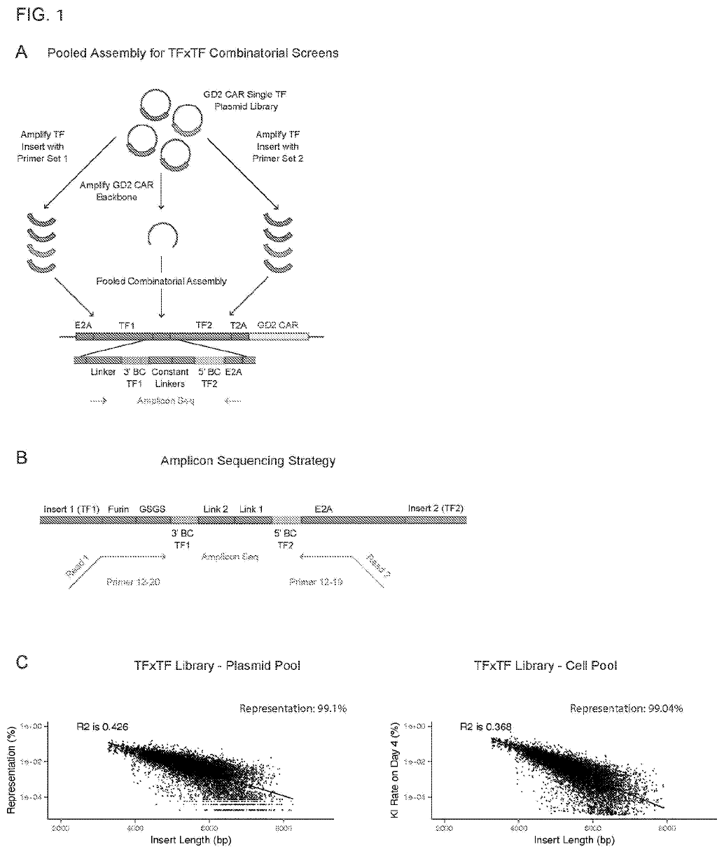

In any of the screening methods described herein, cDNA can be generated from mRNA by reverse transcribing mRNA from the cells using techniques known to those of skill in the art. Any of the screening methods described herein can further comprise amplifying the genomic DNA, or the cDNA, for example, prior to sequencing the genomic DNA or the cDNA. In some methods, the two or more heterologous coding or noncoding nucleic acid sequences are separated by linkers and each heterologous coding or noncoding nucleic acid sequence comprises a unique barcode sequence. In some methods, the barcodes that identify each heterologous coding or noncoding nucleic acid sequence are located in between the heterologous coding or noncoding nucleic acid sequences, for example, in between a first heterologous coding sequence and a second heterologous coding sequence. See, exemplary FIG. 1A, where a nucleic acid sequence comprising a nucleic acid sequence encoding transcription factor 1 (TF1) and a first barcode that identifies TF1 (3′BC TF1), is linked to a nucleic acid sequence comprising a second barcode that identifies transcription factor (5′BC TF2) and a nucleic acid sequence encoding transcription factor 2 (TF2).

In some methods, the DNA template comprises two heterologous nucleic acid sequences that encode two transcription factors. In some methods, the transcription factors are the same. In some methods, the transcription factors are different. In some methods, the DNA template comprises a heterologous nucleic acid sequence encoding a transcription factor and a heterologous nucleic acid sequence encoding a switch receptor.

In some embodiments, the method further comprises determining the relative number of cells in the population having different DNA templates inserted in the target insertion site. In some embodiments, the method further comprises applying a selective pressure to the population of modified cells. In some embodiments, the method further comprises comparing the relative number of cells in the population having different DNA templates inserted in the target insertion site before and after applying the selective pressure to the cells. In some methods, the DNA template is inserted by introducing a viral vector comprising the DNA template into the cell. IN some methods, the population is a population of mammalian cells. In some methods, the mammalian cells are human cells. In some methods, the human cells are T cells. In some methods, T cells are regulatory T cells, effector T cells or naïve T cells. In some methods, the effector T cells are CD8+ T cells or CD4+ T cells. In some methods, the effector T cells are CD8+CD4+ T cells. In some methods, the cells are primary cells. In some methods, the DNA template comprises a nucleic acid encoding two or more heterologous polypeptides.

Disclosed are materials, compositions, and components that can be used for, can be used in conjunction with, can be used in preparation for, or are products of the disclosed methods and compositions. These and other materials are disclosed herein, and it is understood that when combinations, subsets, interactions, groups, etc. of these materials are disclosed that while specific reference of each various individual and collective combinations and permutations of these compounds may not be explicitly disclosed, each is specifically contemplated and described herein. For example, if a method is disclosed and discussed and a number of modifications that can be made to one or more molecules including in the method are discussed, each and every combination and permutation of the method, and the modifications that are possible are specifically contemplated unless specifically indicated to the contrary. Likewise, any subset or combination of these is also specifically contemplated and disclosed. This concept applies to all aspects of this disclosure including, but not limited to, steps in methods using the disclosed compositions. Thus, if there are a variety of additional steps that can be performed, it is understood that each of these additional steps can be performed with any specific method steps or combination of method steps of the disclosed methods, and that each such combination or subset of combinations is specifically contemplated and should be considered disclosed.

Publications cited herein and the material for which they are cited are hereby specifically incorporated by reference in their entireties.

### Example I

### Isolation and Culture of Primary Human T Cells

T cell isolation was done as previously described (Roth et al., Cell 181:728-744 (2020)). Briefly, human T cells were isolated from leukapheresis products (Stemcell, samples collected with approved Stemcell IRB) using EasySep Human T Cell Isolation Kit (Stemcell). The use of human material was approved by the UCSF Committee on Human Research (CHR #13-11950). T cells were cultured in X-VIVO 15 media (Lonza) supplemented with 5% fetal bovine serum (FBS), 50 μM 2mercaptoethanol (ThermoFisher), and 10 mM N-Acetyl-L-Cysteine (VWR). Prior to electroporation, T cells were stimulated for 48 hours at 1e6 cells per ml of media containing 500 U/ml IL-2 (Peprotech), 5 ng/ml IL-7 (ThermoFisher), 5 ng/ml IL-15 (ThermoFisher), and CTS (Cell Therapy Systems) CD3/CD28 Dynabeads (ThermoFisher, bead:cell ratio 1:1). After nucleofection, T cells were cultured in X-VIVO 15 media containing 500 U/ml IL-2.

### Generation of Combinatorial Libraries for Pooled Knock-In

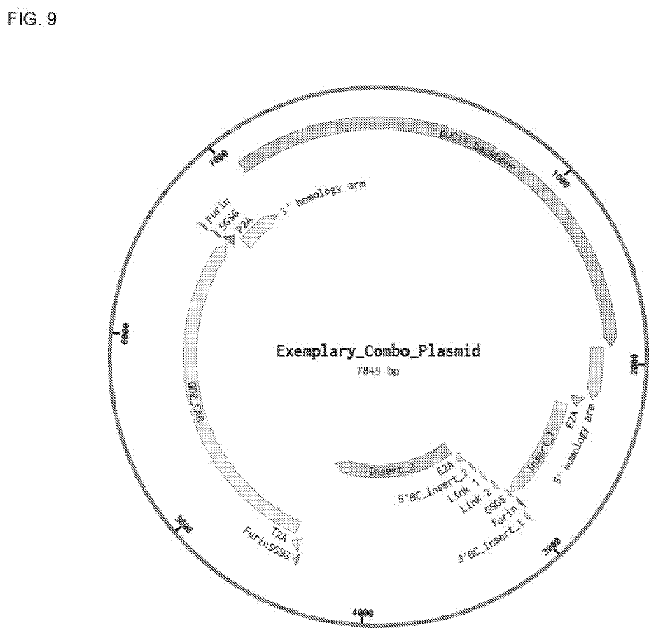

The combinatorial GD2 CAR plasmid libraries were generated by pooled Gibson assembly of the GD2 CAR pUC19 backbone as well as insert 1 and insert 2. The backbone included the published GD2 CAR sequence (Lynn et al., 2019) with CD28 co-stimulation and mutations to increase tonic signaling in the IgG1 CH2 region (Watanabe et al., 2016). The inserts were PCR-amplified out of pre-existing libraries (described in as described in International Patent Application Publication No. WO2022072932) using primers that removed the 5′ barcode of the first insert and the 3′ barcode of the second insert and added a constant linker in between the two combinatorial inserts. An exemplary plasmid map of the final constructs is shown in FIG. 9. For insert 1, the transcription factor library was used as described in International Patent Application Publication No. WO2022072932. For insert 2, the transcription factor library or the switch receptor (SR) library (as described in International Patent Application Publication No. WO2022072932) was used. The GD2 CAR backbone, the pools of insert 1 and the pools of insert 2 were assembled using NEBuilder HiFi DNA Assembly Master Mix (New England Biolabs (NEB)). The assembled product was SPRI purified using Sera-Mag SpeedBeads, transformed into Endura electrocompetent cells (Lucigen) and maxiprepped (Zymo Research) for further use. HDR templates (HDRTs) were produced as previously described (Roth et al., 2020). In brief, the combinatorial GD2 plasmid pools were used as templates for PCR amplification (KAPA HiFi HotStart ReadyMix, Roche) to generate double stranded DNA templates. Templates were SPRI purified and eluted into H2O. The plasmids for arrayed knock-in of the different combinations during the validation stage were generated in a similar way. Instead of libraries, single constructs served as templates for the PCRs to generate insert 1 and insert 2 followed by Gibson assembly together with the GD2 pUC19 backbone. GD2 CAR sequence was provided by Robbie Majzner and Crystal Mackall (Stanford University).

### Cas9 RNP Electroporation

Electroporation was done as previously described (Roth et al., 2020). To produce ribonucleoproteins (RNPs), crRNA and tracrRNA (stock 160 uM, both Dharmacon) were mixed 1:1 by volume, and annealed by incubation at 37° C. for 30 min to form an 80 μM guide RNA (gRNA) solution. Poly-L-glutamic acid (PGA, stock 125 mg/ml, Sigma) was mixed with gRNA at 0.8:1 volume ratio prior to complexing with Cas9-NLS (QB3 Macrolab) for final volume ratio gRNA:PGA:Cas9 of 1:0.8:1. These were incubated at 37° C. for 15 min to form a 14.3 μM RNP solution. RNPs and HDRTs were mixed with T cells before electroporation (3.5 ul of RNP with 1 ug=1 uL of HDRT). Bulk T cells were resuspended in electroporation buffer P3 (Lonza) at 0.75e6 cells per 20 μl (per well) and transferred to a 96-well electroporation plate together with 4.5 uL of RNP/HDRT mix per well. Pulse code EH115 was used. Cells were rescued in X-VIVO 15 without cytokines for 15 min and then cultured in X-VIVO 15 with 500 U/ml IL-2.

### Combinatorial Pooled Knock-In Screens

Primary human T cells were electroporated with combinatorial libraries on day 2 after activation. On day 4 after electroporation, knock-in rates were analyzed by flow cytometry and input population was harvested and transferred to TRI Reagent (Sigma). Cells were cultured in X-VIVO 15 supplemented with 500 U/ml IL-2 for 12 days and split every 2-3 days. Output population was harvested on day 16 after electroporation and transferred to TRI Reagent (Sigma). RNA was isolated from input and output population using Direct-zol RNA kits (Zymo Research) and reverse transcribed into cDNA using Maxima H Minus First Strand cDNA Synthesis Kit (ThermoFisher). The sequencing library was generated by two PCRs. PCR1 was performed using KAPA HiFi HotStart ReadyMix (Roche) for 18 cycles. The primers were designed to add Illumina Read1 and Read2 sequences and included a forward primer binding in the furin/GSGS region upstream of the two barcodes and a reverse primer binding downstream of the two barcodes in the constant E2A region (FIG. 9). Amplicons from PCR1 were SPRI purified. For PCR2, NEB Next Ultra II Q5 polymerase (NEB) was used for 10 cycles to append P5 and P7 Illumina sequencing adaptors. The PCR2 product was SPRI purified, normalized libraries were pooled across samples and sequenced on a NextSeq500 (Illumina). Barcode distribution was analyzed and log 2 fold change of barcode presentation in output vs input population was calculated to detect changes in abundance.

### Competition Assay

After arrayed knock-in of the different combinatorial constructs, cells were sorted on day 7 after electroporation and competition assay was set up on day 8 after electroporation. T cells were cultured at a 50/50 ratio with control T cells in X-VIVO 15 containing 50 U/ml IL-2. The cell ratio was confirmed by flow analysis of the cell mixes and exact percentage of control T cells was determined at baseline level (NGFR expression). Every two days, changes in cell ratios were analyzed by flow, cells were split 1:2 and IL-2 was added to a final concentration of 50 U/ml. Changes in cell ratio were normalized based on percentages on day 0 of the assay.

### Activation Marker and Phenotype Analysis

Activation marker expression (CD25, CD69) was analyzed by flow cytometry on day 8 after electroporation. CD62L/CD45RA expression levels were analyzed by flow cytometry 14 days after electroporation.

### Flow Cytometry and Fluorescence Activated Cell Sorting (FACS)

For flow cytometric analysis, T cells (unless sorted) were first stained for CAR expression using an Anti-Mouse IgG, F(ab′)₂ fragment specific antibody (Jackson ImmunoResearch) at a 1:100 dilution for 10 min at 4 C. Cells were washed and blocked with PBS/2% mouse serum before they were stained with the rest of the extracellular antibodies in flow buffer (PBS/2% FCS). Cells were analyzed on an Attune NxT Flow Cytometer (ThermoFisher, Waltham, Massachusetts, USA) or BD LSRFortessa (BD Biosciences, San Jose, California, USA).

For FACS, cells were stained with Anti-Mouse IgG, F(ab′)₂ fragment specific antibody (Jackson ImmunoResearch) and anti-human TCRab (Biolegend) and sorted for CAR+/TCR− cells on a BD FACSAria Fusion (BD Biosciences, San Jose, California, USA).

### RNA-Sequencing (RNA-Seq)

Edited cells were sorted for CAR+/TCR-expression on day 14. Parts of the sorted population were stored in Tri Reagent, the other part was stimulated with GD2+Nalm-6 cells at a 1:1 E:T ratio. After 24 h, the stimulated T cells were sorted again for CAR+/TCR-cells and stored in Tri Reagent. RNA was isolated using Direct-zol RNA kits (Zymo Research). RNA was prepared for sequencing as previously described (Cortez et al., 2020) by the Functional Genomics Laboratory at UC Berkeley and sequenced by the Vincent J. Coates Genomics Sequencing Laboratory at UC Berkeley. Kallisto was used to map the reads to the human reference transcriptome and genes with zero counts in more than 80% of samples were removed from analysis. DESeq2 R package was used for differential gene expression, fgsea package for gene set enrichment analysis (GSEA) with MSigDB v7.2 hallmark gene sets as reference gene lists.

### In Vitro Killing Assay

For IncuCyte assays, flat bottom 96-well plates were coated with 50 ul of 0.01% poly-L-ornithine (PLO) solution (Sigma) for 1 hr. PLO was removed and plates were dried for 30-60 min. 10,000 GFP+/GD2+ Nalm-6 per well were mixed with sorted T cells in various effector:target (E:T) ratios. The assay media consisted of X-VIVO 15 as described above, supplemented with 500 U/ml IL-2 and 1× Glucose Solution (ThermoFisher). Cell counts (GFP+) were analyzed every six hours using the IncuCyte Live Cell Analysis System (Essen BioScience).

### In Vivo Mouse Model

NOD/SCID/IL2Rg-null (NSG) mice were purchased from Jackson Laboratory. 8-12 weeks old female mice were used and mouse experiments were performed in compliance with ethical regulations per an approved IACUC protocol (UCSF). Mice were injected IV with 0.5e6 Nalm-6/Luciferase/GFP/GD2 cells on day 0. Three days later, edited human T cells were injected IV (1e6 per mouse). T cells were TCR-depleted one day before injection using EasySep Human TCR Alpha/Beta Depletion Kit (Stemcell) to avoid Graft-versus-Host disease in the mice. Knock-in rates were adjusted between groups by adding TCR-negative T cells without CAR knock-in right before injection. For imaging, 200 uL (3 mg) of D-Luciferin Potassium Salt (Gold BioTechnology) were injected IP and mice were imaged using an IVIS Spectrum In Vivo Imaging System (PerkinElmer) once/twice per week.

### Results

Analyses of single transcription factor overexpression in combination with a T cell receptor (TCR) or chimeric antigen receptor (CAR) raised the question whether combinations of two transcription factors (TFs) or one transcription factor with one switch receptor/therapeutic construct (TC) could further ameliorate T cell functionality and overcome exhaustion. A 10,404-member library of 100 transcription factors (plus two controls) combined with 100 transcription factors (plus two controls) in combination with a GD2 CAR. The GD2 CAR pUC19-based backbone as well as the transcription factor library, as described in International Patent Application Publication No. WO2022072932, were PCR amplified for insert position 1 and insert position 2 with distinct primer pairs (FIG. 1A). The PCRs of the two inserts were designed to remove 5′ barcode and constant 5′ linker of the first construct as well as the 3′ barcode and constant 3′ linker of the second construct. By pooled Gibson assembly, a fusion region was created which consisted of the 3′ barcode of the TF in 1st position, a constant linker (linker 2-linker 1 junction) and the 5′ barcode of the TF in 2nd position. Homology directed repair templates (HDRTs) were generated from the plasmid library by PCR and non-viral knock-in of the library into the TCR alpha constant chain (TRAC) locus of primary human T cells was performed. The fusion region between TF1 and TF2 was used to identify the abundance as well as the orientation (insert 1 vs insert 2) of the different combinatorial constructs by amplicon sequencing (FIG. 1B). Amplicon sequencing of the plasmid pool and the cell pool 4 days after electroporation showed a construct size-dependent representation/knock-in rate, but confirmed representation of >99% of the constructs at both steps of the protocol (FIG. 1C).

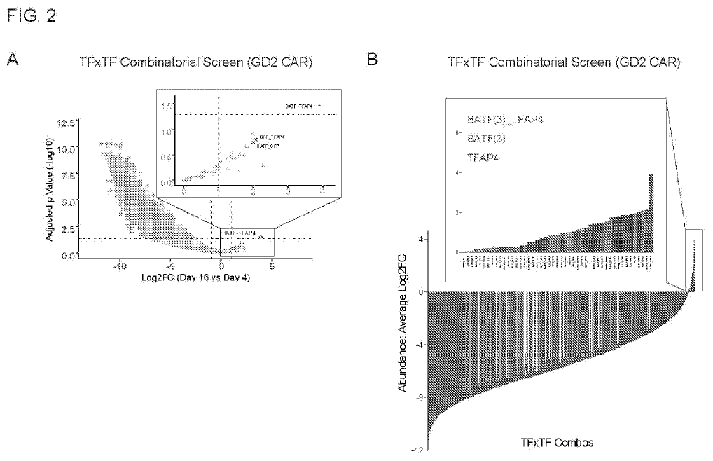

Which TF×TF combinations would enhance T cell expansion and survival in the context of chronic antigen stimulation (tonic signaling GD2 CAR) were tested. The abundance of the >10,000 different TF×TF combinations was analyzed after 16 days of culture and compared to the baseline abundance on day 4 after electroporation. Statistical analysis of two independent donors showed that, while most of the constructs dropped out over time, the combination of TFAP4 and BATF increased abundance in GD2 CAR T cells significantly (FIG. 2A). When further analyzing the constructs with the highest positive log 2 fold change after 16 days compared to 4 days, we found that most of the top performing constructs consisted of either TFAP4 or BATF/BATF3 or a combination of the two suggesting that BATF (3) and TFAP4 are the key transcription factors driving increased fitness in this model (FIG. 2B).

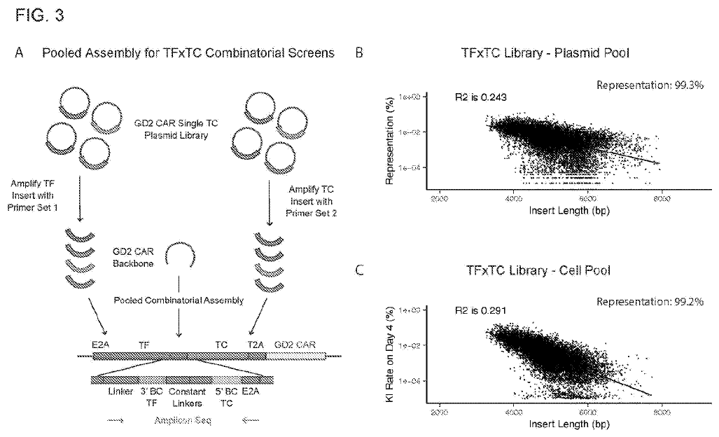

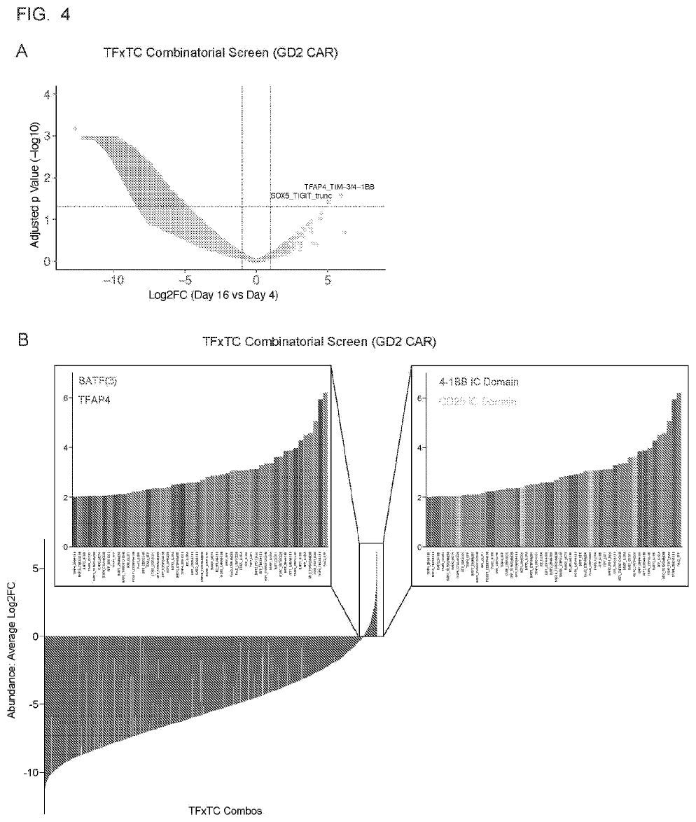

In analogy, a second combinatorial library was generated with a transcription factor in 1st position and a switch receptor/therapeutic construct (TC) (see, International Patent Application Publication No. WO2022072932) in 2nd position resulting in a 13,362-member TF×TC library (FIG. 3A). Again, amplicon sequencing showed insert size-dependent representation/knock-in rates, but confirmed that >99% of the constructs were represented in the plasmid library (FIG. 3B) as well as 4 days after electroporation (FIG. 3C). When calculating the log 2 fold change on day 16 vs day 4 after electroporation of the TF×TC library, we found significantly increased abundance of the combinatorial constructs TFAP4-TIM-3/4-1BB and SOX5-TIGITtrunc (FIG. 4A). Again, we observed that many of the constructs with high fold changes in abundance had either TFAP4 or BATF (3) as 1st position insert (FIG. 4B). Potentially because the GD2 CAR is a CD28-based costimulatory CAR, we found relatively more 4-1BB-based switch receptors compared to CD28-based switch receptors in the top performing hits.

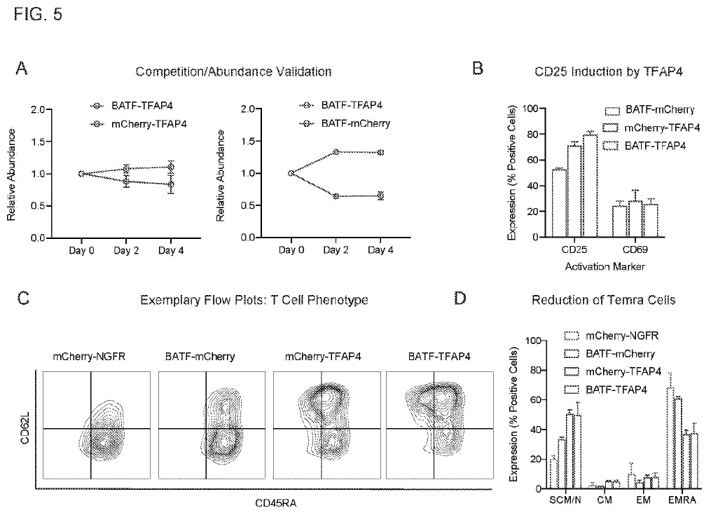

To validate our finding that a combination of BATF and TFAP4 seemed to be beneficial in the tonic signaling model, we next performed single knock-ins of the GD2 CAR and the BATF-TFAP4 combination as well as controls with overexpression of only one (mCherry-TFAP and BATF-mCherry) or no transcription factor (mCherry-NGFR). The combinatorial construct was co-cultured at a 50/50 ratio with constructs only containing one of the transcription factors and abundance was monitored over time (FIG. 5A). Addition of TFAP4 to the construct seemed to have a stronger effect than addition of BATF hinting at the fact that the majority of the proliferative or anti-apoptotic effect is driven by TFAP4. In line with observations we made with single knock-in constructs containing TFAP4, we found increased levels of CD25 expression in TFAP4-containing combinatorial constructs whereas CD69 expression stayed comparable (FIG. 5B). When analyzing the phenotype of GD2 CAR T cells 14 days after electroporation, we observed that control (mCherry-NGFR) and BATF-overexpressing (BATF-mCherry) T cells had high percentages of terminally differentiated Temra cells, whereas the phenotype of TFAP4-overexpressing cells was shifted toward stem cell-like memory stages with significantly reduced percentages of Temra cells (FIG. 5C+D).

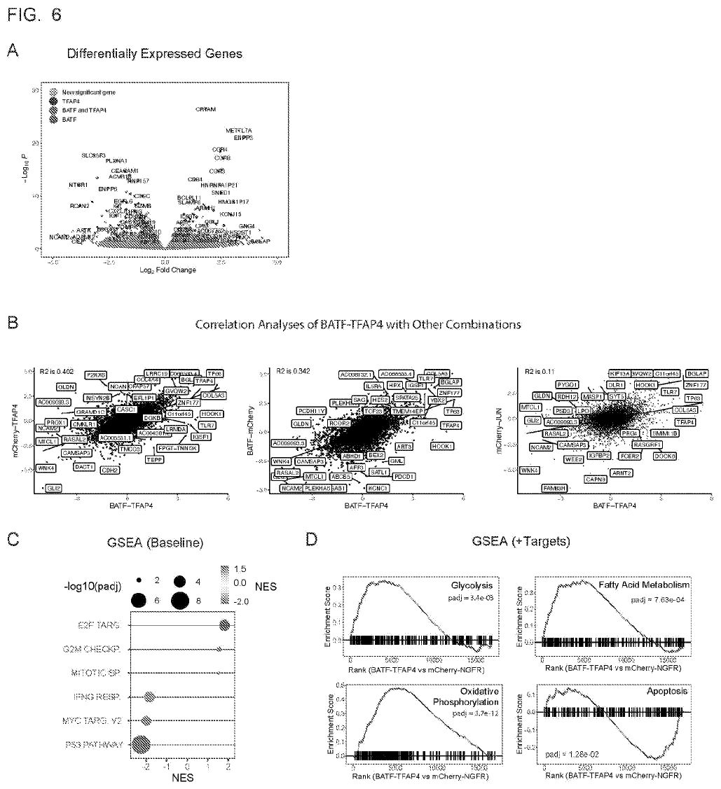

To evaluate the transcriptomic profile driven by overexpression of the BATF-TFAP4 construct, we sorted CAR+/TCR-cells on day 14 after electroporation and performed bulk RNA-seq. Differential gene expression analysis showed that the most differentially expressed gene between BATF-TFAP4 and the mCherry-NGFR condition was TFAP4 (not shown, log 2 FC 5.0, padj 6.03e-77). Moreover, we observed that while BATF-TFAP4 shared many differentially expressed genes such as CCR3, CCR4 and CCR8 with its single knock-in controls (BATF-mCherry and mCherry-TFAP4), the combination of BATF and TFAP4 in addition yielded unique differential expression of a variety of genes highlighted in yellow (FIG. 6A). Correlation analysis of the log 2 fold changes between the respective tested condition and control (mCherry-NGFR) showed that BATF-TFAP4 shares highest similarities with mCherry-TFAP4 followed by correlation with BATF-mCherry (FIG. 6B). BATF-TFAP4 had only little correlation with mCherry-JUN. Gene set enrichment analysis of BATF-TFAP4 vs mCherry-NGFR highlighted increased expression of genes involved in cell cycle regulation on day 14 after electroporation (FIG. 6C). After stimulation with target cells, BATF-TFAP4 cells had increased expression of genes involved in glycolysis, oxidative phosphorylation and fatty acid metabolism while expression of genes involved in apoptosis were decreased (FIG. 6D).

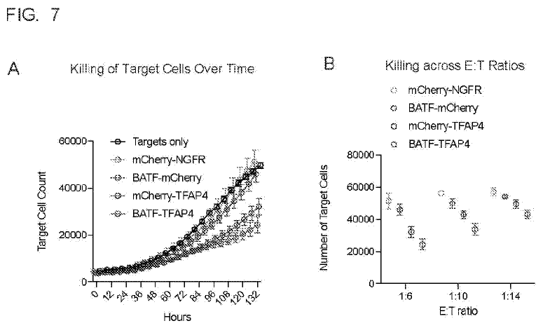

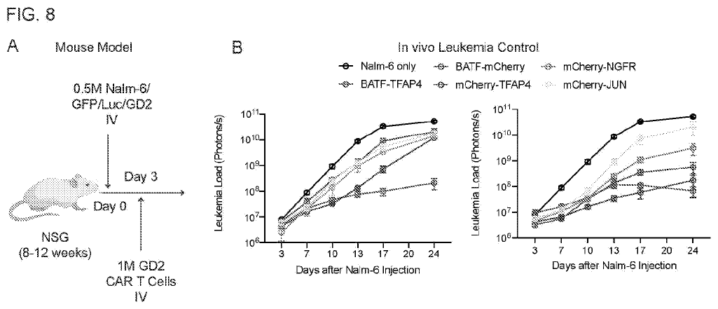

We next analyzed whether addition of TFAP4 and BATF can render GD2 CAR T cells more functional in terms of cancer cell killing. Indeed, when co-cultured with GD2 positive target cells, the combination of BATF and TFAP4 performed best across multiple E:T ratios in an in vitro killing assay (FIG. 7A+B). Lastly, we included the combinatorial cells into a NOD/SCID/IL2Rg-null (NSG) xenograft mouse model of adoptive T cell transfer (FIG. 8A) and observed that while TFAP4 overexpression alone seems to be insufficient in one of the donors, the combination of TFAP4 and BATF shows the largest potential to control leukemia growth (FIG. 8B).

### Example II

Whether specific combinations of TFs that work in concert to enhance T cell fitness in the setting of tonic CAR signaling was examined. Analyzing combinations of 100 different transcription factors requires I) knockin library sizes (˜10,000 members) that have not been tested before in this setting and II) successful knockin of very large constructs, especially when done in combination with a CAR (average construct size ˜5.5 kb plus homology arms) and thus cannot be performed readily with AAV (adeno-associated virus) HDR templates due to viral packaging size limitations.

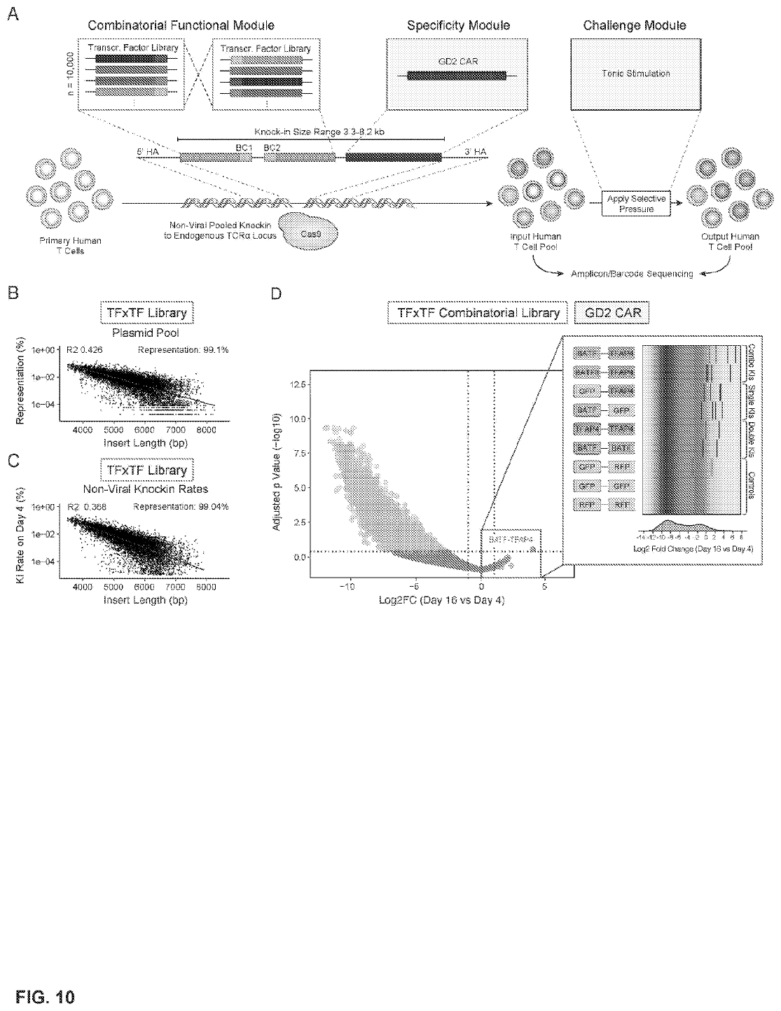

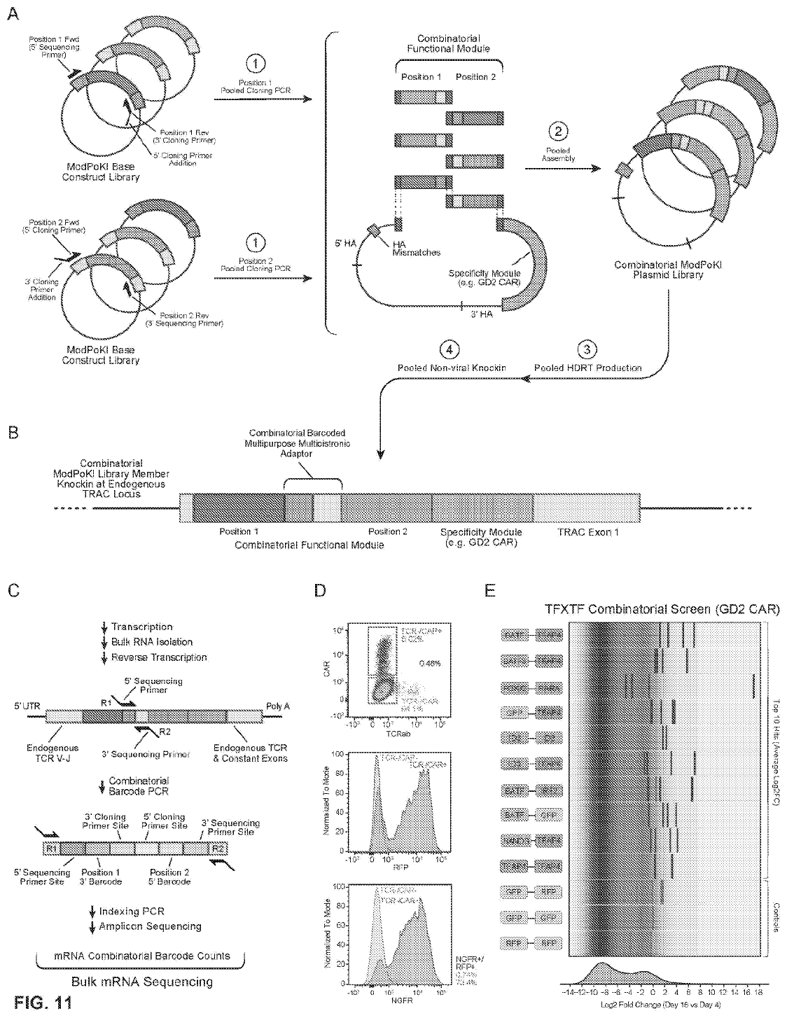

Modular pooled knockin screening (ModPoKI) molecular biology was used to overcome these challenges. The ModPoKI screening platform for large-scale combinatorial pooled knockin screens (FIGS. 10A and 11A) was adapted. A ˜10,000-member library (100 transcription factors plus two controls, combined with 100 transcription factors plus two controls) was created and cloned in constructs with the GD2 CAR. Briefly, to accomplish this, we first PCR-amplified the GD2 CAR pUC19-based backbone (specificity module) including the homology arms to target the human TRAC locus. Next, we created the two transcription factor inserts by PCR amplification off of the existing TF library using distinct primer pairs for position 1 vs position 2 within the functional module (FIG. 11B). The PCRs of the two library inserts were designed to remove the 5′ barcode and constant 5′ linker of the first construct as well as the 3′ barcode and constant 3′ linker of the second construct. By pooled Gibson assembly, a DNA site was created which consisted of the 3′ barcode of the TF in the 1^{st} position, a constant linker (linker 2-linker 1 junction) and the 5′ barcode of the TF in the 2^{nd} position, creating a unique combinatorial barcode for each TF×TF combination. HDR templates were generated from the plasmid library by PCR and non-viral knockin of the library into the TRAC locus of primary human T cells was performed. Notably, the expected knockin templates spanned a large size range from ˜3.3 to ˜8.2 kb (without homology arms). The fusion region between TF1 and TF2 served as a barcode combination to identify the abundance as well as the orientation (TF1 vs TF2) of the different combinatorial constructs by amplicon sequencing (FIG. 11C). Amplicon sequencing of the plasmid pool and the ModPoKI T cell pool 4 days after electroporation confirmed representation of >99% of the constructs at both steps of the protocol, despite the expected construct size-dependent effects on library representation/knockin rate (FIG. 10B-C). We were thus able to generate pooled libraries with thousands of different members and successfully achieved diverse knockins including constructs as large as ˜7.6 kb based on barcode sequencing.

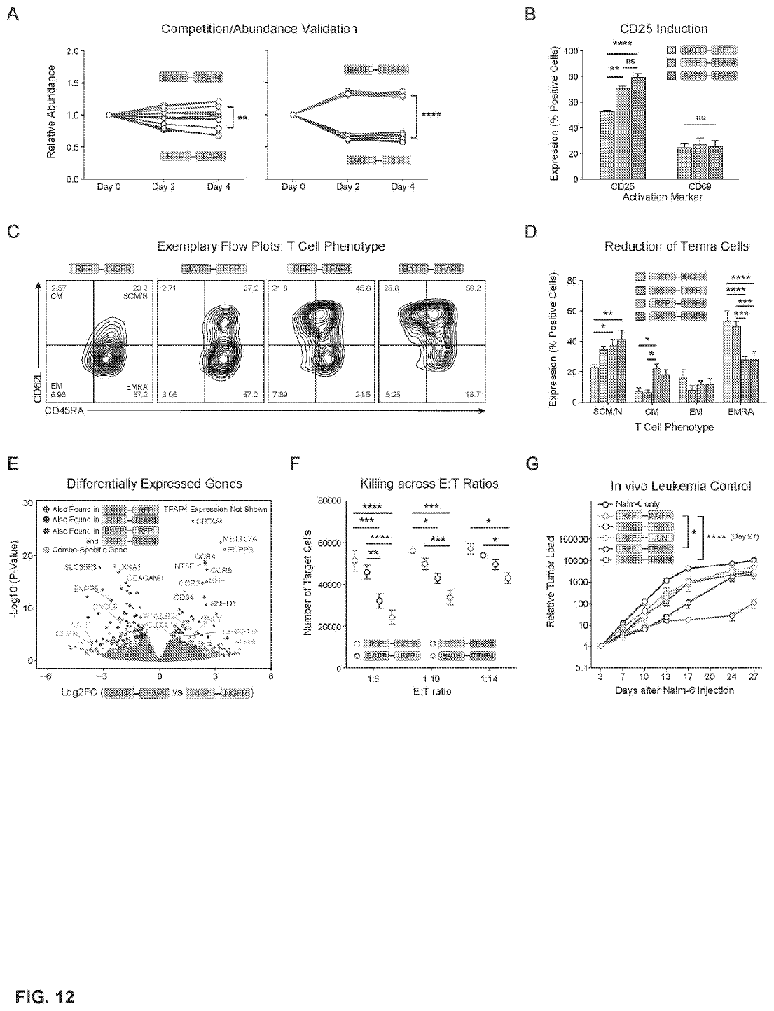

We used combinatorial ModPoKI to test which TF×TF constructs would enhance T cell fitness in the context of tonic CAR signaling. ModPoKI cells expanded in culture due to GD2 CAR tonic signaling. We compared the abundance of each TF×TF combination construct after 16 days in culture to its baseline abundance in the ModPoKI T cell population on day 4 after electroporation. Most TF×TF combinations were depleted from the pool over time, consistent with our previous evidence that major transcriptional changes can be detrimental to fitness (FIG. 12A-B). Analysis of the constructs that increased the most in relative abundance (log 2 fold change) highlighted that several of the top performing constructs included a combination of TFAP4 and BATF (or BATF3) suggesting that TFAP4 and BATF (3) are key transcription factors that can coordinately drive increased T cell fitness during repetitive simulations (FIGS. 10D and 11E). Analysis of screens performed in cells from two human donors identified the TFAP4 and BATF combination construct as the most significantly increased in abundance across the two different barcode combinations/knockin directions (TFAP4-BATF and BATF-TFAP4) (FIG. 10D). In summary, these data show that large-scale combinatorial knockin screens of ˜10,000 different constructs with an average knockin size of ˜5.5 kb is feasible using the ModPoKI screening platform and can help create an atlas of combinatorial KI constructs with potential to enhance therapeutic T cells.

### Combined TFAP4 and BATF KI Induces Favorable States in Therapeutic T Cells

To validate and characterize the benefit of KI constructs combining BATF and TFAP4, we next generated specific knockin constructs with the GD2 CAR and: 1) BATF+TFAP4 combination, 2) single TF+control (RFP-TFAP and BATF-RFP), or 3) control+control (RFP-tNGFR). We performed competitive fitness assays to assess if the combination KI outperformed the individual TFKIs. KI cells with the BATF+TFAP4 combinatorial construct were co-cultured at a ˜50/50 ratio with KI cells with a construct containing only a single TF (along with a control gene), and relative abundance was monitored over time (FIG. 12A). KI cells with the BATF+TFAP4 combinatorial construct outcompeted both the TFAP4+control KI cells and the BATF+control KI cells. The relative benefit of BATF+TFAP4 combination was stronger compared to BATF only than to TFAP4 only, hinting that the majority of fitness benefit (although not all of it) is conferred by TFAP4KI. Consistent with the effects of the single TFAP4 KI constructs, we found increased levels of CD25 expression in TFAP4-containing combinatorial constructs, whereas CD69 levels were not markedly affected (FIG. 12B). When analyzing the phenotype of GD2 CAR T cells 14 days after electroporation, we observed that control (RFP-tNGFR) and BATF KI (BATF-RFP) T cells had high percentages of terminally differentiated Temra cells (CD62L−/CD45RA+), whereas the phenotypes of TFAP4 KI cells (both TFAP4+control and TFAP4+BATF KIs) were shifted toward stem cell-like and central memory states with significantly reduced percentages of Temra cells (FIG. 12C-D).

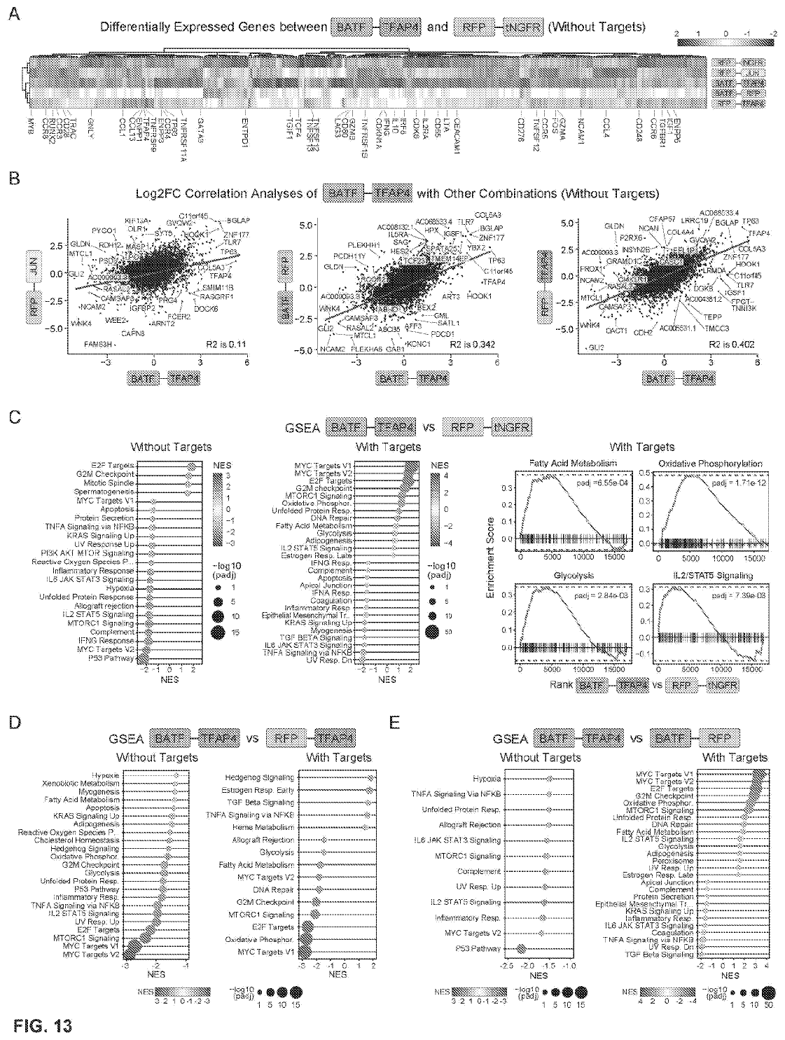

We next evaluated the transcriptional effects of the BATF-TFAP4 combination construct compared to single TF constructs and control constructs. We sorted GD2 CAR+/TCR-cells from each KI population on day 14 after electroporation and performed RNA-seq without addition of target cells (tonic signaling only). Correlation analysis of the log 2 fold changes between the respective tested condition and control (RFP-tNGFR) showed that BATF-TFAP4 KI cells were more similar to RFP-TFAP4 KI cells than to the BATF-RFP KI cells (FIGS. 13A-B). The BATF-TFAP4 KI cells had even less correlation with RFP-JUN KI cells in this setting, suggesting that the transcriptional program promoted by these TFs is divergent from the previously reported program promoted by JUN overexpression. Some genes including CCR3, CCR4 and CCR8 were induced by BATF+TFAP4 KI, BATF-RFP KI and RFP-TFAP4 KI (relative to control KI cells). However, the combined KI of BATF and TFAP4 also promoted differential expression of a variety of genes highlighted in yellow that were not differentially affected by either BATF or TFAP4 KI alone (FIG. 12E) such as TP63 (Tumor Protein 63), GNLY (Granulysin), TNFRSF11A (Tumor necrosis factor receptor superfamily member 11A) and CLECL1 (encoding for the T cell costimulatory protein C-type lectin-like domain family 1). Gene set enrichment analysis of BATF-TFAP4 vs RFP-tNGFR highlighted increased expression of genes involved in cell cycle control (such as E2F targets and G2M checkpoint genes) on day 14 after electroporation, whereas interestingly genes involved in the P53 pathway and IFN-g response seemed to be decreased (FIG. 13C, left panel). After stimulation with target cells, BATF-TFAP4 cells had increased expression of genes involved in glycolysis, oxidative phosphorylation, and fatty acid metabolism (FIG. 13C, middle and right panels and FIGS. 13D-E (comparison with single KIs)). Taken together, these results suggest that combinatorial knockin of BATF and TFAP4 can drive both overlapping but also distinct transcriptional changes compared to single BATF or TFAP4 KIs to promote a fitness advantage in the GD2 CAR model of tonic signaling.

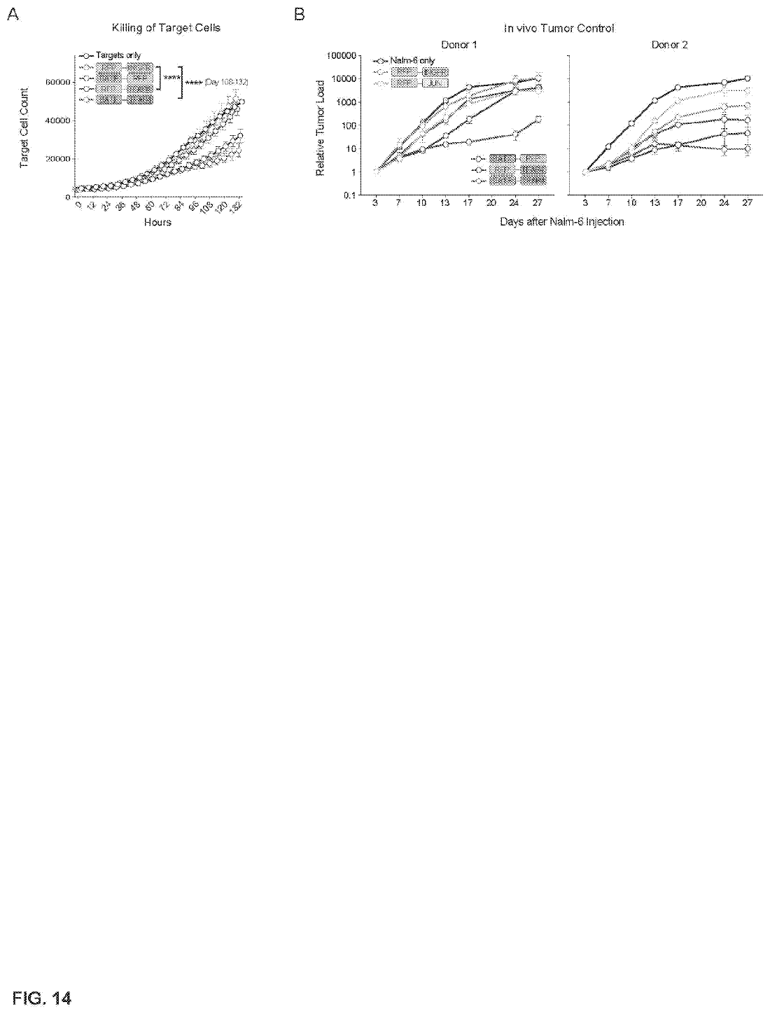

The polycistronic TFAP4 single KI construct had improved cancer killing capacity of GD2 TRAC CAR T cells in vitro and in vivo. We now wanted to assess if the TFAP4 and BATF combinatorial KI construct could further enhance the anti-cancer function of GD2 TRAC CAR T cells since this combination had conferred an added T cell fitness benefit. Indeed, when co-cultured with GD2 positive cancer target cells, BATF+TFAP4 KI GD2 TRAC CAR T cells performed best in an in vitro killing assay across multiple E:T ratios (FIGS. 12F and 14A). Finally, we tested the combinatorial KI cells in the in vivo NSG xenograft mouse model of adoptive T cell transfer (FIG. 12G and FIG. 14B) and observed that KI CAR T cells with the TFAP4+BATF combinatorial construct showed the best ability to control leukemia growth compared to CAR T cells with either single TFKI construct or with control KI constructs. In summary, combinatorial modular pooled knockin screens of thousands of different synthetic gene constructs can inform design of combinatorial genetic programs that promote enhanced persistence and function to improve adoptive T cell therapy.

### Example III

### Generation of Plasmid Libraries for Pooled Single Construct Knockin

The 231 constructs included in the pooled knockin library (see, for example, Blaeschke et al., “Modular Pooled Discovery of Synthetic Knockin Sequences to Program Durable Cell Therapies,” bioRxiv 2022.07.27.501186; doi: https://doi.org/10.1101/2022.07.27.501186) were designed using the Twist Bioscience codon optimization tool and were commercially synthesized and cloned (Twist Bioscience) into a custom pUC19 plasmid containing the NY-ESO-1 TCR sequence (except for HIF1A, JUN and TCF7 constructs that were cloned individually using gBlocks Gene Fragments (Integrated DNA Technologies)). Individual pooled plasmid libraries were created by pooling single construct plasmids into respective libraries (transcription factors, 100 members; surface receptor constructs, 129 members; controls, 2) or in one complete pool. The CD19/28z CAR plasmid pool was created in a pooled assembly fashion by amplifying constructs from the TCR plasmid pool as a DNA template. PCR amplification (Kapa Hot Start polymerase, Roche) produced a pooled library of amplicons with small overhangs homologous to a pUC19 plasmid containing the CD19/28z CAR HDR sequence. The amplicon pool was treated with DpnI restriction enzyme (New England Biolabs, NEB) to remove residual circular TCR plasmids, bead purified (Sera-Mag SpeedBeads), and eluted into H2O. We then used Gibson Assembly (NEB) to construct a plasmid pool containing all 231 library members and knockin controls, plus the new CAR sequence. The CD19/28z CAR plasmid pool was bead purified, transformed into Endura electrocompetent cells (Lucigen) and maxiprepped (Plasmid Plus Midi or Maxi Kit, Qiagen) for further use. The GD2 CAR single knockin library was generated in a similar way. While the NY-ESO-1 TCR libraries were pooled at the plasmid stage (plasmids were synthesized individually), all other plasmid libraries in this project (anti-CD19/28z CAR, anti-GD2 CAR, combinatorial library) were generated by pooled Gibson assembly of the plasmids. The CD19 CAR sequence used in this study was kindly provided by Tobias Feuchtinger, Dr. von Hauner Children's Hospital, University Hospital, LMU Munich. The GD2 CAR sequence was kindly provided by Crystal Mackall and Robbie Majzner, Stanford (Lynn et al., 2019). The assembled product was SPRI purified using Sera-Mag SpeedBeads, transformed into Endura electrocompetent cells (Lucigen) and maxiprepped (Zymo Research) for further use. HDR templates (HDRTs) were produced as previously described (Roth et al., 2020). In brief, the single knockin plasmid pools were used as templates for PCR amplification (KAPA HiFi HotStart ReadyMix, Roche) to generate double stranded DNA templates. Templates were SPRI purified and eluted into H2O. GD2 CAR sequence was provided by Robbie Majzner and Crystal Mackall (Stanford University).

### Repetitive Stimulation Screen

One day prior to the start of the repetitive stimulation screen, A375 cells were counted and transferred to 24-well plates (25,000 cells per well in 1 ml of complete RPMI media) assuming they doubled within 24 hours. One day later, edited T cell pools were counted and 10e6 cells were frozen in TRI Reagent (Sigma-Aldrich) for amplicon sequencing (input population). Media of the A375 cells was removed. 50,000 edited T cells (CAR positive, ˜1:1 effector:target ratio) were transferred to each well of the 24-well plate and co-cultured with the A375 cells in 2 ml of X-VIVO 15 containing supplements plus 50 U/ml IL-2. 24 hours later, fresh A375 cells were plated as described above. One day later, media of the new A375 plate was removed and replaced by 1 ml of fresh X-VIVO 15 plus 1 ml of the T cell suspension from the first plate including 50 U/ml IL-2 calculated on the total volume per well. The rest of the T cells were counted and 10^{e}6 cells were transferred to TRI Reagent (Sigma-Aldrich) for amplicon sequencing. The procedure was repeated every other day for a total number of five stimulations with target cells. Multiple wells of the 24-well plates were used per screen to reach cell coverage. When working with CD19 CARs in combination with A375 cells, CD19 overexpressing A375 cells (SFFV promoter knocked in upstream of endogenous CD19) were used.

### Single Stimulation Screen

One day prior to set-up of the screen, 2.5^{e}6 A375s were plated per T75 flask in complete RPMI media (RPMI plus NEAA, Glutamine, Hepes, Pen/Strep, sodium pyruvate (all Thermo Fisher Scientific) and 10% FCS (Sigma-Aldrich, St. Louis, Missouri, USA)) assuming that they doubled within 24 hours. One day later (=seven days after electroporation), edited T cell pools were counted and washed once. 10°6 T cells were transferred to TRI Reagent (Sigma-Aldrich) representing the input population for amplicon sequencing. 10e6 T cells per screening condition were transferred to one T75 flask in 20 ml of X-VIVO 15 (Lonza Bioscience) supplemented with 5% FCS, 2-Mercaptoethanol (Thermo Fisher Scientific), N-Acetyl-L-Cysteine (VWR) and 50 U/ml IL-2 (Proleukin). cRPMI was removed and flasks were filled up with 20 ml of X-VIVO 15 plus additives and 10e6 T cells. After two days, 10 ml of X-VIVO 15 were added to all conditions including supplements and 50 U/ml IL-2. Another two days later, cells were counted and 10e6 cells were transferred to TRI Reagent (Sigma-Aldrich) for RNA isolation and amplicon sequencing.

### CD25 Sorted Screen

For the sorted screen on CD25 expression, T cells were electroporated with the GD2 CAR TF library and sorted for CD25 expression (pre-gated on CAR+/TCR−) on day 8 after electroporation (top and bottom ˜20% of CD25 expression, 0.5M cells per donor per bin). RNA was isolated from CD25 high and CD25 low populations using Direct-zol RNA kits (Zymo Research) and reverse transcribed into cDNA using Maxima H Minus First Strand cDNA Synthesis Kit (ThermoFisher). The sequencing library was generated by two PCRs. PCR1 was performed using KAPA HiFi HotStart ReadyMix (Roche) for 18 cycles. The primers were designed to add Illumina Read1 and Read2 sequences upstream and downstream of the 3′ barcode region. Amplicons from PCR1 were SPRI purified. For PCR2, NEB Next Ultra II Q5 polymerase (NEB) was used for 10 cycles to append P5 and P7 Illumina sequencing adaptors. The PCR2 product was SPRI purified, normalized libraries were pooled across samples and sequenced on a NextSeq500 (Illumina). Barcode distribution was analyzed and log 2 fold change of barcode presentation in CD25 high vs low bins was calculated.

### Results

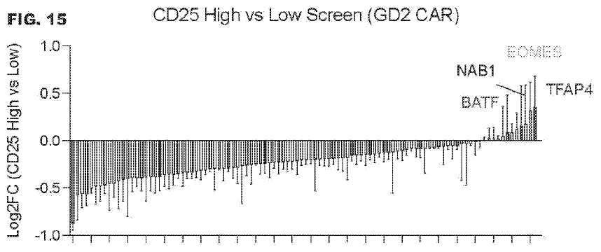

Since increased CD25 expression was observed on GD2 CAR T cells with TFAP4 knockin, a sorted screen on CD25 high vs low expression was performed on day 8 after electroporation with the GD2 CAR single insert TF library. Data shows normalized log 2FC of insert representation in CD25 high vs low bins in 3 individual donors (mean+SEM). The screen revealed a trend toward increased CD25 expression on T cells with TFAP4 or BATF knockin (FIG. 15).

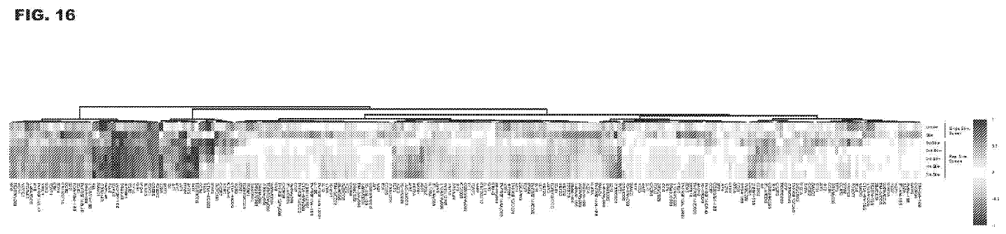

To complement previous datasets, single stimulation screens (A375/CD19 addition vs unstim condition) and repetitive stimulation screens (5× addition of A375/CD19) were performed in a complementary model using a CD19/28z CAR in combination with the full TF and SR single insert library. Normalized Log 2FC of stimulated/unstimulated/repetitively stimulated conditions over input are shown to evaluate changes in abundance. Indeed, a trend toward an increased abundance of hits (e.g., BATF2, IL2RA, TFAP4, BATF, LATI, BATF3, CXCR1, MYC, ID3, ID2, IRF1, TIGIT/ICOS, LTBR/4-1BB, DR5/ICOS, CCR4) was observed in a model complementary to the one used for the combinatorial screens (GD2 CAR) (FIG. 16).

All patents, patent applications, and other published reference materials cited in this specification are hereby incorporated herein by reference in their entirety.

TABLE 1

| Gene/ | | SEQ | | SEQ |
|---|---|---|---|---|
| Protein | | ID | | ID |
| Name | Nucleic acid Sequence | NO: | Amino acid sequence | NO: |
| TFAP4 | ATGGAGTATTTCATGGTGCCCACTCAGAAGGTG | 1. | MEYFMVPTQKVPSLQHFRKTEKEVIGGLCSLANI | 30. |
| | CCCTCTTTGCAACATTTCAGGAAAACAGAGAAA | | PLTPETQRDQERRIRREIANSNERRRMQSINAGF | |
| | GAAGTGATAGGAGGGCTCTGTAGCCTTGCCAAC | | QSLKTLIPHTDGEKLSKAAILQQTAEYIFSLEQE | |
| | ATTCCACTAACCCCCGAGACTCAGCGGGACCAG | | KTRLLQQNTQLKRFIQELSGSSPKRRRAEDKDEG | |
| | GAGCGGCGGATTCGGCGGGAGATCGCCAACAGC | | IGSPDIWEDEKAEDLRREMIELRQQLDKERSVRM | |
| | AACGAGCGGAGACGCATGCAGAGCATCAACGCG | | MLEEQVRSLEAHMYPEKLKVIAQQVQLQQQQEQV | |
| | GGATTCCAGTCCCTCAAGACCCTCATCCCCCAC | | RLLHQEKLEREQQQLRTQLLPPPAPTHHPTVIVP | |
| | ACAGACGGAGAGAAGCTCAGCAAGGCAGCCATT | | APPPPPSHHINVVTMGPSSVINSVSTSRQNLDTI | |
| | CTCCAGCAGACAGCCGAGTACATCTTCTCCCTG | | VQAIQHIEGTQEKQELEEEQRRAVIVKPVRSCPE | |
| | GAGCAGGAGAAGACCAGGCTCTTGCAGCAGAAC | | APTSDTASDSEASDSDAMDQSREEPSGDGELP | |
| | ACACAGCTCAAGCGCTTCATCCAGGAGCTGAGC | | | |
| | GGCTCGTCCCCCAAGCGACGGCGGGCAGAGGAC | | | |
| | AAGGACGAAGGCATAGGCTCCCCGGACATCTGG | | | |
| | GAGGACGAGAAGGCGGAGGACCTGCGGCGGGAG | | | |
| | ATGATTGAGCTGCGGCAGCAGCTGGACAAGGAG | | | |
| | CGCTCGGTGCGCATGATGCTGGAGGAGCAGGTG | | | |
| | CGCTCGCTGGAGGCCCACATGTACCCGGAAAAG | | | |
| | CTCAAGGTGATTGCGCAGCAGGTGCAGCTGCAG | | | |
| | CAGCAGCAGGAACAGGTGAGGCTGCTGCACCAG | | | |
| | GAGAAGCTGGAGCGGGAACAGCAGCAGCTGCGG | | | |
| | ACCCAGCTTCTGCCCCCTCCGGCCCCCACCCAC | | | |
| | CACCCCACGGTGATCGTGCCAGCACCGCCTCCT | | | |
| | CCTCCCTCCCACCACATCAATGTCGTCACCATG | | | |
| | GGCCCCTCCTCGGTCATCAACTCTGTTTCCACA | | | |
| | TCCCGGCAAAATCTGGACACCATCGTGCAGGCA | | | |
| | ATCCAGCACATCGAGGGCACCCAGGAAAAGCAG | | | |
| | GAGCTGGAGGAGGAGCAGCGGCGAGCTGTCATC | | | |
| | GTGAAGCCTGTCCGCAGCTGCCCGGAGGCCCCC | | | |
| | ACCTCTGACACCGCCTCCGACTCCGAGGCCTCA | | | |
| | GACAGTGACGCCATGGACCAGAGCCGGGAGGAG | | | |
| | CCGTCGGGGGACGGGGAGCTTCCC | | | |
| BATF | ATGCCTCACAGCTCCGACAGCAGTGACTCCAGC | 2. | MPHSSDSSDSSFSRSPPPGKQDSSDDVRRVQRRE | 31. |
| | TTCAGCCGCTCTCCTCCCCCTGGCAAACAGGAC | | KNRIAAQKSRQRQTQKADTLHLESEDLEKQNAAL | |
| | TCATCTGATGATGTGAGAAGAGTTCAGAGGAGG | | RKEIKQLTEELKYFTSVLNSHEPLCSVLAASTPS | |
| | GAGAAAAATCGTATTGCCGCCCAGAAGAGCCGA | | PPEVVYSAHAFHQPHVSSPRFQP | |
| | CAGAGGCAGACACAGAAGGCCGACACCCTGCAC | | | |
| | CTGGAGAGCGAAGACCTGGAGAAACAGAACGCG | | | |
| | GCTCTACGCAAGGAGATCAAGCAGCTCACAGAG | | | |
| | GAACTGAAGTACTTCACGTCGGTGCTGAACAGC | | | |
| | CACGAGCCCCTGTGCTCGGTGCTGGCCGCCAGC | | | |
| | ACGCCCTCGCCCCCCGAGGTGGTGTACAGCGCC | | | |
| | CACGCATTCCACCAACCTCATGTCAGCTCCCCG | | | |
| | CGCTTCCAGCCC | | | |
| BATF3 | ATGTCGCAAGGGCTCCCGGCCGCCGGCAGCGTC | 3. | MSQGLPAAGSVLQRSVAAPGNQPQPQPQQQSPED | 32. |
| | CTGCAGAGGAGCGTCGCGGCGCCCGGGAACcag | | DDRKVRRREKNRVAAQRSRKKQTQKADKLHEEYE | |
| | ccgcagccgcagccgcagcagcagAGCCCTGAG | | SLEQENTMLRREIGKLTEELKHLTEALKEHEKMC | |
| | GATGATGACAGGAAGGTCCGAAGGAGAGAAAAA | | PLLLCPMNFVPVPPRPDPVAGCLPR | |
| | AACCGAGTTGCTGCTCAGAGAAGTCGGAAGAAG | | | |
| | CAGACCCAGAAGGCTGACAAGCTCCATGAGGAA | | | |
| | TATGAGAGCCTGGAGCAAGAAAACACCATGCTG | | | |
| | CGGAGAGAGATCGGGAAGCTGACAGAGGAGCTG | | | |
| | AAGCACCTGACAGAGGCACTGAAGGAGCACGAG | | | |
| | GCCCGCTGAAGATGTCTGCTCTGCCCTATGAAC | | | |
| | TTTGTGCCAGTGCCTCCCCGGCCGGACCCTGTG | | | |
| | GCCGGCTGCTTGCCCCGA | | | |
| FOXJ2 | ATGGCTTCTGACCTAGAGAGTAGCCTCACCTCC | 4. | MASDLESSLTSIDWLPQLTLRATIEKLGSASQAG | 33. |
| | ATAGACTGGCTCCCCCAGCTGACCCTCCGAGCT | | PPGSSRKCSPGSPTDPNATLSKDEAAVHQDGKPR | |
| | ACCATTGAGAAGCTTGGAAGTGCCTCCCAGGCT | | YSYATLITYAINSSPAKKMTLSEIYRWICDNFPY | |
| | GGGCCTCCCGGGAGCAGCCGCAAGTGTTCACCA | | YKNAGIGWKNSIRHNLSLNKCFRKVPRPRDDPGK | |
| | GGGTCACCCACAGATCCTAATGCCACCCTGAGC | | GSYWTIDTCPDISRKRRHPPDDDLSQDSPEQEAS | |
| | AAAGACGAGGCAGCAGTGCACCAGGACGGCAAG | | KSPRGGVAGSGEASLPPEGNPQMSLQSPTSIASY | |
| | CCACGATACAGCTATGCCACTCTCATCACCTAT | | SQGTGSVDGGAVAAGASGRESAEGPPPLYNTNHD | |
| | GCCATCAACTCCTCTCCAGCCAAGAAGATGACC | | FKFSYSEINFQDLSWSFRNLYKSMLEKSSSSSQH | |
| | CTCAGCGAGATTTACCGCTGGATCTGTGATAAC | | GFSSLLGDIPPSNNYYMYQQQQPPPPQQQQQQQQ | |
| | TTCCCCTATTACAAGAATGCTGGCATTGGTTGG | | PPQPPPQQSQPQQQQAPAQGPSAVGGAPPLHTPS | |
| | AAGAATTCAATACGGCACAACCTTTCTCTCAAC | | TDGCTPPGGKQAGAEGYGPPPVMAMHPPPLQHGG | |
| | AAGTGTTTCCGGAAGGTGCCCAGACCTCGGGAT | | YHPHQHHPHSHPAQQPPPPQPQAQGQAPINNTGF | |
| | GACCCTGGGAAGGGTTCCTATTGGACAATTGAC | | AFPSDWCSNIDSLKESFKMVNRLNWSSIEQSQFS | |
| | ACCTGCCCTGACATTTCCCGAAAGAGAAGACAC | | ELMESLRQAEQKNWTLDQHHIANLCDSLNHFLTQ | |
| | CCTCCAGATGATGATCTGTCCCAAGACTCACCA | | TGHVPPQGGTHRPPAPARIADSCALTSGKQESAM | |
| | GAACAGGAGGCAAGCAAGAGCCCACGGGGAGGC | | SQVNSYGHPQAPHLYPGPSPMYPIPTQDSAGYNR | |
| | GTTGCAGGGAGTGGAGAAGCCTCACTGCCTCCT | | PAHHMVPRPSVPPPGANEEIPDDFDWDLIT | |
| | GAGGGGAATCCGCAGATGTCACTTCAGAGCCCC | | | |
| | ACATCTATAGCCAGCTACAGCCAGGGCACAGGA | | | |
| | TCTGTGGATGGTGGAGCAGTGGCAGCAGGGGCT | | | |
| | TCAGGCCGAGAAAGTGCTGAGGGTCCCCCTCCC | | | |
| | CTCTATAACACCAACCATGACTTTAAATTCTCC | | | |
| | TACTCAGAGATCAACTTTCAGGATCTAAGCTGG | | | |
| | TCCTTCCGCAACCTCTATAAGTCCATGCTGGAG | | | |
| | AAGTCCTCTTCCTCCTCTCAGCACGGCTTTTCT | | | |
| | TCTCTCCTGGGGGACATCCCACCCTCGAACAAC | | | |
| | TACTACATGTAtcagcagcagcagccaccgcca | | | |
| | cctcaacagcagcagcagcagcagcagccgcca | | | |
| | cagccacctccccagcagtcccagccacagcag | | | |
| | cagcAGGCACCTGCCCAGGGCCCCTCAGCTGTA | | | |
| | GGGGGTGCTCCTCCACTGCACACCCCAAGCACA | | | |
| | GATGGTTGTACCCCACCAGGGGGAAAGCAAGCT | | | |
| | GGGGCGGAAGGCTATGGGCCTCCCCCTGTAATG | | | |
| | GCCATGCATCCACCCCCGCTGCAGCATGGAGGC | | | |
| | TACCACCCTCATCAGCACCATCCCCACTCCCAC | | | |
| | CCTGCCCAGCAGCCACCACCTCCACAGCCACAG | | | |
| | GCACAAGGCCAGGCTCCCATCAACAACACTGGC | | | |
| | TTTGCCTTTCCTTCTGACTGGTGCTCTAATATT | | | |
| | GACTCTTTAAAGGAAAGCTTCAAGATGGTGAAT | | | |
| | CGGCTCAATTGGTCCAGCATTGAGCAGTCACAA | | | |
| | TTCTCAGAACTGATGGAGAGTCTACGACAGGCA | | | |
| | GAGCAGAAGAACTGGACCCTCGACCAGCATCAC | | | |
| | ATTGCCAATCTGTGTGACTCCCTCAACCACTTC | | | |
| | CTTACTCAGACTGGTCACGTGCCCCCTCAAGGG | | | |
| | GGTACCCACCGCCCACCAGCCCCTGCCCGTATT | | | |
| | GCTGACTCCTGTGCCCTCACCAGTGGCAAACAG | | | |
| | GAGTCAGCCATGAGCCAAGTGAACTCTTATGGG | | | |
| | CACCCACAAGCTCCCCACCTCTACCCTGGCCCA | | | |
| | TCACCAATGTACCCAATCCCCACCCAGGACTCA | | | |
| | GCAGGATACAATCGCCCAGCACACCATATGGTC | | | |
| | CCTCGGCCATCAGTGCCACCTCCTGGTGCCAAT | | | |
| | GAGGAGATCCCTGATGACTTCGACTGGGACTTG | | | |
| | ATCACT | | | |
| RARA | ATGGCCAGCAACAGCAGCTCCTGCCCGACACCT | 5. | MASNSSSCPTPGGGHLNGYPVPPYAFFFPPMLGG | 34. |
| | GGGGGCGGGCACCTCAATGGGTACCCGGTGCCT | | LSPPGALTTLQHQLPVSGYSTPSPATIETQSSSS | |
| | CCCTACGCCTTCTTCTTCCCCCCTATGCTGGGT | | EEIVPSPPSPPPLPRIYKPCFVCQDKSSGYHYGV | |
| | GGACTCTCCCCGCCAGGCGCTCTGACCACTCTC | | SACEGCKGFFRRSIQKNMVYTCHRDKNCIINKVT | |
| | CAGCACCAGCTTCCAGTTAGTGGATATAGCACA | | RNRCQYCRLQKCFEVGMSKESVRNDRNKKKKEVP | |
| | CCATCCCCAGCCACCATTGAGACCCAGAGCAGC | | KPECSESYTLTPEVGELIEKVRKAHQETFPALCQ | |
| | AGTTCTGAAGAGATAGTGCCCAGCCCTCCCTCG | | LGKYTTNNSSEQRVSLDIDLWDKFSELSTKCIIK | |
| | CCACCCCCTCTACCCCGCATCTACAAGCCTTGC | | TVEFAKQLPGFTTLTIADQITLLKAACLDILILR | |
| | TTTGTCTGTCAGGACAAGTCCTCAGGCTACCAC | | ICTRYTPEQDTMTFSDGLTLNRTQMHNAGFGPLT | |
| | TATGGGGTCAGCGCCTGTGAGGGCTGCAAGGGC | | DLVFAFANQLLPLEMDDAETGLLSAICLICGDRQ | |
| | TTCTTCCGCCGCAGCATCCAGAAGAACATGGTG | | DLEQPDRVDMLQEPLLEALKVYVRKRRPSRPHMF | |
| | TACACGTGTCACCGGGACAAGAACTGCATCATC | | PKMLMKITDLRSISAKGAERVITLKMEIPGSMPP | |
| | AACAAGGTGACCCGGAACCGCTGCCAGTACTGC | | LIQEMLENSEGLDTLSGQPGGGGRDGGGLAPPPG | |
| | CGACTGCAGAAGTGCTTTGAAGTGGGCATGTCC | | SCSPSLSPSSNRSSPATHSP | |
| | AAGGAGTCTGTGAGAAACGACCGAAACAAGAAG | | | |
| | AAGAAGGAGGTGCCCAAGCCCGAGTGCTCTGAG | | | |
| | AGCTACACGCTGACGCCGGAGGTGGGGGAGCTC | | | |
| | ATTGAGAAGGTGCGCAAAGCGCACCAGGAAACC | | | |
| | TTCCCTGCCCTCTGCCAGCTGGGCAAATACACT | | | |
| | ACGAACAACAGCTCAGAACAACGTGTCTCTCTG | | | |
| | GACATTGACCTCTGGGACAAGTTCAGTGAACTC | | | |
| | TCCACCAAGTGCATCATTAAGACTGTGGAGTTC | | | |
| | GCCAAGCAGCTGCCCGGCTTCACCACCCTCACC | | | |
| | ATCGCCGACCAGATCACCCTCCTCAAGGCTGCC | | | |
| | TGCCTGGACATCCTGATCCTGCGGATCTGCACG | | | |
| | CGGTACACGCCCGAGCAGGACACCATGACCTTC | | | |
| | TCGGACGGGCTGACCCTGAACCGGACCCAGATG | | | |
| | CACAACGCTGGCTTCGGCCCCCTCACCGACCTG | | | |
| | GTCTTTGCCTTCGCCAACCAGCTGCTGCCCCTG | | | |
| | GAGATGGATGATGCGGAGACGGGGCTGCTCAGC | | | |
| | GCCATCTGCCTCATCTGCGGAGACCGCCAGGAC | | | |
| | CTGGAGCAGCCGGACCGGGTGGACATGCTGCAG | | | |
| | GAGCCGCTGCTGGAGGCGCTAAAGGTCTACGTG | | | |
| | CGGAAGCGGAGGCCCAGCCGCCCCCACATGTTC | | | |
| | CCCAAGATGCTAATGAAGATTACTGACCTGCGA | | | |
| | AGCATCAGCGCCAAGGGGGCTGAGCGGGTGATC | | | |
| | ACGCTGAAGATGGAGATCCCGGGCTCCATGCCG | | | |
| | CCTCTCATCCAGGAAATGTTGGAGAACTCAGAG | | | |
| | GGCCTGGACACTCTGAGCGGACAGCCggggggg | | | |
| | gggggcgggacgggggTGGCCTGGCCCCCCCGC | | | |
| | CAGGCAGCTGTAGCCCCAGCCTCAGCCCCAGCT | | | |
| | CCAACAGAAGCAGCCCGGCCACCCACTCCCCG | | | |
| ID3 | ATGAAGGCGCTGAGCCCGGTGCGCGGCTGCTAC | 6. | MKALSPVRGCYEAVCCLSERSLAIARGRGKGPAA | 35. |
| | GAGGCGGTGTGCTGCCTGTCGGAACGCAGTCTG | | EEPLSLLDDMNHCYSRLRELVPGVPRGTQLSQVE | |
| | GCCATCGCCCGGGGCCGAGGGAAGGGCCCGGCA | | ILQRVIDYILDLQVVLAEPAPGPPDGPHLPIQTA | |
| | GCTGAGGAGCCGCTGAGCTTGCTGGACGACATG | | ELTPELVISNDKRSFCH | |
| | AACCACTGCTACTCCCGCCTGCGGGAACTGGTA | | | |
| | CCCGGAGTCCCGAGAGGCACTCAGCTTAGCCAG | | | |
| | GTGGAAATCCTACAGCGCGTCATCGACTACATT | | | |
| | CTCGACCTGCAGGTAGTCCTGGCCGAGCCAGCC | | | |
| | CCTGGACCCCCTGATGGCCCCCACCTTCCCATC | | | |
| | CAGACAGCCGAGCTCACTCCGGAACTTGTCATC | | | |
| | TCCAACGACAAAAGGAGCTTTTGCCAC | | | |
| IRF2 | ATGCCGGTGGAAAGGATGCGCATGCGCCCGTGG | 7. | MPVERMRMRPWLEEQINSNTIPGLKWLNKEKKIF | 36. |
| | CTGGAGGAGCAGATAAACTCCAACACGATCCCG | | QIPWMHAARHGWDVEKDAPLFRNWAIHTGKHQPG | |
| | GGGCTCAAGTGGCTTAACAAGGAAAAGAAGATT | | VDKPDPKTWKANFRCAMNSLPDIEEVKDKSIKKG | |
| | TTTCAGATCCCCTGGATGCATGCGGCTAGACAT | | NNAFRVYRMLPLSERPSKKGKKPKTEKEDKVKHI | |
| | GGGTGGGATGTGGAAAAAGATGCACCACTCTTT | | KQEPVESSLGLSNGVSDLSPEYAVLTSTIKNEVD | |
| | AGAAACTGGGCAATCCATACAGGAAAGCATCAA | | STVNIIVVGQSHLDSNIENQEIVTNPPDICQVVE | |
| | CCAGGAGTAGATAAACCTGATCCCAAAACATGG | | VTTESDEQPVSMSELYPLQISPVSSYAESETTDS | |
| | AAGGCGAATTTCAGATGCGCCATGAATTCCTTG | | VPSDEESAEGRPHWRKRNIEGKQYLSNMGTRGSY | |
| | CCTGATATTGAAGAAGTCAAGGATAAAAGCATA | | LLPGMASFVTSNKPDLQVTIKEESNPVPYNSSWP | |
| | AAGAAAGGAAATAATGCCTTCAGGGTCTACCGA | | PFQDLPLSSSMTPASSSSRPDRETRASVIKKTSD | |
| | ATGCTGCCCCTATCAGAACGGCCTTCTAAGAAA | | ITQARVKSC | |
| | GGAAAGAAACCAAAGACAGAAAAAGAAGACAAA | | | |
| | GTTAAGCACATCAAGCAAGAACCAGTTGAGTCA | | | |
| | TCTCTGGGGCTTAGTAATGGAGTAAGTGATCTT | | | |
| | TCTCCTGAGTATGCGGTCCTGACTTCAACTATA | | | |
| | AAAAATGAAGTGGATAGTACGGTGAACATCATA | | | |
| | GTTGTAGGACAGTCCCATCTGGACAGCAACATT | | | |
| | GAGAATCAAGAGATTGTCACCAATCCGCCAGAC | | | |
| | ATTTGCCAAGTTGTAGAGGTGACCACTGAGAGC | | | |
| | GACGAGCAGCCGGTCAGCATGAGCGAGCTCTAC | | | |
| | CCTCTGCAGATCTCCCCCGTGTCTTCCTATGCA | | | |
| | GAAAGCGAAACGACTGATAGTGTGCCCAGCGAT | | | |
| | GAAGAGAGTGCCGAGGGGCGGCCACACTGGCGG | | | |
| | AAGAGGAATATTGAAGGCAAACAGTACCTCAGC | | | |
| | AACATGGGGACTCGAGGCTCCTACCTGCTGCCC | | | |
| | GGCATGGCGTCCTTCGTCACTTCCAACAAACCG | | | |
| | GACCTCCAGGTCACCATCAAAGAGGAGAGCAAT | | | |
| | CCGGTGCCTTACAACAGCTCCTGGCCCCCTTTT | | | |
| | CAAGACCTCCCCCTTTCTTCCTCCATGACCCCA | | | |
| | GCATCCAGCAGCAGTCGGCCAGACCGGGAGACC | | | |
| | CGGGCCAGCGTCATCAAGAAAACATCGGATATC | | | |
| | ACCCAGGCCCGCGTCAAGAGCTGT | | | |
| NANOG | ATGAGTGTGGATCCAGCTTGTCCCCAAAGCTTG | 8. | MSVDPACPQSLPCFEASDCKESSPMPVICGPEEN | 37. |
| | CCTTGCTTTGAAGCATCCGACTGTAAAGAATCT | | YPSLQMSSAEMPHTETVSPLPSSMDLLIQDSPDS | |
| | TCACCTATGCCTGTGATTTGTGGGCCTGAAGAA | | STSPKGKQPTSAEKSVAKKEDKVPVKKQKTRTVF | |
| | AACTATCCATCCTTGCAAATGTCTTCTGCTGAG | | SSTQLCVLNDRFQRQKYLSLQQMQELSNILNLSY | |
| | ATGCCTCACACGGAGACTGTCTCTCCTCTTCCT | | KQVKTWFQNQRMKSKRWQKNNWPKNSNGVTQKAS | |
| | TCCTCCATGGATCTGCTTATTCAGGACAGCCCT | | APTYPSLYSSYHQGCLVNPTGNLPMWSNQTWNNS | |
| | GATTCTTCCACCAGTCCCAAAGGCAAACAACCC | | TWSNQTQNIQSWSNHSWNTQTWCTQSWNNQAWNS | |
| | ACTTCTGCAGAGAAGAGTGTCGCAAAAAAGGAA | | PFYNCGEESLQSCMQFQPNSPASDLEAALEAAGE | |
| | GACAAGGTCCCGGTCAAGAAACAGAAGACCAGA | | GLNVIQQTTRYFSTPQTMDLFLNYSMNMQPEDV | |
| | ACTGTGTTCTCTTCCACCCAGCTGTGTGTACTC | | | |
| | AATGATAGATTTCAGAGACAGAAATACCTCAGC | | | |
| | CTCCAGCAGATGCAAGAACTCTCCAACATCCTG | | | |
| | AACCTCAGCTACAAACAGGTGAAGACCTGGTTC | | | |
| | CAGAACCAGAGAATGAAATCTAAGAGGTGGCAG | | | |
| | AAAAACAACTGGCCGAAGAATAGCAATGGTGTG | | | |
| | ACGCAGAAGGCCTCAGCACCTACCTACCCCAGC | | | |
| | CTTTACTCTTCCTACCACCAGGGATGCCTGGTG | | | |
| | AACCCGACTGGGAACCTTCCAATGtggagcaac | | | |
| | cagacctggaacaattcaacctggagcaaccag | | | |
| | acccagaacatccagtcctggagcaaccactcc | | | |
| | tggaacactcagacctggtgcacccaatcctgg | | | |
| | aacaatcaggcctggaacaGTCCCTTCTATAAC | | | |
| | TGTGGAGAGGAATCTCTGCAGTCCTGCATGCAG | | | |
| | TTCCAGCCAAATTCTCCTGCCAGTGACTTGGAG | | | |
| | GCTGCCTTGGAAGCTGCTGGGGAAGGCCTTAAT | | | |
| | GTAATACAGCAGACCACTAGGTATTTTAGTACT | | | |
| | CCACAAACCATGGATTTATTCCTAAACTACTCC | | | |
| | ATGAACATGCAACCTGAAGACGTG | | | |
| MAFF | ATGAGCGTTGACCCATTGAGCTCTAAGGCACTG | 9. | MSVDPLSSKALKIKRELSENTPHLSDEALMGLSV | 38. |
| | AAAATTAAAAGAGAATTGAGTGAAAATACACCC | | RELNRHLRGLSAEEVTRLKQRRRTLKNRGYAASC | |
| | CATCTTTCCGATGAAGCACTTATGGGCCTCTCT | | RVKRVCQKEELQKQKSELEREVDKLARENAAMRL | |
| | GTTCGAGAACTCAATCGACACCTTCGAGGACTG | | ELDALRGKCEALQGFARSVAAARGPATLVAPASV | |
| | TCTGCGGAAGAAGTTACTCGCCTGAAACAACGC | | ITIVKSTPGSGSGPAHGPDPAHGPASCS | |
| | AGACGGACTCTGAAGAATCGCGGGTATGCTGCT | | | |
| | TCCTGTCGGGTCAAAAGAGTCTGTCAGAAAGAA | | | |
| | GAACTCCAAAAGCAAAAGTCCGAACTTGAACGG | | | |
| | GAAGTAGATAAATTGGCTAGGGAAAATGCTGCA | | | |
| | ATGAGACTCGAATTGGATGCTCTTAGAGGAAAA | | | |
| | TGTGAAGCTCTCCAAGGATTTGCACGGTCTGTT | | | |
| | GCTGCTGCACGGGGCCCTGCGACACTGGTCGCC | | | |
| | CCTGCATCCGTGATAACGATTGTTAAAAGCACA | | | |
| | CCTGGAAGCGGATCAGGACCTGCTCATGGACCA | | | |
| | GATCCTGCACATGGGCCCGCTTCATGTAGT | | | |
| SATB1 | ATGGATCATTTGAACGAGGCAACTCAGGGGAAA | 10. | MDHLNEATQGKEHSEMSNNVSDPKGPPAKIARLE | 39. |
| | GAACATTCAGAAATGTCTAACAATGTGAGTGAT | | QNGSPLGRGRLGSTGAKMQGVPLKHSGHLMKTNL | |
| | CCGAAGGGTCCACCAGCCAAGATTGCCCGCCTG | | RKGTMLPVFCVVEHYENAIEYDCKEEHAEFVLVR | |
| | GAGCAGAACGGGAGCCCGCTAGGAAGAGGAAGG | | KDMLFNQLIEMALLSLGYSHSSAAQAKGLIQVGK | |
| | CTTGGGAGTACAGGTGCAAAAATGCAGGGAGTG | | WNPVPLSYVTDAPDATVADMLQDVYHVVTLKIQL | |
| | CCTTTAAAACACTCGGGCCATCTGATGAAAACC | | HSCPKLEDLPPEQWSHTTVRNALKDLLKDMNQSS | |
| | AACCTTAGGAAAGGAACCATGCTGCCAGTTTTC | | LAKECPLSQSMISSIVNSTYYANVSAAKCQEFGR | |
| | TGTGTGGTGGAACATTATGAAAACGCCATTGAA | | WYKHFKKTKDMMVEMDSLSELSQQGANHVNFGQQ | |
| | TATGATTGCAAGGAGGAGCATGCAGAATTTGTG | | PVPGNTAEQPPSPAQLSHGSQPSVRTPLPNLHPG | |
| | CTGGTGAGAAAGGATATGCTTTTCAACCAGCTG | | LVSTPISPQLVNQQLVMAQLLNQQYAVNRLLAQQ | |
| | ATCGAAATGGCATTGCTGTCTCTAGGTTATTCA | | SLNQQYLNHPPPVSRSMNKPLEQQVSTNTEVSSE | |
| | CATAGCTCTGCTGCCCAGGCCAAAGGGCTAATC | | IYQWVRDELKRAGISQAVFARVAFNRTQGLLSEI | |
| | CAGGTTGGAAAGTGGAATCCAGTTCCACTGTCT | | LRKEEDPKTASQSLLVNLRAMQNFLQLPEAERDR | |
| | TACGTGACAGATGCCCCTGATGCTACAGTAGCA | | IYQDERERSLNAASAMGPAPLISTPPSRPPQVKT | |
| | GATATGCTTCAAGATGTGTATCATGTGGTCACA | | ATIATERNGKPENNTMNINASIYDEIQQEMKRAK | |
| | TTGAAAATTCAGTTACACAGTTGCCCCAAACTA | | VSQALFAKVAATKSQGWLCELLRWKEDPSPENRT | |
| | GAAGACTTGCCTCCCGAACAATGGTCGCACACC | | LWENLSMIRRFLSLPQPERDAIYEQESNAVHHHG | |
| | ACAGTGAGGAATGCTCTGAAGGACTTACTGAAA | | DRPPHIIHVPAEQIQSPSPTTLGKGESRGVFLPG | |
| | GATATGAATCAGAGTTCATTGGCCAAGGAGTGC | | LPTPAPWLGAAPQQQQQQQQQQQQQQQAPPPPQP | |
| | CCCCTTTCACAGAGTATGATTTCTTCCATTGTG | | QQQPQTGPRLPPRQPTVASPAESDEENRQKTRPR | |
| | AACAGTACTTACTATGCAAATGTCTCAGCAGCA | | TKISVEALGILQSFIQDVGLYPDEEAIQTLSAQL | |
| | AAATGTCAAGAATTTGGAAGGTGGTACAAACAT | | DLPKYTIIKFFQNQRYYLKHHGKLKDNSGLEVDV | |
| | TTCAAGAAGACAAAAGATATGATGGTTGAAATG | | AEYKEEELLKDLEESVQDKNTNTLFSVKLEEELS | |
| | GATAGTCTTTCTGAGCTATCCCAGCAAGGCGCC | | VEGNTDINTDLKD | |
| | AATCATGTCAATTTTGGCCAGCAACCAGTTCCA | | | |
| | GGGAACACAGCCGAGCAGCCTCCATCCCCTGCG | | | |
| | CAGCTCTCCCATGGCAGCCAGCCCTCTGTCCGG | | | |
| | ACACCTCTTCCAAACCTGCACCCTGGGCTCGTA | | | |
| | TCAACACCTATCAGTCCTCAATTGGTCAACCAG | | | |
| | CAGCTGGTGATGGCTCAGCTGCTGAACCAGCAG | | | |
| | TATGCAGTGAATAGACTTTTAGCCCAGCAGTCC | | | |
| | TTAAACCAACAATACTTGAACCACCCTCCCCCT | | | |
| | GTCAGTAGATCTATGAATAAGCCTTTGGAGCAA | | | |
| | CAGGTTTCGACCAACACAGAGGTGTCTTCCGAA | | | |
| | ATCTACCAGTGGGTACGCGATGAACTGAAACGA | | | |
| | GCAGGAATCTCCCAGGCGGTATTTGCACGTGTG | | | |
| | GCTTTTAACAGAACTCAGGGCTTGCTTTCAGAA | | | |
| | ATCCTCCGAAAGGAAGAGGACCCCAAGACTGCA | | | |
| | TCCCAGTCTTTGCTGGTAAACCTTCGGGCTATG | | | |
| | CAGAATTTCTTGCAGTTACCGGAAGCTGAAAGA | | | |
| | GACCGAATATACCAGGACGAAAGGGAAAGGAGC | | | |
| | TTGAATGCTGCCTCGGCCATGGGTCCTGCCCCC | | | |
| | CTCATCAGCACACCACCCAGCCGTCCTCCCCAG | | | |
| | GTGAAAACAGCTACTATTGCCACTGAAAGGAAT | | | |
| | GGGAAACCAGAGAACAATACCATGAACATTAAT | | | |
| | GCTTCCATTTATGATGAGATTCAGCAGGAAATG | | | |
| | AAGCGTGCTAAAGTGTCTCAAGCACTGTTTGCA | | | |
| | AAGGTTGCAGCAACCAAAAGCCAGGGATGGTTG | | | |
| | TGCGAGCTGTTACGCTGGAAAGAAGATCCTTCT | | | |
| | CCAGAAAACAGAACCCTGTGGGAGAACCTCTCC | | | |
| | ATGATCCGAAGGTTCCTCAGTCTTCCTCAGCCA | | | |
| | GAACGTGATGCCATTTATGAACAGGAGAGCAAC | | | |
| | GCGGTGCATCACCATGGCGACAGGCCGCCCCAC | | | |
| | ATTATCCATGTTCCAGCAGAGCAGATTCAGAGC | | | |
| | CCCTCTCCCACCACCCTTGGGAAAGGAGAGTCT | | | |
| | AGAGGCGTTTTCTTACCAGGCCTGCCGACCCCT | | | |
| | GCACCATGGCTCGGTGCTGCTCCTCAGcaacag | | | |
| | cagcagcaacagcaacagcagcagcagcagcag | | | |
| | caggcaccgccgcctccacagccacagcagcag | | | |
| | ccacagACAGGCCCTCGGCTCCCCCCACGGCAA | | | |
| | CCCACGGTGGCCTCTCCAGCAGAGTCAGATGAG | | | |
| | GAAAACCGACAGAAGACCCGGCCACGAACAAAA | | | |
| | ATTTCAGTGGAAGCCTTGGGAATCCTCCAGAGT | | | |
| | TTCATACAAGACGTGGGCCTGTACCCTGACGAA | | | |
| | GAGGCCATCCAGACTCTGTCTGCCCAGCTCGAC | | | |
| | CTTCCCAAGTACACCATCATCAAGTTCTTTCAG | | | |
| | AACCAGCGGTACTATCTCAAGCACCACGGCAAA | | | |
| | CTGAAGGACAATTCCGGTTTAGAGGTCGATGTG | | | |
| | GCAGAATATAAAGAAGAGGAGCTGCTGAAGGAT | | | |
| | TTGGAAGAGAGTGTCCAAGATAAAAATACTAAC | | | |
| | ACCCTTTTTTCAGTGAAACTAGAAGAAGAGCTG | | | |
| | TCAGTGGAAGGAAACACAGACATTAATACTGAT | | | |
| | TTGAAAGAC | | | |
| EOMES | ATGCAACTCGGAGAACAACTGCTCGTTAGTTCT | 11. | MQLGEQLLVSSVNLPGAHFYPLESARGGSGGSAG | 40. |
| | GTCAATCTTCCCGGGGCACATTTCTATCCCCTC | | HLPSAAPSPQKLDLDKASKKFSGSLSCEAVSGEP | |
| | GAATCAGCAAGGGGCGGGTCAGGTGGATCCGCC | | AAASAGAPAAMLSDTDAGDAFASAAAVAKPGPPD | |
| | GGTCATCTGCCTTCTGCTGCTCCTTCCCCTCAA | | GRKGSPCGEEELPSAAAAAAAAAAAAAATARYSM | |
| | AAGCTGGATCTCGATAAGGCTAGCAAGAAATTC | | DSLSSERYYLQSPGPQGSELAAPCSLFPYQAAAG | |
| | AGCGGATCCCTGTCATGTGAAGCAGTATCTGGT | | APHGPVYPAPNGARYPYGSMLPPGGFPAAVCPPG | |
| | GAACCAGCTGCGGCGTCTGCTGGTGCTCCAGCC | | RAQFGPGAGAGSGAGGSSGGGGGPGTYQYSQGAP | |
| | GCAATGTTGAGCGATACTGATGCAGGAGATGCC | | LYGPYPGAAAAGSCGGLGGLGVPGSGFRAHVYLC | |
| | TTCGCAAGTGCAGCAGCTGTCGCTAAACCAGGA | | NRPLWLKFHRHQTEMIITKQGRRMFPFLSFNING | |
| | CCACCCGATGGGAGAAAAGGGAGCCCGTGTGGC | | LNPTAHYNVFVEVVLADPNHWRFQGGKWVTCGKA | |
| | GAAGAAGAATTGCCGTCTGCTGCCGCCGCAGCG | | DNNMQGNKMYVHPESPNTGSHWMRQEISFGKLKL | |
| | GCTGCTGCTGCTGCAGCCGCCGCCGCTACCGCC | | TNNKGANNNNTQMIVLQSLHKYQPRLHIVEVTED | |
| | CGTTATTCTATGGATTCCTTGAGTAGCGAAAGG | | GVEDLNEPSKTQTFTFSETQFIAVTAYQNTDITQ | |
| | TATTATCTTCAAAGTCCTGGCCCGCAAGGTTCT | | LKIDHNPFAKGFRDNYDSSHQIVPGGRYGVQSFF | |
| | GAATTGGCCGCCCCATGTAGCCTGTTTCCTTAT | | PEPFVNTLPQARYYNGERTVPQTNGLLSPQQSEE | |
| | CAAGCCGCTGCCGGCGCTCCTCATGGTCCCGTA | | VANPPQRWLVTPVQQPGTNKLDISSYESEYTSST | |
| | TATCCCGCCCCAAATGGCGCCAGATATCCATAT | | LLPYGIKSLPLQTSHALGYYPDPTFPAMAGWGGR | |
| | GGGTCAATGCTTCCCCCTGGTGGATTTCCTGCT | | GSYQRKMAAGLPWTSRTSPTVFSEDQLSKEKVKE | |
| | GCTGTATGTCCCCCAGGACGGGCCCAATTTGGG | | EIGSSWIETPPSIKSLDSNDSGVYTSACKRRRLS | |
| | CCTGGGGCAGGGGCTGGTTCAGGGGCAGGTGGC | | PSNSSNENSPSIKCEDINAEEYSKDTSKGMGGYY | |
| | TCTTCTGGTGGCGGCGGTGGGCCAGGTACATAC | | AFYTTP | |
| | CAATATTCACAAGGCGCCCCACTGTATGGTCCA | | | |
| | TATCCGGGCGCTGCTGCCGCTGGGAGCTGTGGC | | | |
| | GGCCTCGGCGGGCTTGGCGTGCCTGGAAGCGGT | | | |
| | TTTAGGGCACATGTGTATTTGTGTAATCGACCA | | | |
| | CTTTGGCTGAAGTTTCATAGGCATCAGACGGAA | | | |
| | ATGATAATCACTAAGCAAGGGCGAAGGATGTTC | | | |
| | CCATTTCTGTCCTTTAATATTAATGGTCTGAAC | | | |
| | CCAACCGCACATTATAACGTCTTTGTGGAAGTC | | | |
| | GTCCTTGCAGATCCTAATCATTGGCGGTTTCAA | | | |
| | GGCGGAAAGTGGGTTACGTGCGGAAAGGCGGAT | | | |
| | AACAATATGCAAGGGAATAAGATGTACGTCCAT | | | |
| | CCTGAATCACCGAACACAGGGAGTCATTGGATG | | | |
| | AGGCAAGAAATAAGCTTTGGAAAGCTGAAGCTG | | | |
| | ACGAACAATAAGGGAGCCAACAATAATAATACT | | | |
| | CAAATGATCGTGCTTCAGTCACTTCATAAGTAT | | | |
| | CAGCCAAGGCTTCACATAGTAGAGGTCACGGAA | | | |
| | GACGGGGTCGAAGATCTGAACGAACCATCCAAA | | | |
| | ACACAAACCTTCACATTTTCCGAGACCCAGTTT | | | |
| | ATCGCCGTCACAGCGTATCAGAATACAGACATA | | | |
| | ACCCAGCTCAAAATAGACCACAATCCTTTCGCC | | | |
| | AAGGGATTTCGCGATAATTACGACTCCTCACAC | | | |
| | CAAATAGTGCCCGGCGGCAGGTATGGTGTGCAG | | | |
| | AGTTTCTTTCCAGAACCGTTCGTGAATACATTG | | | |
| | CCCCAGGCACGGTACTACAACGGGGAACGAACA | | | |
| | GTCCCCCAAACTAATGGTTTGCTCAGCCCACAG | | | |
| | CAATCCGAGGAAGTTGCAAATCCGCCACAAAGA | | | |
| | TGGCTCGTAACTCCCGTGCAACAGCCCGGCACG | | | |
| | AATAAGCTGGATATATCTAGCTACGAGTCCGAG | | | |
| | TACACAAGTTCCACCCTTCTTCCGTACGGGATC | | | |
| | AAGAGCCTGCCACTGCAAACCTCACACGCATTG | | | |
| | GGCTACTATCCCGATCCCACATTCCCCGCCATG | | | |
| | GCCGGCTGGGGCGGCAGAGGCTCATATCAACGC | | | |
| | AAAATGGCCGCGGGTTTGCCCTGGACAAGCCGC | | | |
| | ACCAGTCCGACAGTGTTTTCAGAGGACCAACTG | | | |
| | AGTAAAGAAAAGGTAAAGGAAGAGATCGGTTCA | | | |
| | AGTTGGATCGAAACCCCACCATCAATTAAGAGC | | | |
| | CTCGACAGTAACGACAGCGGCGTGTATACTTCC | | | |
| | GCCTGCAAAAGGAGACGTCTCAGCCCCTCTAAT | | | |
| | TCTTCCAACGAGAACTCCCCGAGTATTAAATGC | | | |
| | GAAGATATCAACGCAGAGGAATACAGCAAGGAT | | | |
| | ACATCTAAGGGGATGGGTGGCTACTACGCCTTC | | | |
| | TATACTACACCT | | | |
| IRF1 | ATGCCCATCACTCGGATGCGCATGAGACCCTGG | 12. | MPITRMRMRPWLEMQINSNQIPGLIWINKEEMIF | 41. |
| | CTAGAGATGCAGATTAATTCCAACCAAATCCCG | | QIPWKHAAKHGWDINKDACLFRSWAIHTGRYKAG | |
| | GGGCTCATCTGGATTAATAAAGAGGAGATGATC | | EKEPDPKTWKANFRCAMNSLPDIEEVKDQSRNKG | |
| | TTCCAGATCCCATGGAAGCATGCTGCCAAGCAT | | SSAVRVYRMLPPLTKNQRKERKSKSSRDAKSKAK | |
| | GGCTGGGACATCAACAAGGATGCCTGTTTGTTC | | RKSCGDSSPDTFSDGLSSSTLPDDHSSYTVPGYM | |
| | CGGAGCTGGGCCATTCACACAGGCCGATACAAA | | QDLEVEQALTPALSPCAVSSTLPDWHIPVEVVPD | |
| | GCAGGGGAAAAGGAGCCAGATCCCAAGACGTGG | | STSDLYNFQVSPMPSTSEATTDEDEEGKLPEDIM | |
| | AAGGCCAACTTTCGCTGTGCCATGAACTCCCTG | | KLLEQSEWQPTNVDGKGYLLNEPGVQPTSVYGDF | |
| | CCAGATATCGAGGAGGTGAAAGACCAGAGCAGG | | SCKEEPEIDSPGGDIGLSLQRVFTDLKNMDATWL | |
| | AACAAGGGCAGCTCAGCTGTGCGAGTGTACCGG | | DSLLTPVRLPSIQAIPCAP | |
| | ATGCTTCCACCTCTCACCAAGAACCAGAGAAAA | | | |
| | GAAAGAAAGTCGAAGTCCAGCCGAGATGCTAAG | | | |
| | AGCAAGGCCAAGAGGAAGTCATGTGGGGATTCC | | | |
| | AGCCCTGATACCTTCTCTGATGGACTCAGCAGC | | | |
| | TCCACTCTGCCTGATGACCACAGCAGCTACACA | | | |
| | GTTCCAGGCTACATGCAGGACTTGGAGGTGGAG | | | |
| | CAGGCCCTGACTCCAGCACTGTCGCCATGTGCT | | | |
| | GTCAGCAGCACTCTCCCCGACTGGCACATCCCA | | | |
| | GTGGAAGTTGTGCCGGACAGCACCAGTGATCTG | | | |
| | TACAACTTCCAGGTGTCACCCATGCCCTCCACC | | | |
| | TCTGAAGCTACAACAGATGAGGATGAGGAAGGG | | | |
| | AAATTACCTGAGGACATCATGAAGCTCTTGGAG | | | |
| | CAGTCGGAGTGGCAGCCAACAAACGTGGATGGG | | | |
| | AAGGGGTACCTACTCAATGAACCTGGAGTCCAG | | | |
| | CCCACCTCTGTCTATGGAGACTTTAGCTGTAAG | | | |
| | GAGGAGCCAGAAATTGACAGCCCAGGGGGGGAT | | | |
| | ATTGGGCTGAGTCTACAGCGTGTCTTCACAGAT | | | |
| | CTGAAGAACATGGATGCCACCTGGCTGGACAGC | | | |
| | CTGCTGACCCCAGTCCGGTTGCCCTCCATCCAG | | | |
| | GCCATTCCCTGTGCACCG | | | |
| HOPX | ATGCTCATTTTCCTGGGCTGTTACAGAAGAAGA | 13. | MLIFLGCYRRRLEERAGTMSAETASGPTEDQVEI | 42. |
| | CTGGAAGAGCGCGCAGGGACCATGTCGGCGGAG | | LEYNFNKVDKHPDSTTLCLIAAEAGLSEEETQGS | |
| | ACCGCGAGCGGCCCCACAGAGGACCAGGTGGAA | | DLISRSKIWHPESSPQREGYPHDSLPCLAFDYFS | |
| | ATCCTGGAGTACAACTTCAACAAGGTCGACAAG | | LLPPQCKEMV | |
| | CACCCGGATTCCACCACGCTGTGCCTCATCGCG | | | |
| | GCCGAGGCAGGCCTTTCCGAGGAGGAGACCCAG | | | |
| | GGTAGTGATTTGATCTCCAGGAGCAAAATATGG | | | |
| | CACCCAGAAAGTAGCCCCCAAAGAGAAGGCTAC | | | |
| | CCCCATGATAGTCTGCCGTGCTTGGCTTTCGAT | | | |
| | TACTTTTCTCTACTGCCGCCCCAGTGTAAAGAA | | | |
| | ATGGTT | | | |
| HES2 | ATGGGCTTGCCGAGAAGAGCGGGAGATGCTGCC | 14. | MGLPRRAGDAAELRKSLKPLLEKRRRARINQSLS | 43. |
| | GAACTCCGGAAATCATTGAAACCCCTTCTCGAA | | QLKGLILPLLGRENSNCSKLEKADVLEMTVRFLQ | |
| | AAGCGGAGAAGAGCCAGAATTAATCAATCCCTC | | ELPASSWPTAAPLPCDSYREGYSACVARLARVLP | |
| | AGTCAACTGAAAGGCTTGATACTTCCACTCTTG | | ACRVLEPAVSARLLEHLWRRAASATLDGGRAGDS | |
| | GGTCGAGAAAATTCTAATTGTTCTAAACTCGAG | | SGPSAPAPAPASAPEPASAPVPSPPSPPCGPGLW | |
| | AAAGCTGATGTGTTGGAGATGACAGTCCGGTTT | | RPW | |
| | CTCCAAGAACTCCCCGCCTCTAGCTGGCCAACC | | | |
| | GCTGCACCTCTTCCGTGTGATAGTTATAGAGAA | | | |
| | GGATATTCAGCTTGCGTTGCACGACTTGCTAGG | | | |
| | GTCCTGCCAGCATGTAGGGTATTGGAACCAGCT | | | |
| | GTCAGTGCAAGGTTGCTCGAACATCTTTGGCGA | | | |
| | CGAGCTGCATCTGCTACTTTGGATGGCGGACGT | | | |
| | GCCGGCGACAGCTCCGGTCCTAGTGCTCCTGCT | | | |
| | CCTGCTCCTGCAAGCGCACCTGAACCGGCTTCA | | | |
| | GCACCTGTCCCAAGTCCCCCGTCACCACCTTGT | | | |
| | GGGCCCGGATTGTGGCGTCCATGG | | | |
| ID2 | ATGAAAGCCTTCAGTCCCGTGAGGTCCGTTAGG | 15. | MKAFSPVRSVRKNSLSDHSLGISRSKTPVDDPMS | 44. |
| | AAAAACAGCCTGTCGGACCACAGCCTGGGCATC | | LLYNMNDCYSKLKELVPSIPQNKKVSKMEILQHV | |
| | TCCCGGAGCAAAACCCCTGTGGACGACCCGATG | | IDYILDLQIALDSHPTIVSLHHQRPGQNQASRTP | |
| | AGCCTGCTATACAACATGAACGACTGCTACTCC | | LTTLNTDISILSLQASEFPSELMSNDSKALCG | |
| | AAGCTCAAGGAGCTGGTGCCCAGCATCCCCCAG | | | |
| | AACAAGAAGGTGAGCAAGATGGAAATCCTGCAG | | | |
| | CACGTCATCGACTACATCTTGGACCTGCAGATC | | | |
| | GCCCTGGACTCGCATCCCACTATTGTCAGCCTG | | | |
| | CATCACCAGAGACCCGGGCAGAACCAGGCGTCC | | | |
| | AGGACGCCGCTGACCACCCTCAACACGGATATC | | | |
| | AGCATCCTGTCCTTGCAGGCTTCTGAATTCCCT | | | |
| | TCTGAGTTAATGTCAAATGACAGCAAAGCACTG | | | |
| | TGTGGC | | | |
| TP73 | ATGGCCCAGTCCACCGCCACCTCCCCTGATGGG | 16. | MAQSTATSPDGGTTFEHLWSSLEPDSTYFDLPQS | 45. |
| | GGCACCACGTTTGAGCACCTCTGGAGCTCTCTG | | SRGNNEVVGGTDSSMDVFHLEGMTTSVMAQFNLL | |
| | GAACCAGACAGCACCTACTTCGACCTTCCCCAG | | SSTMDQMSSRAASASPYTPEHAASVPTHSPYAQP | |
| | TCAAGCCGGGGGAATAATGAGGTGGTGGGCGGA | | SSTFDTMSPAPVIPSNTDYPGPHHFEVTFQQSST | |
| | ACGGATTCCAGCATGGACGTCTTCCACCTGGAG | | AKSATWTYSPLLKKLYCQIAKTCPIQIKVSTPPP | |
| | GGCATGACTACATCTGTCATGGCCCAGTTCAAT | | PGTAIRAMPVYKKAEHVIDVVKRCPNHELGRDFN | |
| | CTGCTGAGCAGCACCATGGACCAGATGAGCAGC | | EGQSAPASHLIRVEGNNLSQYVDDPVTGRQSVVV | |
| | CGCGCGGCCTCGGCCAGCCCCTACACCCCAGAG | | PYEPPQVGTEFTTILYNFMCNSSCVGGMNRRPIL | |
| | CACGCCGCCAGCGTGCCCACCCACTCGCCCTAC | | IIITLEMRDGQVLGRRSFEGRICACPGRDRKADE | |
| | GCACAACCCAGCTCCACCTTCGACACCATGTCG | | DHYREQQALNESSAKNGAASKRAFKQSPPAVPAL | |
| | CCGGCGCCTGTCATCCCCTCCAACACCGACTAC | | GAGVKKRRHGDEDTYYLQVRGRENFEILMKLKES | |
| | CCCGGACCCCACCACTTTGAGGTCACTTTCCAG | | LELMELVPQPLVDSYRQQQQLLQRPSHLQPPSYG | |
| | CAGTCCAGCACGGCCAAGTCAGCCACCTGGACG | | PVLSPMNKVHGGMNKLPSVNQLVGQPPPHSSAAT | |
| | TACTCCCCGCTCTTGAAGAAACTCTACTGCCAG | | PNLGPVGPGMLNNHGHAVPANGEMSSSHSAQSMV | |
| | ATCGCCAAGACATGCCCCATCCAGATCAAGGTG | | SGSHCTPPPPYHADPSLVSFLTGLGCPNCIEYFT | |
| | TCCACCCCGCCACCCCCAGGCACCGCCATCCGG | | SQGLQSIYHLQNLTIEDLGALKIPEQYRMTIWRG | |
| | GCCATGCCTGTTTACAAGAAAGCGGAGCACGTG | | LQDLKQGHDYSTAQQLLRSSNAATISIGGSGELQ | |
| | ACCGACGTCGTGAAACGCTGCCCCAACCACGAG | | RQRVMEAVHFRVRHTITIPNRGGPGGGPDEWADF | |
| | CTCGGGAGGGACTTCAACGAAGGACAGTCTGCT | | GFDLPDCKARKQPIKEEFTEAEIH | |
| | CCAGCCAGCCACCTCATCCGCGTGGAAGGCAAT | | | |
| | AATCTCTCGCAGTATGTGGATGACCCTGTCACC | | | |
| | GGCAGGCAGAGCGTCGTGGTGCCCTATGAGCCA | | | |
| | CCACAGGTGGGGACGGAATTCACCACCATCCTG | | | |
| | TACAACTTCATGTGTAACAGCAGCTGTGTAGGG | | | |
| | GGCATGAACCGGCGGCCCATCCTCATCATCATC | | | |
| | ACCCTGGAGATGCGGGATGGGCAGGTGCTGGGC | | | |
| | CGCCGGTCCTTTGAGGGCCGCATCTGCGCCTGT | | | |
| | CCTGGCCGCGACCGAAAAGCTGATGAGGACCAC | | | |
| | TACCGGGAGCAGCAGGCCCTGAACGAGAGCTCC | | | |
| | GCCAAGAACGGGGCCGCCAGCAAGCGTGCCTTC | | | |
| | AAGCAGAGCCCCCCTGCCGTCCCCGCCCTTGGT | | | |
| | GCCGGTGTGAAGAAGCGGCGGCATGGAGACGAG | | | |
| | GACACGTACTACCTTCAGGTGCGAGGCCGGGAG | | | |
| | AACTTTGAGATCCTGATGAAGCTGAAAGAGAGC | | | |
| | CTGGAGCTGATGGAGTTGGTGCCGCAGCCACTG | | | |
| | GTGGACTCCTATCGGCAGCAGCAGCAGCTCCTA | | | |
| | CAGAGGCCGAGTCACCTACAGCCCCCGTCCTAC | | | |
| | GGGCCGGTCCTCTCGCCCATGAACAAGGTGCAC | | | |
| | GGGGGCATGAACAAGCTGCCCTCCGTCAACCAG | | | |
| | CTGGTGGGCCAGCCTCCCCCGCACAGTTCGGCA | | | |
| | GCTACACCCAACCTGGGGCCCGTGGGCCCCGGG | | | |
| | ATGCTCAACAACCATGGCCACGCAGTGCCAGCC | | | |
| | AACGGCGAGATGAGCAGCAGCCACAGCGCCCAG | | | |
| | TCCATGGTCTCGGGGTCCCACTGCACTCCGCCA | | | |
| | CCCCCCTACCACGCCGACCCCAGCCTCGTCAGT | | | |
| | TTTTTAACAGGATTGGGGTGTCCAAACTGCATC | | | |
| | GAGTATTTCACCTCCCAAGGGTTACAGAGCATT | | | |
| | TACCACCTGCAGAACCTGACCATTGAGGACCTG | | | |
| | GGGGCCCTGAAGATCCCCGAGCAGTACCGCATG | | | |
| | ACCATCTGGCGGGGCCTGCAGGACCTGAAGCAG | | | |
| | GGCCACGACTACAGCACCGCGCAGCAGCTGCTC | | | |
| | CGCTCTAGCAACGCGGCCACCATCTCCATCGGC | | | |
| | GGCTCAGGGGAACTGCAGCGCCAGCGGGTCATG | | | |
| | GAGGCCGTGCACTTCCGCGTGCGCCACACCATC | | | |
| | ACCATCCCCAACCGCGGCGGCCCAGGCGGCGGC | | | |
| | CCTGACGAGTGGGCGGACTTCGGCTTCGACCTG | | | |
| | CCCGACTGCAAGGCCCGCAAGCAGCCCATCAAG | | | |
| | GAGGAGTTCACGGAGGCCGAGATCCAC | | | |
| SMAD3 | ATGTCGTCCATCCTGCCTTTCACTCCCCCGATC | 17. | MSSILPFTPPIVKRLLGWKKGEQNGQEEKWCEKA | 46. |
| | GTGAAGCGCCTGCTGGGCTGGAAGAAGGGCGAG | | VKSLVKKLKKTGOLDELEKAITTQNVNTKCITIP | |
| | CAGAACGGGCAGGAGGAGAAATGGTGCGAGAAG | | RSLDGRLQVSHRKGLPHVIYCRLWRWPDLHSHHE | |
| | GCGGTCAAGAGCCTGGTCAAGAAACTCAAGAAG | | LRAMELCEFAFNMKKDEVCVNPYHYQRVETPVLP | |
| | ACGGGGCAGCTGGACGAGCTGGAGAAGGCCATC | | PVLVPRHTEIPAEFPPLDDYSHSIPENTNFPAGI | |
| | ACCACGCAGAACGTCAACACCAAGTGCATCACC | | EPQSNIPETPPPGYLSEDGETSDHQMNHSMDAGS | |
| | ATCCCCAGGTCCCTGGATGGCCGGTTGCAGGTG | | PNLSPNPMSPAHNNLDLQPVTYCEPAFWCSISYY | |
| | TCCCATCGGAAGGGGCTCCCTCATGTCATCTAC | | ELNQRVGETFHASQPSMTVDGFTDPSNSERFCLG | |
| | TGCCGCCTGTGGCGATGGCCAGACCTGCACAGC | | LLSNVNRNAAVELTRRHIGRGVRLYYIGGEVFAE | |
| | CACCACGAGCTACGGGCCATGGAGCTGTGTGAG | | CLSDSAIFVQSPNCNQRYGWHPATVCKIPPGCNL | |
| | TTCGCCTTCAATATGAAGAAGGACGAGGTCTGC | | KIFNNQEFAALLAQSVNQGFEAVYQLTRMCTIRM | |
| | GTGAATCCCTACCACTACCAGAGAGTAGAGACA | | SFVKGWGAEYRRQTVTSTPCWIELHLNGPLQWLD | |
| | CCAGTTCTACCTCCTGTGTTGGTGCCACGCCAC | | KVLTQMGSPSIRCSSVS | |
| | ACAGAGATCCCGGCCGAGTTCCCCCCACTGGAC | | | |
| | GACTACAGCCATTCCATCCCCGAAAACACTAAC | | | |
| | TTCCCCGCAGGCATCGAGCCCCAGAGCAATATT | | | |
| | CCAGAGACCCCACCCCCTGGCTACCTGAGTGAA | | | |
| | GATGGAGAAACCAGTGACCACCAGATGAACCAC | | | |
| | AGCATGGACGCAGGTTCTCCAAACCTATCCCCG | | | |
| | AATCCGATGTCCCCAGCACATAATAACTTGGAC | | | |
| | CTGCAGCCAGTTACCTACTGCGAGCCGGCCTTC | | | |
| | TGGTGCTCCATCTCCTACTACGAGCTGAACCAG | | | |
| | CGCGTCGGGGAGACATTCCACGCCTCGCAGCCA | | | |
| | TCCATGACTGTGGATGGCTTCACCGACCCCTCC | | | |
| | AATTCGGAGCGCTTCTGCCTAGGGCTGCTCTCC | | | |
| | AATGTCAACAGGAATGCAGCAGTGGAGCTGACA | | | |
| | CGGAGACACATCGGAAGAGGCGTGCGGCTCTAC | | | |
| | TACATCGGAGGGGAGGTCTTCGCAGAGTGCCTC | | | |
| | AGTGACAGCGCTATTTTTGTCCAGTCTCCCAAC | | | |
| | TGTAACCAGCGCTATGGCTGGCACCCGGCCACC | | | |
| | GTCTGCAAGATCCCACCAGGATGCAACCTGAAG | | | |
| | ATCTTCAACAACCAGGAGTTCGCTGCCCTCCTG | | | |
| | GCCCAGTCGGTCAACCAGGGCTTTGAGGCTGTC | | | |
| | TACCAGTTGACCCGAATGTGCACCATCCGCATG | | | |
| | AGCTTCGTCAAAGGCTGGGGAGCGGAGTACAGG | | | |
| | AGACAGACTGTGACCAGTACCCCCTGCTGGATT | | | |
| | GAGCTGCACCTGAATGGGCCTTTGCAGTGGCTT | | | |
| | GACAAGGTCCTCACCCAGATGGGCTCCCCAAGC | | | |
| | ATCCGCTGTTCCAGTGTGTCT | | | |
| ATF2 | ATGAAATTCAAGTTACATGTGAATTCTGCCAGG | 18. | MKFKLHVNSARQYKDLWNMSDDKPFLCTAPGCGQ | 47. |
| | CAATACAAGGACCTGTGGAATATGAGTGATGAC | | RFTNEDHLAVHKHKHEMTLKFGPARNDSVIVADQ | |
| | AAACCCTTTCTATGTACTGCGCCTGGATGTGGC | | TPTPTRFLKNCEEVGLFNELASPFENEFKKASED | |
| | CAGCGTTTTACCAACGAGGATCATTTGGCTGTC | | DIKKMPLDLSPLATPIIRSKIEEPSVVETTHQDS | |
| | CATAAACATAAACATGAGATGACACTGAAATTT | | PLPHPESTTSDEKEVPLAQTAQPTSAIVRPASLQ | |
| | GGTCCAGCACGTAATGACAGTGTCATTGTGGCT | | VPNVLLTSSDSSVIIQQAVPSPTSSTVITQAPSS | |
| | GATCAGACCCCAACACCAACAAGATTCTTGAAA | | NRPIVPVPGPFPLLLHLPNGQTMPVAIPASITSS | |
| | AACTGTGAAGAAGTGGGTTTGTTTAATGAGTTG | | NVHVPAAVPLVRPVTMVPSVPGIPGPSSPQPVQS | |
| | GCGAGTCCATTTGAGAATGAATTCAAGAAAGCT | | EAKMRLKAALTQQHPPVTNGDTVKGHGSGLVRTQ | |
| | TCAGAAGATGACATTAAAAAAATGCCTCTAGAT | | SEESRPQSLQQPATSTTETPASPAHTTPQTQSTS | |
| | TTATCCCCTCTTGCAACACCTATCATAAGAAGC | | GRRRRAANEDPDEKRRKFLERNRAAASRCRQKRK | |
| | AAAATTGAGGAGCCTTCTGTTGTAGAAACAACT | | VWVQSLEKKAEDLSSLNGQLQSEVTLLRNEVAQL | |
| | CACCAGGATAGTCCTTTACCTCACCCAGAGTCT | | KQLLLAHKDCPVTAMQKKSGYHTADKDDSSEDIS | |
| | ACTACCAGTGATGAGAAGGAAGTACCATTGGCA | | VPSSPHTEAIQHSSVSTSNGVSSTSKAEAVATSV | |
| | CAAACTGCACAGCCCACATCAGCTATTGTTCGT | | LTQMADQSTEPALSQIVMAPSSQSQPSGS | |
| | CCAGCATCATTACAGGTTCCCAATGTGCTGCTT | | | |
| | ACAAGTTCTGACTCAAGTGTAATTATTCAGCAG | | | |
| | GCAGTACCTTCACCAACCTCAAGTACTGTAATC | | | |
| | ACCCAGGCACCATCCTCTAACAGGCCAATTGTC | | | |
| | CCTGTACCAGGCCCATTTCCTCTTCTGTTACAT | | | |
| | CTTCCTAATGGACAAACCATGCCTGTTGCTATT | | | |
| | CCTGCATCAATTACAAGTTCTAATGTGCATGTT | | | |
| | CCAGCTGCAGTCCCACTCGTTCGACCAGTCACC | | | |
| | ATGGTGCCTAGTGTTCCAGGAATCCCAGGTCCT | | | |
| | TCCTCTCCCCAACCAGTACAGTCAGAAGCAAAA | | | |
| | ATGAGATTAAAAGCTGCTTTGACCCAGCAACAT | | | |
| | CCTCCAGTTACCAATGGTGATACTGTCAAAGGT | | | |
| | CATGGTAGCGGATTGGTTAGGACTCAGTCAGAG | | | |
| | GAATCTCGACCGCAGTCATTACAACAGCCAGCC | | | |
| | ACATCCACTACAGAAACTCCGGCTTCTCCAGCT | | | |
| | CACACAACTCCACAGACCCAAAGTACAAGTGGT | | | |
| | CGTCGGAGAAGAGCAGCTAACGAAGATCCTGAT | | | |
| | GAAAAAAGGAGAAAGTTTTTAGAGCGAAATAGA | | | |
| | GCAGCAGCTTCAAGATGCCGACAAAAAAGGAAA | | | |
| | GTCTGGGTTCAGTCTTTAGAGAAGAAAGCTGAA | | | |
| | GACTTGAGTTCATTAAATGGTCAGCTGCAGAGT | | | |
| | GAAGTCACCCTGCTGAGAAATGAAGTGGCACAG | | | |
| | CTGAAACAGCTTCTTCTGGCTCATAAAGATTGC | | | |
| | CCTGTAACCGCCATGCAGAAGAAATCTGGCTAT | | | |
| | CATACTGCTGATAAAGATGATAGTTCAGAAGAC | | | |
| | ATTTCAGTGCCGAGTAGTCCACATACAGAAGCT | | | |
| | ATACAGCATAGTTCGGTCAGCACATCCAATGGA | | | |
| | GTCAGTTCAACCTCCAAGGCAGAAGCTGTAGCC | | | |
| | ACTTCAGTCCTCACCCAGATGGCGGACCAGAGT | | | |
| | ACAGAGCCTGCTCTTTCACAGATCGTTATGGCT | | | |
| | CCTTCCTCCCAGTCACAGCCCTCAGGAAGT | | | |
| SMAD4 | ATGGACAATATGTCTATTACGAATACACCAACA | 19. | MDNMSITNTPTSNDACLSIVHSLMCHRQGGESET | 48. |
| | AGTAATGATGCCTGTCTGAGCATTGTGCATAGT | | FAKRAIESLVKKLKEKKDELDSLITAITTNGAHP | |
| | TTGATGTGCCATAGACAAGGTGGAGAGAGTGAA | | SKCVTIQRTLDGRLQVAGRKGFPHVIYARLWRWP | |
| | ACATTTGCAAAAAGAGCAATTGAAAGTTTGGTA | | DLHKNELKHVKYCQYAFDLKCDSVCVNPYHYERV | |
| | AAGAAGCTGAAGGAGAAAAAAGATGAATTGGAT | | VSPGIDLSGLTLQSNAPSSMMVKDEYVHDFEGQP | |
| | TCTTTAATAACAGCTATAACTACAAATGGAGCT | | SLSTEGHSIQTIQHPPSNRASTETYSTPALLAPS | |
| | CATCCTAGTAAATGTGTTACCATACAGAGAACA | | ESNATSTANFPNIPVASTSQPASILGGSHSEGLL | |
| | TTGGATGGGAGGCTTCAGGTGGCTGGTCGGAAA | | QIASGPQPGQQQNGFTGQPATYHHNSTTTWTGSR | |
| | GGATTTCCTCATGTGATCTATGCCCGTCTCTGG | | TAPYTPNLPHHQNGHLQHHPPMPPHPGHYWPVHN | |
| | AGGTGGCCTGATCTTCACAAAAATGAACTAAAA | | ELAFQPPISNHPAPEYWCSIAYFEMDVQVGETFK | |
| | CATGTTAAATATTGTCAGTATGCGTTTGACTTA | | VPSSCPIVTVDGYVDPSGGDRFCLGQLSNVHRTE | |
| | AAATGTGATAGTGTCTGTGTGAATCCATATCAC | | AIERARLHIGKGVQLECKGEGDVWVRCLSDHAVF | |
| | TACGAACGAGTTGTATCACCTGGAATTGATCTC | | VQSYYLDREAGRAPGDAVHKIYPSAYIKVFDLRQ | |
| | TCAGGATTAACACTGCAGAGTAATGCTCCATCA | | CHRQMQQQAATAQAAAAAQAAAVAGNIPGPGSVG | |
| | AGTATGATGGTGAAGGATGAATATGTGCATGAC | | GIAPAISLSAAAGIGVDDLRRLCILRMSFVKGWG | |
| | TTTGAGGGACAGCCATCGTTGTCCACTGAAGGA | | PDYPRQSIKETPCWIEIHLHRALQLLDEVLHTMP | |
| | CATTCAATTCAAACCATCCAGCATCCACCAAGT | | IADPQPLD | |
| | AATCGTGCATCGACAGAGACATACAGCACCCCA | | | |
| | GCTCTGTTAGCCCCATCTGAGTCTAATGCTACC | | | |
| | AGCACTGCCAACTTTCCCAACATTCCTGTGGCT | | | |
| | TCCACAAGTCAGCCTGCCAGTATACTGGGGGGC | | | |
| | AGCCATAGTGAAGGACTGTTGCAGATAGCATCA | | | |
| | GGGCCTCAGCCAGGACAGCAGCAGAATGGATTT | | | |
| | ACTGGTCAGCCAGCTACTTACCATCATAACAGC | | | |
| | ACTACCACCTGGACTGGAAGTAGGACTGCACCA | | | |
| | TACACACCTAATTTGCCTCACCACCAAAACGGC | | | |
| | CATCTTCAGCACCACCCGCCTATGCCGCCCCAT | | | |
| | CCCGGACATTACTGGCCTGTTCACAATGAGCTT | | | |
| | GCATTCCAGCCTCCCATTTCCAATCATCCTGCT | | | |
| | CCTGAGTATTGGTGTTCCATTGCTTACTTTGAA | | | |
| | ATGGATGTTCAGGTAGGAGAGACATTTAAGGTT | | | |
| | CCTTCAAGCTGCCCTATTGTTACTGTTGATGGA | | | |
| | TACGTGGACCCTTCTGGAGGAGATCGCTTTTGT | | | |
| | TTGGGTCAACTCTCCAATGTCCACAGGACAGAA | | | |
| | GCCATTGAGAGAGCAAGGTTGCACATAGGCAAA | | | |
| | GGTGTGCAGTTGGAATGTAAAGGTGAAGGTGAT | | | |
| | GTTTGGGTCAGGTGCCTTAGTGACCACGCGGTC | | | |
| | TTTGTACAGAGTTACTACTTAGACAGAGAAGCT | | | |
| | GGGCGTGCACCTGGAGATGCTGTTCATAAGATC | | | |
| | TACCCAAGTGCATATATAAAGGTCTTTGATTTG | | | |
| | CGTCAGTGTCATCGACAGATGCAGCAGCAGGCG | | | |
| | GCTACTGCACAAGCTGCAGCAGCTGCCCAGGCA | | | |
| | GCAGCCGTGGCAGGAAACATCCCTGGCCCAGGA | | | |
| | TCAGTAGGTGGAATAGCTCCAGCTATCAGTCTG | | | |
| | TCAGCTGCTGCTGGAATTGGTGTTGATGACCTT | | | |
| | CGTCGCTTATGCATACTCAGGATGAGTTTTGTG | | | |
| | AAAGGCTGGGGACCGGATTACCCAAGACAGAGC | | | |
| | ATCAAAGAAACACCTTGCTGGATTGAAATTCAC | | | |
| | TTACACCGGGCCCTCCAGCTCCTAGACGAAGTA | | | |
| | CTTCATACCATGCCGATTGCAGACCCACAACCT | | | |
| | TTAGAC | | | |
| FOXP3 | ATGCCCAACCCCAGGCCTGGCAAGCCCTCGGCC | 20. | MPNPRPGKPSAPSLALGPSPGASPSWRAAPKASD | 49. |
| | CCTTCCTTGGCCCTTGGCCCATCCCCAGGAGCC | | LLGARGPGGTFQGRDLRGGAHASSSSLNPMPPSQ | |
| | TCGCCCAGCTGGAGGGCTGCACCCAAAGCCTCA | | LQLPTLPLVMVAPSGARLGPLPHLQALLQDRPHF | |
| | GACCTGCTGGGGGCCCGGGGCCCAGGGGGAACC | | MHQLSTVDAHARTPVLQVHPLESPAMISLTPPTT | |
| | TTCCAGGGCCGAGATCTTCGAGGCGGGGCCCAT | | ATGVFSLKARPGLPPGINVASLEWVSREPALLCT | |
| | GCCTCCTCTTCTTCCTTGAACCCCATGCCACCA | | FPNPSAPRKDSTLSAVPQSSYPLLANGVCKWPGC | |
| | TCGCAGCTGCAGCTGCCCACACTGCCCCTAGTC | | EKVFEEPEDFLKHCQADHLLDEKGRAQCLLQREM | |
| | ATGGTGGCACCCTCCGGGGCACGGCTGGGCCCC | | VQSLEQQLVLEKEKLSAMQAHLAGKMALTKASSV | |
| | TTGCCCCACTTACAGGCACTCCTCCAGGACAGG | | ASSDKGSCCIVAAGSQGPVVPAWSGPREAPDSLF | |
| | CCACATTTCATGCACCAGCTCTCAACGGTGGAT | | AVRRHLWGSHGNSTFPEFLHNMDYFKFHNMRPPF | |
| | GCCCACGCCCGGACCCCTGTGCTGCAGGTGCAC | | TYATLIRWAILEAPEKQRTLNEIYHWFTRMFAFF | |
| | CCCCTGGAGAGCCCAGCCATGATCAGCCTCACA | | RNHPATWKNAIRHNLSLHKCFVRVESEKGAVWTV | |
| | CCACCCACCACCGCCACTGGGGTCTTCTCCCTC | | DELEFRKKRSQRPSRCSNPTPGP | |
| | AAGGCCCGGCCTGGCCTCCCACCTGGGATCAAC | | | |
| | GTGGCCAGCCTGGAATGGGTGTCCAGGGAGCCG | | | |
| | GCACTGCTCTGCACCTTCCCAAATCCCAGTGCA | | | |
| | CCCAGGAAGGACAGCACCCTTTCGGCTGTGCCC | | | |
| | CAGAGCTCCTACCCACTGCTGGCAAATGGTGTC | | | |
| | TGCAAGTGGCCCGGATGTGAGAAGGTCTTCGAA | | | |
| | GAGCCAGAGGACTTCCTCAAGCACTGCCAGGCG | | | |
| | GACCATCTTCTGGATGAGAAGGGCAGGGCACAA | | | |
| | TGTCTCCTCCAGAGAGAGATGGTACAGTCTCTG | | | |
| | GAGCAGCAGCTGGTGCTGGAGAAGGAGAAGCTG | | | |
| | AGTGCCATGCAGGCCCACCTGGCTGGGAAAATG | | | |
| | GCACTGACCAAGGCTTCATCTGTGGCATCATCC | | | |
| | GACAAGGGCTCCTGCTGCATCGTAGCTGCTGGC | | | |
| | AGCCAAGGCCCTGTCGTCCCAGCCTGGTCTGGC | | | |
| | CCCCGGGAGGCCCCTGACAGCCTGTTTGCTGTC | | | |
| | CGGAGGCACCTGTGGGGTAGCCATGGAAACAGC | | | |
| | ACATTCCCAGAGTTCCTCCACAACATGGACTAC | | | |
| | TTCAAGTTCCACAACATGCGACCCCCTTTCACC | | | |
| | TACGCCACGCTCATCCGCTGGGCCATCCTGGAG | | | |
| | GCTCCAGAGAAGCAGCGGACACTCAATGAGATC | | | |
| | TACCACTGGTTCACACGCATGTTTGCCTTCTTC | | | |
| | AGAAACCATCCTGCCACCTGGAAGAACGCCATC | | | |
| | CGCCACAACCTGAGTCTGCACAAGTGCTTTGTG | | | |
| | CGGGTGGAGAGCGAGAAGGGGGCTGTGTGGACC | | | |
| | GTGGATGAGCTGGAGTTCCGCAAGAAACGGAGC | | | |
| | CAGAGGCCCAGCAGGTGTTCCAACCCTACACCT | | | |
| | GGCCCC | | | |
| RELA | ATGGACGAACTGTTCCCCCTCATCTTCCCGGCA | 21. | MDELFPLIFPAEPAQASGPYVEIIEQPKQRGMRF | 50. |
| | GAGCCAGCCCAGGCCTCTGGCCCCTATGTGGAG | | RYKCEGRSAGSIPGERSTDTTKTHPTIKINGYTG | |
| | ATCATTGAGCAGCCCAAGCAGCGGGGCATGCGC | | PGTVRISLVTKDPPHRPHPHELVGKDCRDGFYEA | |
| | TTCCGCTACAAGTGCGAGGGGCGCTCCGCGGGC | | ELCPDRCIHSFQNLGIQCVKKRDLEQAISQRIQT | |
| | AGCATCCCAGGCGAGAGGAGCACAGATACCACC | | NNNPFQVPIEEQRGDYDLNAVRLCFQVTVRDPSG | |
| | AAGACCCACCCCACCATCAAGATCAATGGCTAC | | RPLRLPPVLSHPIFDNRAPNTAELKICRVNRNSG | |
| | ACAGGACCAGGGACAGTGCGCATCTCCCTGGTC | | SCLGGDEIFLLCDKVQKEDIEVYFTGPGWEARGS | |
| | ACCAAGGACCCTCCTCACCGGCCTCACCCCCAC | | FSQADVHRQVAIVFRTPPYADPSLQAPVRVSMQL | |
| | GAGCTTGTAGGAAAGGACTGCCGGGATGGCTTC | | RRPSDRELSEPMEFQYLPDTDDRHRIEEKRKRTY | |
| | TATGAGGCTGAGCTCTGCCCGGACCGCTGCATC | | ETFKSIMKKSPFSGPTDPRPPPRRIAVPSRSSAS | |
| | CACAGTTTCCAGAACCTGGGAATCCAGTGTGTG | | VPKPAPQPYPFTSSLSTINYDEFPTMVFPSGQIS | |
| | AAGAAGCGGGACCTGGAGCAGGCTATCAGTCAG | | QASALAPAPPQVLPQAPAPAPAPAMVSALAQAPA | |
| | CGCATCCAGACCAACAACAACCCCTTCCAAGTT | | PVPVLAPGPPQAVAPPAPKPTQAGEGTLSEALLQ | |
| | CCTATAGAAGAGCAGCGTGGGGACTACGACCTG | | LQFDDEDLGALLGNSTDPAVFTDLASVDNSEFQQ | |
| | AATGCTGTGCGGCTCTGCTTCCAGGTGACAGTG | | LLNQGIPVAPHTTEPMLMEYPEAITRLVTGAQRP | |
| | CGGGACCCATCAGGCAGGCCCCTCCGCCTGCCG | | PDPAPAPLGAPGLPNGLLSGDEDFSSIADMDFSA | |
| | CCTGTCCTTTCTCATCCCATCTTTGACAATCGT | | LLSQISS | |
| | GCCCCCAACACTGCCGAGCTCAAGATCTGCCGA | | | |
| | GTGAACCGAAACTCTGGCAGCTGCCTCGGTGGG | | | |
| | GATGAGATCTTCCTACTGTGTGACAAGGTGCAG | | | |
| | AAAGAGGACATTGAGGTGTATTTCACGGGACCA | | | |
| | GGCTGGGAGGCCCGAGGCTCCTTTTCGCAAGCT | | | |
| | GATGTGCACCGACAAGTGGCCATTGTGTTCCGG | | | |
| | ACCCCTCCCTACGCAGACCCCAGCCTGCAGGCT | | | |
| | CCTGTGCGTGTCTCCATGCAGCTGCGGCGGCCT | | | |
| | TCCGACCGGGAGCTCAGTGAGCCCATGGAATTC | | | |
| | CAGTACCTGCCAGATACAGACGATCGTCACCGG | | | |
| | ATTGAGGAGAAACGTAAAAGGACATATGAGACC | | | |
| | TTCAAGAGCATCATGAAGAAGAGTCCTTTCAGC | | | |
| | GGACCCACCGACCCCCGGCCTCCACCTCGACGC | | | |
| | ATTGCTGTGCCTTCCCGCAGCTCAGCTTCTGTC | | | |
| | CCCAAGCCAGCACCCCAGCCCTATCCCTTTACG | | | |
| | TCATCCCTGAGCACCATCAACTATGATGAGTTT | | | |
| | CCCACCATGGTGTTTCCTTCTGGGCAGATCAGC | | | |
| | CAGGCCTCGGCCTTGGCCCCGGCCCCTCCCCAA | | | |
| | GTcctgccccaggetccagcccctgcccctgct | | | |
| | ccagccATGGTATCAGCTCTGGCCCAGGCCCCA | | | |
| | GCCCCTGTCCCAGTCCTAGCCCCAGGCCCTCCT | | | |
| | CAGGCTGTGGCCCCACCTGCCCCCAAGCCCACC | | | |
| | CAGGCTGGGGAAGGAACGCTGTCagaggccctg | | | |
| | ctgcagctgcagtttgatgatgaagacctgggg | | | |
| | gccttgcttggcaacagcacagacccagctgtg | | | |
| | tTCACAGACCTGGCATCCGTCGACAACTCCGAG | | | |
| | TTTCAGCAGCTGCTGAACCAGGGCATACCTGTG | | | |
| | GCCCCCCACACAACTGAGCCCATGCTGATGGAG | | | |
| | TACCCTGAGGCTATAACTCGCCTAGTGACAGGG | | | |
| | GCCCAGAGGCCCCCCGACCCAGCTCCTGCTCCA | | | |
| | CTGGGGGCCCCGGGGCTCCCCAATGGCCTCCTT | | | |
| | TCAGGAGATGAAGACTTCTCCTCCATTGCGGAC | | | |
| | ATGGACTTCTCAGCCCTGCTGAGTCAGATCAGC | | | |
| | TCC | | | |
| SOX5 | ATGCTTACTGACCCTGATTTACCTCAGGAGTTT | 22. | MLTDPDLPQEFERMSSKRPASPYGEADGEVAMVT | 51. |
| | GAAAGGATGTCTTCCAAGCGACCAGCCTCTCCG | | SRQKVEEEESDGLPAFHLPLHVSFPNKPHSEEFQ | |
| | TATGGGGAAGCAGATGGAGAGGTAGCCATGGTG | | PVSLLTQETCGHRTPTSQHNTMEVDGNKVMSSFA | |
| | ACAAGCAGACAGAAAGTGGAAGAAGAGGAGAGT | | PHNSSTSPQKAEEGGRQSGESLSSTALGTPERRK | |
| | GACGGGCTCCCAGCCTTTCACCTTCCCTTGCAT | | GSLADVVDTLKQRKMEELIKNEPEETPSIEKLLS | |
| | GTGAGTTTTCCCAACAAGCCTCACTCTGAGGAA | | KDWKDKLLAMGSGNFGEIKGTPESLAEKERQLMG | |
| | TTTCAGCCAGTTTCTCTGCTGACGCAAGAGACT | | MINQLTSLREQLLAAHDEQKKLAASQIEKQRQQM | |
| | TGTGGCCATAGGACTCCCACTTCTCAGCACAAT | | ELAKQQQEQIARQQQQLLQQQHKINLLQQQIQVQ | |
| | ACAATGgAAGTTGATGGCAATAAAGTTATGTCT | | GQLPPLMIPVFPPDQRTLAAAAQQGFLLPPGFSY | |
| | TCATTTGCCCCACACAACTCATCTACCTCACCT | | KAGCSDPYPVQLIPTTMAAAAAATPGLGPLQLQQ | |
| | CAGAAGGCAGAAGAAGGTGGGCGACAGAGTGGC | | LYAAQLAAMQVSPGGKLPGIPQGNLGAAVSPTSI | |
| | GAGTCCTTGTCTAGTACAGCCCTGGGAACTCCT | | HTDKSTNSPPPKSKDEVAQPLNLSAKPKTSDGKS | |
| | GAACGGCGCAAGGGCAGTTTAGCTGATGTTGTT | | PTSPTSPHMPALRINSGAGPLKASVPAALASPSA | |
| | GACACCTTGAAGCAGAGGAAAATGGAAGAGCTC | | RVSTIGYLNDHDAVTKAIQEARQMKEQLRREQQV | |
| | ATCAAAAACGAGCCGGAAGAAACCCCCAGTATT | | LDGKVAVVNSLGLNNCRTEKEKTTLESLTQQLAV | |
| | GAAAAACTACTCTCAAAGGACTGGAAAGACAAG | | KQNEEGKFSHAMMDFNLSGDSDGSAGVSESRIYR | |
| | CTTCTTGCAATGGGATCGGGGAACTTTGGCGAA | | ESRGRGSNEPHIKRPMNAFMVWAKDERRKILQAF | |
| | ATAAAAGGGACTCCCGAGAGCTTAGCTGAGAAA | | PDMHNSNISKILGSRWKAMTNLEKQPYYEEQARL | |
| | GAAAGGCAACTCATGGGTATGATCAACCAGCTG | | SKQHLEKYPDYKYKPRPKRTCLVDGKKLRIGEYK | |
| | ACCAGCCTCCGAGAGCAGCTGTTGGCTGCCCAC | | AIMRNRRQEMRQYFNVGQQAQIPIATAGVVYPGA | |
| | GATGAGCAGAAGAAACTAGCTGCCTCTCAGATT | | IAMAGMPSPHLPSEHSSVSSSPEPGMPVIQSTYG | |
| | GAGAAACAGCGTCAGCAAATGGAGCTGGCCAAG | | VKGEEPHIKEEIQAEDINGEIYDEYDEEEDDPDV | |
| | CAGCAACAAGAACAAATTGCAAGACAGCAGCAG | | DYGSDSENHIAGQAN | |
| | CAGCTTCTACAGCAACAACACAAAATCAATTTG | | | |
| | CTCCAGCAACAGATCCAGGTTCAAGGTCAGCTG | | | |
| | CCGCCATTAATGATTCCCGTATTCCCTCCTGAT | | | |
| | CAACGGACACTGGCTGCAGCTGCCCAGCAAGGA | | | |
| | TTCCTCCTCCCTCCAGGCTTCAGCTATAAGGCT | | | |
| | GGATGTAGTGACCCTTACCCTGTTCAGCTGATC | | | |
| | CCAACTACCATGGCAGCTGCTGCCGCAGCAACA | | | |
| | CCAGGCTTAGGCCCACTCCAACTGCAGCAGTTA | | | |
| | TATGCTGCCCAGCTAGCTGCAATGCAGGTATCT | | | |
| | CCAGGAGGGAAGCTGCCAGGCATACCCCAAGGC | | | |
| | AACCTTGGTGCTGCTGTATCTCCTACCAGCATT | | | |
| | CACACAGACAAGAGCACAAACAGCCCACCACCC | | | |
| | AAAAGCAAGGATGAAGTGGCACAGCCACTGAAC | | | |
| | CTATCAGCTAAACCCAAGACCTCTGATGGCAAA | | | |
| | TCACCCACATCACCCACCTCTCCCCATATGCCA | | | |
| | GCTCTGAGAATAAACAGTGGGGCAGGCCCCCTC | | | |
| | AAAGCCTCTGTCCCAGCAGCGTTAGCTAGTCCT | | | |
| | TCAGCCAGAGTTAGCACAATAGGTTACTTAAAT | | | |
| | GACCATGATGCTGTCACCAAGGCAATCCAAGAA | | | |
| | GCTCGGCAAATGAAGGAGCAACTCCGACGGGAA | | | |
| | CAACAGGTGCTTGATGGGAAGGTGGCTGTTGTG | | | |
| | AATAGTCTGGGTCTCAATAACTGCCGAACAGAA | | | |
| | AAGGAAAAAACAACACTGGAGAGTCTGACTCAG | | | |
| | CAACTGGCAGTTAAACAGAATGAAGAAGGAAAA | | | |
| | TTTAGCCATGCAATGATGGATTTCAATCTGAGT | | | |
| | GGAGATTCTGATGGAAGTGCTGGAGTCTCAGAG | | | |
| | TCAAGAATTTATAGGGAATCCCGAGGGCGTGGT | | | |
| | AGCAATGAACCCCACATAAAGCGTCCAATGAAT | | | |
| | GCCTTCATGGTGTGGGCTAAAGATGAACGGAGA | | | |
| | AAGATCCTTCAAGCCTTTCCTGACATGCACAAC | | | |
| | TCCAACATCAGCAAGATATTGGGATCTCGCTGG | | | |
| | AAAGCTATGACAAACCTAGAGAAACAGCCATAT | | | |
| | TATGAGGAGCAAGCCCGTCTCAGCAAGCAGCAC | | | |
| | CTGGAGAAGTACCCTGACTATAAGTACAAGCCC | | | |
| | AGGCCAAAGCGCACCTGCCTGGTGGATGGCAAA | | | |
| | AAGCTGCGCATTGGTGAATACAAGGCAATCATG | | | |
| | CGCAACAGGCGGCAGGAAATGCGGCAGTACTTC | | | |
| | AATGTTGGGCAACAAGCACAGATCCCCATTGCC | | | |
| | ACTGCTGGTGTTGTGTACCCTGGAGCCATCGCC | | | |
| | ATGGCTGGGATGCCCTCCCCTCACCTGCCCTCG | | | |
| | GAGCACTCAAGCGTGTCTAGCAGCCCAGAGCCT | | | |
| | GGGATGCCTGTTATCCAGAGCACTTACGGTGTG | | | |
| | AAAGGAGAGGAGCCACATATCAAAGAAGAGATA | | | |
| | CAGGCCGAGGACATCAATGGAGAAATTTATGAT | | | |
| | GAGTACGACGAGGAAGAGGATGATCCAGATGTA | | | |
| | GATTATGGGAGTGACAGTGAAAACCATATTGCA | | | |
| | GGACAAGCCAAC | | | |
| TIGIT_trunc | ATGCGCTGGTGTCTCCTCCTGATCTGGGCCCAG | 23. | MRWCLLLIWAQGLRQAPLASGMMTGTIETTGNIS | 52. |
| | GGGCTGAGGCAGGCTCCCCTCGCCTCAGGAATG | | AEKGGSIILQCHLSSTTAQVTQVNWEQQDQLLAI | |
| | ATGACAGGCACAATAGAAACAACGGGGAACATT | | CNADLGWHISPSFKDRVAPGPGLGLTLQSLTVND | |
| | TCTGCAGAGAAAGGTGGCTCTATCATCTTACAA | | TGEYFCIYHTYPDGTYTGRIFLEVLESSVAEHGA | |
| | TGTCACCTCTCCTCCACCACGGCACAAGTGACC | | RFQIPLLGAMAATLVVICTAVIVVVALTRKKKA | |
| | CAGGTCAACTGGGAGCAGCAGGACCAGCTTCTG | | | |
| | GCCATTTGTAATGCTGACTTGGGGTGGCACATC | | | |
| | TCCCCATCCTTCAAGGATCGAGTGGCCCCAGGT | | | |
| | CCCGGCCTGGGCCTCACCCTCCAGTCGCTGACC | | | |
| | GTGAACGATACAGGGGAGTACTTCTGCATCTAT | | | |
| | CACACCTACCCTGATGGGACGTACACTGGGAGA | | | |
| | ATCTTCCTGGAGGTCCTAGAAAGCTCAGTGGCT | | | |
| | GAGCACGGTGCCAGGTTCCAGATTCCATTGCTT | | | |
| | GGAGCCATGGCCGCGACGCTGGTGGTCATCTGC | | | |
| | ACAGCAGTCATCGTGGTGGTCGCGTTGACTAGA | | | |
| | AAGAAGAAAGCC | | | |
| MYC | ATGCCCCTCAACGTTAGCTTCACCAACAGGAAC | 24. | MPLNVSFTNRNYDLDYDSVQPYFYCDEEENFYQQ | 53. |
| | TATGACCTCGACTACGACTCGGTGCAGCCGTAT | | QQQSELQPPAPSEDIWKKFELLPTPPLSPSRRSG | |
| | TTCTACTGCGACGAGGAGGAGAACTTCTACCAG | | LCSPSYVAVTPFSLRGDNDGGGGSFSTADQLEMV | |
| | CAGCAGCAGCAGAGCGAGCTGCAGCCCCCGGCG | | TELLGGDMVNQSFICDPDDETFIKNIIIQDCMWS | |
| | CCCAGCGAGGATATCTGGAAGAAATTCGAGCTG | | GFSAAAKLVSEKLASYQAARKDSGSPNPARGHSV | |
| | CTGCCCACCCCGCCCCTGTCCCCTAGCCGCCGC | | CSTSSLYLQDLSAAASECIDPSVVFPYPLNDSSS | |
| | TCCGGGCTCTGCTCGCCCTCCTACGTTGCGGTC | | PKSCASQDSSAFSPSSDSLLSSTESSPQGSPEPL | |
| | ACACCCTTCTCCCTTCGGGGAGACAACGACGGC | | VLHEETPPTTSSDSEEEQEDEEEIDVVSVEKRQA | |
| | GGTGGCGGGAGCTTCTCCACGGCCGACCAGCTG | | PGKRSESGSPSAGGHSKPPHSPLVLKRCHVSTHQ | |
| | GAGATGGTGACCGAGCTGCTGGGAGGAGACATG | | HNYAAPPSTRKDYPAAKRVKLDSVRVLRQISNNR | |
| | GTGAACCAGAGTTTCATCTGCGACCCGGACGAC | | KCTSPRSSDTEENVKRRTHNVLERQRRNELKRSF | |
| | GAGACCTTCATCAAAAACATCATCATCCAGGAC | | FALRDQIPELENNEKAPKVVILKKATAYILSVQA | |
| | TGTATGTGGAGCGGCTTCTCGGCCGCCGCCAAG | | EEQKLISEEDLLRKRREQLKHKLEQLRNSCA | |
| | CTCGTCTCAGAGAAGCTGGCCTCCTACCAGGCT | | | |
| | GCGCGCAAAGACAGCGGCAGCCCGAACCCCGCC | | | |
| | CGCGGCCACAGCGTCTGCTCCACCTCCAGCTTG | | | |
| | TACCTGCAGGATCTGAGCGCCGCCGCCTCAGAG | | | |
| | TGCATCGACCCCTCGGTGGTCTTCCCCTACCCT | | | |
| | CTCAACGACAGCAGCTCGCCCAAGTCCTGCGCC | | | |
| | TCGCAAGACTCCAGCGCCTTCTCTCCGTCCTCG | | | |
| | GATTCTCTGCTCTCCTCGACGGAGTCCTCCCCG | | | |
| | CAGGGCAGCCCCGAGCCCCTGGTGCTCCATGAG | | | |
| | GAGACACCGCCCACCACCAGCAGCGACTCTGAG | | | |
| | GAGGAACAAGAAGATGAGGAAGAAATCGATGTT | | | |
| | GTTTCTGTGGAAAAGAGGCAGGCTCCTGGCAAA | | | |
| | AGGTCAGAGTCTGGATCACCTTCTGCTGGAGGC | | | |
| | CACAGCAAACCTCCTCACAGCCCACTGGTCCTC | | | |
| | AAGAGGTGCCACGTCTCCACACATCAGCACAAC | | | |
| | TACGCAGCGCCTCCCTCCACTCGGAAGGACTAT | | | |
| | CCTGCTGCCAAGAGGGTCAAGTTGGACAGTGTC | | | |
| | AGAGTCCTGAGACAGATCAGCAACAACCGAAAA | | | |
| | TGCACCAGCCCCAGGTCCTCGGACACCGAGGAG | | | |
| | AATGTCAAGAGGCGAACACACAACGTCTTGGAG | | | |
| | CGCCAGAGGAGGAACGAGCTAAAACGGAGCTTT | | | |
| | TTTGCCCTGCGTGACCAGATCCCGGAGTTGGAA | | | |
| | AACAATGAAAAGGCCCCCAAGGTAGTTATCCTT | | | |
| | AAAAAAGCCACAGCATACATCCTGTCCGTCCAA | | | |
| | GCAGAGGAGCAAAAGCTCATTTCTGAAGAGGAC | | | |
| | TTGTTGCGGAAACGACGAGAACAGTTGAAACAC | | | |
| | AAACTTGAACAGCTACGGAACTCTTGTGCG | | | |
| IL2RA | ATGGATTCATACCTGCTGATGTGGGGACTGCTC | 25. | MDSYLLMWGLLTFIMVPGCQAELCDDDPPEIPHA | 54. |
| | ACGTTCATCATGGTGCCTGGCTGCCAGGCAGAG | | TFKAMAYKEGTMLNCECKRGFRRIKSGSLYMLCT | |
| | CTCTGTGACGATGACCCGCCAGAGATCCCACAC | | GNSSHSSWDNQCQCTSSATRNTTKQVTPQPEEQK | |
| | GCCACATTCAAAGCCATGGCCTACAAGGAAGGA | | ERKTTEMQSPMQPVDQASLPGHCREPPPWENEAT | |
| | ACCATGTTGAACTGTGAATGCAAGAGAGGTTTC | | ERIYHFVVGQMVYYQCVQGYRALHRGPAESVCKM | |
| | CGCAGAATAAAAAGCGGGTCACTCTATATGCTC | | THGKTRWTQPQLICTGEMETSQFPGEEKPQASPE | |
| | TGTACAGGAAACTCTAGCCACTCGTCCTGGGAC | | GRPESETSCLVTTTDFQIQTEMAATMETSIFTTE | |
| | AACCAATGTCAATGCACAAGCTCTGCCACTCGG | | YQVAVAGCVFLLISVLLLSGLTWQRRQRKSRRTI | |
| | AACACAACGAAACAAGTGACACCTCAACCTGAA | | | |
| | GAACAGAAAGAAAGGAAAACCACAGAAATGCAA | | | |
| | AGTCCAATGCAGCCAGTGGACCAAGCGAGCCTT | | | |
| | CCAGGTCACTGCAGGGAACCTCCACCATGGGAA | | | |
| | AATGAAGCCACAGAGAGAATTTATCATTTCGTG | | | |
| | GTGGGGCAGATGGTTTATTATCAGTGCGTCCAG | | | |
| | GGATACAGGGCTCTACACAGAGGTCCTGCTGAG | | | |
| | AGCGTCTGCAAAATGACCCACGGGAAGACAAGG | | | |
| | TGGACCCAGCCCCAGCTCATATGCACAGGTGAA | | | |
| | ATGGAGACCAGTCAGTTTCCAGGTGAAGAGAAG | | | |
| | CCTCAGGCAAGCCCCGAAGGCCGTCCTGAGAGT | | | |
| | GAGACTTCCTGCCTCGTCACAACAACAGATTTT | | | |
| | CAAATACAGACAGAAATGGCTGCAACCATGGAG | | | |
| | ACGTCCATATTTACAACAGAGTACCAGGTAGCA | | | |
| | GTGGCCGGCTGTGTTTTCCTGCTGATCAGCGTC | | | |
| | CTCCTCCTGAGTGGGCTCACCTGGCAGCGGAGA | | | |
| | CAGAGGAAGAGTAGAAGAACAATC | | | |
| 4-1BB | ATGGGAAACAGCTGTTACAACATAGTAGCCACT | 26. | MGNSCYNIVATLLLVLNFERTRSLQDPCSNCPAG | 55. |
| (TNFRSF9) | CTGTTGCTGGTCCTCAACTTTGAGAGGACAAGA | | TFCDNNRNQICSPCPPNSFSSAGGQRTCDICRQC | |
| | TCATTGCAGGATCCTTGTAGTAACTGCCCAGCT | | KGVFRTRKECSSTSNAECDCTPGFHCLGAGCSMC | |
| | GGTACATTCTGTGATAATAACAGGAATCAGATT | | EQDCKQGQELTKKGCKDCCFGTFNDQKRGICRPW | |
| | TGCAGTCCCTGTCCTCCAAATAGTTTCTCCAGC | | TNCSLDGKSVLVNGTKERDVVCGPSPADLSPGAS | |
| | GCAGGTGGACAAAGGACCTGTGACATATGCAGG | | SVTPPAPAREPGHSPQIISFFLALTSTALLFLLF | |
| | CAGTGTAAAGGTGTTTTCAGGACCAGGAAGGAG | | FLTLRFSVVKRGRKKLLYIFKQPFMRPVQTTQEE | |
| | TGTTCCTCCACCAGCAATGCAGAGTGTGACTGC | | DGCSCRFPEEEEGGCEL | |
| | ACTCCAGGGTTTCACTGCCTGGGGGCAGGATGC | | | |
| | AGCATGTGTGAACAGGATTGTAAACAAGGTCAA | | | |
| | GAACTGACAAAAAAAGGTTGTAAAGACTGTTGC | | | |
| | TTTGGGACATTTAACGATCAGAAACGTGGCATC | | | |
| | TGTCGACCCTGGACAAACTGTTCTTTGGATGGA | | | |
| | AAGTCTGTGCTTGTGAATGGGACGAAGGAGAGG | | | |
| | GACGTGGTCTGTGGACCATCTCCAGCCGACCTC | | | |
| | TCTCCGGGAGCATCCTCTGTGACCCCGCCTGCC | | | |
| | CCTGCGAGAGAGCCAGGACACTCTCCGCAGATC | | | |
| | ATCTCCTTCTTTCTTGCGCTGACGTCGACTGCG | | | |
| | TTGCTCTTCCTGCTGTTCTTCCTCACGCTCCGT | | | |
| | TTCTCTGTTGTTAAACGGGGCAGAAAGAAACTC | | | |
| | CTGTATATATTCAAACAACCATTTATGAGACCA | | | |
| | GTACAAACTACTCAAGAGGAAGATGGCTGTAGC | | | |
| | TGCCGATTTCCAGAAGAAGAAGAAGGAGGATGT | | | |
| | GAACTG | | | |
| SMAD1 | ATGAATGTGACAAGTTTATTTTCCTTTACAAGT | 27. | MNVTSLFSFTSPAVKRLLGWKQGDEEEKWAEKAV | 56. |
| | CCAGCTGTGAAGAGACTTCTTGGGTGGAAACAG | | DALVKKLKKKKGAMEELEKALSCPGQPSNCVTIP | |
| | GGCGATGAAGAAGAAAAATGGGCAGAGAAAGCT | | RSLDGRLQVSHRKGLPHVIYCRVWRWPDLQSHHE | |
| | GTTGATGCTTTGGTGAAAAAACTGAAGAAAAAG | | LKPLECCEFPFGSKQKEVCINPYHYKRVESPVLP | |
| | AAAGGTGCCATGGAGGAACTGGAAAAGGCCTTG | | PVLVPRHSEYNPQHSLLAQFRNLGQNEPHMPLNA | |
| | AGCTGCCCAGGGCAACCGAGTAACTGTGTCACC | | TFPDSFQQPNSHPFPHSPNSSYPNSPGSSSSTYP | |
| | ATTCCCCGCTCTCTGGATGGCAGGCTGCAAGTC | | HSPTSSDPGSPFQMPADTPPPAYLPPEDPMTQDG | |
| | TCCCACCGGAAGGGACTGCCTCATGTCATTTAC | | SQPMDTNMMAPPLPSEINRGDVQAVAYEEPKHWC | |
| | TGCCGTGTGTGGCGCTGGCCCGATCTTCAGAGC | | SIVYYELNNRVGEAFHASSTSVLVDGFTDPSNNK | |
| | CACCATGAACTAAAACCACTGGAATGCTGTGAG | | NRFCLGLLSNVNRNSTIENTRRHIGKGVHLYYVG | |
| | TTTCCTTTTGGTTCCAAGCAGAAGGAGGTCTGC | | GEVYAECLSDSSIFVQSRNCNYHHGFHPTTVCKI | |
| | ATCAATCCCTACCACTATAAGAGAGTAGAAAGC | | PSGCSLKIFNNQEFAQLLAQSVNHGFETVYELTK | |
| | CCTGTACTTCCTCCTGTGCTGGTTCCAAGACAC | | MCTIRMSFVKGWGAEYHRQDVTSTPCWIEIHLHG | |
| | AGCGAATATAATCCTCAGCACAGCCTCTTAGCT | | PLQWLDKVLTQMGSPHNPISSVS | |
| | CAGTTCCGTAACTTAGGACAAAATGAGCCTCAC | | | |
| | ATGCCACTCAACGCCACTTTTCCAGATTCTTTC | | | |
| | CAGCAACCCAACAGCCACCCGTTTCCTCACTCT | | | |
| | CCCAATAGCAGTTACCCAAACTCTCCTGGGAGC | | | |
| | AGCAGCAGCACCTACCCTCACTCTCCCACCAGC | | | |
| | TCAGACCCAGGAAGCCCTTTCCAGATGCCAGCT | | | |
| | GATACGCCCCCACCTGCTTACCTGCCTCCTGAA | | | |
| | GACCCCATGACCCAGGATGGCTCTCAGCCGATG | | | |
| | GACACAAACATGATGGCGCCTCCCCTGCCCTCA | | | |
| | GAAATCAACAGAGGAGATGTTCAGGCGGTTGCT | | | |
| | TATGAGGAACCAAAACACTGGTGCTCTATTGTC | | | |
| | TACTATGAGCTCAACAATCGTGTGGGTGAAGCG | | | |
| | TTCCATGCCTCCTCCACAAGTGTGTTGGTGGAT | | | |
| | GGTTTCACTGATCCTTCCAACAATAAGAACCGT | | | |
| | TTCTGCCTTGGGCTGCTCTCCAATGTTAACCGG | | | |
| | AATTCCACTATTGAAAACACCAGGCGGCATATT | | | |
| | GGAAAAGGAGTTCATCTTTATTATGTTGGAGGG | | | |
| | GAGGTGTATGCCGAATGCCTTAGTGACAGTAGC | | | |
| | ATCTTTGTGCAAAGTCGGAACTGCAACTACCAT | | | |
| | CATGGATTTCATCCTACTACTGTTTGCAAGATC | | | |
| | CCTAGTGGGTGTAGTCTGAAAATTTTTAACAAC | | | |
| | CAAGAATTTGCTCAGTTATTGGCACAGTCTGTG | | | |
| | AACCATGGATTTGAGACAGTCTATGAGCTTACA | | | |
| | AAAATGTGTACTATACGTATGAGCTTTGTGAAG | | | |
| | GGCTGGGGAGCAGAATACCACCGCCAGGATGTT | | | |
| | ACTAGCACCCCCTGCTGGATTGAGATACATCTG | | | |
| | CACGGCCCCCTCCAGTGGCTGGATAAAGTTCTT | | | |
| | ACTCAAATGGGTTCACCTCATAATCCTATTTCA | | | |
| | TCTGTATCT | | | |
| CXCR1 | ATGTCAAATATTACAGATCCACAGATGTGGGAT | 28. | MSNITDPQMWDFDDLNFTGMPPADEDYSPCMLET | 57. |
| | TTTGATGATCTAAATTTCACTGGCATGCCACCT | | ETLNKYVVIIAYALVFLLSLLGNSLVMLVILYSR | |
| | GCAGATGAAGATTACAGCCCCTGTATGCTAGAA | | VGRSVTDVYLLNLALADLLFALTLPIWAASKVNG | |
| | ACTGAGACACTCAACAAGTATGTTGTGATCATC | | WIFGTFLCKVVSLLKEVNFYSGILLLACISVDRY | |
| | GCCTATGCCCTAGTGTTCCTGCTGAGCCTGCTG | | LAIVHATRTLTQKRHLVKFVCLGCWGLSMNLSLP | |
| | GGAAACTCCCTGGTGATGCTGGTCATCTTATAC | | FFLFRQAYHPNNSSPVCYEVLGNDTAKWRMVLRI | |
| | AGCAGGGTCGGCCGCTCCGTCACTGATGTCTAC | | LPHTFGFIVPLFVMLFCYGFTLRTLFKAHMGQKH | |
| | CTGCTGAACCTGGCCTTGGCCGACCTACTCTTT | | RAMRVIFAVVLIFLLCWLPYNLVLLADTLMRTQV | |
| | GCCCTGACCTTGCCCATCTGGGCCGCCTCCAAG | | IQESCERRNNIGRALDATEILGFLHSCLNPIIYA | |
| | GTGAATGGCTGGATTTTTGGCACATTCCTGTGC | | FIGQNFRHGFLKILAMHGLVSKEFLARHRVTSYT | |
| | AAGGTGGTCTCACTCCTGAAGGAAGTCAACTTC | | SSSVNVSSNL | |
| | TACAGTGGCATCCTGCTGTTGGCCTGCATCAGT | | | |
| | GTGGACCGTTACCTGGCCATTGTCCATGCCACA | | | |
| | CGCACACTGACCCAGAAGCGTCACTTGGTCAAG | | | |
| | TTTGTTTGTCTTGGCTGCTGGGGACTGTCTATG | | | |
| | AATCTGTCCCTGCCCTTCTTCCTTTTCCGCCAG | | | |
| | GCTTACCATCCAAACAATTCCAGTCCAGTTTGC | | | |
| | TATGAGGTCCTGGGAAATGACACAGCAAAATGG | | | |
| | CGGATGGTGTTGCGGATCCTGCCTCACACCTTT | | | |
| | GGCTTCATCGTGCCGCTGTTTGTCATGCTGTTC | | | |
| | TGCTATGGATTCACCCTGCGTACACTGTTTAAG | | | |
| | GCCCACATGGGGCAGAAGCACCGAGCCATGAGG | | | |
| | GTCATCTTTGCTGTCGTCCTCATCTTCCTGCTT | | | |
| | TGCTGGCTGCCCTACAACCTGGTCCTGCTGGCA | | | |
| | GACACCCTCATGAGGACCCAGGTGATCCAGGAG | | | |
| | AGCTGTGAGCGCCGCAACAACATCGGCCGGGCC | | | |
| | CTGGATGCCACTGAGATTCTGGGATTTCTCCAT | | | |
| | AGCTGCCTCAACCCCATCATCTACGCCTTCATC | | | |
| | GGCCAAAATTTTCGCCATGGATTCCTCAAGATC | | | |
| | CTGGCTATGCATGGCCTGGTCAGCAAGGAGTTC | | | |
| | TTGGCACGTCATCGTGTTACCTCCTACACTTCT | | | |
| | TCGTCTGTCAATGTCTCTTCCAACCTC | | | |
| ATF1 | ATGGAAGATTCCCACAAGAGTACCACGTCAGAG | 29. | MEDSHKSTTSETAPQPGSAVQGAHISHIAQQVSS | 58. |
| | ACAGCACCTCAACCTGGTTCAGCAGTTCAGGGA | | LSESEESQDSSDSIGSSQKAHGILARRPSYRKIL | |
| | GCTCACATTTCTCATATTGCTCAACAGGTATCA | | KDLSSEDTRGRKGDGENSGVSAAVTSMSVPTPIY | |
| | TCTTTATCAGAAAGTGAGGAGTCCCAGGACTCA | | QTSSGQYIAIAPNGALQLASPGTDGVQGLQTLTM | |
| | TCCGACAGCATAGGCTCCTCACAGAAAGCCCAC | | TNSGSTQQGTTILQYAQTSDGQQILVPSNQVVVQ | |
| | GGGATCCTAGCACGGCGCCCATCTTACAGAAAA | | TASGDMQTYQIRTTPSATSLPQTVVMTSPVTLTS | |
| | ATTTTGAAAGACTTATCTTCTGAAGATACACGG | | QTTKTDDPQLKREIRLMKNREAARECRRKKKEYV | |
| | GGCAGAAAAGGAGACGGAGAAAATTCTGGAGTT | | KCLENRVAVLENQNKTLIEELKTLKDLYSNKSV | |
| | TCTGCTGCTGTCACTTCTATGTCTGTTCCAACT | | | |
| | CCCATTGCCCCAAATGGAGCCTTACAGTTGGCA | | | |
| | AGTCCAGGCACAGATGGAGTACAGGGACTTCAG | | | |
| | ACATTAACCATGACAAATTCAGGCAGTACTCAG | | | |
| | CAAGGTACAACTATTCTTCAGTATGCACAGACC | | | |
| | TCTGATGGACAGCAGATACTTGTGCCCAGCAAT | | | |
| | CAGGTGGTCGTACAAACTGCATCAGGAGATATG | | | |
| | CAAACATATCAGATCCGAACTACACCTTCAGCT | | | |
| | ACTTCTCTGCCACAAACTGTGGTGATGACATCT | | | |
| | CCTGTGACTCTCACCTCTCAGACAACTAAGACA | | | |
| | GATGACCCCCAATTGAAAAGAGAAATAAGGTTA | | | |
| | ATGAAAAACAGAGAAGCTGCTCGAGAATGTCGC | | | |
| | AGAAAGAAGAAAGAATATGTGAAATGCCTGGAA | | | |
| | AACCGAGTTGCAGTCCTGGAAAATCAAAATAAA | | | |
| | ACTCTAATAGAAGAGTTAAAAACTTTGAAGGAT | | | |
| | CTTTATTCCAATAAAAGTGTT | | | |

TABLE 2

| | | nucleic acid sequence encoding | SEQ ID | amino acid sequence of | SEQ ID |
|---|---|---|---|---|---|
| Domain | Domain | polypeptide | NO: | polypeptide | NO: |
| TIM3 | 4-1BB | ATGTTTTCACATCTTCCCTTTGACTGTGTC | 59. | MFSHLPFDCVLLLLLLLLTRSSEVEYRAEV | 69. |
| (HAVR2) | (TNFRSF9) | CTGCTGCTGCTGCTGCTACTACTTACAAGG | | GQNAYLPCFYTPAAPGNLVPVCWGKGACPV | |
| | | TCCTCAGAAGTGGAATACAGAGCGGAGGTC | | FECGNVVLRTDERDVNYWTSRYWLNGDFRK | |
| | | GGTCAGAATGCCTATCTGCCCTGCTTCTAC | | GDVSLTIENVTLADSGIYCCRIQIPGIMND | |
| | | ACCCCAGCCGCCCCAGGGAACCTCGTGCCC | | EKENLKLVIKPAKVTPAPTRQRDFTAAFPR | |
| | | GTCTGCTGGGGCAAAGGAGCCTGTCCTGTG | | MLTTRGHGPAETQTLGSLPDINLTQISTLA | |
| | | TTTGAATGTGGCAACGTGGTGCTCAGGACT | | NELRDSRLANDLRPAPAREPGHSPQIISFF | |
| | | GATGAAAGGGATGTGAATTATTGGACATCC | | LALTSTALLFLLFFLTLRFSVVKRGRKKLL | |
| | | AGATACTGGCTAAATGGGGATTTCCGCAAA | | YIFKQPFMRPVQTTQEEDGCSCRFPEEEEG | |
| | | GGAGATGTGTCCCTGACCATAGAGAATGTG | | GCEL | |
| | | ACTCTAGCAGACAGTGGGATCTACTGCTGC | | | |
| | | CGGATCCAAATCCCAGGCATAATGAATGAT | | | |
| | | GAAAAATTTAACCTGAAGTTGGTCATCAAA | | | |
| | | CCAGCCAAGGTCACCCCTGCACCGACTCGG | | | |
| | | CAGAGAGACTTCACTGCAGCCTTTCCAAGG | | | |
| | | ATGCTTACCACCAGGGGACATGGCCCAGCA | | | |
| | | GAGACACAGACACTGGGGAGCCTCCCTGAT | | | |
| | | ATAAATCTAACACAAATATCCACATTGGCC | | | |
| | | AATGAGTTACGGGACTCTAGATTGGCCAAT | | | |
| | | GACTTACGGCCTGCCCCTGCGAGAGAGCCA | | | |
| | | GGACACTCTCCGCAGATCATCTCCTTCTTT | | | |
| | | CTTGCGCTGACGTCGACTGCGTTGCTCTTC | | | |
| | | CTGCTGTTCTTCCTCACGCTCCGTTTCTCT | | | |
| | | GTTGTTAAACGGGGCAGAAAGAAACTCCTG | | | |
| | | TATATATTCAAACAACCATTTATGAGACCA | | | |
| | | GTACAAACTACTCAAGAGGAAGATGGCTGT | | | |
| | | AGCTGCCGATTTCCAGAAGAAGAAGAAGGA | | | |
| | | GGATGTGAACTG | | | |
| TGFbR2 | MyD88 | ATGTTTTCACATCTTCCCTTTGACTGTGTC | 60. | MFSHLPFDCVLLLLLLLLTRSSEVEYRAEV | 70. |
| | | CTGCTGCTGCTGCTGCTACTACTTACAAGG | | GQNAYLPCFYTPAAPGNLVPVCWGKGACPV | |
| | | TCCTCAGAAGTGGAATACAGAGCGGAGGTC | | FECGNVVLRTDERDVNYWTSRYWLNGDFRK | |
| | | GGTCAGAATGCCTATCTGCCCTGCTTCTAC | | GDVSLTIENVTLADSGIYCCRIQIPGIMND | |
| | | ACCCCAGCCGCCCCAGGGAACCTCGTGCCC | | EKENLKLVIKPAKVTPAPTRQRDFTAAFPR | |
| | | GTCTGCTGGGGCAAAGGAGCCTGTCCTGTG | | MLTTRGHGPAETQTLGSLPDINLTQISTLA | |
| | | TTTGAATGTGGCAACGTGGTGCTCAGGACT | | NELRDSRLANDLRPAPAREPGHSPQIISFF | |
| | | GATGAAAGGGATGTGAATTATTGGACATCC | | LALTSTALLFLLFFLTLRFSVVKRGRKKLL | |
| | | AGATACTGGCTAAATGGGGATTTCCGCAAA | | YIFKQPFMRPVQTTQEEDGCSCRFPEEEEG | |
| | | GGAGATGTGTCCCTGACCATAGAGAATGTG | | GCEL | |
| | | ACTCTAGCAGACAGTGGGATCTACTGCTGC | | | |
| | | CGGATCCAAATCCCAGGCATAATGAATGAT | | | |
| | | GAAAAATTTAACCTGAAGTTGGTCATCAAA | | | |
| | | CCAGCCAAGGTCACCCCTGCACCGACTCGG | | | |
| | | CAGAGAGACTTCACTGCAGCCTTTCCAAGG | | | |
| | | ATGCTTACCACCAGGGGACATGGCCCAGCA | | | |
| | | GAGACACAGACACTGGGGAGCCTCCCTGAT | | | |
| | | ATAAATCTAACACAAATATCCACATTGGCC | | | |
| | | AATGAGTTACGGGACTCTAGATTGGCCAAT | | | |
| | | GACTTACGGCCTGCCCCTGCGAGAGAGCCA | | | |
| | | GGACACTCTCCGCAGATCATCTCCTTCTTT | | | |
| | | CTTGCGCTGACGTCGACTGCGTTGCTCTTC | | | |
| | | CTGCTGTTCTTCCTCACGCTCCGTTTCTCT | | | |
| | | GTTGTTAAACGGGGCAGAAAGAAACTCCTG | | | |
| | | TATATATTCAAACAACCATTTATGAGACCA | | | |
| | | GTACAAACTACTCAAGAGGAAGATGGCTGT | | | |
| | | AGCTGCCGATTTCCAGAAGAAGAAGAAGGA | | | |
| | | GGATGTGAACTG | | | |
| LTBR | IL-4R | ATGCTCCTGCCTTGGGCCACCTCTGCCCCC | 61. | MLLPWATSAPGLAWGPLVLGLFGLLAASQP | 71. |
| | (IIL4RA) | GGCCTGGCCTGGGGGCCTCTGGTGCTGGGC | | QAVPPYASENQTCRDQEKEYYEPQHRICCS | |
| | | CTCTTCGGGCTCCTGGCAGCATCGCAGCCC | | RCPPGTYVSAKCSRIRDTVCATCAENSYNE | |
| | | CAGGCGGTGCCTCCATATGCGTCGGAGAAC | | HWNYLTICQLCRPCDPVMGLEEIAPCTSKR | |
| | | CAGACCTGCAGGGACCAGGAAAAGGAATAC | | KTQCRCQPGMFCAAWALECTHCELLSDCPP | |
| | | TATGAGCCCCAGCACCGCATCTGCTGCTCC | | GTEAELKDEVGKGNNHCVPCKAGHFQNTSS | |
| | | CGCTGCCCGCCAGGCACCTATGTCTCAGCT | | PSARCQPHTRCENQGLVEAAPGTAQSDTTC | |
| | | AAATGTAGCCGCATCCGGGACACAGTTTGT | | KNPLEPLPPEMSGTMLMLAVLLPLAFFLLL | |
| | | GCCACATGTGCCGAGAATTCCTACAACGAG | | ATVFSCIWKSHPSLCKIKKEWWDQIPNPAR | |
| | | CACTGGAACTACCTGACCATCTGCCAGCTG | | SRLVAIIIQDAQGSQWEKRSRGQEPAKCPH | |
| | | TGCCGCCCCTGTGACCCAGTGATGGGCCTC | | WKNCLTKLLPCFLEHNMKRDEDPHKAAKEM | |
| | | GAGGAGATTGCCCCCTGCACAAGCAAACGG | | PFQGSGKSAWCPVEISKTVLWPESISVVRC | |
| | | AAGACCCAGTGCCGCTGCCAGCCGGGAATG | | VELFEAPVECEEEEEVEEEKGSFCASPESS | |
| | | TTCTGTGCTGCCTGGGCCCTCGAGTGTACA | | RDDFQEGREGIVARLTESLFLDLLGEENGG | |
| | | CACTGCGAGCTACTTTCTGACTGCCCGCCT | | FCQQDMGESCLLPPSGSTSAHMPWDEFPSA | |
| | | GGCACTGAAGCCGAGCTCAAAGATGAAGTT | | GPKEAPPWGKEQPLHLEPSPPASPTQSPDN | |
| | | GGGAAGGGTAACAACCACTGCGTCCCCTGC | | LTCTETPLVIAGNPAYRSFSNSLSQSPCPR | |
| | | AAGGCCGGGCACTTCCAGAATACCTCCTCC | | ELGPDPLLARHLEEVEPEMPCVPQLSEPTT | |
| | | CCCAGCGCCCGCTGCCAGCCCCACACCAGG | | VPQPEPETWEQILRRNVLQHGAAAAPVSAP | |
| | | TGTGAGAACCAAGGTCTGGTGGAGGCAGCT | | TSGYQEFVHAVEQGGTQASAVVGLGPPGEA | |
| | | CCAGGCACTGCCCAGTCCGACACAACCTGC | | GYKAFSSLLASSAVSPEKCGFGASSGEEGY | |
| | | AAAAATCCATTAGAGCCACTGCCCCCAGAG | | KPFQDLIPGCPGDPAPVPVPLFTFGLDREP | |
| | | ATGTCAGGAACCATGCTGATGCTGGCCGTT | | PRSPQSSHLPSSSPEHLGLEPGEKVEDMPK | |
| | | CTGCTGCCACTGGCCTTCTTTCTGCTCCTT | | PPLPQEQATDPLVDSLGSGIVYSALTCHLC | |
| | | GCCACCGTCTTCTCCTGCATCTGGAAGAGC | | GHLKQCHGQEDGGQTPVMASPCCGCCCGDR | |
| | | CACCCTTCTCTCTGCAAGATTAAGAAAGAA | | SSPPTTPLRAPDPSPGGVPLEASLCPASLA | |
| | | TGGTGGGATCAGATTCCCAACCCAGCCCGC | | PSGISEKSKSSSSFHPAPGNAQSSSQTPKI | |
| | | AGCCGCCTCGTGGCTATAATAATCCAGGAT | | VNFVSVGPTYMRVS | |
| | | GCTCAGGGGTCACAGTGGGAGAAGCGGTCC | | | |
| | | CGAGGCCAGGAACCAGCCAAGTGCCCACAC | | | |
| | | TGGAAGAATTGTCTTACCAAGCTCTTGCCC | | | |
| | | TGTTTTCTGGAGCACAACATGAAAAGGGAT | | | |
| | | GAAGATCCTCACAAGGCTGCCAAAGAGATG | | | |
| | | CCTTTCCAGGGCTCTGGAAAATCAGCATGG | | | |
| | | TGCCCAGTGGAGATCAGCAAGACAGTCCTC | | | |
| | | TGGCCAGAGAGCATCAGCGTGGTGCGATGT | | | |
| | | GTGGAGTTGTTTGAGGCCCCGGTGGAGTGT | | | |
| | | GAGGAGGAGGAGGAGGTAGAGGAAGAAAAA | | | |
| | | GGGAGCTTCTGTGCATCGCCTGAGAGCAGC | | | |
| | | AGGGATGACTTCCAGGAGGGAAGGGAGGGC | | | |
| | | ATTGTGGCCCGGCTAACAGAGAGCCTGTTC | | | |
| | | CTGGACCTGCTCGGAGAGGAGAATGGGGGC | | | |
| | | TTTTGCCAGCAGGACATGGGGGAGTCATGC | | | |
| | | CTTCTTCCACCTTCGGGAAGTACGAGTGCT | | | |
| | | CACATGCCCTGGGATGAGTTCCCAAGTGCA | | | |
| | | GGGCCCAAGGAGGCACCTCCCTGGGGCAAG | | | |
| | | GAGCAGCCTCTCCACCTGGAGCCAAGTCCT | | | |
| | | CCTGCCAGCCCGACCCAGAGTCCAGACAAC | | | |
| | | CTGACTTGCACAGAGACGCCCCTCGTCATC | | | |
| | | GCAGGCAACCCTGCTTACCGCAGCTTCAGC | | | |
| | | AACTCCCTGAGCCAGTCACCGTGTCCCAGA | | | |
| | | GAGCTGGGTCCAGACCCACTGCTGGCCAGA | | | |
| | | CACCTGGAGGAAGTAGAACCCGAGATGCCC | | | |
| | | TGTGTCCCCCAGCTCTCTGAGCCAACCACT | | | |
| | | GTGCCCCAACCTGAGCCAGAAACCTGGGAG | | | |
| | | CAGATCCTCCGCCGAAATGTCCTCCAGCAT | | | |
| | | GGGGCAGCTGCAGCCCCCGTCTCGGCCCCC | | | |
| | | ACCAGTGGCTATCAGGAGTTTGTACATGCG | | | |
| | | GTGGAGCAGGGTGGCACCCAGGCCAGTGCG | | | |
| | | GTGGTGGGCTTGGGTCCCCCAGGAGAGGCT | | | |
| | | GGTTACAAGGCCTTCTCAAGCCTGCTTGCC | | | |
| | | AGCAGTGCTGTGTCCCCAGAGAAATGTGGG | | | |
| | | TTTGGGGCTAGCAGTGGGGAAGAGGGGTAT | | | |
| | | AAGCCTTTCCAAGACCTCATTCCTGGCTGC | | | |
| | | CCTGGGGACCCTGCCCCAGTCCCTGTCCCC | | | |
| | | TTGTTCACCTTTGGACTGGACAGGGAGCCA | | | |
| | | CCTCGCAGTCCGCAGAGCTCACATCTCCCA | | | |
| | | AGCAGCTCCCCAGAGCACCTGGGTCTGGAG | | | |
| | | CCGGGGGAAAAGGTAGAGGACATGCCAAAG | | | |
| | | CCCCCACTTCCCCAGGAGCAGGCCACAGAC | | | |
| | | CCCCTTGTGGACAGCCTGGGCAGTGGCATT | | | |
| | | GTCTACTCAGCCCTTACCTGCCACCTGTGC | | | |
| | | GGCCACCTGAAACAGTGTCATGGCCAGGAG | | | |
| | | GATGGTGGCCAGACCCCTGTCATGGCCAGT | | | |
| | | CCTTGCTGTGGCTGCTGCTGTGGAGACAGG | | | |
| | | TCCTCGCCCCCTACAACCCCCCTGAGGGCC | | | |
| | | CCAGACCCCTCTCCAGGTGGGGTTCCACTG | | | |
| | | GAGGCCAGTCTGTGTCCGGCCTCCCTGGCA | | | |
| | | CCCTCGGGCATCTCAGAGAAGAGTAAATCC | | | |
| | | TCATCATCCTTCCATCCTGCCCCTGGCAAT | | | |
| | | GCTCAGAGCTCAAGCCAGACCCCCAAAATC | | | |
| | | GTGAACTTTGTCTCCGTGGGACCCACATAC | | | |
| | | ATGAGGGTCTCT | | | |
| IL4RA | 4-1BB | ATGGGGTGGCTTTGCTCTGGGCTCCTGTTC | 62. | MGWLCSGLLFPVSCLVLLQVASSGNMKVLQ | 72. |
| | | CCTGTGAGCTGCCTGGTCCTGCTGCAGGTG | | EPTCVSDYMSISTCEWKMNGPTNCSTELRL | |
| | | GCAAGCTCTGGGAACATGAAGGTCTTGCAG | | LYQLVFLLSEAHTCIPENNGGAGCVCHLLM | |
| | | GAGCCCACCTGCGTCTCCGACTACATGAGC | | DDVVSADNYTLDLWAGQQLLWKGSFKPSEH | |
| | | ATCTCTACTTGCGAGTGGAAGATGAATGGT | | VKPRAPGNLTVHTNVSDTLLLTWSNPYPPD | |
| | | CCCACCAATTGCAGCACCGAGCTCCGCCTG | | NYLYNHLTYAVNIWSENDPADFRIYNVTYL | |
| | | TTGTACCAGCTGGTTTTTCTGCTCTCCGAA | | EPSLRIAASTLKSGISYRARVRAWAQCYNT | |
| | | GCCCACACGTGTATCCCTGAGAACAACGGA | | TWSEWSPSTKWHNSYREPFEQHLIISFFLA | |
| | | GGCGCGGGGTGCGTGTGCCACCTGCTCATG | | LTSTALLFLLFFLTLRFSVVKRGRKKLLYI | |
| | | GATGACGTGGTCAGTGCGGATAACTATACA | | FKQPFMRPVQTTQEEDGCSCRFPEEEEGGC | |
| | | CTGGACCTGTGGGCTGGGCAGCAGCTGCTG | | EL | |
| | | TGGAAGGGCTCCTTCAAGCCCAGCGAGCAT | | | |
| | | GTGAAACCCAGGGCCCCAGGAAACCTGACA | | | |
| | | GTTCACACCAATGTCTCCGACACTCTGCTG | | | |
| | | CTGACCTGGAGCAACCCGTATCCCCCTGAC | | | |
| | | AATTACCTGTATAATCATCTCACCTATGCA | | | |
| | | GTCAACATTTGGAGTGAAAACGACCCGGCA | | | |
| | | GATTTCAGAATCTATAACGTGACCTACCTA | | | |
| | | GAACCCTCCCTCCGCATCGCAGCCAGCACC | | | |
| | | CTGAAGTCTGGGATTTCCTACAGGGCACGG | | | |
| | | GTGAGGGCCTGGGCTCAGTGCTATAACACC | | | |
| | | ACCTGGAGTGAGTGGAGCCCCAGCACCAAG | | | |
| | | TGGCACAACTCCTACAGGGAGCCCTTCGAG | | | |
| | | CAGCACCTCATCATCTCCTTCTTTCTTGCG | | | |
| | | CTGACGTCGACTGCGTTGCTCTTCCTGCTG | | | |
| | | TTCTTCCTCACGCTCCGTTTCTCTGTTGTT | | | |
| | | AAACGGGGCAGAAAGAAACTCCTGTATATA | | | |
| | | TTCAAACAACCATTTATGAGACCAGTACAA | | | |
| | | ACTACTCAAGAGGAAGATGGCTGTAGCTGC | | | |
| | | CGATTTCCAGAAGAAGAAGAAGGAGGATGT | | | |
| | | GAACTG | | | |
| 2B4 | 4-1BB | ATGCTGGGGCAAGTGGTCACCCTCATACTC | 63. | MLGQVVTLILLLLLKVYQGKGCQGSADHVV | 73. |
| (CD244) | | CTCCTGCTCCTCAAGGTGTATCAGGGCAAA | | SISGVPLQLQPNSIQTKVDSIAWKKLLPSQ | |
| | | GGATGCCAGGGATCAGCTGACCATGTGGTT | | NGFHHILKWENGSLPSNTSNDRFSFIVKNL | |
| | | AGCATCTCGGGAGTGCCTCTTCAGTTACAA | | SLLIKAAQQQDSGLYCLEVTSISGKVQTAT | |
| | | CCAAACAGCATACAGACGAAGGTTGACAGC | | FQVFVFESLLPDKVEKPRLQGQGKILDRGR | |
| | | ATTGCATGGAAGAAGTTGCTGCCCTCACAA | | CQVALSCLVSRDGNVSYAWYRGSKLIQTAG | |
| | | AATGGATTTCATCACATATTGAAGTGGGAG | | NLTYLDEEVDINGTHTYTCNVSNPVSWESH | |
| | | AATGGCTCTTTGCCTTCCAATACTTCCAAT | | TLNLTQDCONPAPAREPGHSPQIISFFLAL | |
| | | GATAGATTCAGTTTTATAGTCAAGAACTTG | | TSTALLFLLFFLTLRFSVVKRGRKKLLYIF | |
| | | AGTCTTCTCATCAAGGCAGCTCAGCAGCAG | | KQPFMRPVQTTQEEDGCSCRFPEEEEGGCE | |
| | | GACAGTGGCCTCTACTGCCTGGAGGTCACC | | L | |
| | | AGTATATCTGGAAAAGTTCAGACAGCCACG | | | |
| | | TTCCAGGTTTTTGTATTTGAATCTCTGCTT | | | |
| | | CCAGATAAAGTTGAGAAACCCCGCCTACAG | | | |
| | | GGGCAGGGGAAGATCCTGGACAGAGGGAGA | | | |
| | | TGCCAAGTGGCTCTGTCTTGCTTGGTCTCC | | | |
| | | AGGGATGGCAATGTGTCCTATGCTTGGTAC | | | |
| | | AGAGGGAGCAAGCTGATCCAGACAGCAGGG | | | |
| | | AACCTCACCTACCTGGACGAGGAGGTTGAC | | | |
| | | ATTAATGGCACTCACACATATACCTGCAAT | | | |
| | | GTCAGCAATCCTGTTAGCTGGGAAAGCCAC | | | |
| | | ACCCTGAATCTCACTCAGGACTGTCAGAAT | | | |
| | | CCTGCCCCTGCGAGAGAGCCAGGACACTCT | | | |
| | | CCGCAGATCATCTCCTTCTTTCTTGCGCTG | | | |
| | | ACGTCGACTGCGTTGCTCTTCCTGCTGTTC | | | |
| | | TTCCTCACGCTCCGTTTCTCTGTTGTTAAA | | | |
| | | CGGGGCAGAAAGAAACTCCTGTATATATTC | | | |
| | | AAACAACCATTTATGAGACCAGTACAAACT | | | |
| | | ACTCAAGAGGAAGATGGCTGTAGCTGCCGA | | | |
| | | TTTCCAGAAGAAGAAGAAGGAGGATGTGAA | | | |
| | | CTG | | | |
| VISTA | CD28 | ATGGGCGTCCCCACGGCCCTGGAGGCCGGC | 64. | MGVPTALEAGSWRWGSLLFALFLAASLGPV | 74. |
| (VSIR) | | AGCTGGCGCTGGGGATCCCTGCTCTTCGCT | | AAFKVATPYSLYVCPEGQNVTLTCRLLGPV | |
| | | CTCTTCCTGGCTGCGTCCCTAGGTCCGGTG | | DKGHDVTFYKTWYRSSRGEVQTCSERRPIR | |
| | | GCAGCCTTCAAGGTCGCCACGCCGTATTCC | | NLTFQDLHLHHGGHQAANTSHDLAQRHGLE | |
| | | CTGTATGTCTGTCCCGAGGGGCAGAACGTC | | SASDHHGNFSITMRNLTLLDSGLYCCLVVE | |
| | | ACCCTCACCTGCAGGCTCTTGGGCCCTGTG | | IRHHHSEHRVHGAMELQVQTGKDAPSNCVV | |
| | | GACAAAGGGCACGATGTGACCTTCTACAAG | | YPSSSCPSPLFPGPSKPFWVLVVVGGVLAC | |
| | | ACGTGGTACCGCAGCTCGAGGGGCGAGGTG | | YSLLVTVAFIIFWVRSKRSRLLHSDYMNMT | |
| | | CAGACCTGCTCAGAGCGCCGGCCCATCCGC | | PRRPGPTRKHYQPYAPPRDFAAYRS | |
| | | AACCTCACGTTCCAGGACCTTCACCTGCAC | | | |
| | | CATGGAGGCCACCAGGCTGCCAACACCAGC | | | |
| | | CACGACCTGGCTCAGCGCCACGGGCTGGAG | | | |
| | | TCGGCCTCCGACCACCATGGCAACTTCTCC | | | |
| | | ATCACCATGCGCAACCTGACCCTGCTGGAT | | | |
| | | AGCGGCCTCTACTGCTGCCTGGTGGTGGAG | | | |
| | | ATCAGGCACCACCACTCGGAGCACAGGGTC | | | |
| | | CATGGTGCCATGGAGCTGCAGGTGCAGACA | | | |
| | | GGCAAAGATGCACCATCCAACTGTGTGGTG | | | |
| | | TACCCATCCTCCTCCTGTCCAAGTCCCCTA | | | |
| | | TTTCCCGGACCTTCTAAGCCCTTTTGGGTG | | | |
| | | CTGGTGGTGGTTGGTGGAGTCCTGGCTTGC | | | |
| | | TATAGCTTGCTAGTAACAGTGGCCTTTATT | | | |
| | | ATTTTCTGGGTGAGGAGTAAGAGGAGCAGG | | | |
| | | CTCCTGCACAGTGACTACATGAACATGACT | | | |
| | | CCCCGCCGCCCCGGGCCCACCCGCAAGCAT | | | |
| | | TACCAGCCCTATGCCCCACCACGCGACTTC | | | |
| | | GCAGCCTATCGCTCC | | | |
| TNFRSF12 | OX40 | ATGGCCCGCGGAAGTCTTCGCCGTTTGCTC | 65. | MARGSLRRLLRLLVLGLWLALLRSVAGEQA | 75. |
| (TNFRSF12A) | (TNFRSF4) | CGTCTTCTTGTTCTCGGCCTGTGGCTCGCT | | PGTAPCSRGSSWSADLDKCMDCASCRARPH | |
| | | CTGCTCCGGAGTGTAGCGGGCGAACAAGCA | | SDFCLGCAAAPPAPFRLLWPILGGALSLTF | |
| | | CCTGGGACTGCACCGTGTTCACGGGGCTCC | | VLGLLSGFLVWRRCRRREALYLLRRDQRLP | |
| | | TCATGGTCCGCCGATCTTGATAAATGTATG | | PDAHKPPGGGSFRTPIQEEQADAHSTLAKI | |
| | | GATTGTGCCAGCTGTAGAGCCAGGCCCCAT | | | |
| | | TCTGATTTCTGTCTTGGGTGTGCCGCTGCC | | | |
| | | CCACCGGCACCTTTTAGACTTCTGTGGCCT | | | |
| | | ATTCTGGGCGGAGCCCTCTCATTGACATTT | | | |
| | | GTCCTTGGACTTCTCTCCGGGTTCCTTGTA | | | |
| | | TGGCGGCGGTGTCGGCGCCGCGAAgccctg | | | |
| | | tACCTGCTCCGGAGGGACCAGAGGCTGCCC | | | |
| | | CCCGATGCCCACAAGCCCCCTGGGGGAGGC | | | |
| | | AGTTTCCGGACCCCCATCCAAGAGGAGCAG | | | |
| | | GCCGACGCCCACTCCACCCTGGCCAAGATC | | | |
| LTBR | CD28 | ATGCTCCTGCCTTGGGCCACCTCTGCCCCC | 66. | MLLPWATSAPGLAWGPLVLGLFGLLAASQP | 76. |
| | | GGCCTGGCCTGGGGGCCTCTGGTGCTGGGC | | QAVPPYASENQTCRDQEKEYYEPQHRICCS | |
| | | CTCTTCGGGCTCCTGGCAGCATCGCAGCCC | | RCPPGTYVSAKCSRIRDTVCATCAENSYNE | |
| | | CAGGCGGTGCCTCCATATGCGTCGGAGAAC | | HWNYLTICQLCRPCDPVMGLEEIAPCTSKR | |
| | | CAGACCTGCAGGGACCAGGAAAAGGAATAC | | KTQCRCQPGMFCAAWALECTHCELLSDCPP | |
| | | TATGAGCCCCAGCACCGCATCTGCTGCTCC | | GTEAELKDEVGKGNNHCVPCKAGHFQNTSS | |
| | | CGCTGCCCGCCAGGCACCTATGTCTCAGCT | | PSARCQPHTRCENQGLVEAAPGTAQSDTTC | |
| | | AAATGTAGCCGCATCCGGGACACAGTTTGT | | KNPLEPLPPEMSGTMLMLAVLLPLAFFLLL | |
| | | GCCACATGTGCCGAGAATTCCTACAACGAG | | ATVFSCIWKSHPSLCRSKRSRLLHSDYMNM | |
| | | CACTGGAACTACCTGACCATCTGCCAGCTG | | TPRRPGPTRKHYQPYAPPRDFAAYRS | |
| | | TGCCGCCCCTGTGACCCAGTGATGGGCCTC | | | |
| | | GAGGAGATTGCCCCCTGCACAAGCAAACGG | | | |
| | | AAGACCCAGTGCCGCTGCCAGCCGGGAATG | | | |
| | | TTCTGTGCTGCCTGGGCCCTCGAGTGTACA | | | |
| | | CACTGCGAGCTACTTTCTGACTGCCCGCCT | | | |
| | | GGCACTGAAGCCGAGCTCAAAGATGAAGTT | | | |
| | | GGGAAGGGTAACAACCACTGCGTCCCCTGC | | | |
| | | AAGGCCGGGCACTTCCAGAATACCTCCTCC | | | |
| | | CCCAGCGCCCGCTGCCAGCCCCACACCAGG | | | |
| | | TGTGAGAACCAAGGTCTGGTGGAGGCAGCT | | | |
| | | CCAGGCACTGCCCAGTCCGACACAACCTGC | | | |
| | | AAAAATCCATTAGAGCCACTGCCCCCAGAG | | | |
| | | ATGTCAGGAACCATGCTGATGCTGGCCGTT | | | |
| | | CTGCTGCCACTGGCCTTCTTTCTGCTCCTT | | | |
| | | GCCACCGTCTTCTCCTGCATCTGGAAGAGC | | | |
| | | CACCCTTCTCTCTGCAGGAGTAAGAGGAGC | | | |
| | | AGGCTCCTGCACAGTGACTACATGAACATG | | | |
| | | ACTCCCCGCCGCCCCGGGCCCACCCGCAAG | | | |
| | | CATTACCAGCCCTATGCCCCACCACGCGAC | | | |
| | | TTCGCAGCCTATCGCTCC | | | |
| LTBR | MyD88 | ATGCTCCTGCCTTGGGCCACCTCTGCCCCC | 67. | MLLPWATSAPGLAWGPLVLGLFGLLAASQP | 77. |
| | | GGCCTGGCCTGGGGGCCTCTGGTGCTGGGC | | QAVPPYASENQTCRDQEKEYYEPQHRICCS | |
| | | CTCTTCGGGCTCCTGGCAGCATCGCAGCCC | | RCPPGTYVSAKCSRIRDTVCATCAENSYNE | |
| | | CAGGCGGTGCCTCCATATGCGTCGGAGAAC | | HWNYLTICQLCRPCDPVMGLEEIAPCTSKR | |
| | | CAGACCTGCAGGGACCAGGAAAAGGAATAC | | KTQCRCQPGMFCAAWALECTHCELLSDCPP | |
| | | TATGAGCCCCAGCACCGCATCTGCTGCTCC | | GTEAELKDEVGKGNNHCVPCKAGHFQNTSS | |
| | | CGCTGCCCGCCAGGCACCTATGTCTCAGCT | | PSARCQPHTRCENQGLVEAAPGTAQSDTTC | |
| | | AAATGTAGCCGCATCCGGGACACAGTTTGT | | KNPLEPLPPEMSGTMLMLAVLLPLAFFLLL | |
| | | GCCACATGTGCCGAGAATTCCTACAACGAG | | ATVFSCIWKSHPSLCTQVAADWTALAEEMD | |
| | | CACTGGAACTACCTGACCATCTGCCAGCTG | | FEYLEIRQLETQADPTGRLLDAWQGRPGAS | |
| | | TGCCGCCCCTGTGACCCAGTGATGGGCCTC | | VGRLLELLTKLGRDDVLLELGPSIEEDCQK | |
| | | GAGGAGATTGCCCCCTGCACAAGCAAACGG | | YILKQQQEEAEKPLQVAAVDSSVPRTA | |
| | | AAGACCCAGTGCCGCTGCCAGCCGGGAATG | | | |
| | | TTCTGTGCTGCCTGGGCCCTCGAGTGTACA | | | |
| | | CACTGCGAGCTACTTTCTGACTGCCCGCCT | | | |
| | | GGCACTGAAGCCGAGCTCAAAGATGAAGTT | | | |
| | | GGGAAGGGTAACAACCACTGCGTCCCCTGC | | | |
| | | AAGGCCGGGCACTTCCAGAATACCTCCTCC | | | |
| | | CCCAGCGCCCGCTGCCAGCCCCACACCAGG | | | |
| | | TGTGAGAACCAAGGTCTGGTGGAGGCAGCT | | | |
| | | CCAGGCACTGCCCAGTCCGACACAACCTGC | | | |
| | | AAAAATCCATTAGAGCCACTGCCCCCAGAG | | | |
| | | ATGTCAGGAACCATGCTGATGCTGGCCGTT | | | |
| | | CTGCTGCCACTGGCCTTCTTTCTGCTCCTT | | | |
| | | GCCACCGTCTTCTCCTGCATCTGGAAGAGC | | | |
| | | CACCCTTCTCTCTGCACACAGGTGGCGGCC | | | |
| | | GACTGGACCGCGCTGGCGGAGGAGATGGAC | | | |
| | | TTTGAGTACTTGGAGATCCGGCAACTGGAG | | | |
| | | ACACAAGCGGACCCCACTGGCAGGCTGCTG | | | |
| | | GACGCCTGGCAGGGACGCCCTGGCGCCTCT | | | |
| | | GTAGGCCGACTGCTCGAGCTGCTTACCAAG | | | |
| | | CTGGGCCGCGACGACGTGCTGCTGGAGCTG | | | |
| | | GGACCCAGCATTGAGGAGGATTGCCAAAAG | | | |
| | | TATATCTTGAAGCAGCAGCAGGAGGAGGCT | | | |
| | | GAGAAGCCTTTACAGGTGGCCGCTGTAGAC | | | |
| | | AGCAGTGTCCCACGGACAGCA | | | |
| BTLA4 | 4-1BB | ATGAAGACATTGCCTGCCATGCTTGGAACT | 68. | MKTLPAMLGTGKLFWVFFLIPYLDIWNIHG | 78. |
| | | GGGAAATTATTTTGGGTCTTCTTCTTAATC | | KESCDVQLYIKRQSEHSILAGDPFELECPV | |
| | | CCATATCTGGACATCTGGAACATCCATGGG | | KYCANRPHVTWCKLNGTTCVKLEDRQTSWK | |
| | | AAAGAATCATGTGATGTACAGCTTTATATA | | EEKNISFFILHFEPVLPNDNGSYRCSANFQ | |
| | | AAGAGACAATCTGAACACTCCATCTTAGCA | | SNLIESHSTTLYVTDVKSASERPSKDEMPA | |
| | | GGAGATCCCTTTGAACTAGAATGCCCTGTG | | PAREPGHSPQIISFFLALTSTALLFLLFFL | |
| | | AAATACTGTGCTAACAGGCCTCATGTGACT | | TLRFSVVKRGRKKLLYIFKQPFMRPVQTTQ | |
| | | TGGTGCAAGCTCAATGGAACAACATGTGTA | | EEDGCSCRFPEEEEGGCEL | |
| | | AAACTTGAAGATAGACAAACAAGTTGGAAG | | | |
| | | GAAGAGAAGAACATTTCATTTTTCATTCTA | | | |
| | | CATTTTGAACCAGTGCTTCCTAATGACAAT | | | |
| | | GGGTCATACCGCTGTTCTGCAAATTTTCAG | | | |
| | | TCTAATCTCATTGAAAGCCACTCAACAACT | | | |
| | | CTTTATGTGACAGATGTAAAAAGTGCCTCA | | | |
| | | GAACGACCCTCCAAGGACGAAATGCCTGCC | | | |
| | | CCTGCGAGAGAGCCAGGACACTCTCCGCAG | | | |
| | | ATCATCTCCTTCTTTCTTGCGCTGACGTCG | | | |
| | | ACTGCGTTGCTCTTCCTGCTGTTCTTCCTC | | | |
| | | ACGCTCCGTTTCTCTGTTGTTAAACGGGGC | | | |
| | | AGAAAGAAACTCCTGTATATATTCAAACAA | | | |
| | | CCATTTATGAGACCAGTACAAACTACTCAA | | | |
| | | GAGGAAGATGGCTGTAGCTGCCGATTTCCA | | | |
| | | GAAGAAGAAGAAGGAGGATGTGAACTG | | | |
| DR5 | ICOS | ATGGAACAACGGGGACAGAACGCCCCGGCC | 163 | MEQRGQNAPAASGARKRHGPGPREARGARP | 160 |
| | | GCTTCGGGGGCCCGGAAAAGGCACGGCCCA | | GPRVPKTLVLVVAAVLLLVSAESALITQQD | |
| | | GGACCCAGGGAGGCGCGGGGAGCCAGGCCT | | LAPQQRAAPQQKRSSPSEGLCPPGHHISED | |
| | | GGGCCCCGGGTCCCCAAGACCCTTGTGCTC | | GRDCISCKYGQDYSTHWNDLLFCLRCTRCD | |
| | | GTTGTCGCCGCGGTCCTGCTGTTGGTCTCA | | SGEVELSPCTTTRNTVCQCEEGTFREEDSP | |
| | | GCTGAGTCTGCTCTGATCACCCAACAAGAC | | EMCRKCRTGCPRGMVKVGDCTPWSDIECVH | |
| | | CTAGCTCCCCAGCAGAGAGCGGCCCCACAA | | KESGTKHSGEVPAVEETVTSSPGTPASPCS | |
| | | CAAAAGAGGTCCAGCCCCTCAGAGGGATTG | | LSGIIIGVTVAAVVLIVAVFVCKSLLWKCW | |
| | | TGTCCACCTGGACACCATATCTCAGAAGAC | | LTKKKYSSSVHDPNGEYMFMRAVNTAKKSR | |
| | | GGTAGAGATTGCATCTCCTGCAAATATGGA | | LTDVTL | |
| | | CAGGACTATAGCACTCACTGGAATGACCTC | | | |
| | | CTTTTCTGCTTGCGCTGCACCAGGTGTGAT | | | |
| | | TCAGGTGAAGTGGAGCTAAGTCCCTGCACC | | | |
| | | ACGACCAGAAACACAGTGTGTCAGTGCGAA | | | |
| | | GAAGGCACCTTCCGGGAAGAAGATTCTCCT | | | |
| | | GAGATGTGCCGGAAGTGCCGCACAGGGTGT | | | |
| | | CCCAGAGGGATGGTCAAGGTCGGTGATTGT | | | |
| | | ACACCCTGGAGTGACATCGAATGTGTCCAC | | | |
| | | AAAGAATCAGGTACAAAGCACAGTGGGGAA | | | |
| | | GTCCCAGCTGTGGAGGAGACGGTGACCTCC | | | |
| | | AGCCCAGGGACTCCTGCCTCTCCCTGTTCT | | | |
| | | CTCTCAGGCATCATCATAGGAGTCACAGTT | | | |
| | | GCAGCCGTAGTCTTGATTGTGGCTGTGTTT | | | |
| | | GTTTGCAAGTCTTTACTGTGGAAGTGTTGG | | | |
| | | CTTACAAAAAAGAAGTATTCATCCAGTGTG | | | |
| | | CACGACCCTAACGGTGAATACATGTTCATG | | | |
| | | AGAGCAGTGAACACAGCCAAAAAATCTAGA | | | |
| | | CTCACAGATGTGACCCTA | | | |
| TIGIT | ICOS | ATGCGCTGGTGTCTCCTCCTGATCTGGGCC | 164 | MRWCLLLIWAQGLRQAPLASGMMTGTIETT | 161 |
| | | CAGGGGCTGAGGCAGGCTCCCCTCGCCTCA | | GNISAEKGGSIILQCHLSSTTAQVTQVNWE | |
| | | GGAATGATGACAGGCACAATAGAAACAACG | | QQDQLLAICNADLGWHISPSFKDRVAPGPG | |
| | | GGGAACATTTCTGCAGAGAAAGGTGGCTCT | | LGLTLQSLTVNDTGEYFCIYHTYPDGTYTG | |
| | | ATCATCTTACAATGTCACCTCTCCTCCACC | | RIFLEVLESSVAHIYESQLCCQLKFWLPIG | |
| | | ACGGCACAAGTGACCCAGGTCAACTGGGAG | | CAAFVVVCILGCILICWLTKKKYSSSVHDP | |
| | | CAGCAGGACCAGCTTCTGGCCATTTGTAAT | | NGEYMFMRAVNTAKKSRLTDVTL | |
| | | GCTGACTTGGGGTGGCACATCTCCCCATCC | | | |
| | | TTCAAGGATCGAGTGGCCCCAGGTCCCGGC | | | |
| | | CTGGGCCTCACCCTCCAGTCGCTGACCGTG | | | |
| | | AACGATACAGGGGAGTACTTCTGCATCTAT | | | |
| | | CACACCTACCCTGATGGGACGTACACTGGG | | | |
| | | AGAATCTTCCTGGAGGTCCTAGAAAGCTCA | | | |
| | | GTGGCTCATATTTATGAATCACAACTTTGT | | | |
| | | TGCCAGCTGAAGTTCTGGTTACCCATAGGA | | | |
| | | TGTGCAGCCTTTGTTGTAGTCTGCATTTTG | | | |
| | | GGATGCATACTTATTTGTTGGCTTACAAAA | | | |
| | | AAGAAGTATTCATCCAGTGTGCACGACCCT | | | |
| | | AACGGTGAATACATGTTCATGAGAGCAGTG | | | |
| | | AACACAGCCAAAAAATCTAGACTCACAGAT | | | |
| | | GTGACCCTA | | | |
| LTBR | 4-1BB | ATGCTCCTGCCTTGGGCCACCTCTGCCCCC | 165 | MLLPWATSAPGLAWGPLVLGLFGLLAASQP | 162 |
| | | GGCCTGGCCTGGGGGCCTCTGGTGCTGGGC | | QAVPPYASENQTCRDQEKEYYEPQHRICCS | |
| | | CTCTTCGGGCTCCTGGCAGCATCGCAGCCC | | RCPPGTYVSAKCSRIRDTVCATCAENSYNE | |
| | | CAGGCGGTGCCTCCATATGCGTCGGAGAAC | | HWNYLTICQLCRPCDPVMGLEEIAPCTSKR | |
| | | CAGACCTGCAGGGACCAGGAAAAGGAATAC | | KTQCRCQPGMFCAAWALECTHCELLSDCPP | |
| | | TATGAGCCCCAGCACCGCATCTGCTGCTCC | | GTEAELKDEVGKGNNHCVPCKAGHFQNTSS | |
| | | CGCTGCCCGCCAGGCACCTATGTCTCAGCT | | PSARCQPHTRCENQGLVEAAPGTAQSDTTC | |
| | | AAATGTAGCCGCATCCGGGACACAGTTTGT | | KNPLEPLPPEMSGTMLMLAVLLPLAFFLLL | |
| | | GCCACATGTGCCGAGAATTCCTACAACGAG | | ATVFSCIWKSHPSLCKRGRKKLLYIFKQPF | |
| | | CACTGGAACTACCTGACCATCTGCCAGCTG | | MRPVQTTQEEDGCSCRFPEEEEGGCEL | |
| | | TGCCGCCCCTGTGACCCAGTGATGGGCCTC | | | |
| | | GAGGAGATTGCCCCCTGCACAAGCAAACGG | | | |
| | | AAGACCCAGTGCCGCTGCCAGCCGGGAATG | | | |
| | | TTCTGTGCTGCCTGGGCCCTCGAGTGTACA | | | |
| | | CACTGCGAGCTACTTTCTGACTGCCCGCCT | | | |
| | | GGCACTGAAGCCGAGCTCAAAGATGAAGTT | | | |
| | | GGGAAGGGTAACAACCACTGCGTCCCCTGC | | | |
| | | AAGGCCGGGCACTTCCAGAATACCTCCTCC | | | |
| | | CCCAGCGCCCGCTGCCAGCCCCACACCAGG | | | |
| | | TGTGAGAACCAAGGTCTGGTGGAGGCAGCT | | | |
| | | CCAGGCACTGCCCAGTCCGACACAACCTGC | | | |
| | | AAAAATCCATTAGAGCCACTGCCCCCAGAG | | | |
| | | ATGTCAGGAACCATGCTGATGCTGGCCGTT | | | |
| | | CTGCTGCCACTGGCCTTCTTTCTGCTCCTT | | | |
| | | GCCACCGTCTTCTCCTGCATCTGGAAGAGC | | | |
| | | CACCCTTCTCTCTGCAAACGGGGCAGAAAG | | | |
| | | AAACTCCTGTATATATTCAAACAACCATTT | | | |
| | | ATGAGACCAGTACAAACTACTCAAGAGGAA | | | |
| | | GATGGCTGTAGCTGCCGATTTCCAGAAGAA | | | |
| | | GAAGAAGGAGGATGTGAACTG | | | |

TABLE 3

| | | | Signal | | | SEQ | | SEQ | | |
|---|---|---|---|---|---|---|---|---|---|---|
| Gene name | Full amino acid sequence | SEQ ID NO: | Peptide | SEQ ID NO: | EC domain | ID NO: | TM domain | ID NO: | IC domain | SEQ ID NO: |
| TIM-3 | MFSHLPFDCVLLLLLLLL | 93. | MFSHLPFD | 166. | SEVEYRAEVGQ | 168. | SEVEYRA | 170. | FKWYSHSKE | 172. |
| | TRSSEVEYRAEVGQNAY | | CVLLLLLL | | NAYLPCFYTPA | | EVGQNAY | | KIQNLSLISL | |
| | LPCFYTPAAPGNLVPVC | | LLTRS | | APGNLVPVCW | | LPCFYTPA | | ANLPPSGLA | |
| | WGKGACPVFECGNVVLR | | | | GKGACPVFECG | | APGNLVP | | NAVAEGIRS | |
| | TDERDVNYWTSRYWLN | | | | NVVLRTDERD | | VCWGKG | | EENIYTIEEN | |
| | GDFRKGDVSLTIENVTLA | | | | VNYWTSRYWL | | ACPVFEC | | VYEVEEPNE | |
| | DSGIYCCRIQIPGIMNDEK | | | | NGDFRKGDVSL | | GNVVLRT | | YYCYVSSRQ | |
| | FNLKLVIKPAKVTPAPTR | | | | TIENVTLADSGI | | DERDVNY | | QPSQPLGCR | |
| | QRDFTAAFPRMLTTRGH | | | | YCCRIQIPGIMN | | WTSRYWL | | FAMP | |
| | GPAETQTLGSLPDINLTQI | | | | DEKFNLKLVIK | | NGDFRKG | | | |
| | STLANELRDSRLANDLRD | | | | PAKVTPAPTRQ | | DVSLTIEN | | | |
| | SGATIRIGIYIGAGICAGL | | | | RDFTAAFPRML | | VTLADSGI | | | |
| | ALALIFGALIFKWYSHSK | | | | TTRGHGPAETQ | | YCCRIQIP | | | |
| | EKIQNLSLISLANLPPSGL | | | | TLGSLPDINLTQ | | GIMNDEK | | | |
| | ANAVAEGIRSEENIYTIEE | | | | ISTLANELRDSR | | FNLKLVIK | | | |
| | NVYEVEEPNEYYCYVSS | | | | LANDLRDSGAT | | PAKVTPAP | | | |
| | RQQPSQPLGCRFAMP | | | | IRIG | | TRQRDFT | | | |
| | | | | | | | AAFPRML | | | |
| | | | | | | | TTRGHGP | | | |
| | | | | | | | AETQTLGS | | | |
| | | | | | | | LPDINLTQ | | | |
| | | | | | | | ISTLANEL | | | |
| | | | | | | | RDSRLAN | | | |
| | | | | | | | DLRDSGA | | | |
| | | | | | | | TIRIG | | | |
| 4-1BB | MGNSCYNIVATLLLVLNF | 94. | MGNSCYN | 167. | LQDPCSNCPAG | 169. | LQDPCSN | 171. | KRGRKKLL | 173. |
| | ERTRSLQDPCSNCPAGTF | | IVATLLLV | | TFCDNNRNQIC | | CPAGTFC | | YIFKQPFMR | |
| | CDNNRNQICSPCPPNSFSS | | LNFERTRS | | SPCPPNSFSSAG | | DNNRNQI | | PVQTTQEED | |
| | AGGQRTCDICRQCKGVF | | | | GQRTCDICRQC | | CSPCPPNS | | GCSCRFPEE | |
| | RTRKECSSTSNAECDCTP | | | | KGVFRTRKECS | | FSSAGGQ | | EEGGCEL | |
| | GFHCLGAGCSMCEQDCK | | | | STSNAECDCTP | | RTCDICRQ | | | |
| | QGQELTKKGCKDCCFGT | | | | GFHCLGAGCS | | CKGVFRT | | | |
| | FNDQKRGICRPWTNCSLD | | | | MCEQDCKQGQ | | RKECSSTS | | | |
| | GKSVLVNGTKERDVVCG | | | | ELTKKGCKDCC | | NAECDCT | | | |
| | PSPADLSPGASSVTPPAPA | | | | FGTFNDQKRGI | | PGFHCLG | | | |
| | REPGHSPQIISFFLALTSTA | | | | CRPWTNCSLDG | | AGCSMCE | | | |
| | LLFLLFFLTLRFSVVKRG | | | | KSVLVNGTKER | | QDCKQGQ | | | |
| | RKKLLYIFKQPFMRPVQT | | | | DVVCGPSPADL | | ELTKKGC | | | |
| | TQEEDGCSCRFPEEEEGG | | | | SPGASSVTPPAP | | KDCCFGT | | | |
| | CEL | | | | AREPGHSPQ | | FNDQKRGI | | | |
| | | | | | | | CRPWTNC | | | |
| | | | | | | | SLDGKSV | | | |
| | | | | | | | LVNGTKE | | | |
| | | | | | | | RDVVCGP | | | |
| | | | | | | | SPADLSPG | | | |
| | | | | | | | ASSVTPPA | | | |
| | | | | | | | PAREPGHS | | | |
| | | | | | | | PQ | | | |
| TIGIT | MRWCLLLIWAQGLRQAP | 95. | MRWCLLL | 106. | MMTGTIETTGN | 119. | MMTGTIE | 132. | LTRKKKALR | 145. |
| | LASGMMTGTIETTGNISA | | IWAQGLR | | ISAEKGGSIILQ | | TTGNISAE | | IHSVEGDLR | |
| | EKGGSIILQCHLSSTTAQV | | QAPLASG | | CHLSSTTAQVT | | KGGSIILQ | | RKSAGQEE | |
| | TQVNWEQQDQLLAICNA | | | | QVNWEQQDQL | | CHLSSTTA | | WSPSAPSPP | |
| | DLGWHISPSFKDRVAPGP | | | | LAICNADLGW | | QVTQVNW | | GSCVQAEA | |
| | GLGLTLQSLTVNDTGEYF | | | | HISPSFKDRVAP | | EQQDQLL | | APAGLCGEQ | |
| | CIYHTYPDGTYTGRIFLEV | | | | GPGLGLTLQSL | | AICNADL | | RGEDCAELH | |
| | LESSVAEHGARFQIPLLG | | | | TVNDTGEYFCI | | GWHISPSF | | DYFNVLSYR | |
| | AMAATLVVICTAVIVVV | | | | YHTYPDGTYTG | | KDRVAPG | | SLGNCSFFT | |
| | ALTRKKKALRIHSVEGDL | | | | RIFLEVLESSVA | | PGLGLTLQ | | ETG | |
| | RRKSAGQEEWSPSAPSPP | | | | EHGARFQIP | | SLTVNDT | | | |
| | GSCVQAEAAPAGLCGEQ | | | | | | GEYFCIYH | | | |
| | RGEDCAELHDYFNVLSY | | | | | | TYPDGTY | | | |
| | RSLGNCSFFTETG | | | | | | TGRIFLEV | | | |
| | | | | | | | LESSVAEH | | | |
| | | | | | | | GARFQIP | | | |
| TGFbR2 | MGRGLLRGLWPLHIVLW | 96. | MGRGLLR | 107. | TIPPHVQKSVN | 120. | TIPPHVQK | 133. | CYRVNRQQ | 146. |
| | TRIASTIPPHVQKSVNND | | GLWPLHI | | NDMIVTDNNG | | SVNNDMI | | KLSSTWETG | |
| | MIVTDNNGAVKFPQLCK | | VLWTRIAS | | AVKFPQLCKFC | | VTDNNGA | | KTRKLMEFS | |
| | FCDVRFSTCDNQKSCMS | | | | DVRFSTCDNQK | | VKFPQLC | | EHCAIILEDD | |
| | NCSITSICEKPQEVCVAV | | | | SCMSNCSITSIC | | KFCDVRFS | | RSDISSTCAN | |
| | WRKNDENITLETVCHDP | | | | EKPQEVCVAV | | TCDNQKS | | NINHNTELL | |
| | KLPYHDFILEDAASPKCI | | | | WRKNDENITLE | | CMSNCSIT | | PIELDTLVG | |
| | MKEKKKPGETFFMCSCSS | | | | TVCHDPKLPYH | | SICEKPQE | | KGRFAEVY | |
| | DECNDNIIFSEEYNTSNPD | | | | DFILEDAASPK | | VCVAVWR | | KAKLKQNTS | |
| | LLLVIFQVTGISLLPPLGV | | | | CIMKEKKKPGE | | KNDENITL | | EQFETVAVK | |
| | AISVIIIFYCYRVNRQQKL | | | | TFFMCSCSSDE | | ETVCHDP | | IFPYEEYAS | |
| | SSTWETGKTRKLMEFSEH | | | | CNDNIIFSEEYN | | KLPYHDFI | | WKTEKDIFS | |
| | CAIILEDDRSDISSTCANNI | | | | TSNPDLLLVIFQ | | LEDAASP | | DINLKHENIL | |
| | NHNTELLPIELDTLVGKG | | | | | | KCIMKEK | | QFLTAEERK | |
| | RFAEVYKAKLKQNTSEQ | | | | | | KKPGETFF | | TELGKQYW | |
| | FETVAVKIFPYEEYASWK | | | | | | MCSCSSD | | LITAFHAKG | |
| | TEKDIFSDINLKHENILQF | | | | | | ECNDNIIF | | NLQEYLTRH | |
| | LTAEERKTELGKQYWLIT | | | | | | SEEYNTSN | | VISWEDLRK | |
| | AFHAKGNLQEYLTRHVIS | | | | | | PDLLLVIF | | LGSSLARGI | |
| | WEDLRKLGSSLARGIAHL | | | | | | Q | | AHLHSDHTP | |
| | HSDHTPCGRPKMPIVHRD | | | | | | | | CGRPKMPIV | |
| | LKSSNILVKNDLTCCLCD | | | | | | | | HRDLKSSNI | |
| | FGLSLRLDPTLSVDDLAN | | | | | | | | LVKNDLTCC | |
| | SGQVGTARYMAPEVLES | | | | | | | | LCDFGLSLR | |
| | RMNLENVESFKQTDVYS | | | | | | | | LDPTLSVDD | |
| | MALVLWEMTSRCNAVG | | | | | | | | LANSGQVGT | |
| | EVKDYEPPFGSKVREHPC | | | | | | | | ARYMAPEV | |
| | VESMKDNVLRDRGRPEIP | | | | | | | | LESRMNLEN | |
| | SFWLNHQGIQMVCETLTE | | | | | | | | VESFKQTDV | |
| | CWDHDPEARLTAQCVAE | | | | | | | | YSMALVLW | |
| | RFSELEHLDRLSGRSCSEE | | | | | | | | EMTSRCNA | |
| | KIPEDGSLNTTK | | | | | | | | VGEVKDYEP | |
| | | | | | | | | | PFGSKVREH | |
| | | | | | | | | | PCVESMKD | |
| | | | | | | | | | NVLRDRGRP | |
| | | | | | | | | | EIPSFWLNH | |
| | | | | | | | | | QGIQMVCET | |
| | | | | | | | | | LTECWDHD | |
| | | | | | | | | | PEARLTAQC | |
| | | | | | | | | | VAERFSELE | |
| | | | | | | | | | HLDRLSGRS | |
| | | | | | | | | | CSEEKIPEDG | |
| | | | | | | | | | SLNTTK | |
| LTBR | MLLPWATSAPGLAWGPL | 97. | MLLPWAT | 108. | QAVPPYASENQ | 121. | QAVPPYA | 134. | KSHPSLCRK | 147. |
| | VLGLFGLLAASQPQAVPP | | SAPGLAW | | TCRDQEKEYYE | | SENQTCR | | LGSLLKRRP | |
| | YASENQTCRDQEKEYYE | | GPLVLGLF | | PQHRICCSRCPP | | DQEKEYY | | QGEGPNPVA | |
| | PQHRICCSRCPPGTYVSA | | GLLAASQ | | GTYVSAKCSRI | | EPQHRICC | | GSWEPPKAH | |
| | KCSRIRDTVCATCAENSY | | P | | RDTVCATCAEN | | SRCPPGTY | | PYFPDLVQP | |
| | NEHWNYLTICQLCRPCDP | | | | SYNEHWNYLTI | | VSAKCSRI | | LLPISGDVSP | |
| | VMGLEEIAPCTSKRKTQC | | | | CQLCRPCDPVM | | RDTVCAT | | VSTGLPAAP | |
| | RCQPGMFCAAWALECTH | | | | GLEEIAPCTSKR | | CAENSYN | | VLEAGVPQQ | |
| | CELLSDCPPGTEAELKDE | | | | KTQCRCQPGM | | EHWNYLT | | QSPLDLTRE | |
| | VGKGNNHCVPCKAGHFQ | | | | FCAAWALECT | | ICQLCRPC | | PQLEPGEQS | |
| | NTSSPSARCQPHTRCENQ | | | | HCELLSDCPPG | | DPVMGLE | | QVAHGTNGI | |
| | GLVEAAPGTAQSDTTCK | | | | TEAELKDEVGK | | EIAPCTSK | | HVTGGSMTI | |
| | NPLEPLPPEMSGTMLMLA | | | | GNNHCVPCKA | | RKTQCRC | | TGNIYIYNG | |
| | VLLPLAFFLLLATVFSCIW | | | | GHFQNTSSPSA | | QPGMFCA | | PVLGGPPGP | |
| | KSHPSLCRKLGSLLKRRP | | | | RCQPHTRCENQ | | AWALECT | | GDLPATPEP | |
| | QGEGPNPVAGSWEPPKA | | | | GLVEAAPGTAQ | | HCELLSDC | | PYPIPEEGDP | |
| | HPYFPDLVQPLLPISGDVS | | | | SDTTCKNPLEP | | PPGTEAEL | | GPPGLSTPH | |
| | PVSTGLPAAPVLEAGVPQ | | | | LPPEMSGTML | | KDEVGKG | | QEDGKAWH | |
| | QQSPLDLTREPQLEPGEQ | | | | M | | NNHCVPC | | LAETEHCGA | |
| | SQVAHGTNGIHVTGGSM | | | | | | KAGHFQN | | TPSNRGPRN | |
| | TITGNIYIYNGPVLGGPPG | | | | | | TSSPSARC | | QFITHD | |
| | PGDLPATPEPPYPIPEEGD | | | | | | QPHTRCE | | | |
| | PGPPGLSTPHQEDGKAW | | | | | | NQGLVEA | | | |
| | HLAETEHCGATPSNRGPR | | | | | | APGTAQS | | | |
| | NQFITHD | | | | | | DTTCKNP | | | |
| | | | | | | | LEPLPPEM | | | |
| | | | | | | | SGTMLM | | | |
| IL-4R | MGWLCSGLLFPVSCLVL | 98. | MGWLCSG | 109. | MKVLQEPTCVS | 122. | MKVLQEP | 135. | KIKKEWWD | 148. |
| | LQVASSGNMKVLQEPTC | | LLFPVSCL | | DYMSISTCEWK | | TCVSDYM | | QIPNPARSRL | |
| | VSDYMSISTCEWKMNGP | | VLLQVAS | | MNGPTNCSTEL | | SISTCEWK | | VAIIIQDAQG | |
| | TNCSTELRLLYQLVFLLS | | SGN | | RLLYQLVFLLS | | MNGPTNC | | SQWEKRSR | |
| | EAHTCIPENNGGAGCVCH | | | | EAHTCIPENNG | | STELRLLY | | GQEPAKCPH | |
| | LLMDDVVSADNYTLDLW | | | | GAGCVCHLLM | | QLVFLLSE | | WKNCLTKL | |
| | AGQQLLWKGSFKPSEHV | | | | DDVVSADNYT | | AHTCIPEN | | LPCFLEHNM | |
| | KPRAPGNLTVHTNVSDTL | | | | LDLWAGQQLL | | NGGAGCV | | KRDEDPHK | |
| | LLTWSNPYPPDNYLYNH | | | | WKGSFKPSEHV | | CHLLMDD | | AAKEMPFQ | |
| | LTYAVNIWSENDPADFRI | | | | KPRAPGNLTVH | | VVSADNY | | GSGKSAWC | |
| | YNVTYLEPSLRIAASTLKS | | | | TNVSDTLLLTW | | TLDLWAG | | PVEISKTVL | |
| | GISYRARVRAWAQCYNT | | | | SNPYPPDNYLY | | QQLLWKG | | WPESISVVR | |
| | TWSEWSPSTKWHNSYRE | | | | NHLTYAVNIWS | | SFKPSEHV | | CVELFEAPV | |
| | PFEQHLLLGVSVSCIVILA | | | | ENDPADFRIYN | | KPRAPGN | | ECEEEEEVE | |
| | VCLLCYVSITKIKKEWWD | | | | VTYLEPSLRIAA | | LTVHTNV | | EEKGSFCAS | |
| | QIPNPARSRLVAIIIQDAQ | | | | STLKSGISYRAR | | SDTLLLT | | PESSRDDFQ | |
| | GSQWEKRSRGQEPAKCP | | | | VRAWAQCYNT | | WSNPYPP | | EGREGIVAR | |
| | HWKNCLTKLLPCFLEHN | | | | TWSEWSPSTK | | DNYLYNH | | LTESLFLDLL | |
| | MKRDEDPHKAAKEMPFQ | | | | WHNSYREPFEQ | | LTYAVNI | | GEENGGFCQ | |
| | GSGKSAWCPVEISKTVL | | | | H | | WSENDPA | | QDMGESCLL | |
| | WPESISVVRCVELFEAPV | | | | | | DFRIYNVT | | PPSGSTSAH | |
| | ECEEEEEVEEEKGSFCASP | | | | | | YLEPSLRI | | MPWDEFPSA | |
| | ESSRDDFQEGREGIVARL | | | | | | AASTLKS | | GPKEAPPWG | |
| | TESLFLDLLGEENGGFCQ | | | | | | GISYRARV | | KEQPLHLEP | |
| | QDMGESCLLPPSGSTSAH | | | | | | RAWAQCY | | SPPASPTQSP | |
| | MPWDEFPSAGPKEAPPW | | | | | | NTTWSEW | | DNLTCTETP | |
| | GKEQPLHLEPSPPASPTQS | | | | | | SPSTKWH | | LVIAGNPAY | |
| | PDNLTCTETPLVIAGNPA | | | | | | NSYREPFE | | RSFSNSLSQS | |
| | YRSFSNSLSQSPCPRELGP | | | | | | QH | | PCPRELGPD | |
| | DPLLARHLEEVEPEMPCV | | | | | | | | PLLARHLEE | |
| | PQLSEPTTVPQPEPETWE | | | | | | | | VEPEMPCVP | |
| | QILRRNVLQHGAAAAPVS | | | | | | | | QLSEPTTVP | |
| | APTSGYQEFVHAVEQGG | | | | | | | | QPEPETWEQ | |
| | TQASAVVGLGPPGEAGY | | | | | | | | ILRRNVLQH | |
| | KAFSSLLASSAVSPEKCG | | | | | | | | GAAAAPVS | |
| | FGASSGEEGYKPFQDLIP | | | | | | | | APTSGYQEF | |
| | GCPGDPAPVPVPLFTFGL | | | | | | | | VHAVEQGG | |
| | DREPPRSPQSSHLPSSSPE | | | | | | | | TQASAVVGL | |
| | HLGLEPGEKVEDMPKPPL | | | | | | | | GPPGEAGYK | |
| | PQEQATDPLVDSLGSGIV | | | | | | | | AFSSLLASS | |
| | YSALTCHLCGHLKQCHG | | | | | | | | AVSPEKCGF | |
| | QEDGGQTPVMASPCCGC | | | | | | | | GASSGEEGY | |
| | CCGDRSSPPTTPLRAPDPS | | | | | | | | KPFQDLIPG | |
| | PGGVPLEASLCPASLAPS | | | | | | | | CPGDPAPVP | |
| | GISEKSKSSSSFHPAPGNA | | | | | | | | VPLFTFGLD | |
| | QSSSQTPKIVNFVSVGPT | | | | | | | | REPPRSPQSS | |
| | YMRVS | | | | | | | | HLPSSSPEHL | |
| | | | | | | | | | GLEPGEKVE | |
| | | | | | | | | | DMPKPPLPQ | |
| | | | | | | | | | EQATDPLVD | |
| | | | | | | | | | SLGSGIVYS | |
| | | | | | | | | | ALTCHLCGH | |
| | | | | | | | | | LKQCHGQE | |
| | | | | | | | | | DGGQTPVM | |
| | | | | | | | | | ASPCCGCCC | |
| | | | | | | | | | GDRSSPPTTP | |
| | | | | | | | | | LRAPDPSPG | |
| | | | | | | | | | GVPLEASLC | |
| | | | | | | | | | PASLAPSGIS | |
| | | | | | | | | | EKSKSSSSFH | |
| | | | | | | | | | PAPGNAQSS | |
| | | | | | | | | | SQTPKIVNF | |
| | | | | | | | | | VSVGPTYM | |
| | | | | | | | | | RVS | |
| 2B4 | MLGQVVTLILLLLLKVYQ | 99. | MLGQVVT | 110. | CQGSADHVVSI | 123. | CQGSADH | 136. | WRRKRKEK | 149. |
| | GKGCQGSADHVVSISGVP | | LILLLLLK | | SGVPLQLQPNSI | | VVSISGVP | | QSETSPKEFL | |
| | LQLQPNSIQTKVDSIAWK | | VYQGKG | | QTKVDSIAWK | | LQLQPNSI | | TIYEDVKDL | |
| | KLLPSQNGFHHILKWENG | | | | KLLPSQNGFHH | | QTKVDSIA | | KTRRNHEQE | |
| | SLPSNTSNDRFSFIVKNLS | | | | ILKWENGSLPS | | WKKLLPS | | QTFPGGGSTI | |
| | LLIKAAQQQDSGLYCLEV | | | | NTSNDRFSFIVK | | QNGFHHIL | | YSMIQSQSS | |
| | TSISGKVQTATFQVFVFES | | | | NLSLLIKAAQQ | | KWENGSL | | APTSQEPAY | |
| | LLPDKVEKPRLQGQGKIL | | | | QDSGLYCLEVT | | PSNTSNDR | | TLYSLIQPSR | |
| | DRGRCQVALSCLVSRDG | | | | SISGKVQTATF | | FSFIVKNL | | KSGSRKRNH | |
| | NVSYAWYRGSKLIQTAG | | | | QVFVFESLLPD | | SLLIKAAQ | | SPSFNSTIYE | |
| | NLTYLDEEVDINGTHTYT | | | | KVEKPRLQGQ | | QQDSGLY | | VIGKSQPKA | |
| | CNVSNPVSWESHTLNLTQ | | | | GKILDRGRCQV | | CLEVTSIS | | QNPARLSRK | |
| | DCQNAHQEFRFWPFLVII | | | | ALSCLVSRDGN | | GKVQTAT | | ELENFDVYS | |
| | VILSALFLGTLACFCVWR | | | | VSYAWYRGSK | | FQVFVFES | | | |
| | RKRKEKQSETSPKEFLTIY | | | | LIQTAGNLTYL | | LLPDKVE | | | |
| | EDVKDLKTRRNHEQEQT | | | | DEEVDINGTHT | | KPRLQGQ | | | |
| | FPGGGSTIYSMIQSQSSAP | | | | YTCNVSNPVS | | GKILDRGR | | | |
| | TSQEPAYTLYSLIQPSRKS | | | | WESHTLNLTQD | | CQVALSC | | | |
| | GSRKRNHSPSFNSTIYEVI | | | | CQNAHQEFRF | | LVSRDGN | | | |
| | GKSQPKAQNPARLSRKEL | | | | WP | | VSYAWYR | | | |
| | ENFDVYS | | | | | | GSKLIQTA | | | |
| | | | | | | | GNLTYLD | | | |
| | | | | | | | EEVDINGT | | | |
| | | | | | | | HTYTCNV | | | |
| | | | | | | | SNPVSWE | | | |
| | | | | | | | SHTLNLTQ | | | |
| | | | | | | | DCQNAHQ | | | |
| | | | | | | | EFRFWP | | | |
| VISTA | MGVPTALEAGSWRWGSL | 100. | MGVPTAL | 111. | FKVATPYSLYV | 124. | FKVATPYS | 137. | YKQRQAAS | 150. |
| | LFALFLAASLGPVAAFKV | | EAGSWRW | | CPEGQNVTLTC | | LYVCPEG | | NRRAQELVR | |
| | ATPYSLYVCPEGQNVTLT | | GSLLFALF | | RLLGPVDKGH | | QNVTLTC | | MDSNIQGIE | |
| | CRLLGPVDKGHDVTFYK | | LAASLGP | | DVTFYKTWYR | | RLLGPVD | | NPGFEASPP | |
| | TWYRSSRGEVQTCSERRP | | VAA | | SSRGEVQTCSE | | KGHDVTF | | AQGIPEAKV | |
| | IRNLTFQDLHLHHGGHQA | | | | RRPIRNLTFQDL | | YKTWYRS | | RHPLSYVAQ | |
| | ANTSHDLAQRHGLESAS | | | | HLHHGGHQAA | | SRGEVQT | | RQPSESGRH | |
| | DHHGNFSITMRNLTLLDS | | | | NTSHDLAQRH | | CSERRPIR | | LLSEPSTPLS | |
| | GLYCCLVVEIRHHHSEHR | | | | GLESASDHHGN | | NLTFQDL | | PPGPGDVFF | |
| | VHGAMELQVQTGKDAPS | | | | FSITMRNLTLL | | HLHHGGH | | PSLDPVPDSP | |
| | NCVVYPSSSQDSENITAA | | | | DSGLYCCLVVE | | QAANTSH | | NFEVI | |
| | ALATGACIVGILCLPLILL | | | | IRHHHSEHRVH | | DLAQRHG | | | |
| | LVYKQRQAASNRRAQEL | | | | GAMELQVQTG | | LESASDH | | | |
| | VRMDSNIQGIENPGFEAS | | | | KDAPSNCVVYP | | HGNFSITM | | | |
| | PPAQGIPEAKVRHPLSYV | | | | SSSQDSENITAA | | RNLTLLDS | | | |
| | AQRQPSESGRHLLSEPSTP | | | | | | GLYCCLV | | | |
| | LSPPGPGDVFFPSLDPVPD | | | | | | VEIRHHHS | | | |
| | SPNFEVI | | | | | | EHRVHGA | | | |
| | | | | | | | MELQVQT | | | |
| | | | | | | | GKDAPSN | | | |
| | | | | | | | CVVYPSSS | | | |
| | | | | | | | QDSENITA | | | |
| | | | | | | | A | | | |
| CD28 | MLRLLLALNLFPSIQVTG | 101. | MLRLLLA | 112. | NKILVKQSPML | 125. | NKILVKQS | 138. | RSKRSRLLH | 151. |
| | NKILVKQSPMLVAYDNA | | LNLFPSIQ | | VAYDNAVNLS | | PMLVAYD | | SDYMNMTP | |
| | VNLSCKYSYNLFSREFRA | | VTG | | CKYSYNLFSRE | | NAVNLSC | | RRPGPTRKH | |
| | SLHKGLDSAVEVCVVYG | | | | FRASLHKGLDS | | KYSYNLFS | | YQPYAPPRD | |
| | NYSQQLQVYSKTGFNCD | | | | AVEVCVVYGN | | REFRASLH | | FAAYRS | |
| | GKLGNESVTFYLQNLYV | | | | YSQQLQVYSKT | | KGLDSAV | | | |
| | NQTDIYFCKIEVMYPPPY | | | | GFNCDGKLGN | | EVCVVYG | | | |
| | LDNEKSNGTIIHVKGKHL | | | | ESVTFYLONLY | | NYSQQLQ | | | |
| | CPSPLFPGPSKPFWVLVV | | | | VNQTDIYFCKIE | | VYSKTGF | | | |
| | VGGVLACYSLLVTVAFIIF | | | | VMYPPPYLDNE | | NCDGKLG | | | |
| | WVRSKRSRLLHSDYMN | | | | KSNGTIIHVKG | | NESVTFYL | | | |
| | MTPRRPGPTRKHYQPYA | | | | KHLCPSPLFPGP | | QNLYVNQ | | | |
| | PPRDFAAYRS | | | | SKP | | TDIYFCKI | | | |
| | | | | | | | EVMYPPP | | | |
| | | | | | | | YLDNEKS | | | |
| | | | | | | | NGTIIHVK | | | |
| | | | | | | | GKHLCPSP | | | |
| | | | | | | | LFPGPSKP | | | |
| TNFRSF12 | MARGSLRRLLRLLVLGL | 102. | MARGSLR | 113. | EQAPGTAPCSR | 126. | EQAPGTA | 139. | RRCRRREKF | 152. |
| | WLALLRSVAGEQAPGTA | | RLLRLLVL | | GSSWSADLDK | | PCSRGSS | | TTPIEETGGE | |
| | PCSRGSSWSADLDKCMD | | GLWLALL | | CMDCASCRAR | | WSADLDK | | GCPAVALIQ | |
| | CASCRARPHSDFCLGCAA | | RSVAG | | PHSDFCLGCAA | | CMDCASC | | | |
| | APPAPFRLLWPILGGALS | | | | APPAPFRLLWP | | RARPHSDF | | | |
| | LTFVLGLLSGFLVWRRCR | | | | | | CLGCAAA | | | |
| | RREKFTTPIEETGGEGCPA | | | | | | PPAPFRLL | | | |
| | VALIQ | | | | | | WP | | | |
| OX40 | MCVGARRLGRGPCAALL | 103. | MCVGARR | 114. | LHCVGDTYPSN | 127. | LHCVGDT | 140. | ALYLLRRDQ | 153. |
| | LLGLGLSTVTGLHCVGDT | | LGRGPCA | | DRCCHECRPGN | | YPSNDRC | | RLPPDAHKP | |
| | YPSNDRCCHECRPGNGM | | ALLLLGL | | GMVSRCSRSQN | | CHECRPG | | PGGGSFRTPI | |
| | VSRCSRSQNTVCRPCGPG | | GLSTVTG | | TVCRPCGPGFY | | NGMVSRC | | QEEQADAHS | |
| | FYNDVVSSKPCKPCTWC | | | | NDVVSSKPCKP | | SRSQNTV | | TLAKI | |
| | NLRSGSERKQLCTATQDT | | | | CTWCNLRSGSE | | CRPCGPGF | | | |
| | VCRCRAGTQPLDSYKPG | | | | RKQLCTATQDT | | YNDVVSS | | | |
| | VDCAPCPPGHFSPGDNQA | | | | VCRCRAGTQPL | | KPCKPCT | | | |
| | CKPWTNCTLAGKHTLQP | | | | DSYKPGVDCAP | | WCNLRSG | | | |
| | ASNSSDAICEDRDPPATQ | | | | CPPGHFSPGDN | | SERKQLCT | | | |
| | PQETQGPPARPITVQPTEA | | | | QACKPWTNCT | | ATQDTVC | | | |
| | WPRTSQGPSTRPVEVPGG | | | | LAGKHTLQPAS | | RCRAGTQ | | | |
| | RAVAAILGLGLVLGLLGP | | | | NSSDAICEDRD | | PLDSYKPG | | | |
| | LAILLALYLLRRDQRLPP | | | | PPATQPQETQG | | VDCAPCPP | | | |
| | DAHKPPGGGSFRTPIQEE | | | | PPARPITVQPTE | | GHFSPGD | | | |
| | QADAHSTLAKI | | | | AWPRTSQGPST | | NOACKPW | | | |
| | | | | | RPVEVPGGRA | | TNCTLAG | | | |
| | | | | | | | KHTLQPA | | | |
| | | | | | | | SNSSDAIC | | | |
| | | | | | | | EDRDPPAT | | | |
| | | | | | | | QPQETQG | | | |
| | | | | | | | PPARPITV | | | |
| | | | | | | | QPTEAWP | | | |
| | | | | | | | RTSQGPST | | | |
| | | | | | | | RPVEVPG | | | |
| | | | | | | | GRA | | | |
| PD-1 | MQIPQAPWPVVWAVLQL | 104. | MQIPQAP | 115. | FLDSPDRPWNP | 128. | FLDSPDRP | 141. | CSRAARGTI | 154. |
| | GWRPGWFLDSPDRPWNP | | WPVVWA | | PTFSPALLVVTE | | WNPPTFSP | | GARRTGQPL | |
| | PTFSPALLVVTEGDNATF | | VLQLGWR | | GDNATFTCSFS | | ALLVVTE | | KEDPSAVPV | |
| | TCSFSNTSESFVLNWYRM | | PGW | | NTSESFVLNWY | | GDNATFT | | FSVDYGELD | |
| | SPSNQTDKLAAFPEDRSQ | | | | RMSPSNQTDKL | | CSFSNTSE | | FQWREKTPE | |
| | PGQDCRFRVTQLPNGRDF | | | | AAFPEDRSQPG | | SFVLNWY | | PPVPCVPEQ | |
| | HMSVVRARRNDSGTYLC | | | | QDCRFRVTQLP | | RMSPSNQ | | TEYATIVFPS | |
| | GAISLAPKAQIKESLRAEL | | | | NGRDFHMSVV | | TDKLAAF | | GMGTSSPAR | |
| | RVTERRAEVPTAHPSPSP | | | | RARRNDSGTYL | | PEDRSQPG | | RGSADGPRS | |
| | RPAGQFQTLVVGVVGGL | | | | CGAISLAPKAQI | | QDCRFRV | | AQPLRPEDG | |
| | LGSLVLLVWVLAVICSRA | | | | KESLRAELRVT | | TQLPNGR | | HCSWPL | |
| | ARGTIGARRTGQPLKEDP | | | | ERRAEVPTAHP | | DFHMSVV | | | |
| | SAVPVFSVDYGELDFQW | | | | SPSPRPAGQFQ | | RARRNDS | | | |
| | REKTPEPPVPCVPEQTEY | | | | TLV | | GTYLCGAI | | | |
| | ATIVFPSGMGTSSPARRG | | | | | | SLAPKAQI | | | |
| | SADGPRSAQPLRPEDGHC | | | | | | KESLRAEL | | | |
| | SWPL | | | | | | RVTERRA | | | |
| | | | | | | | EVPTAHPS | | | |
| | | | | | | | PSPRPAGQ | | | |
| | | | | | | | FQTLV | | | |
| BTLA | MKTLPAMLGTGKLFWVF | 105. | MKTLPAM | 116 | KESCDVQLYIK | 129. | KESCDVQ | 142. | RRHQGKQN | 155. |
| | FLIPYLDIWNIHGKESCDV | | LGTGKLF | | RQSEHSILAGD | | LYIKRQSE | | ELSDTAGRE | |
| | QLYIKRQSEHSILAGDPFE | | WVFFLIPY | | PFELECPVKYC | | HSILAGDP | | INLVDAHLK | |
| | LECPVKYCANRPHVTWC | | LDIWNIHG | | ANRPHVTWCK | | FELECPVK | | SEQTEASTR | |
| | KLNGTTCVKLEDRQTSW | | | | LNGTTCVKLED | | YCANRPH | | QNSQVLLSE | |
| | KEEKNISFFILHFEPVLPN | | | | RQTSWKEEKNI | | VTWCKLN | | TGIYDNDPD | |
| | DNGSYRCSANFQSNLIES | | | | SFFILHFEPVLP | | GTTCVKL | | LCFRMQEGS | |
| | HSTTLYVTDVKSASERPS | | | | NDNGSYRCSA | | EDRQTSW | | EVYSNPCLE | |
| | KDEMASRPWLLYRLLPL | | | | NFQSNLIESHST | | KEEKNISF | | ENKPGIVYA | |
| | GGLPLLITTCFCLFCCLRR | | | | TLYVTDVKSAS | | FILHFEPV | | SLNHSVIGP | |
| | HQGKQNELSDTAGREINL | | | | ERPSKDEMASR | | LPNDNGS | | NSRLARNVK | |
| | VDAHLKSEQTEASTRONS | | | | PWLLYR | | YRCSANF | | EAPTEYASIC | |
| | QVLLSETGIYDNDPDLCF | | | | | | QSNLIESH | | VRS | |
| | RMQEGSEVYSNPCLEEN | | | | | | STTLYVTD | | | |
| | KPGIVYASLNHSVIGPNS | | | | | | VKSASERP | | | |
| | RLARNVKEAPTEYASICV | | | | | | SKDEMAS | | | |
| | RS | | | | | | RPWLLYR | | | |
| ICOS | MKSGLWYFFLFCLRIKVL | 106. | MKSGLWY | 117. | EINGSANYEMF | 130. | FWLPIGCA | 143. | CWLTKKKY | 156. |
| | TGEINGSANYEMFIFHNG | | FFLFCLRI | | IFHNGGVQILC | | AFVVVCIL | | SSSVHDPNG | |
| | GVQILCKYPDIVQQFKMQ | | KVLTG | | KYPDIVQQFKM | | GCILI | | EYMFMRAV | |
| | LLKGGQILCDLTKTKGSG | | | | QLLKGGQILCD | | | | NTAKKSRLT | |
| | NTVSIKSLKFCHSQLSNN | | | | LTKTKGSGNTV | | | | DVTL | |
| | SVSFFLYNLDHSHANYYF | | | | SIKSLKFCHSQL | | | | | |
| | CNLSIFDPPPFKVTLTGGY | | | | SNNSVSFFLYN | | | | | |
| | LHIYESQLCCQLKFWLPI | | | | LDHSHANYYFC | | | | | |
| | GCAAFVVVCILGCILICW | | | | NLSIFDPPPFKV | | | | | |
| | LTKKKYSSSVHDPNGEY | | | | TLTGGYLHIYE | | | | | |
| | MFMRAVNTAKKSRLTDV | | | | SQLCCQLK | | | | | |
| | TL | | | | | | | | | |
| DR5 | MEQRGQNAPAASGARKR | 107. | MEQRGQN | 118. | ITQQDLAPQQR | 131. | LSGIIIGVT | 144. | CKSLLWKK | 157. |
| | HGPGPREARGARPGPRVP | | APAASGA | | AAPQQKRSSPS | | VAAVVLI | | VLPYLKGIC | |
| | KTLVLVVAAVLLLVSAES | | RKRHGPG | | EGLCPPGHHISE | | VAVFV | | SGGGGDPER | |
| | ALITQQDLAPQQRAAPQQ | | PREARGA | | DGRDCISCKYG | | | | VDRSSQRPG | |
| | KRSSPSEGLCPPGHHISED | | RPGPRVPK | | QDYSTHWNDL | | | | AEDNVLNEI | |
| | GRDCISCKYGQDYSTHW | | TLVLVVA | | LFCLRCTRCDS | | | | VSILQPTQVP | |
| | NDLLFCLRCTRCDSGEVE | | AVLLLVS | | GEVELSPCTTT | | | | EQEMEVQEP | |
| | LSPCTTTRNTVCQCEEGT | | AESAL | | RNTVCQCEEGT | | | | AEPTGVNM | |
| | FREEDSPEMCRKCRTGCP | | | | FREEDSPEMCR | | | | LSPGESEHL | |
| | RGMVKVGDCTPWSDIEC | | | | KCRTGCPRGM | | | | LEPAEAERS | |
| | VHKESGTKHSGEVPAVEE | | | | VKVGDCTPWS | | | | QRRRLLVPA | |
| | TVTSSPGTPASPCSLSGIII | | | | DIECVHKESGT | | | | NEGDPTETL | |
| | GVTVAAVVLIVAVFVCK | | | | KHSGEVPAVEE | | | | RQCFDDFAD | |
| | SLLWKKVLPYLKGICSGG | | | | TVTSSPGTPASP | | | | LVPFDSWEP | |
| | GGDPERVDRSSQRPGAE | | | | CS | | | | LMRKLGLM | |
| | DNVLNEIVSILQPTQVPEQ | | | | | | | | DNEIKVAKA | |
| | EMEVQEPAEPTGVNMLS | | | | | | | | EAAGHRDTL | |
| | PGESEHLLEPAEAERSQR | | | | | | | | YTMLIKWV | |
| | RRLLVPANEGDPTETLRQ | | | | | | | | NKTGRDAS | |
| | CFDDFADLVPFDSWEPL | | | | | | | | VHTLLDALE | |
| | MRKLGLMDNEIKVAKAE | | | | | | | | TLGERLAKQ | |
| | AAGHRDTLYTMLIKWVN | | | | | | | | KIEDHLLSS | |
| | KTGRDASVHTLLDALETL | | | | | | | | GKFMYLEG | |
| | GERLAKQKIEDHLLSSGK | | | | | | | | NADSAMS | |
| | FMYLEGNADSAMS | | | | | | | | | |

### Additional Sequences

RORC
(SEQ ID NO: 79)
MDRAPQRQHRASRELLAAKKTHTSQIEVIPCKICGDKSSGIHYGVITCEGCKGFFRRSQRCNAAYSCTRQQNC
PIDRTSRNRCQHCRLQKCLALGMSRDAVKFGRMSKKQRDSLHAEVQKQLQQRQQQQQEPVVKTPPAGAQ
GADTLTYTLGLPDGQLPLGSSPDLPEASACPPGLLKASGSGPSYSNNLAKAGLNGASCHLEYSPERGKAEGRE
SFYSTGSQLTPDRCGLRFEEHRHPGLGELGQGPDSYGSPSFRSTPEAPYASLTEIEHLVQSVCKSYRETCQLRLE
DLLRQRSNIFSREEVTGYQRKSMWEMWERCAHHLTEAIQYVVEFAKRLSGFMELCQNDQIVLLKAGAMEV
VLVRMCRAYNADNRTVFFEGKYGGMELFRALGCSELISSIFDFSHSLSALHFSEDEIALYTALVLINAHRPGLQE
KRKVEQLQYNLELAFHHHLCKTHRQSILAKLPPKGKLRSLCSQHVERLQIFQHLHPIVVQAAFPPLYKELFSTET
ESPVGLSK
BATF2
(SEQ ID NO: 80)
MHLCGGNGLLTQTDPKEQQRQLKKQKNRAAAQRSRQKHTDKADALHQQHESLEKDNLALRKEIQSLQAEL
AWWSRTLHVHERLCPMDCASCSAPGLLGCWDQAEGLLGPGPQGQHGCREQLELFQTPGSCYPAQPLSPG
PQPHDSPSLLQCPLPSLSLGPAVVAEPPVQLSPSPLLFASHTGSSLQGSSSKLSALQPSLTAQTAPPQPLELEHP
TRGKLGSSPDNPSSALGLARLOSREHKPALSAATWQGLVVDPSPHPLLAFPLLSSAQVHF
PD-1 truncated
(SEQ ID NO: 81)
MQIPQAPWPVVWAVLQLGWRPGWFLDSPDRPWNPPTFSPALLVVTEGDNATFTCSFSNTSESFVLNWYR
MSPSNQTDKLAAFPEDRSQPGQDCRFRVTQLPNGRDFHMSVVRARRNDSGTYLCGAISLAPKAQIKESLRA
ELRVTERRAEVPTAHPSPSPRPAGQFQTLVVGVVGGLLGSLVLLVWVLAVICSRAARGTIG
TCF7
(SEQ ID NO: 82)
MPQLDSGGGGAGGGDDLGAPDELLAFQDEGEEQDDKSRDSAAGPERDLAELKSSLVNESEGAAGGAGIPG
VPGAGAGARGEAEALGREHAAQRLFPDKLPEPLEDGLKAPECTSGMYKETVYSAFNLLMHYPPPSGAGQHP
QPQPPLHKANQPPHGVPQLSLYEHFNSPHPTPAPADISQKQVHRPLQTPDLSGFYSLTSGSMGQLPHTVSW
FTHPSLMLGSGVPGHPAAIPHPAIVPPSGKQELQPFDRNLKTQAESKAEKEAKKPTIKKPLNAFMLYMKEMR
AKVIAECTLKESAAINQILGRRWHALSREEQAKYYELARKERQLHMQLYPGWSARDNYGKKKRRSREKHQES
TTGGKRNAFGTYPEKAAAPAPFLPMTVL
LAT1
(SEQ ID NO: 83)
MAGAGPKRRALAAPAAEEKEEAREKMLAAKSADGSAPAGEGEGVTLQRNITLLNGVAIIVGTIIGSGIFVTPT
GVLKEAGSPGLALVVWAACGVFSIVGALCYAELGTTISKSGGDYAYMLEVYGSLPAFLKLWIELLIIRPSSQYIV
ALVFATYLLKPLFPTCPVPEEAAKLVACLCVLLLTAVNCYSVKAATRVQDAFAAAKLLALALIILLGFVQIGKGDV
SNLDPNFSFEGTKLDVGNIVLALYSGLFAYGGWNYLNFVTEEMINPYRNLPLAIIISLPIVTLVYVLTNLAYFTTL
STEQMLSSEAVAVDFGNYHLGVMSWIIPVFVGLSCFGSVNGSLFTSSRLFFVGSREGHLPSILSMIHPQLLTPV
PSLVFTCVMTLLYAFSKDIFSVINFFSFFNWLCVALAIIGMIWLRHRKPELERPIKVNLALPVFFILACLFLIAVSF
WKTPVECGIGFTIILSGLPVYFFGVWWKNKPKWLLQGIFSTTVLCQKLMQVVPQET
STAT5
(SEQ ID NO: 84)
MAGWIQAQQLQGDALRQMQVLYGQHFPIEVRHYLAQWIESQPWDAIDLDNPQDRAQATQLLEGLVQEL
QKKAEHQVGEDGFLLKIKLGHYATQLQKTYDRCPLELVRCIRHILYNEQRLVREANNCSSPAGILVDAMSQKH
LQINQTFEELRLVTQDTENELKKLQQTQEYFIIQYQESLRIQAQFAQLAQLSPQERLSRETALQQKQVSLEAWL
QREAQTLQQYRVELAEKHQKTLQLLRKQQTIILDDELIQWKRRQQLAGNGGPPEGSLDVLQSWCEKLAEIIW
QNRQQIRRAEHLCQQLPIPGPVEEMLAEVNATITDIISALVTSTFIIEKQPPQVLKTQTKFAATVRLLVGGKLNV
HMNPPQVKATIISEQQAKSLLKNENTRNECSGEILNNCCVMEYHQATGTLSAHFRNMSLKRIKRADRRGAES
VTEEKFTVLFESQFSVGSNELVFQVKTLSLPVVVIVHGSQDHNATATVLWDNAFAEPGRVPFAVPDKVLWP
QLCEALNMKFKAEVQSNRGLTKENLVFLAQKLFNNSSSHLEDYSGLSVSWSQFNRENLPGWNYTFWQWFD
GVMEVLKKHHKPHWNDGAILGFVNKQQAHDLLINKPDGTFLLRFSDSEIGGITIAWKFDSPERNLWNLKPFT
TRDFSIRSLADRLGDLSYLIYVFPDRPKDEVFSKYYTPVLAKAVDGYVKPQIKQVVPEFVNASADAGGSSATYM
DQAPSPAVCPQAPYNMYPQNPDHVLDQDGEFDLDETMDVARHVEELLRRPMDSLDSRLSPPAGLFTSARG
SLS
MCT4 (SLC16A3)
(SEQ ID NO:85)
MGGAVVDEGPTGVKAPDGGWGWAVLFGCFVITGFSYAFPKAVSVFFKELIQEFGIGYSDTAWISSILLAMLY
GTGPLCSVCVNRFGCRPVMLVGGLFASLGMVAASFCRSIIQVYLTTGVITGLGLALNFQPSLIMLNRYFSKRRP
MANGLAAAGSPVFLCALSPLGQLLQDRYGWRGGFLILGGLLLNCCVCAALMRPLVVTAQPGSGPPRPSRRL
LDLSVFRDRGFVLYAVAASVMVLGLFVPPVFVVSYAKDLGVPDTKAAFLLTILGFIDIFARPAAGFVAGLGKVR
PYSVYLFSFSMFFNGLADLAGSTAGDYGGLVVFCIFFGISYGMVGALQFEVLMAIVGTHKFSSAIGLVLLMEA
VAVLVGPPSGGKLLDATHVYMYVFILAGAEVLTSSLILLLGNFFCIRKKPKEPQPEVAAAEEEKLHKPPADSGV
DLREVEHFLKAEPEKNGEVVHTPETSV
PD_1 truncated
(SEQ ID NO:86)
ATGCAAATTCCCCAAGCCCCATGGCCAGTTGTATGGGCAGTTCTCCAGCTCGGGTGGCGGCCCGGCTGG
TTTCTCGATAGCCCGGATCGGCCTTGGAATCCACCAACATTTAGTCCTGCACTTCTGGTCGTTACAGAGG
GTGATAATGCAACATTTACATGTTCCTTTAGCAATACCTCAGAATCCTTTGTTTTGAATTGGTATCGAATG
TCTCCAAGTAATCAAACCGATAAACTCGCGGCATTTCCAGAAGATAGGTCCCAACCGGGTCAAGATTGT
AGGTTTAGAGTGACTCAGCTTCCAAATGGTCGGGATTTTCATATGTCTGTCGTGCGCGCTCGCAGAAAC
GATTCAGGTACATATCTTTGCGGCGCTATAAGTCTCGCGCCGAAAGCCCAAATAAAGGAATCTCTTAGG
GCCGAACTGCGAGTTACGGAAAGGCGGGCCGAGGTTCCTACTGCTCATCCTTCTCCTAGCCCTCGCCCTG
CAGGGCAATTTCAGACTCTTGTCGTGGGCGTAGTAGGCGGTTTGCTTGGGTCACTTGTATTGCTCGTTTG
GGTGCTCGCTGTGATTTGTTCTAGGGCAGCCAGAGGAACTATCGGC
BATF2
(SEQ ID NO:87)
ATGCACCTCTGTGGGGGCAATGGGCTGCTGACCCAGACAGACCCCAAGGAGCAACAAAGGCAGCT
GAAGAAGCAGAAGAACCGGGCAGCCGCCCAGCGAAGCCGGCAGAAGCACACAGACAAGGCAGAC
GCCCTGCACCAGCAGCACGAGTCTCTGGAAAAAGACAACCTCGCCCTGCGGAAGGAGATCCAGTCC
CTGCAGGCCGAGCTGGCGTGGTGGAGCCGGACCCTGCACGTGCATGAGCGCCTGTGCCCCATGGA
TTGTGCCTCCTGCTCAGCTCCAGGGCTCCTGGGCTGCTGGGACCAGGCTGAGGGGCTCCTGGGCC
CTGGCCCACAGGGACAACATGGCTGCCGGGAGCAGCTGGAGCTGTTCCAGACCCCGGGTTCCTGT
TACCCAGCTCAGCCGCTCTCTCCAGGTCCACAGCCTCATGATTCTCCCAGCCTCCTCCAGTGCCCC
CTGCCCTCACTGTCCCTTGGCCCCGCTGTGGTTGCTGAACCTCCTGTCCAGCTGTCCCCCAGCCCT
CTCCTGTTTGCCTCGCACACTGGTTCCAGCCTGCAGGGGTCTTCCTCTAAGCTCAGTGCCCTCCAGC
CCAGCCTCACGGCCCAAACTGCCCCTCCACAGCCCCTCGAGCTGGAGCATCCCACCAGAGGGAAG
CTGGGGTCCTCTCCCGACAACCCTTCCTCTGCCCTGGGGCTTGCACGTCTGCAGAGCAGGGAGCAC
AAACCTGCTCTCTCAGCAGCCACTTGGCAAGGGCTGGTTGTGGATCCCAGCCCTCACCCTCTCCTG
GCCTTTCCTCTGCTCTCCTCTGCTCAAGTCCACTTC
RORC
(SEQ ID NO:88)
ATGGACAGGGCCCCACAGAGACAGCACCGAGCCTCACGGGAGCTGCTGGCTGCAAAGAAGACCCA
CACCTCACAAATTGAAGTGATCCCTTGCAAAATCTGTGGGGACAAGTCGTCTGGGATCCACTACGGG
GTTATCACCTGTGAGGGGTGCAAGGGCTTCTTCCGCCGGAGCCAGCGCTGTAACGCGGCCTACTCC
TGCACCCGTCAGCAGAACTGCCCCATCGACCGCACCAGCCGAAACCGATGCCAGCACTGCCGCCT
GCAGAAATGCCTGGCGCTGGGCATGTCCCGAGATGCTGTCAAGTTCGGCCGCATGTCCAAGAAGCA
GAGGGACAGCCTGCATGCAGAAGTGCAGAAACAGCTGCAGCAGCGGCAACAGCAGCAACAGGAAC
CAGTGGTCAAGACCCCTCCAGCAGGGGCCCAAGGAGCAGATACCCTCACCTACACCTTGGGGCTCC
CAGACGGGCAGCTGCCCCTGGGCTCCTCGCCTGACCTGCCTGAGGCTTCTGCCTGTCCCCCTGGC
CTCCTGAAAGCCTCAGGCTCTGGGCCCTCATATTCCAACAACTTGGCCAAGGCAGGGCTCAATGGG
GCCTCATGCCACCTTGAATACAGCCCTGAGCGGGGCAAGGCTGAGGGCAGAGAGAGCTTCTATAGC
ACAGGCAGCCAGCTGACCCCTGACCGATGTGGACTTCGTTTTGAGGAACACAGGCATCCTGGGCTT
GGGGAACTGGGACAGGGCCCAGACAGCTACGGCAGCCCCAGTTTCCGCAGCACACCGGAGGCACC
CTATGCCTCCCTGACAGAGATAGAGCACCTGGTGCAGAGCGTCTGCAAGTCCTACAGGGAGACATG
CCAGCTGCGGCTGGAGGACCTGCTGCGGCAGCGCTCCAACATCTTCTCCCGGGAGGAAGTGACTG
GCTACCAGAGGAAGTCCATGTGGGAGATGTGGGAACGGTGTGCCCACCACCTCACCGAGGCCATTC
AGTACGTGGTGGAGTTCGCCAAGAGGCTCTCAGGCTTTATGGAGCTCTGCCAGAATGACCAGATTG
TGCTTCTCAAAGCAGGAGCAATGGAAGTGGTGCTGGTTAGGATGTGCCGGGCCTACAATGCTGACA
ACCGCACGGTCTTTTTTGAAGGCAAATACGGTGGCATGGAGCTGTTCCGAGCCTTGGGCTGCAGCG
AGCTCATCAGCTCCATCTTTGACTTCTCCCACTCCCTAAGTGCCTTGCACTTTTCCGAGGATGAGATT
GCCCTCTACACAGCCCTTGTTCTCATCAATGCCCATCGGCCAGGGCTCCAAGAGAAAAGGAAAGTA
GAACAGCTGCAGTACAATCTGGAGCTGGCCTTTCATCATCATCTCTGCAAGACTCATCGCCAAAGCA
TCCTGGCAAAGCTGCCACCCAAGGGGAAGCTTCGGAGCCTGTGTAGCCAGCATGTGGAAAGGCTG
CAGATCTTCCAGCACCTCCACCCCATCGTGGTCCAAGCCGCTTTCCCTCCACTCTACAAGGAGCTCT
TCAGCACTGAAACCGAGTCACCTGTGGGGCTGTCCAAG
TCF7
(SEQ ID NO:89)
ATGCCGCAGCTGGATTCTggcGGAGGAGGAgcgggcGGAGGAGATGATctcggcgcgccgGATGAGCTGCT
GGCCTTCCAGGATGAAGGCGAGGAGCAGGATGACAAGAGCCGAGATAGCGCCGCCGGTCCTGAGC
GCGACCTGGCCGAGCTCAAGTCGTCGCTTGTGAACGAATCCGAGGGAGCAGCCGGAGGAGCAGGA
ATCCCGGGAGTTCCGGGAGCCGGCGCCGGAGCCCGAGGCGAGGCCGAAGCTCTTGGACGAGAAC
ACGCTGCGCAGAGACTCTTCCCGGACAAACTTCCAGAGCCCCTGGAGGACGGCCTGAAGGCCCCG
GAGTGCACCAGCGGCATGTACAAAGAGACCGTCTACTCCGCCTTCAATCTGCTCATGCATTACCCAC
CTCCTTCGGGAGCAGGACAGCACCCTCAGCCGCAGCCTCCGCTGCACAAGGCCAATCAGCCTCCTC
ACGGTGTCCCTCAACTCTCTCTCTACGAACATTTCAACAGCCCACATCCCACCCCTGCACCTGCGGA
CATCAGCCAGAAGCAAGTTCACAGGCCTCTGCAGACCCCTGACCTCTCTGGCTTCTACTCCCTGACC
TCAGGCAGCATGGGGCAGCTCCCCCACACTGTGAGCTGGTTCACCCACCCATCCTTGATGCTAGGT
TCTGGTGTACCTGGTCACCCAGCAGCCATCCCTCACCCGGCCATTGTGCCTCCTTCAGGGAAGCAG
GAGCTGCAGCCTTTCGACCGCAACCTGAAGACACAAGCAGAGTCCAAGGCAGAGAAGGAGGCCAA
GAAGCCAACCATCAAGAAGCCCCTCAATGCCTTCATGCTGTACATGAAGGAGATGAGAGCCAAGGT
CATTGCAGAGTGCACACTTAAGGAGAGCGCTGCCATCAACCAGATCCTGGGCCGCAGGTGGCACGC
GCTGTCGCGAGAAGAGCAGGCCAAGTACTATGAGCTGGCCCGCAAGGAGAGGCAGCTGCACATGC
AGCTATACCCAGGCTGGTCAGCGCGGGACAACTACGGGAAGAAGAAGAGGCGGTCGAGGGAAAAG
CACCAAGAATCCACCACAGGAGGAAAAAGAAATGCATTCGGTACTTACCCGGAGAAGGCCGCTGCC
CCAGCCCCGTTCCTTCCGATGACAGTGCTC
STAT5
(SEQ ID NO:90)
ATGGCGGGCTGGATCCAGGCCCAACAACTCCAGGGAGACGCGCTGCGCCAGATGCAGGTGCTGTA
CGGCCAGCACTTCCCCATCGAGGTCCGGCACTACTTGGCCCAGTGGATTGAGAGCCAGCCATGGG
ATGCCATTGACTTGGACAATCCCCAGGACAGAGCCCAAGCCACCCAGCTCCTGGAGGGCCTGGTGC
AGGAGCTGCAGAAGAAGGCGGAGCACCAGGTGGGGGAAGATGGGTTTTTACTGAAGATCAAGCTG
GGGCACTACGCCACGCAGCTCCAGAAAACATATGACCGCTGCCCCCTGGAGCTGGTCCGCTGCATC
CGGCACATTCTGTACAATGAACAGAGGCTGGTCCGAGAAGCCAACAATTGCAGCTCTCCGGCTGGG
ATCCTGGTTGACGCCATGTCCCAGAAGCACCTTCAGATCAACCAGACATTTGAGGAGCTGCGACTG
GTCACGCAGGACACAGAGAATGAGCTGAAGAAACTGCAGCAGACTCAGGAGTACTTCATCATCCAG
TACCAGGAGAGCCTGAGGATCCAAGCTCAGTTTGCCCAGCTGGCCCAGCTGAGCCCCCAGGAGCG
TCTGAGCCGGGAGACGGCCCTCCAGCAGAAGCAGGTGTCTCTGGAGGCCTGGTTGCAGCGTGAGG
CACAGACACTGCAGCAGTACCGCGTGGAGCTGGCCGAGAAGCACCAGAAGACCCTGCAGCTGCTG
CGGAAGCAGCAGACCATCATCCTGGATGACGAGCTGATCCAGTGGAAGCGGCGGCAGCAGCTGGC
CGGGAACGGCGGGCCCCCCGAGGGCAGCCTGGACGTGCTACAGTCCTGGTGTGAGAAGTTGGCC
GAGATCATCTGGCAGAACCGGCAGCAGATCCGCAGGGCTGAGCACCTCTGCCAGCAGCTGCCCAT
CCCCGGCCCAGTGGAGGAGATGCTGGCCGAGGTCAACGCCACCATCACGGACATTATCTCAGCCCT
GGTGACCAGCACATTCATCATTGAGAAGCAGCCTCCTCAGGTCCTGAAGACCCAGACCAAGTTTGCA
GCCACCGTACGCCTGCTGGTGGGCGGGAAGCTGAACGTGCACATGAATCCCCCCCAGGTGAAGGC
CACCATCATCAGTGAGCAGCAGGCCAAGTCTCTGCTTAAAAATGAGAACACCCGCAACGAGTGCAGT
GGTGAGATCCTGAACAACTGCTGCGTGATGGAGTACCACCAAGCCACGGGCACCCTCAGTGCCCAC
TTCAGGAACATGTCACTGAAGAGGATCAAGCGTGCTGACCGGCGGGGTGCAGAGTCCGTGACAGA
GGAGAAGTTCACAGTCCTGTTTGAGTCTCAGTTCAGTGTTGGCAGCAATGAGCTTGTGTTCCAGGTG
AAGACTCTGTCCCTACCTGTGGTTGTCATCGTCCACGGCAGCCAGGACCACAATGCCACGGCTACT
GTGCTGTGGGACAATGCCTTTGCTGAGCCGGGCAGGGTGCCATTTGCCGTGCCTGACAAAGTGCTG
TGGCCGCAGCTGTGTGAGGCGCTCAACATGAAATTCAAGGCCGAAGTGCAGAGCAACCGGGGCCT
GACCAAGGAGAACCTCGTGTTCCTGGCGCAGAAACTGTTCAACAACAGCAGCAGCCACCTGGAGGA
CTACAGTGGCCTGTCCGTGTCCTGGTCCCAGTTCAACAGGGAGAACTTGCCGGGCTGGAACTACAC
CTTCTGGCAGTGGTTTGACGGGGTGATGGAGGTGTTGAAGAAGCACCACAAGCCCCACTGGAATGA
TGGGGCCATCCTAGGTTTTGTGAATAAGCAACAGGCCCACGACCTGCTCATCAACAAGCCCGACGG
GACCTTCTTGTTGCGCTTTAGTGACTCAGAAATCGGGGGCATCACCATCGCCTGGAAGTTTGACTCC
CCGGAACGCAACCTGTGGAACCTGAAACCATTCACCACGCGGGATTTCTCCATCAGGTCCCTGGCT
GACCGGCTGGGGGACCTGAGCTATCTCATCTATGTGTTTCCTGACCGCCCCAAGGATGAGGTCTTC
TCCAAGTACTACACTCCTGTGCTGGCTAAAGCTGTTGATGGATATGTGAAACCACAGATCAAGCAAG
TGGTCCCTGAGTTTGTGAATGCATCTGCAGATGCTGGGGGCAGCAGCGCCACGTACATGGACCAGG
CCCCCTCCCCAGCTGTGTGCCCCCAGGCTCCCTATAACATGTACCCACAGAACCCTGACCATGTACT
CGATCAGGATGGAGAATTCGACCTGGATGAGACCATGGATGTGGCCAGGCACGTGGAGGAACTCTT
ACGCCGACCAATGGACAGTCTTGACTCCCGCCTCTCGCCCCCTGCCGGTCTTTTCACCTCTGCCAG
AGGCTCCCTCTCA
MCT4 (SLC16A3)
(SEQ ID NO:91)
ATGGGAGGGGCCGTGGTGGACGAGGGCCCCACAGGCGTCAAGGCCCCTGACGGCGGCTGGGGCTGG
GCCGTGCTCTTCGGCTGTTTCGTCATCACTGGCTTCTCCTACGCCTTCCCCAAGGCCGTCAGTGTCTTCTT
CAAGGAGCTCATACAGGAGTTTGGGATCGGCTACAGCGACACAGCCTGGATCTCCTCCATCCTGCTGGC
CATGCTCTACGGGACAGGTCCGCTCTGCAGTGTGTGCGTGAACCGCTTTGGCTGCCGGCCCGTCATGCT
TGTGGGGGGTCTCTTTGCGTCGCTGGGCATGGTGGCTGCGTCCTTTTGCCGGAGCATCATCCAGGTCTA
CCTCACCACTGGGGTCATCACGGGGTTGGGTTTGGCACTCAACTTCCAGCCCTCGCTCATCATGCTGAAC
CGCTACTTCAGCAAGCGGCGCCCCATGGCCAACGGGCTGGCGGCAGCAGGTAGCCCTGTCTTCCTGTGT
GCCCTGAGCCCGCTGGGGCAGCTGCTGCAGGACCGCTACGGCTGGCGGGGCGGCTTCCTCATCCTGGG
CGGCCTGCTGCTCAACTGCTGCGTGTGTGCCGCACTCATGAGGCCCCTGGTGGTCACGGCCCAGCCGGG
CTCGGGGCCGCCGCGACCCTCCCGGCGCCTGCTAGACCTGAGCGTCTTCCGGGACCGCGGCTTTGTGCT
TTACGCCGTGGCCGCCTCGGTCATGGTGCTGGGGCTCTTCGTCCCGCCCGTGTTCGTGGTGAGCTACGC
CAAGGACCTGGGCGTGCCCGACACCAAGGCCGCCTTCCTGCTCACCATCCTGGGCTTCATTGACATCTTC
GCGCGGCCGGCCGCGGGCTTCGTGGCGGGGCTTGGGAAGGTGCGGCCCTACTCCGTCTACCTCTTCAG
CTTCTCCATGTTCTTCAACGGCCTCGCGGACCTGGCGGGTTCTACGGCGGGCGACTACGGCGGCCTCGT
GGTCTTCTGCATCTTCTTTGGCATCTCCTACGGCATGGTGGGGGCCCTGCAGTTCGAGGTGCTCATGGCC
ATCGTGGGCACCCACAAGTTCTCCAGTGCCATTGGCCTGGTGCTGCTGATGGAGGCGGTGGCCGTGCTC
GTCGGGCCCCCTTCGGGAGGCAAACTCCTGGATGCGACCCACGTCTACATGTACGTGTTCATCCTGGCG
GGGGCCGAGGTGCTCACCTCCTCCCTGATTTTGCTGCTGGGCAACTTCTTCTGCATTAGGAAGAAGCCCA
AAGAGCCACAGCCTGAGGTGGCGGCCGCGGAGGAGGAGAAGCTCCACAAGCCTCCTGCAGACTCGGG
GGTGGACTTGCGGGAGGTGGAGCATTTCCTGAAGGCTGAGCCTGAGAAAAACGGGGAGGTGGTTCAC
ACCCCGGAAACAAGTGTC
LAT1
(SEQ ID NO: 92)
ATGGCGGGTGCGGGCCCGAAGCGGCGCGCGCTAGCGGCGCCGGCGGCCGAGGAGAAGGAAGAGGCG
CGGGAGAAGATGCTGGCCGCCAAGAGCGCGGACGGCTCGGCGCCGGCAGGCGAGGGCGAGGGCGTG
ACCCTGCAGCGGAACATCACGCTGCTCAACGGCGTGGCCATCATCGTGGGGACCATTATCGGCTCGGGC
ATCTTCGTGACGCCCACGGGCGTGCTCAAGGAGGCAGGCTCGCCGGGGCTGGCGCTGGTGGTGTGGGC
CGCGTGCGGCGTCTTCTCCATCGTGGGCGCGCTCTGCTACGCGGAGCTCGGCACCACCATCTCCAAATC
GGGCGGCGACTACGCCTACATGCTGGAGGTCTACGGCTCGCTGCCCGCCTTCCTCAAGCTCTGGATCGA
GCTGCTCATCATCCGGCCTTCATCGCAGTACATCGTGGCCCTGGTCTTCGCCACCTACCTGCTCAAGCCGC
TCTTCCCCACCTGCCCGGTGCCCGAGGAGGCAGCCAAGCTCGTGGCCTGCCTCTGCGTGCTGCTGCTCAC
GGCCGTGAACTGCTACAGCGTGAAGGCCGCCACCCGGGTCCAGGATGCCTTTGCCGCCGCCAAGCTCCT
GGCCCTGGCCCTGATCATCCTGCTGGGCTTCGTCCAGATCGGGAAGGGTGATGTGTCCAATCTAGATCC
CAACTTCTCATTTGAAGGCACCAAACTGGATGTGGGGAACATTGTGCTGGCATTATACAGCGGCCTCTTT
GCCTATGGAGGATGGAATTACTTGAATTTCGTCACAGAGGAAATGATCAACCCCTACAGAAACCTGCCC
CTGGCCATCATCATCTCCCTGCCCATCGTGACGCTGGTGTACGTGCTGACCAACCTGGCCTACTTCACCAC
CCTGTCCACCGAGCAGATGCTGTCGTCCGAGGCCGTGGCCGTGGACTTCGGGAACTATCACCTGGGCGT
CATGTCCTGGATCATCCCCGTCTTCGTGGGCCTGTCCTGCTTCGGCTCCGTCAATGGGTCCCTGTTCACAT
CCTCCAGGCTCTTCTTCGTGGGGTCCCGGGAAGGCCACCTGCCCTCCATCCTCTCCATGATCCACCCACA
GCTCCTCACCCCCGTGCCGTCCCTCGTGTTCACGTGTGTGATGACGCTGCTCTACGCCTTCTCCAAGGAC
ATCTTCTCCGTCATCAACTTCTTCAGCTTCTTCAACTGGCTCTGCGTGGCCCTGGCCATCATCGGCATGAT
CTGGCTGCGCCACAGAAAGCCTGAGCTTGAGCGGCCCATCAAGGTGAACCTGGCCCTGCCTGTGTTCTT
CATCCTGGCCTGCCTCTTCCTGATCGCCGTCTCCTTCTGGAAGACACCCGTGGAGTGTGGCATCGGCTTC
ACCATCATCCTCAGCGGGCTGCCCGTCTACTTCTTCGGGGTCTGGTGGAAAAACAAGCCCAAGTGGCTC
CTCCAGGGCATCTTCTCCACGACCGTCCTGTGTCAGAAGCTCATGCAGGTGGTCCCCCAGGAGACA
CCR4
(SEQ ID NO: 174)
MNPTDIADTTLDESIYSNYYLYESIPKPCTKEGIKAFGELFLPPLYSLVFVFGLLGNSVVVLVLFKYKRLRSMTDV
YLLNLAISDLLFVFSLPFWGYYAADQWVFGLGLCKMISWMYLVGFYSGIFFVMLMSIDRYLAIVHAVFSLRAR
TLTYGVITSLATWSVAVFASLPGFLFSTCYTERNHTYCKTKYSLNSTTWKVLSSLEINILGLVIPLGIMLFCYSMII
RTLQHCKNEKKNKAVKMIFAVVVLFLGFWTPYNIVLFLETLVELEVLQDCTFERYLDYAIQATETLAFVHCCLN
PIIYFFLGEKFRKYILQLFKTCRGLFVLCQYCGLLQIYSADTPSSSYTQSTMDHDLHDAL

1. A human T cell that heterologously expresses:
a polypeptide comprising a TFAP4 protein, and a polypeptide comprising a BATF protein;
a polypeptide comprising a BATF3 protein, and a polypeptide comprising a TFAP4 protein;
a polypeptide comprising a FOXJ2 protein, and a polypeptide comprising a RARA protein;
a polypeptide comprising an ID3 protein, and a polypeptide comprising TFAP4 protein;
a polypeptide comprising a BATF protein, and a polypeptide comprising IRF2 protein;
a polypeptide comprising a NANOG protein, and a polypeptide comprising TFAP4 protein;
a polypeptide comprising a MAFF protein, and a polypeptide comprising SATB1 protein;
a polypeptide comprising a BATF protein, and a polypeptide comprising IRF1 protein;
a polypeptide comprising a TFAP4 protein and a polypeptide comprising HOPX protein;
a polypeptide comprising a BATF protein, and a polypeptide comprising HES2 protein;
a polypeptide comprising an ATF2 protein, and a polypeptide comprising BATF protein;
a polypeptide comprising a BATF3 protein, and a polypeptide comprising SMAD4 protein;
a polypeptide comprising a FOXP3 protein, and a polypeptide comprising RELA protein;
a polypeptide comprising an ID3 protein, and a polypeptide comprising BATF3 protein;
a polypeptide comprising an ID2 protein, and a polypeptide comprising a TP73 protein;
a polypeptide comprising a TIGIT protein, and a polypeptide comprising an ICOS protein;
a polypeptide comprising a LTBR protein, and a polypeptide comprising a 4-1BB protein;
a polypeptide comprising a DR5 protein, and a polypeptide comprising a ICOS protein;
a polypeptide comprising a SMAD3 protein, and a polypeptide comprising a BATF3 protein;
a polypeptide comprising a HES2 protein;
a polypeptide comprising a FOXJ2 protein;
a polypeptide comprising a CCR4 protein;
a polypeptide comprising a human TFAP4 protein, and a polypeptide comprising a human TIM3 extracellular domain or a portion thereof linked to a human 4-1BB intracellular domain (and optionally about 1-15 (e.g., 12) amino acids of the human 4-1BB extracellular domain) via a transmembrane domain;
a polypeptide comprising a human SOX5 protein, and a truncated TIGIT protein comprising the human TIGIT extracellular domain or a portion thereof, the TIGIT transmembrane domain, and about 1-10 (e.g., 7) amino acids of the human TIGIT intracellular domain;
a polypeptide comprising a human MYC protein, and a polypeptide comprising a human TGFbR2 extracellular domain or a portion thereof linked to amino acids 41-142 of human MyD88 (and optionally 1-10 (e.g. 7) amino acids of the TGFbR2 intracellular domain) via a transmembrane domain;
a polypeptide comprising a BATF3 protein and a polypeptide comprising 4-1BB protein;
a polypeptide comprising a TFAP4 protein, and a polypeptide comprising a human LTBR extracellular domain or a portion thereof linked to the intracellular domain of human IL-4R (and optionally 1-10 (e.g. 7) amino acids of the LTBR intracellular domain) via a transmembrane domain;
a polypeptide comprising a ATF1 protein, and a polypeptide comprising a human IL4RA extracellular domain or a portion thereof linked to the intracellular domain of human 4-1BB;
a polypeptide comprising a BATF protein, and a polypeptide comprising a human 2B4 extracellular domain or a portion thereof linked to the intracellular domain of human 4-1BB, and optionally about 1-15 (e.g., 12) amino acids of the human 4-1BB extracellular domain;
a polypeptide comprising a RORC protein, and a polypeptide comprising a VISTA extracellular domain or a portion thereof linked to the intracellular domain of human CD28, and optionally about 1-15 (e.g., 12) amino acids of the human CD28 extracellular domain;
a polypeptide comprising a BATF protein and a polypeptide comprising CXCR 1 protein;
a polypeptide comprising a MAFF protein and a polypeptide comprising IL2RA protein;
a polypeptide comprising a ATF1 protein, and a polypeptide comprising a human TNFRSF12 extracellular domain or a portion thereof linked to the intracellular domain of human OX40 (and optionally 1-10 (e.g. 7) amino acids of the TNFRSF12 intracellular domain) via a transmembrane domain;
a polypeptide comprising a human TIM3 extracellular domain or a portion thereof linked to the intracellular domain of human 4-1BB (and optionally about 1-15 (e.g., 12) amino acids of the human 4-1BB extracellular domain) via a transmembrane domain;
a polypeptide comprising a human BATF2 extracellular domain or a portion thereof and a truncated PD-1 comprising the human PD-1 extracellular domain or a portion thereof, the PD-1 transmembrane domain and about 1-12 (e.g., 10) amino acids of the human PD-1 intracellular domain;
a polypeptide comprising a human TCF7 protein and a polypeptide comprising a human LAT1 protein;
a polypeptide comprising a human 4-1BB protein;
a polypeptide comprising a human STAT5 protein and a human IL-2RA protein;
a polypeptide comprising a human FOXJ2 protein and a polypeptide comprising a LTBR extracellular domain or a portion thereof linked to the intracellular domain of human CD28 (and optionally 1-10 (e.g. 7) amino acids of the LTBR intracellular domain) via a transmembrane domain;
a polypeptide comprising a human FOXJ2 protein and a polypeptide comprising a LTBR extracellular domain or a portion thereof linked to amino acids 41-142 of human MyD88 (and optionally 1-10 (e.g. 7) amino acids of the LTBR intracellular domain) via a transmembrane domain;
a polypeptide comprising a human HOPX protein and a polypeptide comprising a IL4RA extracellular domain or a portion thereof linked to the intracellular domain of human 41-BB via a transmembrane domain;
a polypeptide comprising a ID2 protein, and a polypeptide comprising a BLTA extracellular domain or a portion thereof linked to the intracellular domain of human 4-1BB, and optionally about 1-15 (e.g., 12) amino acids of the human 4-1BB extracellular domain;
a polypeptide comprising a human SMAD1 protein, and a polypeptide comprising a MCT4 protein;
a polypeptide comprising a human SMAD1 protein and a polypeptide comprising a LTBR extracellular domain or a portion thereof linked to the intracellular domain of human IL-4R (and optionally 1-10 (e.g. 7) amino acids of the LTBR intracellular domain) via a transmembrane domain,
a polypeptide comprising about 1-12 amino acids (e.g., 7 amino acids) of the DR5 intracellular domain and a polypeptide comprising the intracellular domain of human ICOS via a transmembrane domain;
a polypeptide comprising a TIGIT extracellular domain or a portion thereof linked to the intracellular domain of human ICOS (and optionally about 1-15 (e.g., 12) amino acids of the human ICOS extracellular domain) via a transmembrane domain;
a polypeptide comprising a LTBR extracellular domain or a portion thereof linked to the intracellular domain of human 4-1BB (and optionally about 1-15 (e.g., 7) amino acids of the human LTBR intracellular domain) via a transmembrane domain,
wherein the one or more polypeptides are encoded by a heterologous nucleic acid construct inserted into a target genomic locus of the cell, optionally wherein the target genomic locus is the T-cell receptor (TCR) locus of the cell, optionally wherein the heterologous nucleic acid construct is non-virally inserted.

2. The human T cell of claim 1, wherein the T cell heterologously expresses one or more polypeptides, wherein each polypeptide comprises an amino acid sequence that is at least 95% identical to an amino acid sequence selected from the group consisting of SEQ ID NO: 30-SEQ ID NO: 58, SEQ ID NO: 69-85, SEQ ID NO:160, SEQ ID NO:161 and SEQ ID NO:162.

3. The human T cell of claim 1, wherein the target insertion site is in exon 1 of a TCR-alpha subunit constant gene (TRAC) or in exon 1 of a TCR-beta subunit constant gene (TRBC).

4. (canceled)

5. The human T cell of claim 1, wherein the TRBC is TRBC1 or TRBC2.

6. The human T cell of claim 1, wherein the heterologous nucleic acid construct comprises one or more nucleic acid sequences, wherein each nucleic acid is at least 95% identical to a nucleic acid sequence selected from the consisting of SEQ ID NO: 1-29, and 59-68.

7. The human T cell of claim 1, wherein the T cell expresses an antigen-specific T-cell receptor (TCR) or synthetic antigen receptor that recognizes a target antigen.

8-11. (canceled)

12. The human T cell of claim 1, wherein the T cell is a primary cell.

13-17. (canceled)

18. A nucleic sequence encoding:
a TFAP polypeptide comprising an amino acid sequence at least 95% identical to SEQ ID NO: 30, and a BATF polypeptide comprising an amino acid sequence at least 95% identical to SEQ ID NO: 31;
a BATF3 polypeptide comprising an amino acid sequence at least 95% identical to SEQ ID NO: 32, and a TFAP4 polypeptide comprising an amino acid sequence at least 95% identical to SEQ ID NO: 30;
a FOXJ2 polypeptide comprising an amino acid sequence at least 95% identical to SEQ ID NO: 33, and a RARA polypeptide comprising an amino acid sequence at least 95% identical to SEQ ID NO: 34; 7a ID3 polypeptide comprising an amino acid sequence at least 95% identical to SEQ ID NO: 35, and a TFAP4 polypeptide comprising an amino acid sequence at least 95% identical to SEQ ID NO: 30;
a BATF polypeptide comprising an amino acid sequence at least 95% identical to SEQ ID NO: 31, and a IRF2 polypeptide comprising an amino acid sequence at least 95% identical to SEQ ID NO: 36;
a NANOG polypeptide comprising an amino acid sequence at least 95% identical to SEQ ID NO: 37, and a TFAP4 polypeptide comprising an amino acid sequence at least 95% identical to SEQ ID NO: 30;
a MAFF polypeptide comprising an amino acid sequence at least 95% identical to SEQ ID NO: 38, and a SATB1 polypeptide comprising an amino acid sequence at least 95% identical to SEQ ID NO: 39;
a BATF polypeptide comprising an amino acid sequence at least 95% identical to SEQ ID NO: 32, and a IRF1 polypeptide comprising an amino acid sequence at least 95% identical to SEQ ID NO: 36;
a TFAP4 polypeptide comprising an amino acid sequence at least 95% identical to SEQ ID NO: 39, and a HOPX polypeptide comprising an amino acid sequence at least 95% identical to SEQ ID NO: 51;
a TFAP4 polypeptide comprising an amino acid sequence at least 95% identical to SEQ ID NO: 30, and a HOPX polypeptide comprising an amino acid sequence at least 95% identical to SEQ ID NO: 42;
a BATF polypeptide comprising an amino acid sequence at least 95% identical to SEQ ID NO: 31, and a HES2 polypeptide comprising an amino acid sequence at least 95% identical to SEQ ID NO: 43;
a ATF2 polypeptide comprising an amino acid sequence at least 95% identical to SEQ ID NO: 47, and a BATF polypeptide comprising an amino acid sequence at least 95% identical to SEQ ID NO: 31;
a BATF3 polypeptide comprising an amino acid sequence at least 95% identical to SEQ ID NO: 32, and a SMAD4 polypeptide comprising an amino acid sequence at least 95% identical to SEQ ID NO: 48;
a FOXP3 polypeptide comprising an amino acid sequence at least 95% identical to SEQ ID NO: 49, and a RELA polypeptide comprising an amino acid sequence at least 95% identical to SEQ ID NO: 50;
a ID3 polypeptide comprising an amino acid sequence at least 95% identical to SEQ ID NO: 35, and a BATF3 polypeptide comprising an amino acid sequence at least 95% identical to SEQ ID NO: 32;
a TFAP4 polypeptide comprising an amino acid sequence at least 95% identical to SEQ ID NO: 30, and a TIM-3/4-1BB polypeptide comprising an amino acid sequence at least 95% identical to SEQ ID NO: 69;
a SOX5 polypeptide comprising an amino acid sequence at least 95% identical to SEQ ID NO: 51, and a TIM-3/4-1BB polypeptide comprising an amino acid sequence at least 95% identical to SEQ ID NO: 69;
a MYC polypeptide comprising an amino acid sequence at least 95% identical to SEQ ID NO: 53, and a TGFbR2/MyD88 polypeptide comprising an amino acid sequence at least 95% identical to SEQ ID NO: 70;
a BATF3 polypeptide comprising an amino acid sequence at least 95% identical to SEQ ID NO: 32, and a 4-1BB polypeptide comprising an amino acid sequence at least 95% identical to SEQ ID NO: 55;
a TFAP4 polypeptide comprising an amino acid sequence at least 95% identical to SEQ ID NO: 30, and a LTBR-IL-4R polypeptide comprising an amino acid sequence at least 95% identical to SEQ ID NO: 71;
a ATF1 polypeptide comprising an amino acid sequence at least 95% identical to SEQ ID NO: 58, and a IL4RA/4-1BB polypeptide comprising an amino acid sequence at least 95% identical to SEQ ID NO: 72;
a BATF polypeptide comprising an amino acid sequence at least 95% identical to SEQ ID NO: 31, and a 2B4/4-1BB polypeptide comprising an amino acid sequence at least 95% identical to SEQ ID NO: 73;
a RORC polypeptide comprising an amino acid sequence at least 95% identical to SEQ ID NO: 79, and a VISTA/CD28 polypeptide comprising an amino acid sequence at least 95% identical to SEQ ID NO: 74;
a BATF polypeptide comprising an amino acid sequence at least 95% identical to SEQ ID NO: 31, and a CXCR1 polypeptide comprising an amino acid sequence at least 95% identical to SEQ ID NO: 57;
a MAFF polypeptide comprising an amino acid sequence at least 95% identical to SEQ ID NO: 38, and a IL-2RA polypeptide comprising an amino acid sequence at least 95% identical to SEQ ID NO: 54;
a ATF1 polypeptide comprising an amino acid sequence at least 95% identical to SEQ ID NO: 58, and a TNFRSF12-OX40 polypeptide comprising an amino acid sequence at least 95% identical to SEQ ID NO: 75;
a TIM-3/4-1BB polypeptide comprising an amino acid sequence at least 95% identical to SEQ ID NO: 69,
a BATF2 polypeptide comprising an amino acid sequence at least 95% identical to SEQ ID NO: 80 and a truncated PD1 polypeptide comprising an amino acid sequence at least 95% identical to SEQ ID NO: 81;
a TCF7 polypeptide comprising an amino acid sequence at least 95% identical to SEQ ID NO: 82, and a LAT polypeptide comprising an amino acid sequence at least 95% identical to SEQ ID NO: 83;
a STAT5 polypeptide comprising an amino acid sequence at least 95% identical to SEQ ID NO: 84, and a IL-2RA polypeptide comprising an amino acid sequence at least 95% identical to SEQ ID NO: 54;
a FOXJ2 polypeptide comprising an amino acid sequence at least 95% identical to SEQ ID NO: 33, and a LTBR-CD28 polypeptide comprising an amino acid sequence at least 95% identical to SEQ ID NO: 76;
a FOXJ2 polypeptide comprising an amino acid sequence at least 95% identical to SEQ ID NO: 33, and a LTBR-MyD88 polypeptide comprising an amino acid sequence at least 95% identical to SEQ ID NO: 77;
a HOPX polypeptide comprising an amino acid sequence at least 95% identical to SEQ ID NO: 42, and a IL4RA/4-1BB polypeptide comprising an amino acid sequence at least 95% identical to SEQ ID NO: 72;
a ID2 polypeptide comprising an amino acid sequence at least 95% identical to SEQ ID NO: 44, and a BTLA/4-1BB polypeptide comprising an amino acid sequence at least 95% identical to SEQ ID NO: 78;
a SMAD1 polypeptide comprising an amino acid sequence at least 95% identical to SEQ ID NO: 56, and a MCT4 polypeptide comprising an amino acid sequence at least 95% identical to SEQ ID NO: 85;
a SMAD1 polypeptide comprising an amino acid sequence at least 95% identical to SEQ ID NO: 56, and a LTBR-IL-4R polypeptide comprising an amino acid sequence at least 95% identical to SEQ ID NO: 71;
a TIGIT polypeptide comprising an amino acid sequence at least 95% identical to SEQ ID NO: 121, and an ICOS polypeptide comprising an amino acid sequence at least 95% identical to SEQ ID NO: 159;
a LTBR polypeptide comprising an amino acid sequence at least 95% identical to SEQ ID NO: 97, and an 4-1BB polypeptide comprising an amino acid sequence at least 95% identical to SEQ ID NO: 146;
a DR5 polypeptide comprising an amino acid sequence at least 95% identical to SEQ ID NO: 133, and an ICOS polypeptide comprising an amino acid sequence at least 95% identical to SEQ ID NO: 159.

19. (canceled)

20. A human T cell comprising the nucleic acid sequence of claim 18.

21-22. (canceled)

23. A method of modifying a human T cell comprising
(a) introducing into the human T cell
(i) a targeted nuclease that cleaves a target region in the TCR locus of a human T cell to create a target insertion site in the genome of the cell; and
(ii) a nucleic acid construct encoding: a polypeptide comprising a TFAP4 protein and a BATF protein; a polypeptide comprising a BATF3 protein and a TFAP4 protein; a polypeptide comprising a FOXJ2 protein and a RARA protein; a polypeptide comprising a ID3 protein and a TFAP4 protein; a polypeptide comprising a BATF protein and a IRF2 protein; a polypeptide comprising a NANOG protein and a TFAP4 protein; a polypeptide comprising a MAFF protein and a SATB1 protein; a polypeptide comprising a BATF protein and a IRF1 protein; a polypeptide comprising a TFAP4 protein and a HOPX protein; a polypeptide comprising a BATF protein and a HES2 protein; a polypeptide comprising a ATF2 protein and a BATF protein; a polypeptide comprising a BATF3 protein and a SMAD4 protein; a polypeptide comprising a FOXP3 protein and a RELA protein; a polypeptide comprising a ID3 protein and a BATF3 protein; a polypeptide comprising a EOMES protein; a polypeptide comprising a HES2 protein; a polypeptide comprising a FOXJ2 protein;
a polypeptide comprising (a) a human TFAP4 protein, and (b) a polypeptide comprising a human TIM3 extracellular domain or a portion thereof linked to a human 4-1BB intracellular domain (and optionally about 1-15 (e.g., 12) amino acids of the human 4-1BB extracellular domain) via a transmembrane domain;
a polypeptide comprising (a) a human SOX5 protein, and (b) a truncated TIGIT protein comprising the human TIGIT extracellular domain or a portion thereof, the TIGIT transmembrane domain and about 1-10 (e.g., 7) amino acids of the human TIGIT intracellular domain;
a polypeptide comprising (a) a human MYC protein, and (b) a polypeptide comprising a human TGFbR2 extracellular domain or a portion thereof linked to amino acids 41-142 of human MyD88 (and optionally 1-10 (e.g. 7) amino acids of the TGFbR2 intracellular domain) via a transmembrane domain;
a polypeptide comprising a BATF3 protein and a 4-1BB protein;
a polypeptide comprising (a) a TFAP4 protein, and (b) a polypeptide comprising a human LTBR extracellular domain or a portion thereof linked to the intracellular domain of human IL-4R (and optionally 1-10 (e.g. 7) amino acids of the LTBR intracellular domain) via a transmembrane domain;
a polypeptide comprising (a) a ATF1 protein, and (b) a polypeptide comprising a human IL4RA extracellular domain or a portion thereof linked to the intracellular domain of human 4-1BB;
a polypeptide comprising (a) a BATF protein, and (b) a polypeptide comprising a human 2B4 extracellular domain or a portion thereof linked to the intracellular domain of human 4-1BB, and optionally about 1-15 (e.g., 12) amino acids of the human 4-1BB extracellular domain;
a polypeptide comprising (a) a RORC protein, and (b) a polypeptide comprising a VISTA extracellular domain or a portion thereof linked to the intracellular domain of human CD28, and optionally about 1-15 (e.g., 12) amino acids of the human CD28 extracellular domain;
a polypeptide comprising a BATF protein and a CXCR 1 protein;
a polypeptide comprising a MAFF protein and a IL2RA protein;
a polypeptide comprising (a) a ATF1 protein, and (b) a polypeptide comprising a human TNFRSF12 extracellular domain or a portion thereof linked to the intracellular domain of human OX40 (and optionally 1-10 (e.g. 7) amino acids of the TNFRSF12 intracellular domain) via a transmembrane domain;
a polypeptide comprising a human TIM3 extracellular domain or a portion thereof linked to the intracellular domain of human 4-1BB (and optionally about 1-15 (e.g., 12) amino acids of the human 4-1BB extracellular domain) via a transmembrane domain;
a polypeptide comprising a human BATF2 extracellular domain or a portion thereof linked to a truncated PD-1 comprising the human PD-1 extracellular domain or a portion thereof, the PD-1 transmembrane domain and about 1-12 (e.g., 10) amino acids of the human PD-1 intracellular domain;
a polypeptide comprising a human TCF7 protein and a human LAT1 protein;
a polypeptide comprising a human 4-1BB protein;
a polypeptide comprising a human STAT5 protein and a human IL-2RA protein;
a polypeptide comprising (a) a human FOXJ2 protein and (b) a polypeptide comprising a LTBR extracellular domain or a portion thereof linked to the intracellular domain of human CD28 (and optionally 1-10 (e.g. 7) amino acids of the LTBR intracellular domain) via a transmembrane domain;
a polypeptide comprising (a) a human FOXJ2 protein and (b) a polypeptide comprising a LTBR extracellular domain or a portion thereof linked to amino acids 41-142 of human MyD88 (and optionally 1-10 (e.g. 7) amino acids of the LTBR intracellular domain) via a transmembrane domain;
a polypeptide comprising (a) a human HOPX protein and (b) a polypeptide comprising a IL4RA extracellular domain or a portion thereof linked to the intracellular domain of human 41-BB via a transmembrane domain;
a polypeptide comprising (a) a ID2 protein, and (b) a polypeptide comprising a BLTA extracellular domain or a portion thereof linked to the intracellular domain of human 4-1BB, and optionally about 1-15 (e.g., 12) amino acids of the human 4-1BB extracellular domain;
a polypeptide comprising a human SMAD1 protein and a MCT4 protein; and
a polypeptide comprising (a) a human SMAD1 protein and (b) a LTBR extracellular domain or a portion thereof linked to amino acids the intracellular domain of human IL-4R (and optionally 1-10 (e.g. 7) amino acids of the LTBR intracellular domain) via a transmembrane domain; and
(b) allowing recombination to occur, thereby inserting the nucleic acid construct in the target insertion site to generate a modified human T cell.

24. The method of claim 23, wherein the polypeptide comprises an amino acid sequence that is at least 95% identical to an amino acid sequence selected from the group consisting of SEQ ID NO: 30-SEQ ID NO: 58, and SEQ ID NO: 69-85, 160, 161 and 162.

25. (canceled)

26. The method of claim 23, wherein the target insertion site is in exon 1 of a TCR-alpha subunit constant gene (TRAC) or in exon 1 of a TCR-beta subunit constant gene (TRBC).

27-32. (canceled)

33. The method of claim 23 wherein the cell is a primary cell.

34. A modified T cell produced by the method of claim 23

35. A method of enhancing an immune response in a human subject comprising administering the T cell of claim 1 to the subject.

36. The method of claim 35, wherein the T cell expresses an antigen-specific TCR or synthetic antigen receptor that recognizes a target antigen in the subject.

37. The method of claim 36, wherein the human subject has cancer and the target antigen is a cancer-specific antigen.

38. (canceled)

39. The method of claim 36 wherein the human subject has an infection.

40. The method of claim 36, wherein the human subject has an autoimmune disorder and the antigen is an antigen associated with the autoimmune disorder, an allergic disorder or transplant rejection.

41-43. (canceled)

44. A method for identifying a targeted insertion in the genome of a cell comprising:
(a) introducing into a population of cells
(i) a targeted nuclease that cleaves a target region in the genome of the cell to create a target insertion site; and
(ii) a plurality of DNA templates that are different by sequence from each other, wherein each DNA template comprises:
i. two or more heterologous coding or noncoding nucleic acid sequences; and
ii. a unique barcode nucleotide sequence that indicates the identity of each heterologous coding or noncoding nucleic acid sequence;
(b) allowing recombination to occur, thereby creating a population of modified cells;
(c) (i) isolating genomic DNA; or
(ii) isolating mRNA from the cells and generating cDNA from the mRNA;
(d) sequencing the genomic DNA or the cDNA to identify a DNA template inserted into the target insertion site for a cell.

45.-62. (canceled)


SEQUENCE LISTING (176 sequences)

SEQ ID NO: 1 - DNA, 1014 bp, synthetic construct
  source 1..1014: mol_type = other DNA
atggagtatttcatggtgcccactcagaaggtgccctctttgcaacatttcaggaaaacagagaaagaagtgataggagggctctgtagccttgccaacattccactaacccccgagactcagcgggaccaggagcggcggattcggcgggagatcgccaacagcaacgagcggagacgcatgcagagcatcaacgcgggattccagtccctcaagaccctcatcccccacacagacggagagaagctcagcaaggcagccattctccagcagacagccgagtacatcttctccctggagcaggagaagaccaggctcttgcagcagaacacacagctcaagcgcttcatccaggagctgagcggctcgtcccccaagcgacggcgggcagaggacaaggacgaaggcataggctccccggacatctgggaggacgagaaggcggaggacctgcggcgggagatgattgagctgcggcagcagctggacaaggagcgctcggtgcgcatgatgctggaggagcaggtgcgctcgctggaggcccacatgtacccggaaaagctcaaggtgattgcgcagcaggtgcagctgcagcagcagcaggaacaggtgaggctgctgcaccaggagaagctggagcgggaacagcagcagctgcggacccagcttctgccccctccggcccccacccaccaccccacggtgatcgtgccagcaccgcctcctcctccctcccaccacatcaatgtcgtcaccatgggcccctcctcggtcatcaactctgtttccacatcccggcaaaatctggacaccatcgtgcaggcaatccagcacatcgagggcacccaggaaaagcaggagctggaggaggagcagcggcgagctgtcatcgtgaagcctgtccgcagctgcccggaggcccccacctctgacaccgcctccgactccgaggcctcagacagtgacgccatggaccagagccgggaggagccgtcgggggacggggagcttccc

SEQ ID NO: 2 - DNA, 375 bp, synthetic construct
  source 1..375: mol_type = other DNA
atgcctcacagctccgacagcagtgactccagcttcagccgctctcctccccctggcaaacaggactcatctgatgatgtgagaagagttcagaggagggagaaaaatcgtattgccgcccagaagagccgacagaggcagacacagaaggccgacaccctgcacctggagagcgaagacctggagaaacagaacgcggctctacgcaaggagatcaagcagctcacagaggaactgaagtacttcacgtcggtgctgaacagccacgagcccctgtgctcggtgctggccgccagcacgccctcgccccccgaggtggtgtacagcgcccacgcattccaccaacctcatgtcagctccccgcgcttccagccc

SEQ ID NO: 3 - DNA, 381 bp, synthetic construct
  source 1..381: mol_type = other DNA
atgtcgcaagggctcccggccgccggcagcgtcctgcagaggagcgtcgcggcgcccgggaaccagccgcagccgcagccgcagcagcagagccctgaggatgatgacaggaaggtccgaaggagagaaaaaaaccgagttgctgctcagagaagtcggaagaagcagacccagaaggctgacaagctccatgaggaatatgagagcctggagcaagaaaacaccatgctgcggagagagatcgggaagctgacagaggagctgaagcacctgacagaggcactgaaggagcacgagaagatgtgcccgctgctgctctgccctatgaactttgtgccagtgcctccccggccggaccctgtggccggctgcttgccccga

SEQ ID NO: 4 - DNA, 1722 bp, synthetic construct
  source 1..1722: mol_type = other DNA
atggcttctgacctagagagtagcctcacctccatagactggctcccccagctgaccctccgagctaccattgagaagcttggaagtgcctcccaggctgggcctcccgggagcagccgcaagtgttcaccagggtcacccacagatcctaatgccaccctgagcaaagacgaggcagcagtgcaccaggacggcaagccacgatacagctatgccactctcatcacctatgccatcaactcctctccagccaagaagatgaccctcagcgagatttaccgctggatctgtgataacttcccctattacaagaatgctggcattggttggaagaattcaatacggcacaacctttctctcaacaagtgtttccggaaggtgcccagacctcgggatgaccctgggaagggttcctattggacaattgacacctgccctgacatttcccgaaagagaagacaccctccagatgatgatctgtcccaagactcaccagaacaggaggcaagcaagagcccacggggaggcgttgcagggagtggagaagcctcactgcctcctgaggggaatccgcagatgtcacttcagagccccacatctatagccagctacagccagggcacaggatctgtggatggtggagcagtggcagcaggggcttcaggccgagaaagtgctgagggtccccctcccctctataacaccaaccatgactttaaattctcctactcagagatcaactttcaggatctaagctggtccttccgcaacctctataagtccatgctggagaagtcctcttcctcctctcagcacggcttttcttctctcctgggggacatcccaccctcgaacaactactacatgtatcagcagcagcagccaccgccacctcaacagcagcagcagcagcagcagccgccacagccacctccccagcagtcccagccacagcagcagcaggcacctgcccagggcccctcagctgtagggggtgctcctccactgcacaccccaagcacagatggttgtaccccaccagggggaaagcaagctggggcggaaggctatgggcctccccctgtaatggccatgcatccacccccgctgcagcatggaggctaccaccctcatcagcaccatccccactcccaccctgcccagcagccaccacctccacagccacaggcacaaggccaggctcccatcaacaacactggctttgcctttccttctgactggtgctctaatattgactctttaaaggaaagcttcaagatggtgaatcggctcaattggtccagcattgagcagtcacaattctcagaactgatggagagtctacgacaggcagagcagaagaactggaccctcgaccagcatcacattgccaatctgtgtgactccctcaaccacttccttactcagactggtcacgtgccccctcaagggggtacccaccgcccaccagcccctgcccgtattgctgactcctgtgccctcaccagtggcaaacaggagtcagccatgagccaagtgaactcttatgggcacccacaagctccccacctctaccctggcccatcaccaatgtacccaatccccacccaggactcagcaggatacaatcgcccagcacaccatatggtccctcggccatcagtgccacctcctggtgccaatgaggagatccctgatgacttcgactgggacttgatcact

SEQ ID NO: 5 - DNA, 1386 bp, synthetic construct
  source 1..1386: mol_type = other DNA
atggccagcaacagcagctcctgcccgacacctgggggcgggcacctcaatgggtacccggtgcctccctacgccttcttcttcccccctatgctgggtggactctccccgccaggcgctctgaccactctccagcaccagcttccagttagtggatatagcacaccatccccagccaccattgagacccagagcagcagttctgaagagatagtgcccagccctccctcgccaccccctctaccccgcatctacaagccttgctttgtctgtcaggacaagtcctcaggctaccactatggggtcagcgcctgtgagggctgcaagggcttcttccgccgcagcatccagaagaacatggtgtacacgtgtcaccgggacaagaactgcatcatcaacaaggtgacccggaaccgctgccagtactgccgactgcagaagtgctttgaagtgggcatgtccaaggagtctgtgagaaacgaccgaaacaagaagaagaaggaggtgcccaagcccgagtgctctgagagctacacgctgacgccggaggtgggggagctcattgagaaggtgcgcaaagcgcaccaggaaaccttccctgccctctgccagctgggcaaatacactacgaacaacagctcagaacaacgtgtctctctggacattgacctctgggacaagttcagtgaactctccaccaagtgcatcattaagactgtggagttcgccaagcagctgcccggcttcaccaccctcaccatcgccgaccagatcaccctcctcaaggctgcctgcctggacatcctgatcctgcggatctgcacgcggtacacgcccgagcaggacaccatgaccttctcggacgggctgaccctgaaccggacccagatgcacaacgctggcttcggccccctcaccgacctggtctttgccttcgccaaccagctgctgcccctggagatggatgatgcggagacggggctgctcagcgccatctgcctcatctgcggagaccgccaggacctggagcagccggaccgggtggacatgctgcaggagccgctgctggaggcgctaaaggtctacgtgcggaagcggaggcccagccgcccccacatgttccccaagatgctaatgaagattactgacctgcgaagcatcagcgccaagggggctgagcgggtgatcacgctgaagatggagatcccgggctccatgccgcctctcatccaggaaatgttggagaactcagagggcctggacactctgagcggacagccggggggtggggggcgggacgggggtggcctggcccccccgccaggcagctgtagccccagcctcagccccagctccaacagaagcagcccggccacccactccccg

SEQ ID NO: 6 - DNA, 357 bp, synthetic construct
  source 1..357: mol_type = other DNA
atgaaggcgctgagcccggtgcgcggctgctacgaggcggtgtgctgcctgtcggaacgcagtctggccatcgcccggggccgagggaagggcccggcagctgaggagccgctgagcttgctggacgacatgaaccactgctactcccgcctgcgggaactggtacccggagtcccgagaggcactcagcttagccaggtggaaatcctacagcgcgtcatcgactacattctcgacctgcaggtagtcctggccgagccagcccctggaccccctgatggcccccaccttcccatccagacagccgagctcactccggaacttgtcatctccaacgacaaaaggagcttttgccac

SEQ ID NO: 7 - DNA, 1047 bp, synthetic construct
  source 1..1047: mol_type = other DNA
atgccggtggaaaggatgcgcatgcgcccgtggctggaggagcagataaactccaacacgatcccggggctcaagtggcttaacaaggaaaagaagatttttcagatcccctggatgcatgcggctagacatgggtgggatgtggaaaaagatgcaccactctttagaaactgggcaatccatacaggaaagcatcaaccaggagtagataaacctgatcccaaaacatggaaggcgaatttcagatgcgccatgaattccttgcctgatattgaagaagtcaaggataaaagcataaagaaaggaaataatgccttcagggtctaccgaatgctgcccctatcagaacggccttctaagaaaggaaagaaaccaaagacagaaaaagaagacaaagttaagcacatcaagcaagaaccagttgagtcatctctggggcttagtaatggagtaagtgatctttctcctgagtatgcggtcctgacttcaactataaaaaatgaagtggatagtacggtgaacatcatagttgtaggacagtcccatctggacagcaacattgagaatcaagagattgtcaccaatccgccagacatttgccaagttgtagaggtgaccactgagagcgacgagcagccggtcagcatgagcgagctctaccctctgcagatctcccccgtgtcttcctatgcagaaagcgaaacgactgatagtgtgcccagcgatgaagagagtgccgaggggcggccacactggcggaagaggaatattgaaggcaaacagtacctcagcaacatggggactcgaggctcctacctgctgcccggcatggcgtccttcgtcacttccaacaaaccggacctccaggtcaccatcaaagaggagagcaatccggtgccttacaacagctcctggcccccttttcaagacctccccctttcttcctccatgaccccagcatccagcagcagtcggccagaccgggagacccgggccagcgtcatcaagaaaacatcggatatcacccaggcccgcgtcaagagctgt

SEQ ID NO: 8 - DNA, 915 bp, synthetic construct
  source 1..915: mol_type = other DNA
atgagtgtggatccagcttgtccccaaagcttgccttgctttgaagcatccgactgtaaagaatcttcacctatgcctgtgatttgtgggcctgaagaaaactatccatccttgcaaatgtcttctgctgagatgcctcacacggagactgtctctcctcttccttcctccatggatctgcttattcaggacagccctgattcttccaccagtcccaaaggcaaacaacccacttctgcagagaagagtgtcgcaaaaaaggaagacaaggtcccggtcaagaaacagaagaccagaactgtgttctcttccacccagctgtgtgtactcaatgatagatttcagagacagaaatacctcagcctccagcagatgcaagaactctccaacatcctgaacctcagctacaaacaggtgaagacctggttccagaaccagagaatgaaatctaagaggtggcagaaaaacaactggccgaagaatagcaatggtgtgacgcagaaggcctcagcacctacctaccccagcctttactcttcctaccaccagggatgcctggtgaacccgactgggaaccttccaatgtggagcaaccagacctggaacaattcaacctggagcaaccagacccagaacatccagtcctggagcaaccactcctggaacactcagacctggtgcacccaatcctggaacaatcaggcctggaacagtcccttctataactgtggagaggaatctctgcagtcctgcatgcagttccagccaaattctcctgccagtgacttggaggctgccttggaagctgctggggaaggccttaatgtaatacagcagaccactaggtattttagtactccacaaaccatggatttattcctaaactactccatgaacatgcaacctgaagacgtg

SEQ ID NO: 9 - DNA, 492 bp, synthetic construct
  source 1..492: mol_type = other DNA
atgagcgttgacccattgagctctaaggcactgaaaattaaaagagaattgagtgaaaatacaccccatctttccgatgaagcacttatgggcctctctgttcgagaactcaatcgacaccttcgaggactgtctgcggaagaagttactcgcctgaaacaacgcagacggactctgaagaatcgcgggtatgctgcttcctgtcgggtcaaaagagtctgtcagaaagaagaactccaaaagcaaaagtccgaacttgaacgggaagtagataaattggctagggaaaatgctgcaatgagactcgaattggatgctcttagaggaaaatgtgaagctctccaaggatttgcacggtctgttgctgctgcacggggccctgcgacactggtcgcccctgcatccgtgataacgattgttaaaagcacacctggaagcggatcaggacctgctcatggaccagatcctgcacatgggcccgcttcatgtagt

SEQ ID NO: 10 - DNA, 2385 bp, synthetic construct
  source 1..2385: mol_type = other DNA
atggatcatttgaacgaggcaactcaggggaaagaacattcagaaatgtctaacaatgtgagtgatccgaagggtccaccagccaagattgcccgcctggagcagaacgggagcccgctaggaagaggaaggcttgggagtacaggtgcaaaaatgcagggagtgcctttaaaacactcgggccatctgatgaaaaccaaccttaggaaaggaaccatgctgccagttttctgtgtggtggaacattatgaaaacgccattgaatatgattgcaaggaggagcatgcagaatttgtgctggtgagaaaggatatgcttttcaaccagctgatcgaaatggcattgctgtctctaggttattcacatagctctgctgcccaggccaaagggctaatccaggttggaaagtggaatccagttccactgtcttacgtgacagatgcccctgatgctacagtagcagatatgcttcaagatgtgtatcatgtggtcacattgaaaattcagttacacagttgccccaaactagaagacttgcctcccgaacaatggtcgcacaccacagtgaggaatgctctgaaggacttactgaaagatatgaatcagagttcattggccaaggagtgccccctttcacagagtatgatttcttccattgtgaacagtacttactatgcaaatgtctcagcagcaaaatgtcaagaatttggaaggtggtacaaacatttcaagaagacaaaagatatgatggttgaaatggatagtctttctgagctatcccagcaaggcgccaatcatgtcaattttggccagcaaccagttccagggaacacagccgagcagcctccatcccctgcgcagctctcccatggcagccagccctctgtccggacacctcttccaaacctgcaccctgggctcgtatcaacacctatcagtcctcaattggtcaaccagcagctggtgatggctcagctgctgaaccagcagtatgcagtgaatagacttttagcccagcagtccttaaaccaacaatacttgaaccaccctccccctgtcagtagatctatgaataagcctttggagcaacaggtttcgaccaacacagaggtgtcttccgaaatctaccagtgggtacgcgatgaactgaaacgagcaggaatctcccaggcggtatttgcacgtgtggcttttaacagaactcagggcttgctttcagaaatcctccgaaaggaagaggaccccaagactgcatcccagtctttgctggtaaaccttcgggctatgcagaatttcttgcagttaccggaagctgaaagagaccgaatataccaggacgaaagggaaaggagcttgaatgctgcctcggccatgggtcctgcccccctcatcagcacaccacccagccgtcctccccaggtgaaaacagctactattgccactgaaaggaatgggaaaccagagaacaataccatgaacattaatgcttccatttatgatgagattcagcaggaaatgaagcgtgctaaagtgtctcaagcactgtttgcaaaggttgcagcaaccaaaagccagggatggttgtgcgagctgttacgctggaaagaagatccttctccagaaaacagaaccctgtgggagaacctctccatgatccgaaggttcctcagtcttcctcagccagaacgtgatgccatttatgaacaggagagcaacgcggtgcatcaccatggcgacaggccgccccacattatccatgttccagcagagcagattcagagcccctctcccaccacccttgggaaaggagagtctagaggcgttttcttaccaggcctgccgacccctgcaccatggctcggtgctgctcctcagcaacagcagcagcaacagcaacagcagcagcagcagcagcaggcaccgccgcctccacagccacagcagcagccacagacaggccctcggctccccccacggcaacccacggtggcctctccagcagagtcagatgaggaaaaccgacagaagacccggccacgaacaaaaatttcagtggaagccttgggaatcctccagagtttcatacaagacgtgggcctgtaccctgacgaagaggccatccagactctgtctgcccagctcgaccttcccaagtacaccatcatcaagttctttcagaaccagcggtactatctcaagcaccacggcaaactgaaggacaattccggtttagaggtcgatgtggcagaatataaagaagaggagctgctgaaggatttggaagagagtgtccaagataaaaatactaacacccttttttcagtgaaactagaagaagagctgtcagtggaaggaaacacagacattaatactgatttgaaagac

SEQ ID NO: 11 - DNA, 2058 bp, synthetic construct
  source 1..2058: mol_type = other DNA
atgcaactcggagaacaactgctcgttagttctgtcaatcttcccggggcacatttctatcccctcgaatcagcaaggggcgggtcaggtggatccgccggtcatctgccttctgctgctccttcccctcaaaagctggatctcgataaggctagcaagaaattcagcggatccctgtcatgtgaagcagtatctggtgaaccagctgcggcgtctgctggtgctccagccgcaatgttgagcgatactgatgcaggagatgccttcgcaagtgcagcagctgtcgctaaaccaggaccacccgatgggagaaaagggagcccgtgtggcgaagaagaattgccgtctgctgccgccgcagcggctgctgctgctgcagccgccgccgctaccgcccgttattctatggattccttgagtagcgaaaggtattatcttcaaagtcctggcccgcaaggttctgaattggccgccccatgtagcctgtttccttatcaagccgctgccggcgctcctcatggtcccgtatatcccgccccaaatggcgccagatatccatatgggtcaatgcttccccctggtggatttcctgctgctgtatgtcccccaggacgggcccaatttgggcctggggcaggggctggttcaggggcaggtggctcttctggtggcggcggtgggccaggtacataccaatattcacaaggcgccccactgtatggtccatatccgggcgctgctgccgctgggagctgtggcggcctcggcgggcttggcgtgcctggaagcggttttagggcacatgtgtatttgtgtaatcgaccactttggctgaagtttcataggcatcagacggaaatgataatcactaagcaagggcgaaggatgttcccatttctgtcctttaatattaatggtctgaacccaaccgcacattataacgtctttgtggaagtcgtccttgcagatcctaatcattggcggtttcaaggcggaaagtgggttacgtgcggaaaggcggataacaatatgcaagggaataagatgtacgtccatcctgaatcaccgaacacagggagtcattggatgaggcaagaaataagctttggaaagctgaagctgacgaacaataagggagccaacaataataatactcaaatgatcgtgcttcagtcacttcataagtatcagccaaggcttcacatagtagaggtcacggaagacggggtcgaagatctgaacgaaccatccaaaacacaaaccttcacattttccgagacccagtttatcgccgtcacagcgtatcagaatacagacataacccagctcaaaatagaccacaatcctttcgccaagggatttcgcgataattacgactcctcacaccaaatagtgcccggcggcaggtatggtgtgcagagtttctttccagaaccgttcgtgaatacattgccccaggcacggtactacaacggggaacgaacagtcccccaaactaatggtttgctcagcccacagcaatccgaggaagttgcaaatccgccacaaagatggctcgtaactcccgtgcaacagcccggcacgaataagctggatatatctagctacgagtccgagtacacaagttccacccttcttccgtacgggatcaagagcctgccactgcaaacctcacacgcattgggctactatcccgatcccacattccccgccatggccggctggggcggcagaggctcatatcaacgcaaaatggccgcgggtttgccctggacaagccgcaccagtccgacagtgttttcagaggaccaactgagtaaagaaaaggtaaaggaagagatcggttcaagttggatcgaaaccccaccatcaattaagagcctcgacagtaacgacagcggcgtgtatacttccgcctgcaaaaggagacgtctcagcccctctaattcttccaacgagaactccccgagtattaaatgcgaagatatcaacgcagaggaatacagcaaggatacatctaaggggatgggtggctactacgccttctatactacacct

SEQ ID NO: 12 - DNA, 975 bp, synthetic construct
  source 1..975: mol_type = other DNA
atgcccatcactcggatgcgcatgagaccctggctagagatgcagattaattccaaccaaatcccggggctcatctggattaataaagaggagatgatcttccagatcccatggaagcatgctgccaagcatggctgggacatcaacaaggatgcctgtttgttccggagctgggccattcacacaggccgatacaaagcaggggaaaaggagccagatcccaagacgtggaaggccaactttcgctgtgccatgaactccctgccagatatcgaggaggtgaaagaccagagcaggaacaagggcagctcagctgtgcgagtgtaccggatgcttccacctctcaccaagaaccagagaaaagaaagaaagtcgaagtccagccgagatgctaagagcaaggccaagaggaagtcatgtggggattccagccctgataccttctctgatggactcagcagctccactctgcctgatgaccacagcagctacacagttccaggctacatgcaggacttggaggtggagcaggccctgactccagcactgtcgccatgtgctgtcagcagcactctccccgactggcacatcccagtggaagttgtgccggacagcaccagtgatctgtacaacttccaggtgtcacccatgccctccacctctgaagctacaacagatgaggatgaggaagggaaattacctgaggacatcatgaagctcttggagcagtcggagtggcagccaacaaacgtggatgggaaggggtacctactcaatgaacctggagtccagcccacctctgtctatggagactttagctgtaaggaggagccagaaattgacagcccagggggggatattgggctgagtctacagcgtgtcttcacagatctgaagaacatggatgccacctggctggacagcctgctgaccccagtccggttgccctccatccaggccattccctgtgcaccg

SEQ ID NO: 13 - DNA, 336 bp, synthetic construct
  source 1..336: mol_type = other DNA
atgctcattttcctgggctgttacagaagaagactggaagagcgcgcagggaccatgtcggcggagaccgcgagcggccccacagaggaccaggtggaaatcctggagtacaacttcaacaaggtcgacaagcacccggattccaccacgctgtgcctcatcgcggccgaggcaggcctttccgaggaggagacccagggtagtgatttgatctccaggagcaaaatatggcacccagaaagtagcccccaaagagaaggctacccccatgatagtctgccgtgcttggctttcgattacttttctctactgccgccccagtgtaaagaaatggtt

SEQ ID NO: 14 - DNA, 519 bp, synthetic construct
  source 1..519: mol_type = other DNA
atgggcttgccgagaagagcgggagatgctgccgaactccggaaatcattgaaaccccttctcgaaaagcggagaagagccagaattaatcaatccctcagtcaactgaaaggcttgatacttccactcttgggtcgagaaaattctaattgttctaaactcgagaaagctgatgtgttggagatgacagtccggtttctccaagaactccccgcctctagctggccaaccgctgcacctcttccgtgtgatagttatagagaaggatattcagcttgcgttgcacgacttgctagggtcctgccagcatgtagggtattggaaccagctgtcagtgcaaggttgctcgaacatctttggcgacgagctgcatctgctactttggatggcggacgtgccggcgacagctccggtcctagtgctcctgctcctgctcctgcaagcgcacctgaaccggcttcagcacctgtcccaagtcccccgtcaccaccttgtgggcccggattgtggcgtccatgg

SEQ ID NO: 15 - DNA, 402 bp, synthetic construct
  source 1..402: mol_type = other DNA
atgaaagccttcagtcccgtgaggtccgttaggaaaaacagcctgtcggaccacagcctgggcatctcccggagcaaaacccctgtggacgacccgatgagcctgctatacaacatgaacgactgctactccaagctcaaggagctggtgcccagcatcccccagaacaagaaggtgagcaagatggaaatcctgcagcacgtcatcgactacatcttggacctgcagatcgccctggactcgcatcccactattgtcagcctgcatcaccagagacccgggcagaaccaggcgtccaggacgccgctgaccaccctcaacacggatatcagcatcctgtccttgcaggcttctgaattcccttctgagttaatgtcaaatgacagcaaagcactgtgtggc

SEQ ID NO: 16 - DNA, 1908 bp, synthetic construct
  source 1..1908: mol_type = other DNA
atggcccagtccaccgccacctcccctgatgggggcaccacgtttgagcacctctggagctctctggaaccagacagcacctacttcgaccttccccagtcaagccgggggaataatgaggtggtgggcggaacggattccagcatggacgtcttccacctggagggcatgactacatctgtcatggcccagttcaatctgctgagcagcaccatggaccagatgagcagccgcgcggcctcggccagcccctacaccccagagcacgccgccagcgtgcccacccactcgccctacgcacaacccagctccaccttcgacaccatgtcgccggcgcctgtcatcccctccaacaccgactaccccggaccccaccactttgaggtcactttccagcagtccagcacggccaagtcagccacctggacgtactccccgctcttgaagaaactctactgccagatcgccaagacatgccccatccagatcaaggtgtccaccccgccacccccaggcaccgccatccgggccatgcctgtttacaagaaagcggagcacgtgaccgacgtcgtgaaacgctgccccaaccacgagctcgggagggacttcaacgaaggacagtctgctccagccagccacctcatccgcgtggaaggcaataatctctcgcagtatgtggatgaccctgtcaccggcaggcagagcgtcgtggtgccctatgagccaccacaggtggggacggaattcaccaccatcctgtacaacttcatgtgtaacagcagctgtgtagggggcatgaaccggcggcccatcctcatcatcatcaccctggagatgcgggatgggcaggtgctgggccgccggtcctttgagggccgcatctgcgcctgtcctggccgcgaccgaaaagctgatgaggaccactaccgggagcagcaggccctgaacgagagctccgccaagaacggggccgccagcaagcgtgccttcaagcagagcccccctgccgtccccgcccttggtgccggtgtgaagaagcggcggcatggagacgaggacacgtactaccttcaggtgcgaggccgggagaactttgagatcctgatgaagctgaaagagagcctggagctgatggagttggtgccgcagccactggtggactcctatcggcagcagcagcagctcctacagaggccgagtcacctacagcccccgtcctacgggccggtcctctcgcccatgaacaaggtgcacgggggcatgaacaagctgccctccgtcaaccagctggtgggccagcctcccccgcacagttcggcagctacacccaacctggggcccgtgggccccgggatgctcaacaaccatggccacgcagtgccagccaacggcgagatgagcagcagccacagcgcccagtccatggtctcggggtcccactgcactccgccacccccctaccacgccgaccccagcctcgtcagttttttaacaggattggggtgtccaaactgcatcgagtatttcacctcccaagggttacagagcatttaccacctgcagaacctgaccattgaggacctgggggccctgaagatccccgagcagtaccgcatgaccatctggcggggcctgcaggacctgaagcagggccacgactacagcaccgcgcagcagctgctccgctctagcaacgcggccaccatctccatcggcggctcaggggaactgcagcgccagcgggtcatggaggccgtgcacttccgcgtgcgccacaccatcaccatccccaaccgcggcggcccaggcggcggccctgacgagtgggcggacttcggcttcgacctgcccgactgcaaggcccgcaagcagcccatcaaggaggagttcacggaggccgagatccac

SEQ ID NO: 17 - DNA, 1275 bp, synthetic construct
  source 1..1275: mol_type = other DNA
atgtcgtccatcctgcctttcactcccccgatcgtgaagcgcctgctgggctggaagaagggcgagcagaacgggcaggaggagaaatggtgcgagaaggcggtcaagagcctggtcaagaaactcaagaagacggggcagctggacgagctggagaaggccatcaccacgcagaacgtcaacaccaagtgcatcaccatccccaggtccctggatggccggttgcaggtgtcccatcggaaggggctccctcatgtcatctactgccgcctgtggcgatggccagacctgcacagccaccacgagctacgggccatggagctgtgtgagttcgccttcaatatgaagaaggacgaggtctgcgtgaatccctaccactaccagagagtagagacaccagttctacctcctgtgttggtgccacgccacacagagatcccggccgagttccccccactggacgactacagccattccatccccgaaaacactaacttccccgcaggcatcgagccccagagcaatattccagagaccccaccccctggctacctgagtgaagatggagaaaccagtgaccaccagatgaaccacagcatggacgcaggttctccaaacctatccccgaatccgatgtccccagcacataataacttggacctgcagccagttacctactgcgagccggccttctggtgctccatctcctactacgagctgaaccagcgcgtcggggagacattccacgcctcgcagccatccatgactgtggatggcttcaccgacccctccaattcggagcgcttctgcctagggctgctctccaatgtcaacaggaatgcagcagtggagctgacacggagacacatcggaagaggcgtgcggctctactacatcggaggggaggtcttcgcagagtgcctcagtgacagcgctatttttgtccagtctcccaactgtaaccagcgctatggctggcacccggccaccgtctgcaagatcccaccaggatgcaacctgaagatcttcaacaaccaggagttcgctgccctcctggcccagtcggtcaaccagggctttgaggctgtctaccagttgacccgaatgtgcaccatccgcatgagcttcgtcaaaggctggggagcggagtacaggagacagactgtgaccagtaccccctgctggattgagctgcacctgaatgggcctttgcagtggcttgacaaggtcctcacccagatgggctccccaagcatccgctgttccagtgtgtct

SEQ ID NO: 18 - DNA, 1515 bp, synthetic construct
  source 1..1515: mol_type = other DNA
atgaaattcaagttacatgtgaattctgccaggcaatacaaggacctgtggaatatgagtgatgacaaaccctttctatgtactgcgcctggatgtggccagcgttttaccaacgaggatcatttggctgtccataaacataaacatgagatgacactgaaatttggtccagcacgtaatgacagtgtcattgtggctgatcagaccccaacaccaacaagattcttgaaaaactgtgaagaagtgggtttgtttaatgagttggcgagtccatttgagaatgaattcaagaaagcttcagaagatgacattaaaaaaatgcctctagatttatcccctcttgcaacacctatcataagaagcaaaattgaggagccttctgttgtagaaacaactcaccaggatagtcctttacctcacccagagtctactaccagtgatgagaaggaagtaccattggcacaaactgcacagcccacatcagctattgttcgtccagcatcattacaggttcccaatgtgctgcttacaagttctgactcaagtgtaattattcagcaggcagtaccttcaccaacctcaagtactgtaatcacccaggcaccatcctctaacaggccaattgtccctgtaccaggcccatttcctcttctgttacatcttcctaatggacaaaccatgcctgttgctattcctgcatcaattacaagttctaatgtgcatgttccagctgcagtcccactcgttcgaccagtcaccatggtgcctagtgttccaggaatcccaggtccttcctctccccaaccagtacagtcagaagcaaaaatgagattaaaagctgctttgacccagcaacatcctccagttaccaatggtgatactgtcaaaggtcatggtagcggattggttaggactcagtcagaggaatctcgaccgcagtcattacaacagccagccacatccactacagaaactccggcttctccagctcacacaactccacagacccaaagtacaagtggtcgtcggagaagagcagctaacgaagatcctgatgaaaaaaggagaaagtttttagagcgaaatagagcagcagcttcaagatgccgacaaaaaaggaaagtctgggttcagtctttagagaagaaagctgaagacttgagttcattaaatggtcagctgcagagtgaagtcaccctgctgagaaatgaagtggcacagctgaaacagcttcttctggctcataaagattgccctgtaaccgccatgcagaagaaatctggctatcatactgctgataaagatgatagttcagaagacatttcagtgccgagtagtccacatacagaagctatacagcatagttcggtcagcacatccaatggagtcagttcaacctccaaggcagaagctgtagccacttcagtcctcacccagatggcggaccagagtacagagcctgctctttcacagatcgttatggctccttcctcccagtcacagccctcaggaagt

SEQ ID NO: 19 - DNA, 1656 bp, synthetic construct
  source 1..1656: mol_type = other DNA
atggacaatatgtctattacgaatacaccaacaagtaatgatgcctgtctgagcattgtgcatagtttgatgtgccatagacaaggtggagagagtgaaacatttgcaaaaagagcaattgaaagtttggtaaagaagctgaaggagaaaaaagatgaattggattctttaataacagctataactacaaatggagctcatcctagtaaatgtgttaccatacagagaacattggatgggaggcttcaggtggctggtcggaaaggatttcctcatgtgatctatgcccgtctctggaggtggcctgatcttcacaaaaatgaactaaaacatgttaaatattgtcagtatgcgtttgacttaaaatgtgatagtgtctgtgtgaatccatatcactacgaacgagttgtatcacctggaattgatctctcaggattaacactgcagagtaatgctccatcaagtatgatggtgaaggatgaatatgtgcatgactttgagggacagccatcgttgtccactgaaggacattcaattcaaaccatccagcatccaccaagtaatcgtgcatcgacagagacatacagcaccccagctctgttagccccatctgagtctaatgctaccagcactgccaactttcccaacattcctgtggcttccacaagtcagcctgccagtatactggggggcagccatagtgaaggactgttgcagatagcatcagggcctcagccaggacagcagcagaatggatttactggtcagccagctacttaccatcataacagcactaccacctggactggaagtaggactgcaccatacacacctaatttgcctcaccaccaaaacggccatcttcagcaccacccgcctatgccgccccatcccggacattactggcctgttcacaatgagcttgcattccagcctcccatttccaatcatcctgctcctgagtattggtgttccattgcttactttgaaatggatgttcaggtaggagagacatttaaggttccttcaagctgccctattgttactgttgatggatacgtggacccttctggaggagatcgcttttgtttgggtcaactctccaatgtccacaggacagaagccattgagagagcaaggttgcacataggcaaaggtgtgcagttggaatgtaaaggtgaaggtgatgtttgggtcaggtgccttagtgaccacgcggtctttgtacagagttactacttagacagagaagctgggcgtgcacctggagatgctgttcataagatctacccaagtgcatatataaaggtctttgatttgcgtcagtgtcatcgacagatgcagcagcaggcggctactgcacaagctgcagcagctgcccaggcagcagccgtggcaggaaacatccctggcccaggatcagtaggtggaatagctccagctatcagtctgtcagctgctgctggaattggtgttgatgaccttcgtcgcttatgcatactcaggatgagttttgtgaaaggctggggaccggattacccaagacagagcatcaaagaaacaccttgctggattgaaattcacttacaccgggccctccagctcctagacgaagtacttcataccatgccgattgcagacccacaacctttagac

SEQ ID NO: 20 - DNA, 1293 bp, synthetic construct
  source 1..1293: mol_type = other DNA
atgcccaaccccaggcctggcaagccctcggccccttccttggcccttggcccatccccaggagcctcgcccagctggagggctgcacccaaagcctcagacctgctgggggcccggggcccagggggaaccttccagggccgagatcttcgaggcggggcccatgcctcctcttcttccttgaaccccatgccaccatcgcagctgcagctgcccacactgcccctagtcatggtggcaccctccggggcacggctgggccccttgccccacttacaggcactcctccaggacaggccacatttcatgcaccagctctcaacggtggatgcccacgcccggacccctgtgctgcaggtgcaccccctggagagcccagccatgatcagcctcacaccacccaccaccgccactggggtcttctccctcaaggcccggcctggcctcccacctgggatcaacgtggccagcctggaatgggtgtccagggagccggcactgctctgcaccttcccaaatcccagtgcacccaggaaggacagcaccctttcggctgtgccccagagctcctacccactgctggcaaatggtgtctgcaagtggcccggatgtgagaaggtcttcgaagagccagaggacttcctcaagcactgccaggcggaccatcttctggatgagaagggcagggcacaatgtctcctccagagagagatggtacagtctctggagcagcagctggtgctggagaaggagaagctgagtgccatgcaggcccacctggctgggaaaatggcactgaccaaggcttcatctgtggcatcatccgacaagggctcctgctgcatcgtagctgctggcagccaaggccctgtcgtcccagcctggtctggcccccgggaggcccctgacagcctgtttgctgtccggaggcacctgtggggtagccatggaaacagcacattcccagagttcctccacaacatggactacttcaagttccacaacatgcgaccccctttcacctacgccacgctcatccgctgggccatcctggaggctccagagaagcagcggacactcaatgagatctaccactggttcacacgcatgtttgccttcttcagaaaccatcctgccacctggaagaacgccatccgccacaacctgagtctgcacaagtgctttgtgcgggtggagagcgagaagggggctgtgtggaccgtggatgagctggagttccgcaagaaacggagccagaggcccagcaggtgttccaaccctacacctggcccc

SEQ ID NO: 21 - DNA, 1653 bp, synthetic construct
  source 1..1653: mol_type = other DNA
atggacgaactgttccccctcatcttcccggcagagccagcccaggcctctggcccctatgtggagatcattgagcagcccaagcagcggggcatgcgcttccgctacaagtgcgaggggcgctccgcgggcagcatcccaggcgagaggagcacagataccaccaagacccaccccaccatcaagatcaatggctacacaggaccagggacagtgcgcatctccctggtcaccaaggaccctcctcaccggcctcacccccacgagcttgtaggaaaggactgccgggatggcttctatgaggctgagctctgcccggaccgctgcatccacagtttccagaacctgggaatccagtgtgtgaagaagcgggacctggagcaggctatcagtcagcgcatccagaccaacaacaaccccttccaagttcctatagaagagcagcgtggggactacgacctgaatgctgtgcggctctgcttccaggtgacagtgcgggacccatcaggcaggcccctccgcctgccgcctgtcctttctcatcccatctttgacaatcgtgcccccaacactgccgagctcaagatctgccgagtgaaccgaaactctggcagctgcctcggtggggatgagatcttcctactgtgtgacaaggtgcagaaagaggacattgaggtgtatttcacgggaccaggctgggaggcccgaggctccttttcgcaagctgatgtgcaccgacaagtggccattgtgttccggacccctccctacgcagaccccagcctgcaggctcctgtgcgtgtctccatgcagctgcggcggccttccgaccgggagctcagtgagcccatggaattccagtacctgccagatacagacgatcgtcaccggattgaggagaaacgtaaaaggacatatgagaccttcaagagcatcatgaagaagagtcctttcagcggacccaccgacccccggcctccacctcgacgcattgctgtgccttcccgcagctcagcttctgtccccaagccagcaccccagccctatccctttacgtcatccctgagcaccatcaactatgatgagtttcccaccatggtgtttccttctgggcagatcagccaggcctcggccttggccccggcccctccccaagtcctgccccaggctccagcccctgcccctgctccagccatggtatcagctctggcccaggccccagcccctgtcccagtcctagccccaggccctcctcaggctgtggccccacctgcccccaagcccacccaggctggggaaggaacgctgtcagaggccctgctgcagctgcagtttgatgatgaagacctgggggccttgcttggcaacagcacagacccagctgtgttcacagacctggcatccgtcgacaactccgagtttcagcagctgctgaaccagggcatacctgtggccccccacacaactgagcccatgctgatggagtaccctgaggctataactcgcctagtgacaggggcccagaggccccccgacccagctcctgctccactgggggccccggggctccccaatggcctcctttcaggagatgaagacttctcctccattgcggacatggacttctcagccctgctgagtcagatcagctcc

SEQ ID NO: 22 - DNA, 2289 bp, synthetic construct
  source 1..2289: mol_type = other DNA
atgcttactgaccctgatttacctcaggagtttgaaaggatgtcttccaagcgaccagcctctccgtatggggaagcagatggagaggtagccatggtgacaagcagacagaaagtggaagaagaggagagtgacgggctcccagcctttcaccttcccttgcatgtgagttttcccaacaagcctcactctgaggaatttcagccagtttctctgctgacgcaagagacttgtggccataggactcccacttctcagcacaatacaatggaagttgatggcaataaagttatgtcttcatttgccccacacaactcatctacctcacctcagaaggcagaagaaggtgggcgacagagtggcgagtccttgtctagtacagccctgggaactcctgaacggcgcaagggcagtttagctgatgttgttgacaccttgaagcagaggaaaatggaagagctcatcaaaaacgagccggaagaaacccccagtattgaaaaactactctcaaaggactggaaagacaagcttcttgcaatgggatcggggaactttggcgaaataaaagggactcccgagagcttagctgagaaagaaaggcaactcatgggtatgatcaaccagctgaccagcctccgagagcagctgttggctgcccacgatgagcagaagaaactagctgcctctcagattgagaaacagcgtcagcaaatggagctggccaagcagcaacaagaacaaattgcaagacagcagcagcagcttctacagcaacaacacaaaatcaatttgctccagcaacagatccaggttcaaggtcagctgccgccattaatgattcccgtattccctcctgatcaacggacactggctgcagctgcccagcaaggattcctcctccctccaggcttcagctataaggctggatgtagtgacccttaccctgttcagctgatcccaactaccatggcagctgctgccgcagcaacaccaggcttaggcccactccaactgcagcagttatatgctgcccagctagctgcaatgcaggtatctccaggagggaagctgccaggcataccccaaggcaaccttggtgctgctgtatctcctaccagcattcacacagacaagagcacaaacagcccaccacccaaaagcaaggatgaagtggcacagccactgaacctatcagctaaacccaagacctctgatggcaaatcacccacatcacccacctctccccatatgccagctctgagaataaacagtggggcaggccccctcaaagcctctgtcccagcagcgttagctagtccttcagccagagttagcacaataggttacttaaatgaccatgatgctgtcaccaaggcaatccaagaagctcggcaaatgaaggagcaactccgacgggaacaacaggtgcttgatgggaaggtggctgttgtgaatagtctgggtctcaataactgccgaacagaaaaggaaaaaacaacactggagagtctgactcagcaactggcagttaaacagaatgaagaaggaaaatttagccatgcaatgatggatttcaatctgagtggagattctgatggaagtgctggagtctcagagtcaagaatttatagggaatcccgagggcgtggtagcaatgaaccccacataaagcgtccaatgaatgccttcatggtgtgggctaaagatgaacggagaaagatccttcaagcctttcctgacatgcacaactccaacatcagcaagatattgggatctcgctggaaagctatgacaaacctagagaaacagccatattatgaggagcaagcccgtctcagcaagcagcacctggagaagtaccctgactataagtacaagcccaggccaaagcgcacctgcctggtggatggcaaaaagctgcgcattggtgaatacaaggcaatcatgcgcaacaggcggcaggaaatgcggcagtacttcaatgttgggcaacaagcacagatccccattgccactgctggtgttgtgtaccctggagccatcgccatggctgggatgccctcccctcacctgccctcggagcactcaagcgtgtctagcagcccagagcctgggatgcctgttatccagagcacttacggtgtgaaaggagaggagccacatatcaaagaagagatacaggccgaggacatcaatggagaaatttatgatgagtacgacgaggaagaggatgatccagatgtagattatgggagtgacagtgaaaaccatattgcaggacaagccaac

SEQ ID NO: 23 - DNA, 507 bp, synthetic construct
  source 1..507: mol_type = other DNA
atgcgctggtgtctcctcctgatctgggcccaggggctgaggcaggctcccctcgcctcaggaatgatgacaggcacaatagaaacaacggggaacatttctgcagagaaaggtggctctatcatcttacaatgtcacctctcctccaccacggcacaagtgacccaggtcaactgggagcagcaggaccagcttctggccatttgtaatgctgacttggggtggcacatctccccatccttcaaggatcgagtggccccaggtcccggcctgggcctcaccctccagtcgctgaccgtgaacgatacaggggagtacttctgcatctatcacacctaccctgatgggacgtacactgggagaatcttcctggaggtcctagaaagctcagtggctgagcacggtgccaggttccagattccattgcttggagccatggccgcgacgctggtggtcatctgcacagcagtcatcgtggtggtcgcgttgactagaaagaagaaagcc

SEQ ID NO: 24 - DNA, 1317 bp, synthetic construct
  source 1..1317: mol_type = other DNA
atgcccctcaacgttagcttcaccaacaggaactatgacctcgactacgactcggtgcagccgtatttctactgcgacgaggaggagaacttctaccagcagcagcagcagagcgagctgcagcccccggcgcccagcgaggatatctggaagaaattcgagctgctgcccaccccgcccctgtcccctagccgccgctccgggctctgctcgccctcctacgttgcggtcacacccttctcccttcggggagacaacgacggcggtggcgggagcttctccacggccgaccagctggagatggtgaccgagctgctgggaggagacatggtgaaccagagtttcatctgcgacccggacgacgagaccttcatcaaaaacatcatcatccaggactgtatgtggagcggcttctcggccgccgccaagctcgtctcagagaagctggcctcctaccaggctgcgcgcaaagacagcggcagcccgaaccccgcccgcggccacagcgtctgctccacctccagcttgtacctgcaggatctgagcgccgccgcctcagagtgcatcgacccctcggtggtcttcccctaccctctcaacgacagcagctcgcccaagtcctgcgcctcgcaagactccagcgccttctctccgtcctcggattctctgctctcctcgacggagtcctccccgcagggcagccccgagcccctggtgctccatgaggagacaccgcccaccaccagcagcgactctgaggaggaacaagaagatgaggaagaaatcgatgttgtttctgtggaaaagaggcaggctcctggcaaaaggtcagagtctggatcaccttctgctggaggccacagcaaacctcctcacagcccactggtcctcaagaggtgccacgtctccacacatcagcacaactacgcagcgcctccctccactcggaaggactatcctgctgccaagagggtcaagttggacagtgtcagagtcctgagacagatcagcaacaaccgaaaatgcaccagccccaggtcctcggacaccgaggagaatgtcaagaggcgaacacacaacgtcttggagcgccagaggaggaacgagctaaaacggagcttttttgccctgcgtgaccagatcccggagttggaaaacaatgaaaaggcccccaaggtagttatccttaaaaaagccacagcatacatcctgtccgtccaagcagaggagcaaaagctcatttctgaagaggacttgttgcggaaacgacgagaacagttgaaacacaaacttgaacagctacggaactcttgtgcg

SEQ ID NO: 25 - DNA, 816 bp, synthetic construct
  source 1..816: mol_type = other DNA
atggattcatacctgctgatgtggggactgctcacgttcatcatggtgcctggctgccaggcagagctctgtgacgatgacccgccagagatcccacacgccacattcaaagccatggcctacaaggaaggaaccatgttgaactgtgaatgcaagagaggtttccgcagaataaaaagcgggtcactctatatgctctgtacaggaaactctagccactcgtcctgggacaaccaatgtcaatgcacaagctctgccactcggaacacaacgaaacaagtgacacctcaacctgaagaacagaaagaaaggaaaaccacagaaatgcaaagtccaatgcagccagtggaccaagcgagccttccaggtcactgcagggaacctccaccatgggaaaatgaagccacagagagaatttatcatttcgtggtggggcagatggtttattatcagtgcgtccagggatacagggctctacacagaggtcctgctgagagcgtctgcaaaatgacccacgggaagacaaggtggacccagccccagctcatatgcacaggtgaaatggagaccagtcagtttccaggtgaagagaagcctcaggcaagccccgaaggccgtcctgagagtgagacttcctgcctcgtcacaacaacagattttcaaatacagacagaaatggctgcaaccatggagacgtccatatttacaacagagtaccaggtagcagtggccggctgtgttttcctgctgatcagcgtcctcctcctgagtgggctcacctggcagcggagacagaggaagagtagaagaacaatc

SEQ ID NO: 26 - DNA, 765 bp, synthetic construct
  source 1..765: mol_type = other DNA
atgggaaacagctgttacaacatagtagccactctgttgctggtcctcaactttgagaggacaagatcattgcaggatccttgtagtaactgcccagctggtacattctgtgataataacaggaatcagatttgcagtccctgtcctccaaatagtttctccagcgcaggtggacaaaggacctgtgacatatgcaggcagtgtaaaggtgttttcaggaccaggaaggagtgttcctccaccagcaatgcagagtgtgactgcactccagggtttcactgcctgggggcaggatgcagcatgtgtgaacaggattgtaaacaaggtcaagaactgacaaaaaaaggttgtaaagactgttgctttgggacatttaacgatcagaaacgtggcatctgtcgaccctggacaaactgttctttggatggaaagtctgtgcttgtgaatgggacgaaggagagggacgtggtctgtggaccatctccagccgacctctctccgggagcatcctctgtgaccccgcctgcccctgcgagagagccaggacactctccgcagatcatctccttctttcttgcgctgacgtcgactgcgttgctcttcctgctgttcttcctcacgctccgtttctctgttgttaaacggggcagaaagaaactcctgtatatattcaaacaaccatttatgagaccagtacaaactactcaagaggaagatggctgtagctgccgatttccagaagaagaagaaggaggatgtgaactg

SEQ ID NO: 27 - DNA, 1395 bp, synthetic construct
  source 1..1395: mol_type = other DNA
atgaatgtgacaagtttattttcctttacaagtccagctgtgaagagacttcttgggtggaaacagggcgatgaagaagaaaaatgggcagagaaagctgttgatgctttggtgaaaaaactgaagaaaaagaaaggtgccatggaggaactggaaaaggccttgagctgcccagggcaaccgagtaactgtgtcaccattccccgctctctggatggcaggctgcaagtctcccaccggaagggactgcctcatgtcatttactgccgtgtgtggcgctggcccgatcttcagagccaccatgaactaaaaccactggaatgctgtgagtttccttttggttccaagcagaaggaggtctgcatcaatccctaccactataagagagtagaaagccctgtacttcctcctgtgctggttccaagacacagcgaatataatcctcagcacagcctcttagctcagttccgtaacttaggacaaaatgagcctcacatgccactcaacgccacttttccagattctttccagcaacccaacagccacccgtttcctcactctcccaatagcagttacccaaactctcctgggagcagcagcagcacctaccctcactctcccaccagctcagacccaggaagccctttccagatgccagctgatacgcccccacctgcttacctgcctcctgaagaccccatgacccaggatggctctcagccgatggacacaaacatgatggcgcctcccctgccctcagaaatcaacagaggagatgttcaggcggttgcttatgaggaaccaaaacactggtgctctattgtctactatgagctcaacaatcgtgtgggtgaagcgttccatgcctcctccacaagtgtgttggtggatggtttcactgatccttccaacaataagaaccgtttctgccttgggctgctctccaatgttaaccggaattccactattgaaaacaccaggcggcatattggaaaaggagttcatctttattatgttggaggggaggtgtatgccgaatgccttagtgacagtagcatctttgtgcaaagtcggaactgcaactaccatcatggatttcatcctactactgtttgcaagatccctagtgggtgtagtctgaaaatttttaacaaccaagaatttgctcagttattggcacagtctgtgaaccatggatttgagacagtctatgagcttacaaaaatgtgtactatacgtatgagctttgtgaagggctggggagcagaataccaccgccaggatgttactagcaccccctgctggattgagatacatctgcacggccccctccagtggctggataaagttcttactcaaatgggttcacctcataatcctatttcatctgtatct

SEQ ID NO: 28 - DNA, 1050 bp, synthetic construct
  source 1..1050: mol_type = other DNA
atgtcaaatattacagatccacagatgtgggattttgatgatctaaatttcactggcatgccacctgcagatgaagattacagcccctgtatgctagaaactgagacactcaacaagtatgttgtgatcatcgcctatgccctagtgttcctgctgagcctgctgggaaactccctggtgatgctggtcatcttatacagcagggtcggccgctccgtcactgatgtctacctgctgaacctggccttggccgacctactctttgccctgaccttgcccatctgggccgcctccaaggtgaatggctggatttttggcacattcctgtgcaaggtggtctcactcctgaaggaagtcaacttctacagtggcatcctgctgttggcctgcatcagtgtggaccgttacctggccattgtccatgccacacgcacactgacccagaagcgtcacttggtcaagtttgtttgtcttggctgctggggactgtctatgaatctgtccctgcccttcttccttttccgccaggcttaccatccaaacaattccagtccagtttgctatgaggtcctgggaaatgacacagcaaaatggcggatggtgttgcggatcctgcctcacacctttggcttcatcgtgccgctgtttgtcatgctgttctgctatggattcaccctgcgtacactgtttaaggcccacatggggcagaagcaccgagccatgagggtcatctttgctgtcgtcctcatcttcctgctttgctggctgccctacaacctggtcctgctggcagacaccctcatgaggacccaggtgatccaggagagctgtgagcgccgcaacaacatcggccgggccctggatgccactgagattctgggatttctccatagctgcctcaaccccatcatctacgccttcatcggccaaaattttcgccatggattcctcaagatcctggctatgcatggcctggtcagcaaggagttcttggcacgtcatcgtgttacctcctacacttcttcgtctgtcaatgtctcttccaacctc

SEQ ID NO: 29 - DNA, 813 bp, synthetic construct
  source 1..813: mol_type = other DNA
atggaagattcccacaagagtaccacgtcagagacagcacctcaacctggttcagcagttcagggagctcacatttctcatattgctcaacaggtatcatctttatcagaaagtgaggagtcccaggactcatccgacagcataggctcctcacagaaagcccacgggatcctagcacggcgcccatcttacagaaaaattttgaaagacttatcttctgaagatacacggggcagaaaaggagacggagaaaattctggagtttctgctgctgtcacttctatgtctgttccaactcccatctatcagactagcagcggacagtacattgccattgccccaaatggagccttacagttggcaagtccaggcacagatggagtacagggacttcagacattaaccatgacaaattcaggcagtactcagcaaggtacaactattcttcagtatgcacagacctctgatggacagcagatacttgtgcccagcaatcaggtggtcgtacaaactgcatcaggagatatgcaaacatatcagatccgaactacaccttcagctacttctctgccacaaactgtggtgatgacatctcctgtgactctcacctctcagacaactaagacagatgacccccaattgaaaagagaaataaggttaatgaaaaacagagaagctgctcgagaatgtcgcagaaagaagaaagaatatgtgaaatgcctggaaaaccgagttgcagtcctggaaaatcaaaataaaactctaatagaagagttaaaaactttgaaggatctttattccaataaaagtgtt

SEQ ID NO: 30 - protein, 338 aa, synthetic construct
MEYFMVPTQKVPSLQHFRKTEKEVIGGLCSLANIPLTPETQRDQERRIRREIANSNERRRMQSINAGFQSLKTLIPHTDGEKLSKAAILQQTAEYIFSLEQEKTRLLQQNTQLKRFIQELSGSSPKRRRAEDKDEGIGSPDIWEDEKAEDLRREMIELRQQLDKERSVRMMLEEQVRSLEAHMYPEKLKVIAQQVQLQQQQEQVRLLHQEKLEREQQQLRTQLLPPPAPTHHPTVIVPAPPPPPSHHINVVTMGPSSVINSVSTSRQNLDTIVQAIQHIEGTQEKQELEEEQRRAVIVKPVRSCPEAPTSDTASDSEASDSDAMDQSREEPSGDGELP

SEQ ID NO: 31 - protein, 125 aa, synthetic construct
MPHSSDSSDSSFSRSPPPGKQDSSDDVRRVQRREKNRIAAQKSRQRQTQKADTLHLESEDLEKQNAALRKEIKQLTEELKYFTSVLNSHEPLCSVLAASTPSPPEVVYSAHAFHQPHVSSPRFQP

SEQ ID NO: 32 - protein, 127 aa, synthetic construct
MSQGLPAAGSVLQRSVAAPGNQPQPQPQQQSPEDDDRKVRRREKNRVAAQRSRKKQTQKADKLHEEYESLEQENTMLRREIGKLTEELKHLTEALKEHEKMCPLLLCPMNFVPVPPRPDPVAGCLPR

SEQ ID NO: 33 - protein, 574 aa, synthetic construct
MASDLESSLTSIDWLPQLTLRATIEKLGSASQAGPPGSSRKCSPGSPTDPNATLSKDEAAVHQDGKPRYSYATLITYAINSSPAKKMTLSEIYRWICDNFPYYKNAGIGWKNSIRHNLSLNKCFRKVPRPRDDPGKGSYWTIDTCPDISRKRRHPPDDDLSQDSPEQEASKSPRGGVAGSGEASLPPEGNPQMSLQSPTSIASYSQGTGSVDGGAVAAGASGRESAEGPPPLYNTNHDFKFSYSEINFQDLSWSFRNLYKSMLEKSSSSSQHGFSSLLGDIPPSNNYYMYQQQQPPPPQQQQQQQQPPQPPPQQSQPQQQQAPAQGPSAVGGAPPLHTPSTDGCTPPGGKQAGAEGYGPPPVMAMHPPPLQHGGYHPHQHHPHSHPAQQPPPPQPQAQGQAPINNTGFAFPSDWCSNIDSLKESFKMVNRLNWSSIEQSQFSELMESLRQAEQKNWTLDQHHIANLCDSLNHFLTQTGHVPPQGGTHRPPAPARIADSCALTSGKQESAMSQVNSYGHPQAPHLYPGPSPMYPIPTQDSAGYNRPAHHMVPRPSVPPPGANEEIPDDFDWDLIT

SEQ ID NO: 34 - protein, 462 aa, synthetic construct
MASNSSSCPTPGGGHLNGYPVPPYAFFFPPMLGGLSPPGALTTLQHQLPVSGYSTPSPATIETQSSSSEEIVPSPPSPPPLPRIYKPCFVCQDKSSGYHYGVSACEGCKGFFRRSIQKNMVYTCHRDKNCIINKVTRNRCQYCRLQKCFEVGMSKESVRNDRNKKKKEVPKPECSESYTLTPEVGELIEKVRKAHQETFPALCQLGKYTTNNSSEQRVSLDIDLWDKFSELSTKCIIKTVEFAKQLPGFTTLTIADQITLLKAACLDILILRICTRYTPEQDTMTFSDGLTLNRTQMHNAGFGPLTDLVFAFANQLLPLEMDDAETGLLSAICLICGDRQDLEQPDRVDMLQEPLLEALKVYVRKRRPSRPHMFPKMLMKITDLRSISAKGAERVITLKMEIPGSMPPLIQEMLENSEGLDTLSGQPGGGGRDGGGLAPPPGSCSPSLSPSSNRSSPATHSP

SEQ ID NO: 35 - protein, 119 aa, synthetic construct
MKALSPVRGCYEAVCCLSERSLAIARGRGKGPAAEEPLSLLDDMNHCYSRLRELVPGVPRGTQLSQVEILQRVIDYILDLQVVLAEPAPGPPDGPHLPIQTAELTPELVISNDKRSFCH

SEQ ID NO: 36 - protein, 349 aa, synthetic construct
MPVERMRMRPWLEEQINSNTIPGLKWLNKEKKIFQIPWMHAARHGWDVEKDAPLFRNWAIHTGKHQPGVDKPDPKTWKANFRCAMNSLPDIEEVKDKSIKKGNNAFRVYRMLPLSERPSKKGKKPKTEKEDKVKHIKQEPVESSLGLSNGVSDLSPEYAVLTSTIKNEVDSTVNIIVVGQSHLDSNIENQEIVTNPPDICQVVEVTTESDEQPVSMSELYPLQISPVSSYAESETTDSVPSDEESAEGRPHWRKRNIEGKQYLSNMGTRGSYLLPGMASFVTSNKPDLQVTIKEESNPVPYNSSWPPFQDLPLSSSMTPASSSSRPDRETRASVIKKTSDITQARVKSC

SEQ ID NO: 37 - protein, 305 aa, synthetic construct
MSVDPACPQSLPCFEASDCKESSPMPVICGPEENYPSLQMSSAEMPHTETVSPLPSSMDLLIQDSPDSSTSPKGKQPTSAEKSVAKKEDKVPVKKQKTRTVFSSTQLCVLNDRFQRQKYLSLQQMQELSNILNLSYKQVKTWFQNQRMKSKRWQKNNWPKNSNGVTQKASAPTYPSLYSSYHQGCLVNPTGNLPMWSNQTWNNSTWSNQTQNIQSWSNHSWNTQTWCTQSWNNQAWNSPFYNCGEESLQSCMQFQPNSPASDLEAALEAAGEGLNVIQQTTRYFSTPQTMDLFLNYSMNMQPEDV

SEQ ID NO: 38 - protein, 164 aa, synthetic construct
MSVDPLSSKALKIKRELSENTPHLSDEALMGLSVRELNRHLRGLSAEEVTRLKQRRRTLKNRGYAASCRVKRVCQKEELQKQKSELEREVDKLARENAAMRLELDALRGKCEALQGFARSVAAARGPATLVAPASVITIVKSTPGSGSGPAHGPDPAHGPASCS

SEQ ID NO: 39 - protein, 795 aa, synthetic construct
MDHLNEATQGKEHSEMSNNVSDPKGPPAKIARLEQNGSPLGRGRLGSTGAKMQGVPLKHSGHLMKTNLRKGTMLPVFCVVEHYENAIEYDCKEEHAEFVLVRKDMLFNQLIEMALLSLGYSHSSAAQAKGLIQVGKWNPVPLSYVTDAPDATVADMLQDVYHVVTLKIQLHSCPKLEDLPPEQWSHTTVRNALKDLLKDMNQSSLAKECPLSQSMISSIVNSTYYANVSAAKCQEFGRWYKHFKKTKDMMVEMDSLSELSQQGANHVNFGQQPVPGNTAEQPPSPAQLSHGSQPSVRTPLPNLHPGLVSTPISPQLVNQQLVMAQLLNQQYAVNRLLAQQSLNQQYLNHPPPVSRSMNKPLEQQVSTNTEVSSEIYQWVRDELKRAGISQAVFARVAFNRTQGLLSEILRKEEDPKTASQSLLVNLRAMQNFLQLPEAERDRIYQDERERSLNAASAMGPAPLISTPPSRPPQVKTATIATERNGKPENNTMNINASIYDEIQQEMKRAKVSQALFAKVAATKSQGWLCELLRWKEDPSPENRTLWENLSMIRRFLSLPQPERDAIYEQESNAVHHHGDRPPHIIHVPAEQIQSPSPTTLGKGESRGVFLPGLPTPAPWLGAAPQQQQQQQQQQQQQQQAPPPPQPQQQPQTGPRLPPRQPTVASPAESDEENRQKTRPRTKISVEALGILQSFIQDVGLYPDEEAIQTLSAQLDLPKYTIIKFFQNQRYYLKHHGKLKDNSGLEVDVAEYKEEELLKDLEESVQDKNTNTLFSVKLEEELSVEGNTDINTDLKD

SEQ ID NO: 40 - protein, 686 aa, synthetic construct
MQLGEQLLVSSVNLPGAHFYPLESARGGSGGSAGHLPSAAPSPQKLDLDKASKKFSGSLSCEAVSGEPAAASAGAPAAMLSDTDAGDAFASAAAVAKPGPPDGRKGSPCGEEELPSAAAAAAAAAAAAAATARYSMDSLSSERYYLQSPGPQGSELAAPCSLFPYQAAAGAPHGPVYPAPNGARYPYGSMLPPGGFPAAVCPPGRAQFGPGAGAGSGAGGSSGGGGGPGTYQYSQGAPLYGPYPGAAAAGSCGGLGGLGVPGSGFRAHVYLCNRPLWLKFHRHQTEMIITKQGRRMFPFLSFNINGLNPTAHYNVFVEVVLADPNHWRFQGGKWVTCGKADNNMQGNKMYVHPESPNTGSHWMRQEISFGKLKLTNNKGANNNNTQMIVLQSLHKYQPRLHIVEVTEDGVEDLNEPSKTQTFTFSETQFIAVTAYQNTDITQLKIDHNPFAKGFRDNYDSSHQIVPGGRYGVQSFFPEPFVNTLPQARYYNGERTVPQTNGLLSPQQSEEVANPPQRWLVTPVQQPGTNKLDISSYESEYTSSTLLPYGIKSLPLQTSHALGYYPDPTFPAMAGWGGRGSYQRKMAAGLPWTSRTSPTVFSEDQLSKEKVKEEIGSSWIETPPSIKSLDSNDSGVYTSACKRRRLSPSNSSNENSPSIKCEDINAEEYSKDTSKGMGGYYAFYTTP

SEQ ID NO: 41 - protein, 325 aa, synthetic construct
MPITRMRMRPWLEMQINSNQIPGLIWINKEEMIFQIPWKHAAKHGWDINKDACLFRSWAIHTGRYKAGEKEPDPKTWKANFRCAMNSLPDIEEVKDQSRNKGSSAVRVYRMLPPLTKNQRKERKSKSSRDAKSKAKRKSCGDSSPDTFSDGLSSSTLPDDHSSYTVPGYMQDLEVEQALTPALSPCAVSSTLPDWHIPVEVVPDSTSDLYNFQVSPMPSTSEATTDEDEEGKLPEDIMKLLEQSEWQPTNVDGKGYLLNEPGVQPTSVYGDFSCKEEPEIDSPGGDIGLSLQRVFTDLKNMDATWLDSLLTPVRLPSIQAIPCAP

SEQ ID NO: 42 - protein, 112 aa, synthetic construct
MLIFLGCYRRRLEERAGTMSAETASGPTEDQVEILEYNFNKVDKHPDSTTLCLIAAEAGLSEEETQGSDLISRSKIWHPESSPQREGYPHDSLPCLAFDYFSLLPPQCKEMV

SEQ ID NO: 43 - protein, 173 aa, synthetic construct
MGLPRRAGDAAELRKSLKPLLEKRRRARINQSLSQLKGLILPLLGRENSNCSKLEKADVLEMTVRFLQELPASSWPTAAPLPCDSYREGYSACVARLARVLPACRVLEPAVSARLLEHLWRRAASATLDGGRAGDSSGPSAPAPAPASAPEPASAPVPSPPSPPCGPGLWRPW

SEQ ID NO: 44 - protein, 134 aa, synthetic construct
MKAFSPVRSVRKNSLSDHSLGISRSKTPVDDPMSLLYNMNDCYSKLKELVPSIPQNKKVSKMEILQHVIDYILDLQIALDSHPTIVSLHHQRPGQNQASRTPLTTLNTDISILSLQASEFPSELMSNDSKALCG

SEQ ID NO: 45 - protein, 636 aa, synthetic construct
MAQSTATSPDGGTTFEHLWSSLEPDSTYFDLPQSSRGNNEVVGGTDSSMDVFHLEGMTTSVMAQFNLLSSTMDQMSSRAASASPYTPEHAASVPTHSPYAQPSSTFDTMSPAPVIPSNTDYPGPHHFEVTFQQSSTAKSATWTYSPLLKKLYCQIAKTCPIQIKVSTPPPPGTAIRAMPVYKKAEHVTDVVKRCPNHELGRDFNEGQSAPASHLIRVEGNNLSQYVDDPVTGRQSVVVPYEPPQVGTEFTTILYNFMCNSSCVGGMNRRPILIIITLEMRDGQVLGRRSFEGRICACPGRDRKADEDHYREQQALNESSAKNGAASKRAFKQSPPAVPALGAGVKKRRHGDEDTYYLQVRGRENFEILMKLKESLELMELVPQPLVDSYRQQQQLLQRPSHLQPPSYGPVLSPMNKVHGGMNKLPSVNQLVGQPPPHSSAATPNLGPVGPGMLNNHGHAVPANGEMSSSHSAQSMVSGSHCTPPPPYHADPSLVSFLTGLGCPNCIEYFTSQGLQSIYHLQNLTIEDLGALKIPEQYRMTIWRGLQDLKQGHDYSTAQQLLRSSNAATISIGGSGELQRQRVMEAVHFRVRHTITIPNRGGPGGGPDEWADFGFDLPDCKARKQPIKEEFTEAEIH

SEQ ID NO: 46 - protein, 425 aa, synthetic construct
MSSILPFTPPIVKRLLGWKKGEQNGQEEKWCEKAVKSLVKKLKKTGQLDELEKAITTQNVNTKCITIPRSLDGRLQVSHRKGLPHVIYCRLWRWPDLHSHHELRAMELCEFAFNMKKDEVCVNPYHYQRVETPVLPPVLVPRHTEIPAEFPPLDDYSHSIPENTNFPAGIEPQSNIPETPPPGYLSEDGETSDHQMNHSMDAGSPNLSPNPMSPAHNNLDLQPVTYCEPAFWCSISYYELNQRVGETFHASQPSMTVDGFTDPSNSERFCLGLLSNVNRNAAVELTRRHIGRGVRLYYIGGEVFAECLSDSAIFVQSPNCNQRYGWHPATVCKIPPGCNLKIFNNQEFAALLAQSVNQGFEAVYQLTRMCTIRMSFVKGWGAEYRRQTVTSTPCWIELHLNGPLQWLDKVLTQMGSPSIRCSSVS

SEQ ID NO: 47 - protein, 505 aa, synthetic construct
MKFKLHVNSARQYKDLWNMSDDKPFLCTAPGCGQRFTNEDHLAVHKHKHEMTLKFGPARNDSVIVADQTPTPTRFLKNCEEVGLFNELASPFENEFKKASEDDIKKMPLDLSPLATPIIRSKIEEPSVVETTHQDSPLPHPESTTSDEKEVPLAQTAQPTSAIVRPASLQVPNVLLTSSDSSVIIQQAVPSPTSSTVITQAPSSNRPIVPVPGPFPLLLHLPNGQTMPVAIPASITSSNVHVPAAVPLVRPVTMVPSVPGIPGPSSPQPVQSEAKMRLKAALTQQHPPVTNGDTVKGHGSGLVRTQSEESRPQSLQQPATSTTETPASPAHTTPQTQSTSGRRRRAANEDPDEKRRKFLERNRAAASRCRQKRKVWVQSLEKKAEDLSSLNGQLQSEVTLLRNEVAQLKQLLLAHKDCPVTAMQKKSGYHTADKDDSSEDISVPSSPHTEAIQHSSVSTSNGVSSTSKAEAVATSVLTQMADQSTEPALSQIVMAPSSQSQPSGS

SEQ ID NO: 48 - protein, 552 aa, synthetic construct
MDNMSITNTPTSNDACLSIVHSLMCHRQGGESETFAKRAIESLVKKLKEKKDELDSLITAITTNGAHPSKCVTIQRTLDGRLQVAGRKGFPHVIYARLWRWPDLHKNELKHVKYCQYAFDLKCDSVCVNPYHYERVVSPGIDLSGLTLQSNAPSSMMVKDEYVHDFEGQPSLSTEGHSIQTIQHPPSNRASTETYSTPALLAPSESNATSTANFPNIPVASTSQPASILGGSHSEGLLQIASGPQPGQQQNGFTGQPATYHHNSTTTWTGSRTAPYTPNLPHHQNGHLQHHPPMPPHPGHYWPVHNELAFQPPISNHPAPEYWCSIAYFEMDVQVGETFKVPSSCPIVTVDGYVDPSGGDRFCLGQLSNVHRTEAIERARLHIGKGVQLECKGEGDVWVRCLSDHAVFVQSYYLDREAGRAPGDAVHKIYPSAYIKVFDLRQCHRQMQQQAATAQAAAAAQAAAVAGNIPGPGSVGGIAPAISLSAAAGIGVDDLRRLCILRMSFVKGWGPDYPRQSIKETPCWIEIHLHRALQLLDEVLHTMPIADPQPLD

SEQ ID NO: 49 - protein, 431 aa, synthetic construct
MPNPRPGKPSAPSLALGPSPGASPSWRAAPKASDLLGARGPGGTFQGRDLRGGAHASSSSLNPMPPSQLQLPTLPLVMVAPSGARLGPLPHLQALLQDRPHFMHQLSTVDAHARTPVLQVHPLESPAMISLTPPTTATGVFSLKARPGLPPGINVASLEWVSREPALLCTFPNPSAPRKDSTLSAVPQSSYPLLANGVCKWPGCEKVFEEPEDFLKHCQADHLLDEKGRAQCLLQREMVQSLEQQLVLEKEKLSAMQAHLAGKMALTKASSVASSDKGSCCIVAAGSQGPVVPAWSGPREAPDSLFAVRRHLWGSHGNSTFPEFLHNMDYFKFHNMRPPFTYATLIRWAILEAPEKQRTLNEIYHWFTRMFAFFRNHPATWKNAIRHNLSLHKCFVRVESEKGAVWTVDELEFRKKRSQRPSRCSNPTPGP

SEQ ID NO: 50 - protein, 551 aa, synthetic construct
MDELFPLIFPAEPAQASGPYVEIIEQPKQRGMRFRYKCEGRSAGSIPGERSTDTTKTHPTIKINGYTGPGTVRISLVTKDPPHRPHPHELVGKDCRDGFYEAELCPDRCIHSFQNLGIQCVKKRDLEQAISQRIQTNNNPFQVPIEEQRGDYDLNAVRLCFQVTVRDPSGRPLRLPPVLSHPIFDNRAPNTAELKICRVNRNSGSCLGGDEIFLLCDKVQKEDIEVYFTGPGWEARGSFSQADVHRQVAIVFRTPPYADPSLQAPVRVSMQLRRPSDRELSEPMEFQYLPDTDDRHRIEEKRKRTYETFKSIMKKSPFSGPTDPRPPPRRIAVPSRSSASVPKPAPQPYPFTSSLSTINYDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISS

SEQ ID NO: 51 - protein, 763 aa, synthetic construct [sequence omitted]

SEQ ID NO: 52 - protein, 169 aa, synthetic construct
MRWCLLLIWAQGLRQAPLASGMMTGTIETTGNISAEKGGSIILQCHLSSTTAQVTQVNWEQQDQLLAICNADLGWHISPSFKDRVAPGPGLGLTLQSLTVNDTGEYFCIYHTYPDGTYTGRIFLEVLESSVAEHGARFQIPLLGAMAATLVVICTAVIVVVALTRKKKA

SEQ ID NO: 53 - protein, 439 aa, synthetic construct
MPLNVSFTNRNYDLDYDSVQPYFYCDEEENFYQQQQQSELQPPAPSEDIWKKFELLPTPPLSPSRRSGLCSPSYVAVTPFSLRGDNDGGGGSFSTADQLEMVTELLGGDMVNQSFICDPDDETFIKNIIIQDCMWSGFSAAAKLVSEKLASYQAARKDSGSPNPARGHSVCSTSSLYLQDLSAAASECIDPSVVFPYPLNDSSSPKSCASQDSSAFSPSSDSLLSSTESSPQGSPEPLVLHEETPPTTSSDSEEEQEDEEEIDVVSVEKRQAPGKRSESGSPSAGGHSKPPHSPLVLKRCHVSTHQHNYAAPPSTRKDYPAAKRVKLDSVRVLRQISNNRKCTSPRSSDTEENVKRRTHNVLERQRRNELKRSFFALRDQIPELENNEKAPKVVILKKATAYILSVQAEEQKLISEEDLLRKRREQLKHKLEQLRNSCA

SEQ ID NO: 54 - protein, 272 aa, synthetic construct
MDSYLLMWGLLTFIMVPGCQAELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSCLVTTTDFQIQTEMAATMETSIFTTEYQVAVAGCVFLLISVLLLSGLTWQRRQRKSRRTI

SEQ ID NO: 55 - protein, 255 aa, synthetic construct
MGNSCYNIVATLLLVLNFERTRSLQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLVNGTKERDVVCGPSPADLSPGASSVTPPAPAREPGHSPQIISFFLALTSTALLFLLFFLTLRFSVVKRGRKKLLYIFKQPFMRPVQTTQEEDGCSCRFPEEEEGGCEL

SEQ ID NO: 56 - protein, 465 aa, synthetic construct
MNVTSLFSFTSPAVKRLLGWKQGDEEEKWAEKAVDALVKKLKKKKGAMEELEKALSCPGQPSNCVTIPRSLDGRLQVSHRKGLPHVIYCRVWRWPDLQSHHELKPLECCEFPFGSKQKEVCINPYHYKRVESPVLPPVLVPRHSEYNPQHSLLAQFRNLGQNEPHMPLNATFPDSFQQPNSHPFPHSPNSSYPNSPGSSSSTYPHSPTSSDPGSPFQMPADTPPPAYLPPEDPMTQDGSQPMDTNMMAPPLPSEINRGDVQAVAYEEPKHWCSIVYYELNNRVGEAFHASSTSVLVDGFTDPSNNKNRFCLGLLSNVNRNSTIENTRRHIGKGVHLYYVGGEVYAECLSDSSIFVQSRNCNYHHGFHPTTVCKIPSGCSLKIFNNQEFAQLLAQSVNHGFETVYELTKMCTIRMSFVKGWGAEYHRQDVTSTPCWIEIHLHGPLQWLDKVLTQMGSPHNPISSVS

SEQ ID NO: 57 - protein, 350 aa, synthetic construct
MSNITDPQMWDFDDLNFTGMPPADEDYSPCMLETETLNKYVVIIAYALVFLLSLLGNSLVMLVILYSRVGRSVTDVYLLNLALADLLFALTLPIWAASKVNGWIFGTFLCKVVSLLKEVNFYSGILLLACISVDRYLAIVHATRTLTQKRHLVKFVCLGCWGLSMNLSLPFFLFRQAYHPNNSSPVCYEVLGNDTAKWRMVLRILPHTFGFIVPLFVMLFCYGFTLRTLFKAHMGQKHRAMRVIFAVVLIFLLCWLPYNLVLLADTLMRTQVIQESCERRNNIGRALDATEILGFLHSCLNPIIYAFIGQNFRHGFLKILAMHGLVSKEFLARHRVTSYTSSSVNVSSNL

SEQ ID NO: 58 - protein, 271 aa, synthetic construct
MEDSHKSTTSETAPQPGSAVQGAHISHIAQQVSSLSESEESQDSSDSIGSSQKAHGILARRPSYRKILKDLSSEDTRGRKGDGENSGVSAAVTSMSVPTPIYQTSSGQYIAIAPNGALQLASPGTDGVQGLQTLTMTNSGSTQQGTTILQYAQTSDGQQILVPSNQVVVQTASGDMQTYQIRTTPSATSLPQTVVMTSPVTLTSQTTKTDDPQLKREIRLMKNREAARECRRKKKEYVKCLENRVAVLENQNKTLIEELKTLKDLYSNKSV

SEQ ID NO: 59 - DNA, 822 bp, synthetic construct
  source 1..822: mol_type = other DNA
atgttttcacatcttccctttgactgtgtcctgctgctgctgctgctactacttacaaggtcctcagaagtggaatacagagcggaggtcggtcagaatgcctatctgccctgcttctacaccccagccgccccagggaacctcgtgcccgtctgctggggcaaaggagcctgtcctgtgtttgaatgtggcaacgtggtgctcaggactgatgaaagggatgtgaattattggacatccagatactggctaaatggggatttccgcaaaggagatgtgtccctgaccatagagaatgtgactctagcagacagtgggatctactgctgccggatccaaatcccaggcataatgaatgatgaaaaatttaacctgaagttggtcatcaaaccagccaaggtcacccctgcaccgactcggcagagagacttcactgcagcctttccaaggatgcttaccaccaggggacatggcccagcagagacacagacactggggagcctccctgatataaatctaacacaaatatccacattggccaatgagttacgggactctagattggccaatgacttacggcctgcccctgcgagagagccaggacactctccgcagatcatctccttctttcttgcgctgacgtcgactgcgttgctcttcctgctgttcttcctcacgctccgtttctctgttgttaaacggggcagaaagaaactcctgtatatattcaaacaaccatttatgagaccagtacaaactactcaagaggaagatggctgtagctgccgatttccagaagaagaagaaggaggatgtgaactg

SEQ ID NO: 60 - DNA, 822 bp, synthetic construct; identical to SEQ ID NO: 59
  source 1..822: mol_type = other DNA

SEQ ID NO: 61 - DNA, 2472 bp, synthetic construct
  source 1..2472: mol_type = other DNA
atgctcctgccttgggccacctctgcccccggcctggcctgggggcctctggtgctgggcctcttcgggctcctggcagcatcgcagccccaggcggtgcctccatatgcgtcggagaaccagacctgcagggaccaggaaaaggaatactatgagccccagcaccgcatctgctgctcccgctgcccgccaggcacctatgtctcagctaaatgtagccgcatccgggacacagtttgtgccacatgtgccgagaattcctacaacgagcactggaactacctgaccatctgccagctgtgccgcccctgtgacccagtgatgggcctcgaggagattgccccctgcacaagcaaacggaagacccagtgccgctgccagccgggaatgttctgtgctgcctgggccctcgagtgtacacactgcgagctactttctgactgcccgcctggcactgaagccgagctcaaagatgaagttgggaagggtaacaaccactgcgtcccctgcaaggccgggcacttccagaatacctcctcccccagcgcccgctgccagccccacaccaggtgtgagaaccaaggtctggtggaggcagctccaggcactgcccagtccgacacaacctgcaaaaatccattagagccactgcccccagagatgtcaggaaccatgctgatgctggccgttctgctgccactggccttctttctgctccttgccaccgtcttctcctgcatctggaagagccacccttctctctgcaagattaagaaagaatggtgggatcagattcccaacccagcccgcagccgcctcgtggctataataatccaggatgctcaggggtcacagtgggagaagcggtcccgaggccaggaaccagccaagtgcccacactggaagaattgtcttaccaagctcttgccctgttttctggagcacaacatgaaaagggatgaagatcctcacaaggctgccaaagagatgcctttccagggctctggaaaatcagcatggtgcccagtggagatcagcaagacagtcctctggccagagagcatcagcgtggtgcgatgtgtggagttgtttgaggccccggtggagtgtgaggaggaggaggaggtagaggaagaaaaagggagcttctgtgcatcgcctgagagcagcagggatgacttccaggagggaagggagggcattgtggcccggctaacagagagcctgttcctggacctgctcggagaggagaatgggggcttttgccagcaggacatgggggagtcatgccttcttccaccttcgggaagtacgagtgctcacatgccctgggatgagttcccaagtgcagggcccaaggaggcacctccctggggcaaggagcagcctctccacctggagccaagtcctcctgccagcccgacccagagtccagacaacctgacttgcacagagacgcccctcgtcatcgcaggcaaccctgcttaccgcagcttcagcaactccctgagccagtcaccgtgtcccagagagctgggtccagacccactgctggccagacacctggaggaagtagaacccgagatgccctgtgtcccccagctctctgagccaaccactgtgccccaacctgagccagaaacctgggagcagatcctccgccgaaatgtcctccagcatggggcagctgcagcccccgtctcggcccccaccagtggctatcaggagtttgtacatgcggtggagcagggtggcacccaggccagtgcggtggtgggcttgggtcccccaggagaggctggttacaaggccttctcaagcctgcttgccagcagtgctgtgtccccagagaaatgtgggtttggggctagcagtggggaagaggggtataagcctttccaagacctcattcctggctgccctggggaccctgccccagtccctgtccccttgttcacctttggactggacagggagccacctcgcagtccgcagagctcacatctcccaagcagctccccagagcacctgggtctggagccgggggaaaaggtagaggacatgccaaagcccccacttccccaggagcaggccacagacccccttgtggacagcctgggcagtggcattgtctactcagcccttacctgccacctgtgcggccacctgaaacagtgtcatggccaggaggatggtggccagacccctgtcatggccagtccttgctgtggctgctgctgtggagacaggtcctcgccccctacaacccccctgagggccccagacccctctccaggtggggttccactggaggccagtctgtgtccggcctccctggcaccctcgggcatctcagagaagagtaaatcctcatcatccttccatcctgcccctggcaatgctcagagctcaagccagacccccaaaatcgtgaactttgtctccgtgggacccacatacatgagggtctct

SEQ ID NO: 62 - DNA, 906 bp, synthetic construct
  source 1..906: mol_type = other DNA
atggggtggctttgctctgggctcctgttccctgtgagctgcctggtcctgctgcaggtggcaagctctgggaacatgaaggtcttgcaggagcccacctgcgtctccgactacatgagcatctctacttgcgagtggaagatgaatggtcccaccaattgcagcaccgagctccgcctgttgtaccagctggtttttctgctctccgaagcccacacgtgtatccctgagaacaacggaggcgcggggtgcgtgtgccacctgctcatggatgacgtggtcagtgcggataactatacactggacctgtgggctgggcagcagctgctgtggaagggctccttcaagcccagcgagcatgtgaaacccagggccccaggaaacctgacagttcacaccaatgtctccgacactctgctgctgacctggagcaacccgtatccccctgacaattacctgtataatcatctcacctatgcagtcaacatttggagtgaaaacgacccggcagatttcagaatctataacgtgacctacctagaaccctccctccgcatcgcagccagcaccctgaagtctgggatttcctacagggcacgggtgagggcctgggctcagtgctataacaccacctggagtgagtggagccccagcaccaagtggcacaactcctacagggagcccttcgagcagcacctcatcatctccttctttcttgcgctgacgtcgactgcgttgctcttcctgctgttcttcctcacgctccgtttctctgttgttaaacggggcagaaagaaactcctgtatatattcaaacaaccatttatgagaccagtacaaactactcaagaggaagatggctgtagctgccgatttccagaagaagaagaaggaggatgtgaactg

SEQ ID NO: 63 - DNA, 903 bp, synthetic construct
  source 1..903: mol_type = other DNA
atgctggggcaagtggtcaccctcatactcctcctgctcctcaaggtgtatcagggcaaaggatgccagggatcagctgaccatgtggttagcatctcgggagtgcctcttcagttacaaccaaacagcatacagacgaaggttgacagcattgcatggaagaagttgctgccctcacaaaatggatttcatcacatattgaagtgggagaatggctctttgccttccaatacttccaatgatagattcagttttatagtcaagaacttgagtcttctcatcaaggcagctcagcagcaggacagtggcctctactgcctggaggtcaccagtatatctggaaaagttcagacagccacgttccaggtttttgtatttgaatctctgcttccagataaagttgagaaaccccgcctacaggggcaggggaagatcctggacagagggagatgccaagtggctctgtcttgcttggtctccagggatggcaatgtgtcctatgcttggtacagagggagcaagctgatccagacagcagggaacctcacctacctggacgaggaggttgacattaatggcactcacacatatacctgcaatgtcagcaatcctgttagctgggaaagccacaccctgaatctcactcaggactgtcagaatcctgcccctgcgagagagccaggacactctccgcagatcatctccttctttcttgcgctgacgtcgactgcgttgctcttcctgctgttcttcctcacgctccgtttctctgttgttaaacggggcagaaagaaactcctgtatatattcaaacaaccatttatgagaccagtacaaactactcaagaggaagatggctgtagctgccgatttccagaagaagaagaaggaggatgtgaactg

SEQ ID NO: 64 - DNA, 795 bp, synthetic construct
  source 1..795: mol_type = other DNA
atgggcgtccccacggccctggaggccggcagctggcgctggggatccctgctcttcgctctcttcctggctgcgtccctaggtccggtggcagccttcaaggtcgccacgccgtattccctgtatgtctgtcccgaggggcagaacgtcaccctcacctgcaggctcttgggccctgtggacaaagggcacgatgtgaccttctacaagacgtggtaccgcagctcgaggggcgaggtgcagacctgctcagagcgccggcccatccgcaacctcacgttccaggaccttcacctgcaccatggaggccaccaggctgccaacaccagccacgacctggctcagcgccacgggctggagtcggcctccgaccaccatggcaacttctccatcaccatgcgcaacctgaccctgctggatagcggcctctactgctgcctggtggtggagatcaggcaccaccactcggagcacagggtccatggtgccatggagctgcaggtgcagacaggcaaagatgcaccatccaactgtgtggtgtacccatcctcctcctgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctcc

SEQ ID NO: 65 - DNA, 450 bp, synthetic construct
  source 1..450: mol_type = other DNA
atggcccgcggaagtcttcgccgtttgctccgtcttcttgttctcggcctgtggctcgctctgctccggagtgtagcgggcgaacaagcacctgggactgcaccgtgttcacggggctcctcatggtccgccgatcttgataaatgtatggattgtgccagctgtagagccaggccccattctgatttctgtcttgggtgtgccgctgccccaccggcaccttttagacttctgtggcctattctgggcggagccctctcattgacatttgtccttggacttctctccgggttccttgtatggcggcggtgtcggcgccgcgaagccctgtacctgctccggagggaccagaggctgccccccgatgcccacaagccccctgggggaggcagtttccggacccccatccaagaggagcaggccgacgcccactccaccctggccaagatc

SEQ ID NO: 66 - DNA, 888 bp, synthetic construct
  source 1..888: mol_type = other DNA
atgctcctgccttgggccacctctgcccccggcctggcctgggggcctctggtgctgggcctcttcgggctcctggcagcatcgcagccccaggcggtgcctccatatgcgtcggagaaccagacctgcagggaccaggaaaaggaatactatgagccccagcaccgcatctgctgctcccgctgcccgccaggcacctatgtctcagctaaatgtagccgcatccgggacacagtttgtgccacatgtgccgagaattcctacaacgagcactggaactacctgaccatctgccagctgtgccgcccctgtgacccagtgatgggcctcgaggagattgccccctgcacaagcaaacggaagacccagtgccgctgccagccgggaatgttctgtgctgcctgggccctcgagtgtacacactgcgagctactttctgactgcccgcctggcactgaagccgagctcaaagatgaagttgggaagggtaacaaccactgcgtcccctgcaaggccgggcacttccagaatacctcctcccccagcgcccgctgccagccccacaccaggtgtgagaaccaaggtctggtggaggcagctccaggcactgcccagtccgacacaacctgcaaaaatccattagagccactgcccccagagatgtcaggaaccatgctgatgctggccgttctgctgccactggccttctttctgctccttgccaccgtcttctcctgcatctggaagagccacccttctctctgcaggagtaagaggagcaggctcctgcacagtgactacatgaacatgactccccgccgccccgggcccacccgcaagcattaccagccctatgccccaccacgcgacttcgcagcctatcgctcc

SEQ ID NO: 67 - DNA, 1071 bp, synthetic construct
  source 1..1071: mol_type = other DNA
atgctcctgccttgggccacctctgcccccggcctggcctgggggcctctggtgctgggcctcttcgggctcctggcagcatcgcagccccaggcggtgcctccatatgcgtcggagaaccagacctgcagggaccaggaaaaggaatactatgagccccagcaccgcatctgctgctcccgctgcccgccaggcacctatgtctcagctaaatgtagccgcatccgggacacagtttgtgccacatgtgccgagaattcctacaacgagcactggaactacctgaccatctgccagctgtgccgcccctgtgacccagtgatgggcctcgaggagattgccccctgcacaagcaaacggaagacccagtgccgctgccagccgggaatgttctgtgctgcctgggccctcgagtgtacacactgcgagctactttctgactgcccgcctggcactgaagccgagctcaaagatgaagttgggaagggtaacaaccactgcgtcccctgcaaggccgggcacttccagaatacctcctcccccagcgcccgctgccagccccacaccaggtgtgagaaccaaggtctggtggaggcagctccaggcactgcccagtccgacacaacctgcaaaaatccattagagccactgcccccagagatgtcaggaaccatgctgatgctggccgttctgctgccactggccttctttctgctccttgccaccgtcttctcctgcatctggaagagccacccttctctctgcacacaggtggcggccgactggaccgcgctggcggaggagatggactttgagtacttggagatccggcaactggagacacaagcggaccccactggcaggctgctggacgcctggcagggacgccctggcgcctctgtaggccgactgctcgagctgcttaccaagctgggccgcgacgacgtgctgctggagctgggacccagcattgaggaggattgccaaaagtatatcttgaagcagcagcaggaggaggctgagaagcctttacaggtggccgctgtagacagcagtgtcccacggacagca

SEQ ID NO: 68 - DNA, 687 bp, synthetic construct
  source 1..687: mol_type = other DNA
atgaagacattgcctgccatgcttggaactgggaaattattttgggtcttcttcttaatcccatatctggacatctggaacatccatgggaaagaatcatgtgatgtacagctttatataaagagacaatctgaacactccatcttagcaggagatccctttgaactagaatgccctgtgaaatactgtgctaacaggcctcatgtgacttggtgcaagctcaatggaacaacatgtgtaaaacttgaagatagacaaacaagttggaaggaagagaagaacatttcatttttcattctacattttgaaccagtgcttcctaatgacaatgggtcataccgctgttctgcaaattttcagtctaatctcattgaaagccactcaacaactctttatgtgacagatgtaaaaagtgcctcagaacgaccctccaaggacgaaatgcctgcccctgcgagagagccaggacactctccgcagatcatctccttctttcttgcgctgacgtcgactgcgttgctcttcctgctgttcttcctcacgctccgtttctctgttgttaaacggggcagaaagaaactcctgtatatattcaaacaaccatttatgagaccagtacaaactactcaagaggaagatggctgtagctgccgatttccagaagaagaagaaggaggatgtgaactg

SEQ ID NO: 69 - protein, 274 aa, synthetic construct
MFSHLPFDCVLLLLLLLLTRSSEVEYRAEVGQNAYLPCFYTPAAPGNLVPVCWGKGACPVFECGNVVLRTDERDVNYWTSRYWLNGDFRKGDVSLTIENVTLADSGIYCCRIQIPGIMNDEKFNLKLVIKPAKVTPAPTRQRDFTAAFPRMLTTRGHGPAETQTLGSLPDINLTQISTLANELRDSRLANDLRPAPAREPGHSPQIISFFLALTSTALLFLLFFLTLRFSVVKRGRKKLLYIFKQPFMRPVQTTQEEDGCSCRFPEEEEGGCEL

SEQ ID NO: 70 - protein, 274 aa, synthetic construct; identical to SEQ ID NO: 69

SEQ ID NO: 71 - protein, 824 aa, synthetic construct
MLLPWATSAPGLAWGPLVLGLFGLLAASQPQAVPPYASENQTCRDQEKEYYEPQHRICCSRCPPGTYVSAKCSRIRDTVCATCAENSYNEHWNYLTICQLCRPCDPVMGLEEIAPCTSKRKTQCRCQPGMFCAAWALECTHCELLSDCPPGTEAELKDEVGKGNNHCVPCKAGHFQNTSSPSARCQPHTRCENQGLVEAAPGTAQSDTTCKNPLEPLPPEMSGTMLMLAVLLPLAFFLLLATVFSCIWKSHPSLCKIKKEWWDQIPNPARSRLVAIIIQDAQGSQWEKRSRGQEPAKCPHWKNCLTKLLPCFLEHNMKRDEDPHKAAKEMPFQGSGKSAWCPVEISKTVLWPESISVVRCVELFEAPVECEEEEEVEEEKGSFCASPESSRDDFQEGREGIVARLTESLFLDLLGEENGGFCQQDMGESCLLPPSGSTSAHMPWDEFPSAGPKEAPPWGKEQPLHLEPSPPASPTQSPDNLTCTETPLVIAGNPAYRSFSNSLSQSPCPRELGPDPLLARHLEEVEPEMPCVPQLSEPTTVPQPEPETWEQILRRNVLQHGAAAAPVSAPTSGYQEFVHAVEQGGTQASAVVGLGPPGEAGYKAFSSLLASSAVSPEKCGFGASSGEEGYKPFQDLIPGCPGDPAPVPVPLFTFGLDREPPRSPQSSHLPSSSPEHLGLEPGEKVEDMPKPPLPQEQATDPLVDSLGSGIVYSALTCHLCGHLKQCHGQEDGGQTPVMASPCCGCCCGDRSSPPTTPLRAPDPSPGGVPLEASLCPASLAPSGISEKSKSSSSFHPAPGNAQSSSQTPKIVNFVSVGPTYMRVS

SEQ ID NO: 72 - protein, 302 aa, synthetic construct
MGWLCSGLLFPVSCLVLLQVASSGNMKVLQEPTCVSDYMSISTCEWKMNGPTNCSTELRLLYQLVFLLSEAHTCIPENNGGAGCVCHLLMDDVVSADNYTLDLWAGQQLLWKGSFKPSEHVKPRAPGNLTVHTNVSDTLLLTWSNPYPPDNYLYNHLTYAVNIWSENDPADFRIYNVTYLEPSLRIAASTLKSGISYRARVRAWAQCYNTTWSEWSPSTKWHNSYREPFEQHLIISFFLALTSTALLFLLFFLTLRFSVVKRGRKKLLYIFKQPFMRPVQTTQEEDGCSCRFPEEEEGGCEL

SEQ ID NO: 73 - protein, 301 aa, synthetic construct
MLGQVVTLILLLLLKVYQGKGCQGSADHVVSISGVPLQLQPNSIQTKVDSIAWKKLLPSQNGFHHILKWENGSLPSNTSNDRFSFIVKNLSLLIKAAQQQDSGLYCLEVTSISGKVQTATFQVFVFESLLPDKVEKPRLQGQGKILDRGRCQVALSCLVSRDGNVSYAWYRGSKLIQTAGNLTYLDEEVDINGTHTYTCNVSNPVSWESHTLNLTQDCQNPAPAREPGHSPQIISFFLALTSTALLFLLFFLTLRFSVVKRGRKKLLYIFKQPFMRPVQTTQEEDGCSCRFPEEEEGGCEL

SEQ ID NO: 74 - protein, 265 aa, synthetic construct
MGVPTALEAGSWRWGSLLFALFLAASLGPVAAFKVATPYSLYVCPEGQNVTLTCRLLGPVDKGHDVTFYKTWYRSSRGEVQTCSERRPIRNLTFQDLHLHHGGHQAANTSHDLAQRHGLESASDHHGNFSITMRNLTLLDSGLYCCLVVEIRHHHSEHRVHGAMELQVQTGKDAPSNCVVYPSSSCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 75 - protein, 150 aa, synthetic construct
MARGSLRRLLRLLVLGLWLALLRSVAGEQAPGTAPCSRGSSWSADLDKCMDCASCRARPHSDFCLGCAAAPPAPFRLLWPILGGALSLTFVLGLLSGFLVWRRCRRREALYLLRRDQRLPPDAHKPPGGGSFRTPIQEEQADAHSTLAKI

SEQ ID NO: 76 - protein, 296 aa, synthetic construct
MLLPWATSAPGLAWGPLVLGLFGLLAASQPQAVPPYASENQTCRDQEKEYYEPQHRICCSRCPPGTYVSAKCSRIRDTVCATCAENSYNEHWNYLTICQLCRPCDPVMGLEEIAPCTSKRKTQCRCQPGMFCAAWALECTHCELLSDCPPGTEAELKDEVGKGNNHCVPCKAGHFQNTSSPSARCQPHTRCENQGLVEAAPGTAQSDTTCKNPLEPLPPEMSGTMLMLAVLLPLAFFLLLATVFSCIWKSHPSLCRSKRSRLLHSDYMNMTPRRPGPTRKHYQPYAPPRDFAAYRS

SEQ ID NO: 77 - protein, 357 aa, synthetic construct
MLLPWATSAPGLAWGPLVLGLFGLLAASQPQAVPPYASENQTCRDQEKEYYEPQHRICCSRCPPGTYVSAKCSRIRDTVCATCAENSYNEHWNYLTICQLCRPCDPVMGLEEIAPCTSKRKTQCRCQPGMFCAAWALECTHCELLSDCPPGTEAELKDEVGKGNNHCVPCKAGHFQNTSSPSARCQPHTRCENQGLVEAAPGTAQSDTTCKNPLEPLPPEMSGTMLMLAVLLPLAFFLLLATVFSCIWKSHPSLCTQVAADWTALAEEMDFEYLEIRQLETQADPTGRLLDAWQGRPGASVGRLLELLTKLGRDDVLLELGPSIEEDCQKYILKQQQEEAEKPLQVAAVDSSVPRTA

SEQ ID NO: 78 - protein, 229 aa, synthetic construct
MKTLPAMLGTGKLFWVFFLIPYLDIWNIHGKESCDVQLYIKRQSEHSILAGDPFELECPVKYCANRPHVTWCKLNGTTCVKLEDRQTSWKEEKNISFFILHFEPVLPNDNGSYRCSANFQSNLIESHSTTLYVTDVKSASERPSKDEMPAPAREPGHSPQIISFFLALTSTALLFLLFFLTLRFSVVKRGRKKLLYIFKQPFMRPVQTTQEEDGCSCRFPEEEEGGCEL

SEQ ID NO: 79 - protein, 518 aa, synthetic construct
MDRAPQRQHRASRELLAAKKTHTSQIEVIPCKICGDKSSGIHYGVITCEGCKGFFRRSQRCNAAYSCTRQQNCPIDRTSRNRCQHCRLQKCLALGMSRDAVKFGRMSKKQRDSLHAEVQKQLQQRQQQQQEPVVKTPPAGAQGADTLTYTLGLPDGQLPLGSSPDLPEASACPPGLLKASGSGPSYSNNLAKAGLNGASCHLEYSPERGKAEGRESFYSTGSQLTPDRCGLRFEEHRHPGLGELGQGPDSYGSPSFRSTPEAPYASLTEIEHLVQSVCKSYRETCQLRLEDLLRQRSNIFSREEVTGYQRKSMWEMWERCAHHLTEAIQYVVEFAKRLSGFMELCQNDQIVLLKAGAMEVVLVRMCRAYNADNRTVFFEGKYGGMELFRALGCSELISSIFDFSHSLSALHFSEDEIALYTALVLINAHRPGLQEKRKVEQLQYNLELAFHHHLCKTHRQSILAKLPPKGKLRSLCSQHVERLQIFQHLHPIVVQAAFPPLYKELFSTETESPVGLSK

SEQ ID NO: 80 - protein, 274 aa, synthetic construct
MHLCGGNGLLTQTDPKEQQRQLKKQKNRAAAQRSRQKHTDKADALHQQHESLEKDNLALRKEIQSLQAELAWWSRTLHVHERLCPMDCASCSAPGLLGCWDQAEGLLGPGPQGQHGCREQLELFQTPGSCYPAQPLSPGPQPHDSPSLLQCPLPSLSLGPAVVAEPPVQLSPSPLLFASHTGSSLQGSSSKLSALQPSLTAQTAPPQPLELEHPTRGKLGSSPDNPSSALGLARLQSREHKPALSAATWQGLVVDPSPHPLLAFPLLSSAQVHF

SEQ ID NO: 81 - protein, 201 aa, synthetic construct
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIG

SEQ ID NO: 82 - protein, 384 aa, synthetic construct
MPQLDSGGGGAGGGDDLGAPDELLAFQDEGEEQDDKSRDSAAGPERDLAELKSSLVNESEGAAGGAGIPGVPGAGAGARGEAEALGREHAAQRLFPDKLPEPLEDGLKAPECTSGMYKETVYSAFNLLMHYPPPSGAGQHPQPQPPLHKANQPPHGVPQLSLYEHFNSPHPTPAPADISQKQVHRPLQTPDLSGFYSLTSGSMGQLPHTVSWFTHPSLMLGSGVPGHPAAIPHPAIVPPSGKQELQPFDRNLKTQAESKAEKEAKKPTIKKPLNAFMLYMKEMRAKVIAECTLKESAAINQILGRRWHALSREEQAKYYELARKERQLHMQLYPGWSARDNYGKKKRRSREKHQESTTGGKRNAFGTYPEKAAAPAPFLPMTVL

SEQ ID NO: 83 - protein, 507 aa, synthetic construct
MAGAGPKRRALAAPAAEEKEEAREKMLAAKSADGSAPAGEGEGVTLQRNITLLNGVAIIVGTIIGSGIFVTPTGVLKEAGSPGLALVVWAACGVFSIVGALCYAELGTTISKSGGDYAYMLEVYGSLPAFLKLWIELLIIRPSSQYIVALVFATYLLKPLFPTCPVPEEAAKLVACLCVLLLTAVNCYSVKAATRVQDAFAAAKLLALALIILLGFVQIGKGDVSNLDPNFSFEGTKLDVGNIVLALYSGLFAYGGWNYLNFVTEEMINPYRNLPLAIIISLPIVTLVYVLTNLAYFTTLSTEQMLSSEAVAVDFGNYHLGVMSWIIPVFVGLSCFGSVNGSLFTSSRLFFVGSREGHLPSILSMIHPQLLTPVPSLVFTCVMTLLYAFSKDIFSVINFFSFFNWLCVALAIIGMIWLRHRKPELERPIKVNLALPVFFILACLFLIAVSFWKTPVECGIGFTIILSGLPVYFFGVWWKNKPKWLLQGIFSTTVLCQKLMQVVPQET

SEQ ID NO: 84 - protein, 794 aa, synthetic construct
MAGWIQAQQLQGDALRQMQVLYGQHFPIEVRHYLAQWIESQPWDAIDLDNPQDRAQATQLLEGLVQELQKKAEHQVGEDGFLLKIKLGHYATQLQKTYDRCPLELVRCIRHILYNEQRLVREANNCSSPAGILVDAMSQKHLQINQTFEELRLVTQDTENELKKLQQTQEYFIIQYQESLRIQAQFAQLAQLSPQERLSRETALQQKQVSLEAWLQREAQTLQQYRVELAEKHQKTLQLLRKQQTIILDDELIQWKRRQQLAGNGGPPEGSLDVLQSWCEKLAEIIWQNRQQIRRAEHLCQQLPIPGPVEEMLAEVNATITDIISALVTSTFIIEKQPPQVLKTQTKFAATVRLLVGGKLNVHMNPPQVKATIISEQQAKSLLKNENTRNECSGEILNNCCVMEYHQATGTLSAHFRNMSLKRIKRADRRGAESVTEEKFTVLFESQFSVGSNELVFQVKTLSLPVVVIVHGSQDHNATATVLWDNAFAEPGRVPFAVPDKVLWPQLCEALNMKFKAEVQSNRGLTKENLVFLAQKLFNNSSSHLEDYSGLSVSWSQFNRENLPGWNYTFWQWFDGVMEVLKKHHKPHWNDGAILGFVNKQQAHDLLINKPDGTFLLRFSDSEIGGITIAWKFDSPERNLWNLKPFTTRDFSIRSLADRLGDLSYLIYVFPDRPKDEVFSKYYTPVLAKAVDGYVKPQIKQVVPEFVNASADAGGSSATYMDQAPSPAVCPQAPYNMYPQNPDHVLDQDGEFDLDETMDVARHVEELLRRPMDSLDSRLSPPAGLFTSARGSLS

SEQ ID NO: 85 - protein, 465 aa, synthetic construct
MGGAVVDEGPTGVKAPDGGWGWAVLFGCFVITGFSYAFPKAVSVFFKELIQEFGIGYSDTAWISSILLAMLYGTGPLCSVCVNRFGCRPVMLVGGLFASLGMVAASFCRSIIQVYLTTGVITGLGLALNFQPSLIMLNRYFSKRRPMANGLAAAGSPVFLCALSPLGQLLQDRYGWRGGFLILGGLLLNCCVCAALMRPLVVTAQPGSGPPRPSRRLLDLSVFRDRGFVLYAVAASVMVLGLFVPPVFVVSYAKDLGVPDTKAAFLLTILGFIDIFARPAAGFVAGLGKVRPYSVYLFSFSMFFNGLADLAGSTAGDYGGLVVFCIFFGISYGMVGALQFEVLMAIVGTHKFSSAIGLVLLMEAVAVLVGPPSGGKLLDATHVYMYVFILAGAEVLTSSLILLLGNFFCIRKKPKEPQPEVAAAEEEKLHKPPADSGVDLREVEHFLKAEPEKNGEVVHTPETSV

SEQ ID NO: 86 - DNA, 603 bp, synthetic construct
  source 1..603: mol_type = other DNA
atgcaaattccccaagccccatggccagttgtatgggcagttctccagctcgggtggcggcccggctggtttctcgatagcccggatcggccttggaatccaccaacatttagtcctgcacttctggtcgttacagagggtgataatgcaacatttacatgttcctttagcaatacctcagaatcctttgttttgaattggtatcgaatgtctccaagtaatcaaaccgataaactcgcggcatttccagaagataggtcccaaccgggtcaagattgtaggtttagagtgactcagcttccaaatggtcgggattttcatatgtctgtcgtgcgcgctcgcagaaacgattcaggtacatatctttgcggcgctataagtctcgcgccgaaagcccaaataaaggaatctcttagggccgaactgcgagttacggaaaggcgggccgaggttcctactgctcatccttctcctagccctcgccctgcagggcaatttcagactcttgtcgtgggcgtagtaggcggtttgcttgggtcacttgtattgctcgtttgggtgctcgctgtgatttgttctagggcagccagaggaactatcggc

SEQ ID NO: 87 - DNA, 822 bp, synthetic construct
  source 1..822: mol_type = other DNA
atgcacctctgtgggggcaatgggctgctgacccagacagaccccaaggagcaacaaaggcagctgaagaagcagaagaaccgggcagccgcccagcgaagccggcagaagcacacagacaaggcagacgccctgcaccagcagcacgagtctctggaaaaagacaacctcgccctgcggaaggagatccagtccctgcaggccgagctggcgtggtggagccggaccctgcacgtgcatgagcgcctgtgccccatggattgtgcctcctgctcagctccagggctcctgggctgctgggaccaggctgaggggctcctgggccctggcccacagggacaacatggctgccgggagcagctggagctgttccagaccccgggttcctgttacccagctcagccgctctctccaggtccacagcctcatgattctcccagcctcctccagtgccccctgccctcactgtcccttggccccgctgtggttgctgaacctcctgtccagctgtcccccagccctctcctgtttgcctcgcacactggttccagcctgcaggggtcttcctctaagctcagtgccctccagcccagcctcacggcccaaactgcccctccacagcccctcgagctggagcatcccaccagagggaagctggggtcctctcccgacaacccttcctctgccctggggcttgcacgtctgcagagcagggagcacaaacctgctctctcagcagccacttggcaagggctggttgtggatcccagccctcaccctctcctggcctttcctctgctctcctctgctcaagtccacttc

SEQ ID NO: 88 - DNA, 1554 bp, synthetic construct
  source 1..1554: mol_type = other DNA
atggacagggccccacagagacagcaccgagcctcacgggagctgctggctgcaaagaagacccacacctcacaaattgaagtgatcccttgcaaaatctgtggggacaagtcgtctgggatccactacggggttatcacctgtgaggggtgcaagggcttcttccgccggagccagcgctgtaacgcggcctactcctgcacccgtcagcagaactgccccatcgaccgcaccagccgaaaccgatgccagcactgccgcctgcagaaatgcctggcgctgggcatgtcccgagatgctgtcaagttcggccgcatgtccaagaagcagagggacagcctgcatgcagaagtgcagaaacagctgcagcagcggcaacagcagcaacaggaaccagtggtcaagacccctccagcaggggcccaaggagcagataccctcacctacaccttggggctcccagacgggcagctgcccctgggctcctcgcctgacctgcctgaggcttctgcctgtccccctggcctcctgaaagcctcaggctctgggccctcatattccaacaacttggccaaggcagggctcaatggggcctcatgccaccttgaatacagccctgagcggggcaaggctgagggcagagagagcttctatagcacaggcagccagctgacccctgaccgatgtggacttcgttttgaggaacacaggcatcctgggcttggggaactgggacagggcccagacagctacggcagccccagtttccgcagcacaccggaggcaccctatgcctccctgacagagatagagcacctggtgcagagcgtctgcaagtcctacagggagacatgccagctgcggctggaggacctgctgcggcagcgctccaacatcttctcccgggaggaagtgactggctaccagaggaagtccatgtgggagatgtgggaacggtgtgcccaccacctcaccgaggccattcagtacgtggtggagttcgccaagaggctctcaggctttatggagctctgccagaatgaccagattgtgcttctcaaagcaggagcaatggaagtggtgctggttaggatgtgccgggcctacaatgctgacaaccgcacggtcttttttgaaggcaaatacggtggcatggagctgttccgagccttgggctgcagcgagctcatcagctccatctttgacttctcccactccctaagtgccttgcacttttccgaggatgagattgccctctacacagcccttgttctcatcaatgcccatcggccagggctccaagagaaaaggaaagtagaacagctgcagtacaatctggagctggcctttcatcatcatctctgcaagactcatcgccaaagcatcctggcaaagctgccacccaaggggaagcttcggagcctgtgtagccagcatgtggaaaggctgcagatcttccagcacctccaccccatcgtggtccaagccgctttccctccactctacaaggagctcttcagcactgaaaccgagtcacctgtggggctgtccaag

SEQ ID NO: 89 - DNA, 1152 bp, synthetic construct
  source 1..1152: mol_type = other DNA
atgccgcagctggattctggcggaggaggagcgggcggaggagatgatctcggcgcgccggatgagctgctggccttccaggatgaaggcgaggagcaggatgacaagagccgagatagcgccgccggtcctgagcgcgacctggccgagctcaagtcgtcgcttgtgaacgaatccgagggagcagccggaggagcaggaatcccgggagttccgggagccggcgccggagcccgaggcgaggccgaagctcttggacgagaacacgctgcgcagagactcttcccggacaaacttccagagcccctggaggacggcctgaaggccccggagtgcaccagcggcatgtacaaagagaccgtctactccgccttcaatctgctcatgcattacccacctccttcgggagcaggacagcaccctcagccgcagcctccgctgcacaaggccaatcagcctcctcacggtgtccctcaactctctctctacgaacatttcaacagcccacatcccacccctgcacctgcggacatcagccagaagcaagttcacaggcctctgcagacccctgacctctctggcttctactccctgacctcaggcagcatggggcagctcccccacactgtgagctggttcacccacccatccttgatgctaggttctggtgtacctggtcacccagcagccatccctcacccggccattgtgcctccttcagggaagcaggagctgcagcctttcgaccgcaacctgaagacacaagcagagtccaaggcagagaaggaggccaagaagccaaccatcaagaagcccctcaatgccttcatgctgtacatgaaggagatgagagccaaggtcattgcagagtgcacacttaaggagagcgctgccatcaaccagatcctgggccgcaggtggcacgcgctgtcgcgagaagagcaggccaagtactatgagctggcccgcaaggagaggcagctgcacatgcagctatacccaggctggtcagcgcgggacaactacgggaagaagaagaggcggtcgagggaaaagcaccaagaatccaccacaggaggaaaaagaaatgcattcggtacttacccggagaaggccgctgccccagccccgttccttccgatgacagtgctc

SEQ ID NO: 90 - DNA, 2382 bp, synthetic construct
  source 1..2382: mol_type = other DNA
atggcgggctggatccaggcccaacaactccagggagacgcgctgcgccagatgcaggtgctgtacggccagcacttccccatcgaggtccggcactacttggcccagtggattgagagccagccatgggatgccattgacttggacaatccccaggacagagcccaagccacccagctcctggagggcctggtgcaggagctgcagaagaaggcggagcaccaggtgggggaagatgggtttttactgaagatcaagctggggcactacgccacgcagctccagaaaacatatgaccgctgccccctggagctggtccgctgcatccggcacattctgtacaatgaacagaggctggtccgagaagccaacaattgcagctctccggctgggatcctggttgacgccatgtcccagaagcaccttcagatcaaccagacatttgaggagctgcgactggtcacgcaggacacagagaatgagctgaagaaactgcagcagactcaggagtacttcatcatccagtaccaggagagcctgaggatccaagctcagtttgcccagctggcccagctgagcccccaggagcgtctgagccgggagacggccctccagcagaagcaggtgtctctggaggcctggttgcagcgtgaggcacagacactgcagcagtaccgcgtggagctggccgagaagcaccagaagaccctgcagctgctgcggaagcagcagaccatcatcctggatgacgagctgatccagtggaagcggcggcagcagctggccgggaacggcgggccccccgagggcagcctggacgtgctacagtcctggtgtgagaagttggccgagatcatctggcagaaccggcagcagatccgcagggctgagcacctctgccagcagctgcccatccccggcccagtggaggagatgctggccgaggtcaacgccaccatcacggacattatctcagccctggtgaccagcacattcatcattgagaagcagcctcctcaggtcctgaagacccagaccaagtttgcagccaccgtacgcctgctggtgggcgggaagctgaacgtgcacatgaatcccccccaggtgaaggccaccatcatcagtgagcagcaggccaagtctctgcttaaaaatgagaacacccgcaacgagtgcagtggtgagatcctgaacaactgctgcgtgatggagtaccaccaagccacgggcaccctcagtgcccacttcaggaacatgtcactgaagaggatcaagcgtgctgaccggcggggtgcagagtccgtgacagaggagaagttcacagtcctgtttgagtctcagttcagtgttggcagcaatgagcttgtgttccaggtgaagactctgtccctacctgtggttgtcatcgtccacggcagccaggaccacaatgccacggctactgtgctgtgggacaatgcctttgctgagccgggcagggtgccatttgccgtgcctgacaaagtgctgtggccgcagctgtgtgaggcgctcaacatgaaattcaaggccgaagtgcagagcaaccggggcctgaccaaggagaacctcgtgttcctggcgcagaaactgttcaacaacagcagcagccacctggaggactacagtggcctgtccgtgtcctggtcccagttcaacagggagaacttgccgggctggaactacaccttctggcagtggtttgacggggtgatggaggtgttgaagaagcaccacaagccccactggaatgatggggccatcctaggttttgtgaataagcaacaggcccacgacctgctcatcaacaagcccgacgggaccttcttgttgcgctttagtgactcagaaatcgggggcatcaccatcgcctggaagtttgactccccggaacgcaacctgtggaacctgaaaccattcaccacgcgggatttctccatcaggtccctggctgaccggctgggggacctgagctatctcatctatgtgtttcctgaccgccccaaggatgaggtcttctccaagtactacactcctgtgctggctaaagctgttgatggatatgtgaaaccacagatcaagcaagtggtccctgagtttgtgaatgcatctgcagatgctgggggcagcagcgccacgtacatggaccaggccccctccccagctgtgtgcccccaggctccctataacatgtacccacagaaccctgaccatgtactcgatcaggatggagaattcgacctggatgagaccatggatgtggccaggcacgtggaggaactcttacgccgaccaatggacagtcttgactcccgcctctcgccccctgccggtcttttcacctctgccagaggctccctctca

SEQ ID NO: 91 - DNA, 1395 bp, synthetic construct
  source 1..1395: mol_type = other DNA
atgggaggggccgtggtggacgagggccccacaggcgtcaaggcccctgacggcggctggggctgggccgtgctcttcggctgtttcgtcatcactggcttctcctacgccttccccaaggccgtcagtgtcttcttcaaggagctcatacaggagtttgggatcggctacagcgacacagcctggatctcctccatcctgctggccatgctctacgggacaggtccgctctgcagtgtgtgcgtgaaccgctttggctgccggcccgtcatgcttgtggggggtctctttgcgtcgctgggcatggtggctgcgtccttttgccggagcatcatccaggtctacctcaccactggggtcatcacggggttgggtttggcactcaacttccagccctcgctcatcatgctgaaccgctacttcagcaagcggcgccccatggccaacgggctggcggcagcaggtagccctgtcttcctgtgtgccctgagcccgctggggcagctgctgcaggaccgctacggctggcggggcggcttcctcatcctgggcggcctgctgctcaactgctgcgtgtgtgccgcactcatgaggcccctggtggtcacggcccagccgggctcggggccgccgcgaccctcccggcgcctgctagacctgagcgtcttccgggaccgcggctttgtgctttacgccgtggccgcctcggtcatggtgctggggctcttcgtcccgcccgtgttcgtggtgagctacgccaaggacctgggcgtgcccgacaccaaggccgccttcctgctcaccatcctgggcttcattgacatcttcgcgcggccggccgcgggcttcgtggcggggcttgggaaggtgcggccctactccgtctacctcttcagcttctccatgttcttcaacggcctcgcggacctggcgggttctacggcgggcgactacggcggcctcgtggtcttctgcatcttctttggcatctcctacggcatggtgggggccctgcagttcgaggtgctcatggccatcgtgggcacccacaagttctccagtgccattggcctggtgctgctgatggaggcggtggccgtgctcgtcgggcccccttcgggaggcaaactcctggatgcgacccacgtctacatgtacgtgttcatcctggcgggggccgaggtgctcacctcctccctgattttgctgctgggcaacttcttctgcattaggaagaagcccaaagagccacagcctgaggtggcggccgcggaggaggagaagctccacaagcctcctgcagactcgggggtggacttgcgggaggtggagcatttcctgaaggctgagcctgagaaaaacggggaggtggttcacaccccggaaacaagtgtc

SEQ ID NO: 92 - DNA, 1521 bp, synthetic construct
  source 1..1521: mol_type = other DNA
atggcgggtgcgggcccgaagcggcgcgcgctagcggcgccggcggccgaggagaaggaagaggcgcgggagaagatgctggccgccaagagcgcggacggctcggcgccggcaggcgagggcgagggcgtgaccctgcagcggaacatcacgctgctcaacggcgtggccatcatcgtggggaccattatcggctcgggcatcttcgtgacgcccacgggcgtgctcaaggaggcaggctcgccggggctggcgctggtggtgtgggccgcgtgcggcgtcttctccatcgtgggcgcgctctgctacgcggagctcggcaccaccatctccaaatcgggcggcgactacgcctacatgctggaggtctacggctcgctgcccgccttcctcaagctctggatcgagctgctcatcatccggccttcatcgcagtacatcgtggccctggtcttcgccacctacctgctcaagccgctcttccccacctgcccggtgcccgaggaggcagccaagctcgtggcctgcctctgcgtgctgctgctcacggccgtgaactgctacagcgtgaaggccgccacccgggtccaggatgcctttgccgccgccaagctcctggccctggccctgatcatcctgctgggcttcgtccagatcgggaagggtgatgtgtccaatctagatcccaacttctcatttgaaggcaccaaactggatgtggggaacattgtgctggcattatacagcggcctctttgcctatggaggatggaattacttgaatttcgtcacagaggaaatgatcaacccctacagaaacctgcccctggccatcatcatctccctgcccatcgtgacgctggtgtacgtgctgaccaacctggcctacttcaccaccctgtccaccgagcagatgctgtcgtccgaggccgtggccgtggacttcgggaactatcacctgggcgtcatgtcctggatcatccccgtcttcgtgggcctgtcctgcttcggctccgtcaatgggtccctgttcacatcctccaggctcttcttcgtggggtcccgggaaggccacctgccctccatcctctccatgatccacccacagctcctcacccccgtgccgtccctcgtgttcacgtgtgtgatgacgctgctctacgccttctccaaggacatcttctccgtcatcaacttcttcagcttcttcaactggctctgcgtggccctggccatcatcggcatgatctggctgcgccacagaaagcctgagcttgagcggcccatcaaggtgaacctggccctgcctgtgttcttcatcctggcctgcctcttcctgatcgccgtctccttctggaagacacccgtggagtgtggcatcggcttcaccatcatcctcagcgggctgcccgtctacttcttcggggtctggtggaaaaacaagcccaagtggctcctccagggcatcttctccacgaccgtcctgtgtcagaagctcatgcaggtggtcccccaggagaca

SEQ ID NO: 93 - protein, 301 aa, synthetic construct
MFSHLPFDCVLLLLLLLLTRSSEVEYRAEVGQNAYLPCFYTPAAPGNLVPVCWGKGACPVFECGNVVLRTDERDVNYWTSRYWLNGDFRKGDVSLTIENVTLADSGIYCCRIQIPGIMNDEKFNLKLVIKPAKVTPAPTRQRDFTAAFPRMLTTRGHGPAETQTLGSLPDINLTQISTLANELRDSRLANDLRDSGATIRIGIYIGAGICAGLALALIFGALIFKWYSHSKEKIQNLSLISLANLPPSGLANAVAEGIRSEENIYTIEENVYEVEEPNEYYCYVSSRQQPSQPLGCRFAMP

SEQ ID NO: 94 - protein, 255 aa, synthetic construct; identical to SEQ ID NO: 55

SEQ ID NO: 95 - protein, 244 aa, synthetic construct
MRWCLLLIWAQGLRQAPLASGMMTGTIETTGNISAEKGGSIILQCHLSSTTAQVTQVNWEQQDQLLAICNADLGWHISPSFKDRVAPGPGLGLTLQSLTVNDTGEYFCIYHTYPDGTYTGRIFLEVLESSVAEHGARFQIPLLGAMAATLVVICTAVIVVVALTRKKKALRIHSVEGDLRRKSAGQEEWSPSAPSPPGSCVQAEAAPAGLCGEQRGEDCAELHDYFNVLSYRSLGNCSFFTETG

SEQ ID NO: 96 - protein, 567 aa, synthetic construct
MGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIFYCYRVNRQQKLSSTWETGKTRKLMEFSEHCAIILEDDRSDISSTCANNINHNTELLPIELDTLVGKGRFAEVYKAKLKQNTSEQFETVAVKIFPYEEYASWKTEKDIFSDINLKHENILQFLTAEERKTELGKQYWLITAFHAKGNLQEYLTRHVISWEDLRKLGSSLARGIAHLHSDHTPCGRPKMPIVHRDLKSSNILVKNDLTCCLCDFGLSLRLDPTLSVDDLANSGQVGTARYMAPEVLESRMNLENVESFKQTDVYSMALVLWEMTSRCNAVGEVKDYEPPFGSKVREHPCVESMKDNVLRDRGRPEIPSFWLNHQGIQMVCETLTECWDHDPEARLTAQCVAERFSELEHLDRLSGRSCSEEKIPEDGSLNTTK

SEQ ID NO: 97 - protein, 435 aa, synthetic construct
MLLPWATSAPGLAWGPLVLGLFGLLAASQPQAVPPYASENQTCRDQEKEYYEPQHRICCSRCPPGTYVSAKCSRIRDTVCATCAENSYNEHWNYLTICQLCRPCDPVMGLEEIAPCTSKRKTQCRCQPGMFCAAWALECTHCELLSDCPPGTEAELKDEVGKGNNHCVPCKAGHFQNTSSPSARCQPHTRCENQGLVEAAPGTAQSDTTCKNPLEPLPPEMSGTMLMLAVLLPLAFFLLLATVFSCIWKSHPSLCRKLGSLLKRRPQGEGPNPVAGSWEPPKAHPYFPDLVQPLLPISGDVSPVSTGLPAAPVLEAGVPQQQSPLDLTREPQLEPGEQSQVAHGTNGIHVTGGSMTITGNIYIYNGPVLGGPPGPGDLPATPEPPYPIPEEGDPGPPGLSTPHQEDGKAWHLAETEHCGATPSNRGPRNQFITHD

SEQ ID NO: 98 - protein, 825 aa, synthetic construct
MGWLCSGLLFPVSCLVLLQVASSGNMKVLQEPTCVSDYMSISTCEWKMNGPTNCSTELRLLYQLVFLLSEAHTCIPENNGGAGCVCHLLMDDVVSADNYTLDLWAGQQLLWKGSFKPSEHVKPRAPGNLTVHTNVSDTLLLTWSNPYPPDNYLYNHLTYAVNIWSENDPADFRIYNVTYLEPSLRIAASTLKSGISYRARVRAWAQCYNTTWSEWSPSTKWHNSYREPFEQHLLLGVSVSCIVILAVCLLCYVSITKIKKEWWDQIPNPARSRLVAIIIQDAQGSQWEKRSRGQEPAKCPHWKNCLTKLLPCFLEHNMKRDEDPHKAAKEMPFQGSGKSAWCPVEISKTVLWPESISVVRCVELFEAPVECEEEEEVEEEKGSFCASPESSRDDFQEGREGIVARLTESLFLDLLGEENGGFCQQDMGESCLLPPSGSTSAHMPWDEFPSAGPKEAPPWGKEQPLHLEPSPPASPTQSPDNLTCTETPLVIAGNPAYRSFSNSLSQSPCPRELGPDPLLARHLEEVEPEMPCVPQLSEPTTVPQPEPETWEQILRRNVLQHGAAAAPVSAPTSGYQEFVHAVEQGGTQASAVVGLGPPGEAGYKAFSSLLASSAVSPEKCGFGASSGEEGYKPFQDLIPGCPGDPAPVPVPLFTFGLDREPPRSPQSSHLPSSSPEHLGLEPGEKVEDMPKPPLPQEQATDPLVDSLGSGIVYSALTCHLCGHLKQCHGQEDGGQTPVMASPCCGCCCGDRSSPPTTPLRAPDPSPGGVPLEASLCPASLAPSGISEKSKSSSSFHPAPGNAQSSSQTPKIVNFVSVGPTYMRVS

SEQ ID NO: 99 - protein, 370 aa, synthetic construct
MLGQVVTLILLLLLKVYQGKGCQGSADHVVSISGVPLQLQPNSIQTKVDSIAWKKLLPSQNGFHHILKWENGSLPSNTSNDRFSFIVKNLSLLIKAAQQQDSGLYCLEVTSISGKVQTATFQVFVFESLLPDKVEKPRLQGQGKILDRGRCQVALSCLVSRDGNVSYAWYRGSKLIQTAGNLTYLDEEVDINGTHTYTCNVSNPVSWESHTLNLTQDCQNAHQEFRFWPFLVIIVILSALFLGTLACFCVWRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYS

SEQ ID NO: 100 - protein, 311 aa, synthetic construct
MGVPTALEAGSWRWGSLLFALFLAASLGPVAAFKVATPYSLYVCPEGQNVTLTCRLLGPVDKGHDVTFYKTWYRSSRGEVQTCSERRPIRNLTFQDLHLHHGGHQAANTSHDLAQRHGLESASDHHGNFSITMRNLTLLDSGLYCCLVVEIRHHHSEHRVHGAMELQVQTGKDAPSNCVVYPSSSQDSENITAAALATGACIVGILCLPLILLLVYKQRQAASNRRAQELVRMDSNIQGIENPGFEASPPAQGIPEAKVRHPLSYVAQRQPSESGRHLLSEPSTPLSPPGPGDVFFPSLDPVPDSPNFEVI

SEQ ID NO: 101 - protein, 220 aa, synthetic construct
MLRLLLALNLFPSIQVTGNKILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 102 - protein, 129 aa, synthetic construct
MARGSLRRLLRLLVLGLWLALLRSVAGEQAPGTAPCSRGSSWSADLDKCMDCASCRARPHSDFCLGCAAAPPAPFRLLWPILGGALSLTFVLGLLSGFLVWRRCRRREKFTTPIEETGGEGCPAVALIQ

SEQ ID NO: 103 - protein, 277 aa, synthetic construct
MCVGARRLGRGPCAALLLLGLGLSTVTGLHCVGDTYPSNDRCCHECRPGNGMVSRCSRSQNTVCRPCGPGFYNDVVSSKPCKPCTWCNLRSGSERKQLCTATQDTVCRCRAGTQPLDSYKPGVDCAPCPPGHFSPGDNQACKPWTNCTLAGKHTLQPASNSSDAICEDRDPPATQPQETQGPPARPITVQPTEAWPRTSQGPSTRPVEVPGGRAVAAILGLGLVLGLLGPLAILLALYLLRRDQRLPPDAHKPPGGGSFRTPIQEEQADAHSTLAKI

SEQ ID NO: 104 - protein, 288 aa, synthetic construct
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 105 - protein, 289 aa, synthetic construct
MKTLPAMLGTGKLFWVFFLIPYLDIWNIHGKESCDVQLYIKRQSEHSILAGDPFELECPVKYCANRPHVTWCKLNGTTCVKLEDRQTSWKEEKNISFFILHFEPVLPNDNGSYRCSANFQSNLIESHSTTLYVTDVKSASERPSKDEMASRPWLLYRLLPLGGLPLLITTCFCLFCCLRRHQGKQNELSDTAGREINLVDAHLKSEQTEASTRQNSQVLLSETGIYDNDPDLCFRMQEGSEVYSNPCLEENKPGIVYASLNHSVIGPNSRLARNVKEAPTEYASICVRS

SEQ ID NO: 106 - protein, 199 aa, synthetic construct
MKSGLWYFFLFCLRIKVLTGEINGSANYEMFIFHNGGVQILCKYPDIVQQFKMQLLKGGQILCDLTKTKGSGNTVSIKSLKFCHSQLSNNSVSFFLYNLDHSHANYYFCNLSIFDPPPFKVTLTGGYLHIYESQLCCQLKFWLPIGCAAFVVVCILGCILICWLTKKKYSSSVHDPNGEYMFMRAVNTAKKSRLTDVTL

SEQ ID NO: 107 - protein, 440 aa, synthetic construct
MEQRGQNAPAASGARKRHGPGPREARGARPGPRVPKTLVLVVAAVLLLVSAESALITQQDLAPQQRAAPQQKRSSPSEGLCPPGHHISEDGRDCISCKYGQDYSTHWNDLLFCLRCTRCDSGEVELSPCTTTRNTVCQCEEGTFREEDSPEMCRKCRTGCPRGMVKVGDCTPWSDIECVHKESGTKHSGEVPAVEETVTSSPGTPASPCSLSGIIIGVTVAAVVLIVAVFVCKSLLWKKVLPYLKGICSGGGGDPERVDRSSQRPGAEDNVLNEIVSILQPTQVPEQEMEVQEPAEPTGVNMLSPGESEHLLEPAEAERSQRRRLLVPANEGDPTETLRQCFDDFADLVPFDSWEPLMRKLGLMDNEIKVAKAEAAGHRDTLYTMLIKWVNKTGRDASVHTLLDALETLGERLAKQKIEDHLLSSGKFMYLEGNADSAMS

SEQ ID NO: 108 - protein, 21 aa, synthetic construct
MRWCLLLIWAQGLRQAPLASG

SEQ ID NO: 109 - protein, 22 aa, synthetic construct
MGRGLLRGLWPLHIVLWTRIAS

SEQ ID NO: 110 - protein, 30 aa, synthetic construct
MLLPWATSAPGLAWGPLVLGLFGLLAASQP

SEQ ID NO: 111 - protein, 25 aa, synthetic construct
MGWLCSGLLFPVSCLVLLQVASSGN

SEQ ID NO: 112 - protein, 21 aa, synthetic construct
MLGQVVTLILLLLLKVYQGKG

SEQ ID NO: 113 - protein, 32 aa, synthetic construct
MGVPTALEAGSWRWGSLLFALFLAASLGPVAA

SEQ ID NO: 114 - protein, 18 aa, synthetic construct
MLRLLLALNLFPSIQVTG

SEQ ID NO: 115 - protein, 27 aa, synthetic construct
MARGSLRRLLRLLVLGLWLALLRSVAG

SEQ ID NO: 116 - protein, 28 aa, synthetic construct
MCVGARRLGRGPCAALLLLGLGLSTVTG

SEQ ID NO: 117 - protein, 23 aa, synthetic construct
MQIPQAPWPVVWAVLQLGWRPGW

SEQ ID NO: 118 - protein, 30 aa, synthetic construct
MKTLPAMLGTGKLFWVFFLIPYLDIWNIHG

SEQ ID NO: 119 - protein, 20 aa, synthetic construct
MKSGLWYFFLFCLRIKVLTG

SEQ ID NO: 120 - protein, 55 aa, synthetic construct
MEQRGQNAPAASGARKRHGPGPREARGARPGPRVPKTLVLVVAAVLLLVSAESAL

SEQ ID NO: 121 - protein, 120 aa, synthetic construct
MMTGTIETTGNISAEKGGSIILQCHLSSTTAQVTQVNWEQQDQLLAICNADLGWHISPSFKDRVAPGPGLGLTLQSLTVNDTGEYFCIYHTYPDGTYTGRIFLEVLESSVAEHGARFQIP

SEQ ID NO: 122 - protein, 144 aa, synthetic construct
TIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQ

SEQ ID NO: 123 - protein, 197 aa, synthetic construct
QAVPPYASENQTCRDQEKEYYEPQHRICCSRCPPGTYVSAKCSRIRDTVCATCAENSYNEHWNYLTICQLCRPCDPVMGLEEIAPCTSKRKTQCRCQPGMFCAAWALECTHCELLSDCPPGTEAELKDEVGKGNNHCVPCKAGHFQNTSSPSARCQPHTRCENQGLVEAAPGTAQSDTTCKNPLEPLPPEMSGTMLM

SEQ ID NO: 124 - protein, 207 aa, synthetic construct
MKVLQEPTCVSDYMSISTCEWKMNGPTNCSTELRLLYQLVFLLSEAHTCIPENNGGAGCVCHLLMDDVVSADNYTLDLWAGQQLLWKGSFKPSEHVKPRAPGNLTVHTNVSDTLLLTWSNPYPPDNYLYNHLTYAVNIWSENDPADFRIYNVTYLEPSLRIAASTLKSGISYRARVRAWAQCYNTTWSEWSPSTKWHNSYREPFEQH

SEQ ID NO: 125 - protein, 208 aa, synthetic construct
CQGSADHVVSISGVPLQLQPNSIQTKVDSIAWKKLLPSQNGFHHILKWENGSLPSNTSNDRFSFIVKNLSLLIKAAQQQDSGLYCLEVTSISGKVQTATFQVFVFESLLPDKVEKPRLQGQGKILDRGRCQVALSCLVSRDGNVSYAWYRGSKLIQTAGNLTYLDEEVDINGTHTYTCNVSNPVSWESHTLNLTQDCQNAHQEFRFWP

SEQ ID NO: 126 - protein, 162 aa, synthetic construct
FKVATPYSLYVCPEGQNVTLTCRLLGPVDKGHDVTFYKTWYRSSRGEVQTCSERRPIRNLTFQDLHLHHGGHQAANTSHDLAQRHGLESASDHHGNFSITMRNLTLLDSGLYCCLVVEIRHHHSEHRVHGAMELQVQTGKDAPSNCVVYPSSSQDSENITAA

SEQ ID NO: 127 - protein, 134 aa, synthetic construct
NKILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKP

SEQ ID NO: 128 - protein, 53 aa, synthetic construct
EQAPGTAPCSRGSSWSADLDKCMDCASCRARPHSDFCLGCAAAPPAPFRLLWP

SEQ ID NO: 129 - protein, 186 aa, synthetic construct [sequence omitted]

SEQ ID NO: 130 - protein, 147 aa, synthetic construct
FLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLV

SEQ ID NO: 131 - protein, 127 aa, synthetic construct
KESCDVQLYIKRQSEHSILAGDPFELECPVKYCANRPHVTWCKLNGTTCVKLEDRQTSWKEEKNISFFILHFEPVLPNDNGSYRCSANFQSNLIESHSTTLYVTDVKSASERPSKDEMASRPWLLYR

SEQ ID NO: 132 - protein, 120 aa, synthetic construct
EINGSANYEMFIFHNGGVQILCKYPDIVQQFKMQLLKGGQILCDLTKTKGSGNTVSIKSLKFCHSQLSNNSVSFFLYNLDHSHANYYFCNLSIFDPPPFKVTLTGGYLHIYESQLCCQLK

SEQ ID NO: 133 - protein, 155 aa, synthetic construct
ITQQDLAPQQRAAPQQKRSSPSEGLCPPGHHISEDGRDCISCKYGQDYSTHWNDLLFCLRCTRCDSGEVELSPCTTTRNTVCQCEEGTFREEDSPEMCRKCRTGCPRGMVKVGDCTPWSDIECVHKESGTKHSGEVPAVEETVTSSPGTPASPCS

SEQ ID NO: 134 - protein, 120 aa, synthetic construct; identical to SEQ ID NO: 121

SEQ ID NO: 135 - protein, 144 aa, synthetic construct; identical to SEQ ID NO: 122

SEQ ID NO: 136 - protein, 197 aa, synthetic construct; identical to SEQ ID NO: 123

SEQ ID NO: 137 - protein, 207 aa, synthetic construct; identical to SEQ ID NO: 124

SEQ ID NO: 138 - protein, 208 aa, synthetic construct; identical to SEQ ID NO: 125

SEQ ID NO: 139 - protein, 162 aa, synthetic construct; identical to SEQ ID NO: 126

SEQ ID NO: 140 - protein, 134 aa, synthetic construct; identical to SEQ ID NO: 127

SEQ ID NO: 141 - protein, 53 aa, synthetic construct; identical to SEQ ID NO: 128

SEQ ID NO: 142 - protein, 186 aa, synthetic construct [sequence omitted]

SEQ ID NO: 143 - protein, 147 aa, synthetic construct; identical to SEQ ID NO: 130

SEQ ID NO: 144 - protein, 127 aa, synthetic construct; identical to SEQ ID NO: 131

SEQ ID NO: 145 - protein, 21 aa, synthetic construct
FWLPIGCAAFVVVCILGCILI

SEQ ID NO: 146 - protein, 21 aa, synthetic construct
LSGIIIGVTVAAVVLIVAVFV

SEQ ID NO: 147 - protein, 82 aa, synthetic construct
LTRKKKALRIHSVEGDLRRKSAGQEEWSPSAPSPPGSCVQAEAAPAGLCGEQRGEDCAELHDYFNVLSYRSLGNCSFFTETG

SEQ ID NO: 148 - protein, 380 aa, synthetic construct
CYRVNRQQKLSSTWETGKTRKLMEFSEHCAIILEDDRSDISSTCANNINHNTELLPIELDTLVGKGRFAEVYKAKLKQNTSEQFETVAVKIFPYEEYASWKTEKDIFSDINLKHENILQFLTAEERKTELGKQYWLITAFHAKGNLQEYLTRHVISWEDLRKLGSSLARGIAHLHSDHTPCGRPKMPIVHRDLKSSNILVKNDLTCCLCDFGLSLRLDPTLSVDDLANSGQVGTARYMAPEVLESRMNLENVESFKQTDVYSMALVLWEMTSRCNAVGEVKDYEPPFGSKVREHPCVESMKDNVLRDRGRPEIPSFWLNHQGIQMVCETLTECWDHDPEARLTAQCVAERFSELEHLDRLSGRSCSEEKIPEDGSLNTTK

SEQ ID NO: 149 - protein, 187 aa, synthetic construct
KSHPSLCRKLGSLLKRRPQGEGPNPVAGSWEPPKAHPYFPDLVQPLLPISGDVSPVSTGLPAAPVLEAGVPQQQSPLDLTREPQLEPGEQSQVAHGTNGIHVTGGSMTITGNIYIYNGPVLGGPPGPGDLPATPEPPYPIPEEGDPGPPGLSTPHQEDGKAWHLAETEHCGATPSNRGPRNQFITHD

SEQ ID NO: 150 - protein, 569 aa, synthetic construct
KIKKEWWDQIPNPARSRLVAIIIQDAQGSQWEKRSRGQEPAKCPHWKNCLTKLLPCFLEHNMKRDEDPHKAAKEMPFQGSGKSAWCPVEISKTVLWPESISVVRCVELFEAPVECEEEEEVEEEKGSFCASPESSRDDFQEGREGIVARLTESLFLDLLGEENGGFCQQDMGESCLLPPSGSTSAHMPWDEFPSAGPKEAPPWGKEQPLHLEPSPPASPTQSPDNLTCTETPLVIAGNPAYRSFSNSLSQSPCPRELGPDPLLARHLEEVEPEMPCVPQLSEPTTVPQPEPETWEQILRRNVLQHGAAAAPVSAPTSGYQEFVHAVEQGGTQASAVVGLGPPGEAGYKAFSSLLASSAVSPEKCGFGASSGEEGYKPFQDLIPGCPGDPAPVPVPLFTFGLDREPPRSPQSSHLPSSSPEHLGLEPGEKVEDMPKPPLPQEQATDPLVDSLGSGIVYSALTCHLCGHLKQCHGQEDGGQTPVMASPCCGCCCGDRSSPPTTPLRAPDPSPGGVPLEASLCPASLAPSGISEKSKSSSSFHPAPGNAQSSSQTPKIVNFVSVGPTYMRVS

SEQ ID NO: 151 - protein, 120 aa, synthetic construct
WRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYS

SEQ ID NO: 152 - protein, 96 aa, synthetic construct
YKQRQAASNRRAQELVRMDSNIQGIENPGFEASPPAQGIPEAKVRHPLSYVAQRQPSESGRHLLSEPSTPLSPPGPGDVFFPSLDPVPDSPNFEVI

SEQ ID NO: 153 - protein, 41 aa, synthetic construct
RSKRSRLLHSDYMNMTPRRPGPTRKHYQPYAPPRDFAAYRS

SEQ ID NO: 154 - protein, 28 aa, synthetic construct
RRCRRREKFTTPIEETGGEGCPAVALIQ

SEQ ID NO: 155 - protein, 42 aa, synthetic construct
ALYLLRRDQRLPPDAHKPPGGGSFRTPIQEEQADAHSTLAKI

SEQ ID NO: 156 - protein, 97 aa, synthetic construct
CSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 157 - protein, 111 aa, synthetic construct
RRHQGKQNELSDTAGREINLVDAHLKSEQTEASTRQNSQVLLSETGIYDNDPDLCFRMQEGSEVYSNPCLEENKPGIVYASLNHSVIGPNSRLARNVKEAPTEYASICVRS

SEQ ID NO: 158 - protein, 38 aa, synthetic construct
CWLTKKKYSSSVHDPNGEYMFMRAVNTAKKSRLTDVTL

SEQ ID NO: 159 - protein, 209 aa, synthetic construct
CKSLLWKKVLPYLKGICSGGGGDPERVDRSSQRPGAEDNVLNEIVSILQPTQVPEQEMEVQEPAEPTGVNMLSPGESEHLLEPAEAERSQRRRLLVPANEGDPTETLRQCFDDFADLVPFDSWEPLMRKLGLMDNEIKVAKAEAAGHRDTLYTMLIKWVNKTGRDASVHTLLDALETLGERLAKQKIEDHLLSSGKFMYLEGNADSAMS

SEQ ID NO: 160 - protein, 276 aa, synthetic construct
MEQRGQNAPAASGARKRHGPGPREARGARPGPRVPKTLVLVVAAVLLLVSAESALITQQDLAPQQRAAPQQKRSSPSEGLCPPGHHISEDGRDCISCKYGQDYSTHWNDLLFCLRCTRCDSGEVELSPCTTTRNTVCQCEEGTFREEDSPEMCRKCRTGCPRGMVKVGDCTPWSDIECVHKESGTKHSGEVPAVEETVTSSPGTPASPCSLSGIIIGVTVAAVVLIVAVFVCKSLLWKCWLTKKKYSSSVHDPNGEYMFMRAVNTAKKSRLTDVTL

SEQ ID NO: 161 - protein, 203 aa, synthetic construct
MRWCLLLIWAQGLRQAPLASGMMTGTIETTGNISAEKGGSIILQCHLSSTTAQVTQVNWEQQDQLLAICNADLGWHISPSFKDRVAPGPGLGLTLQSLTVNDTGEYFCIYHTYPDGTYTGRIFLEVLESSVAHIYESQLCCQLKFWLPIGCAAFVVVCILGCILICWLTKKKYSSSVHDPNGEYMFMRAVNTAKKSRLTDVTL

SEQ ID NO: 162 - protein, 297 aa, synthetic construct
MLLPWATSAPGLAWGPLVLGLFGLLAASQPQAVPPYASENQTCRDQEKEYYEPQHRICCSRCPPGTYVSAKCSRIRDTVCATCAENSYNEHWNYLTICQLCRPCDPVMGLEEIAPCTSKRKTQCRCQPGMFCAAWALECTHCELLSDCPPGTEAELKDEVGKGNNHCVPCKAGHFQNTSSPSARCQPHTRCENQGLVEAAPGTAQSDTTCKNPLEPLPPEMSGTMLMLAVLLPLAFFLLLATVFSCIWKSHPSLCKRGRKKLLYIFKQPFMRPVQTTQEEDGCSCRFPEEEEGGCEL

SEQ ID NO: 163 - DNA, 828 bp, synthetic construct
  source 1..828: mol_type = other DNA
atggaacaacggggacagaacgccccggccgcttcgggggcccggaaaaggcacggcccaggacccagggaggcgcggggagccaggcctgggccccgggtccccaagacccttgtgctcgttgtcgccgcggtcctgctgttggtctcagctgagtctgctctgatcacccaacaagacctagctccccagcagagagcggccccacaacaaaagaggtccagcccctcagagggattgtgtccacctggacaccatatctcagaagacggtagagattgcatctcctgcaaatatggacaggactatagcactcactggaatgacctccttttctgcttgcgctgcaccaggtgtgattcaggtgaagtggagctaagtccctgcaccacgaccagaaacacagtgtgtcagtgcgaagaaggcaccttccgggaagaagattctcctgagatgtgccggaagtgccgcacagggtgtcccagagggatggtcaaggtcggtgattgtacaccctggagtgacatcgaatgtgtccacaaagaatcaggtacaaagcacagtggggaagtcccagctgtggaggagacggtgacctccagcccagggactcctgcctctccctgttctctctcaggcatcatcataggagtcacagttgcagccgtagtcttgattgtggctgtgtttgtttgcaagtctttactgtggaagtgttggcttacaaaaaagaagtattcatccagtgtgcacgaccctaacggtgaatacatgttcatgagagcagtgaacacagccaaaaaatctagactcacagatgtgacccta

SEQ ID NO: 164 - DNA, 609 bp, synthetic construct
  source 1..609: mol_type = other DNA
atgcgctggtgtctcctcctgatctgggcccaggggctgaggcaggctcccctcgcctcaggaatgatgacaggcacaatagaaacaacggggaacatttctgcagagaaaggtggctctatcatcttacaatgtcacctctcctccaccacggcacaagtgacccaggtcaactgggagcagcaggaccagcttctggccatttgtaatgctgacttggggtggcacatctccccatccttcaaggatcgagtggccccaggtcccggcctgggcctcaccctccagtcgctgaccgtgaacgatacaggggagtacttctgcatctatcacacctaccctgatgggacgtacactgggagaatcttcctggaggtcctagaaagctcagtggctcatatttatgaatcacaactttgttgccagctgaagttctggttacccataggatgtgcagcctttgttgtagtctgcattttgggatgcatacttatttgttggcttacaaaaaagaagtattcatccagtgtgcacgaccctaacggtgaatacatgttcatgagagcagtgaacacagccaaaaaatctagactcacagatgtgacccta

SEQ ID NO: 165 - DNA, 891 bp, synthetic construct
  source 1..891: mol_type = other DNA
atgctcctgccttgggccacctctgcccccggcctggcctgggggcctctggtgctgggcctcttcgggctcctggcagcatcgcagccccaggcggtgcctccatatgcgtcggagaaccagacctgcagggaccaggaaaaggaatactatgagccccagcaccgcatctgctgctcccgctgcccgccaggcacctatgtctcagctaaatgtagccgcatccgggacacagtttgtgccacatgtgccgagaattcctacaacgagcactggaactacctgaccatctgccagctgtgccgcccctgtgacccagtgatgggcctcgaggagattgccccctgcacaagcaaacggaagacccagtgccgctgccagccgggaatgttctgtgctgcctgggccctcgagtgtacacactgcgagctactttctgactgcccgcctggcactgaagccgagctcaaagatgaagttgggaagggtaacaaccactgcgtcccctgcaaggccgggcacttccagaatacctcctcccccagcgcccgctgccagccccacaccaggtgtgagaaccaaggtctggtggaggcagctccaggcactgcccagtccgacacaacctgcaaaaatccattagagccactgcccccagagatgtcaggaaccatgctgatgctggccgttctgctgccactggccttctttctgctccttgccaccgtcttctcctgcatctggaagagccacccttctctctgcaaacggggcagaaagaaactcctgtatatattcaaacaaccatttatgagaccagtacaaactactcaagaggaagatggctgtagctgccgatttccagaagaagaagaaggaggatgtgaactg

SEQ ID NO: 166 - protein, 21 aa, synthetic construct
MFSHLPFDCVLLLLLLLLTRS

SEQ ID NO: 167 - protein, 23 aa, synthetic construct
MGNSCYNIVATLLLVLNFERTRS

SEQ ID NO: 168 - protein, 181 aa, synthetic construct
SEVEYRAEVGQNAYLPCFYTPAAPGNLVPVCWGKGACPVFECGNVVLRTDERDVNYWTSRYWLNGDFRKGDVSLTIENVTLADSGIYCCRIQIPGIMNDEKFNLKLVIKPAKVTPAPTRQRDFTAAFPRMLTTRGHGPAETQTLGSLPDINLTQISTLANELRDSRLANDLRDSGATIRIG

SEQ ID NO: 169 - protein, 163 aa, synthetic construct
LQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLVNGTKERDVVCGPSPADLSPGASSVTPPAPAREPGHSPQ

SEQ ID NO: 170 - protein, 181 aa, synthetic construct; identical to SEQ ID NO: 168

SEQ ID NO: 171 - protein, 163 aa, synthetic construct; identical to SEQ ID NO: 169

SEQ ID NO: 172 - protein, 78 aa, synthetic construct
FKWYSHSKEKIQNLSLISLANLPPSGLANAVAEGIRSEENIYTIEENVYEVEEPNEYYCYVSSRQQPSQPLGCRFAMP

SEQ ID NO: 173 - protein, 42 aa, synthetic construct
KRGRKKLLYIFKQPFMRPVQTTQEEDGCSCRFPEEEEGGCEL

SEQ ID NO: 174 - protein, 360 aa, synthetic construct
MNPTDIADTTLDESIYSNYYLYESIPKPCTKEGIKAFGELFLPPLYSLVFVFGLLGNSVVVLVLFKYKRLRSMTDVYLLNLAISDLLFVFSLPFWGYYAADQWVFGLGLCKMISWMYLVGFYSGIFFVMLMSIDRYLAIVHAVFSLRARTLTYGVITSLATWSVAVFASLPGFLFSTCYTERNHTYCKTKYSLNSTTWKVLSSLEINILGLVIPLGIMLFCYSMIIRTLQHCKNEKKNKAVKMIFAVVVLFLGFWTPYNIVLFLETLVELEVLQDCTFERYLDYAIQATETLAFVHCCLNPIIYFFLGEKFRKYILQLFKTCRGLFVLCQYCGLLQIYSADTPSSSYTQSTMDHDLHDAL

SEQ ID NO: 175 - protein, 4 aa, synthetic construct
SGSG

SEQ ID NO: 176 - protein, 4 aa, synthetic construct
GSGS